(19) United States
(12) Patent Application Publication
(10) Pub. No.: US 2024/0293318 A1
(43) Pub. Date: Sep. 5, 2024

(54) CLEAVABLE LINKER-CONTAINING IONIZABLE LIPIDS AND LIPID CARRIERS FOR THERAPEUTIC COMPOSITIONS

(71) Applicant: Flagship Pioneering Innovations VII, LLC, Cambridge, MA (US)

(72) Inventors: Timothy Ray Blake, Cambridge, MA (US); Dean Peter Stamos, Lexington, MA (US)

(21) Appl. No.: 18/440,188

(22) Filed: Feb. 13, 2024

Related U.S. Application Data
(60) Provisional application No. 63/445,266, filed on Feb. 13, 2023.

(51) Int. Cl. A61K 9/127; A61K 9/51; A61K 45/06; C07C 211/03; C07C 307/06; C07D 231/38; C07D 241/08; C07D 241/20; C07D 249/14; C07D 251/10; C07D 265/32; C07D 285/10; C07D 285/16; C07D 305/12; C07D 307/33; C07D 309/30; C07D 319/12; C07F 9/24

(52) U.S. Cl. CPC A61K 9/1271; A61K 9/5123; A61K 45/06; C07C 211/03; C07C 307/06; C07D 231/38; C07D 241/08; C07D 241/20; C07D 249/14; C07D 251/10; C07D 265/32; C07D 285/10; C07D 285/16; C07D 305/12; C07D 307/33; C07D 309/30; C07D 319/12; C07F 9/2458

(57) ABSTRACT

The present disclosure relates to a lipid compound of formula (Ia) or (AL-GI):

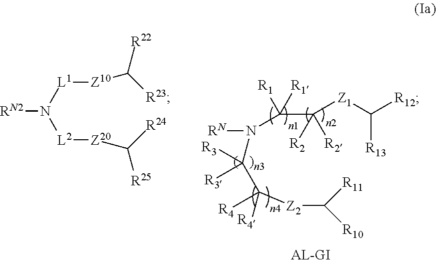

having various cleavable linkers defined by the variables Z₁ and Z₂ and Z¹⁰ and Z²⁰. The present disclosure also relates to a lipid carrier or lipid nanoformulation employing the lipid compound, and the use of the lipid compound in a pharmaceutical composition as well as for a method of delivering an effector, e.g., a therapeutic agent.

### RELATED APPLICATIONS

This application claims the benefit of priority to U.S. Provisional Patent Application Ser. No. 63/445,266, filed Feb. 13, 2023, the contents of which are incorporated herein by reference.

### FIELD OF THE INVENTION

The present disclosure generally relates to novel lipids, lipid-based carriers, pharmaceutical compositions, and methods.

### BACKGROUND

There continues to be a need in the art for improved lipid carriers employing novel lipids for delivering therapeutic agents, such as nucleic acid molecules, proteins, and small molecule drugs.

### SUMMARY OF THE INVENTION

In some embodiments, the present disclosure relates to a compound having the structure of formula (Ia):

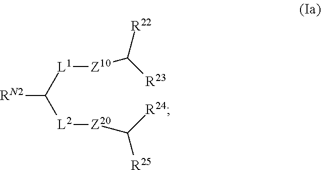

or a salt thereof;
wherein:
R^{N2} is —(CH₂)ₘ(NH)ₙQ²;
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3;
n is an integer from 0-1;
L¹ and L² are each independently (C₁-C₁₅)alkylene;
Z¹⁰ and Z²⁰ are each independently

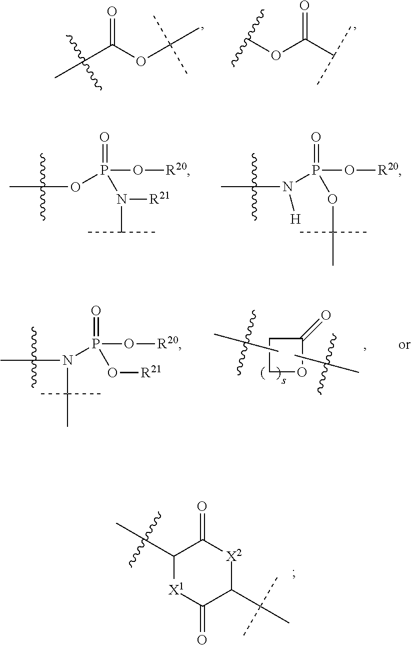

each of X¹ and X² is independently O, S, or N(R²¹)
R²⁰ is branched (C₁-C₁₅)alkyl or unbranched (C₁-C₁₅)alkyl;
R²¹ is H, (C₁-C₅)alkyl, or (C₃-C₈)cycloalkyl;
s is an integer from 1 to 4;
   in Z¹⁰, [symbol] indicates the point of attachment to L¹;
   in Z²⁰, [symbol] indicates the point of attachment to L²;
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H;
wherein when Q² is —OH, then at least one of (i) or (ii) applies:
   (i) at least one of Z¹⁰ and Z²⁰ is

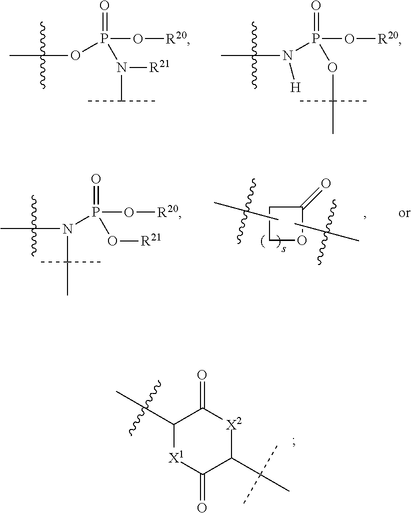

(ii) L¹ and L² are not identical.

In some embodiments, the present disclosure relates to a compound of formula (AL-GI):

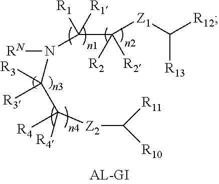

or a salt thereof;
wherein:
R^{N} is substituted or unsubstituted C₁-C₆ alkyl or C₃-C₈ cycloalkyl;
each of R₁, R_{1′}, R₂, R_{2′}, R₃, R_{3′}, R₄, and R_{4′}, independently for each occurrence, is H, branched C₁-C₃ alkyl, unbranched C₁-C₃ alkyl, branched C₂-C₃ alkenyl, or unbranched C₂-C₃ alkenyl;
each of R₁₀, R₁₁, R₁₂, and R₁₃ is independently H or a substituted or unsubstituted branched C₁-C₁₅ alkyl or unbranched C₁-C₁₅ alkyl; provided that at least one of R₁₀ and R₁₁ is not H, and at least one of R₁₂ and R₁₃ is not H;
each of Z₁ and Z₂ is independently

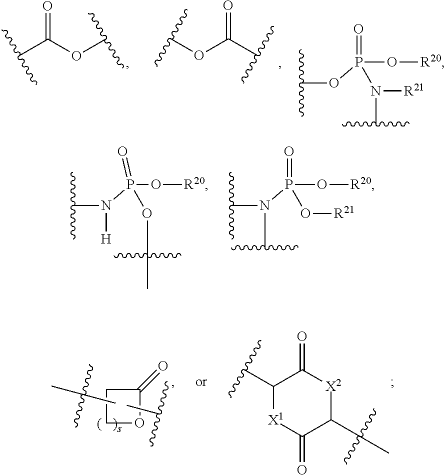

each of X¹ and X² is independently O, S, or N(R₂₁);
R₂₀ is a substituted or unsubstituted branched C₁-C₁₅ alkyl or unbranched C₁-C₁₅ alkyl;
R₂₁ is H, substituted or unsubstituted C₁-C₅ alkyl, or substituted or unsubstituted C₃-C₈ cycloalkyl;
s is an integer from 1 to 4; and
each of n₁, n₂, n₃, and n₄ is independently an integer from 0 to 15, wherein n₁, n₂ ranges from 1 to 15 and n₃+n₄ ranges from 1 to 15;
wherein when R^{N} is C₁-C₆ alkyl substituted with hydroxy, then at least one of (i) or (ii) applies:
   (i) at least one of Z₁ and Z₂ is

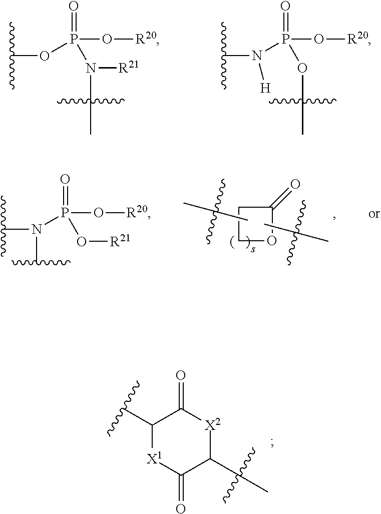

or
(ii) the sum of n₁ and n₂ does not equal the sum of n₃ and n₄.

In some embodiments, the present disclosure relates to a compound having the structure of formula (I-w):

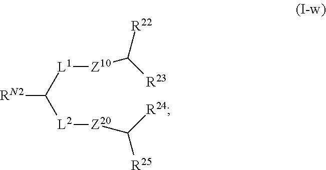

or a salt thereof;
wherein:
R^{N2} is

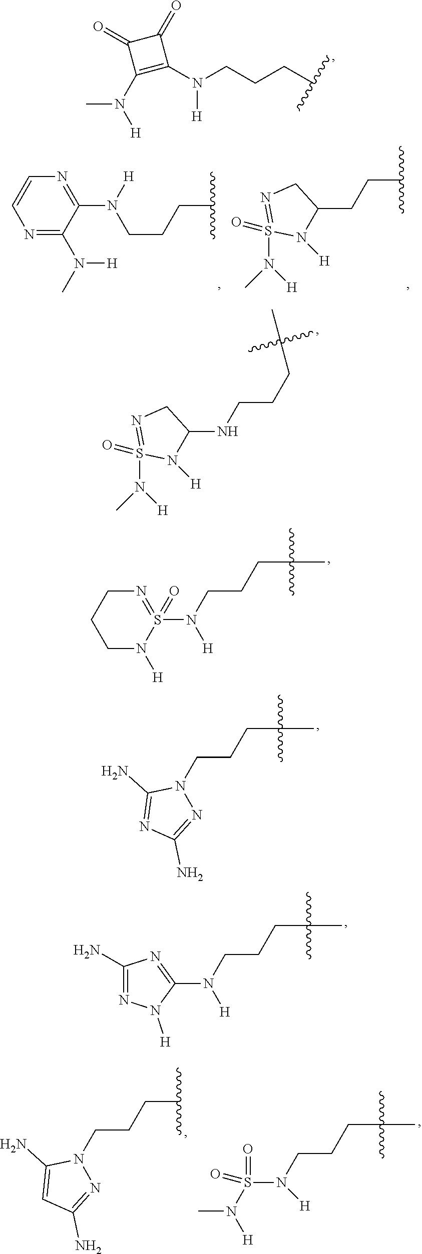

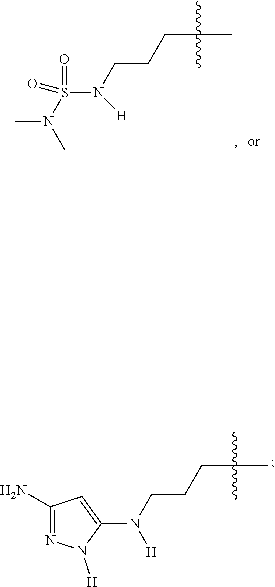 [SMILES: *CCCNS(=O)(=O)N(C)C ; *CCCNc1cc(N)n[nH]1]

L¹ and L² are each independently (C₁-C₁₅)alkylene;
Z¹⁰ and Z²⁰ are each independently

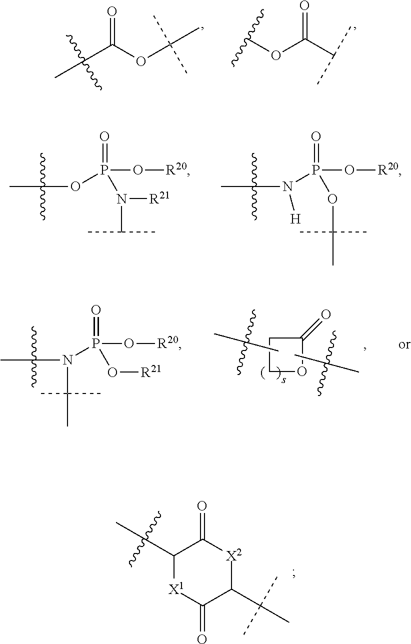

each of X¹ and X² is independently O, S, or N(R²¹);
R²⁰ is branched (C₁-C₁₅)alkyl or unbranched (C₁-C₁₅)alkyl;
R²¹ is H, (C₁-C₅)alkyl, or (C₃-C₈)cycloalkyl;
s is an integer from 1 to 4;
   in Z¹⁰, [symbol] indicates the point of attachment to L¹;
   in Z²⁰, [symbol] indicates the point of attachment to L²; and
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H.

In some embodiments, the present disclosure relates to a compound having the structure of formula (I-w4), (I-w-5), or (I-w-6):

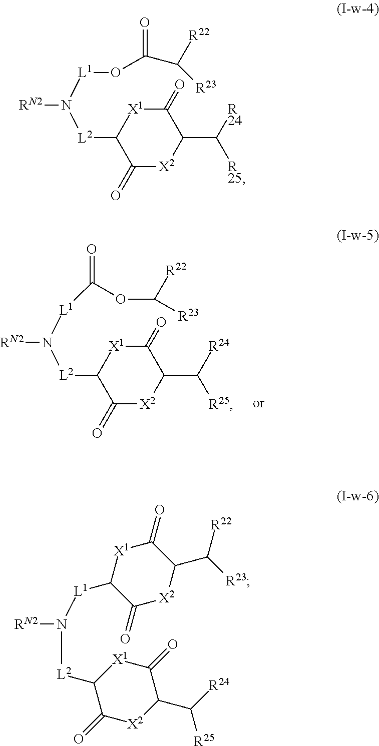

or a salt thereof;
wherein:
R^{N2} is —(CH₂)ₘ(NH)ₙQ²;
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3;
n is an integer from 0-1;
L¹ and L² are each independently (C₁-C₁₅)alkylene;
each of X¹ and X² is independently O, S, or N(R²¹);
R²¹ is H, (C₁-C₅)alkyl, or (C₃-C₈)cycloalkyl; and
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H.

In some embodiments, the present disclosure relates to a compound having the structure of formula (I-w-7), (I-w-8), or (I-w-9):

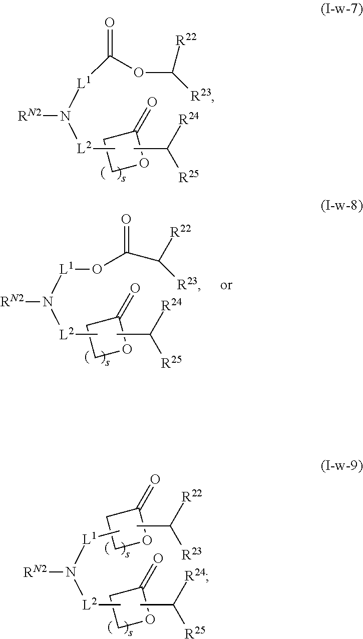

or a salt thereof;
wherein:
R^{N2} is —(CH₂)ₘ(NH)ₙQ²,
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3:
n is an integer from 0-1;
L¹ and L² are each independently (C₁-C₁₅)alkylene;
s is an integer from 1 to 4; and
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R⁴ and R²⁵ is not H.

In certain embodiments, the present disclosure provides a compound having the structure of formula (I-w-10), (1-w-11), (1-w-12), (I-w-13), (I-w-14), or (I-w-15):

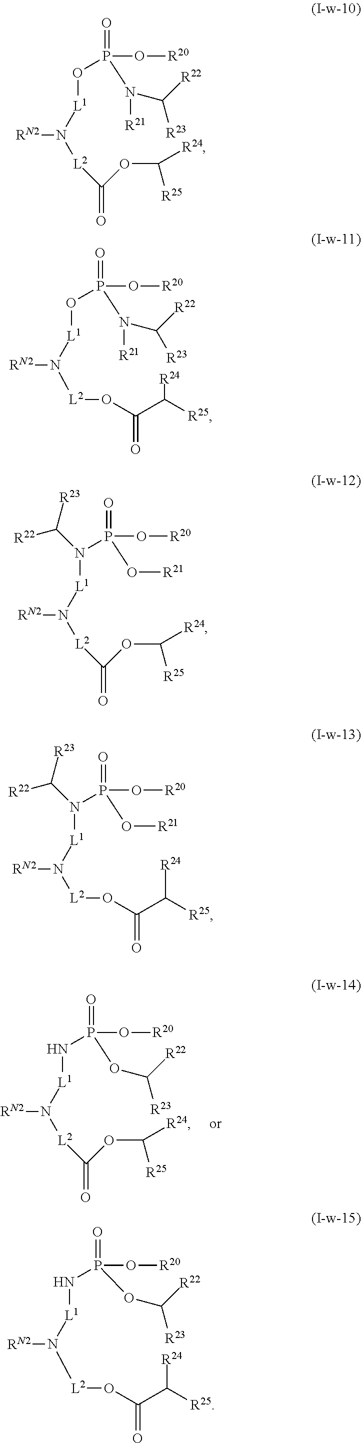

or a salt thereof;
wherein:
R^{N2} is —(CH₂)ₘ(NH)ₙQ²;
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3;
n is an integer from 0-1;
L¹ and L² are each independently (C₁-C₁₅)alkylene;
R²⁰ is branched (C₁-C₁₅)alkyl or unbranched (C₁-C₁₅)alkyl;
R²¹ is H, (C₁-C₅)alkyl, or (C₃-C₈)cycloalkyl; and
each of R²², R^{2′}, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H.

In some embodiments, the present disclosure provides a compound, or a salt thereof, having one of the following structures:

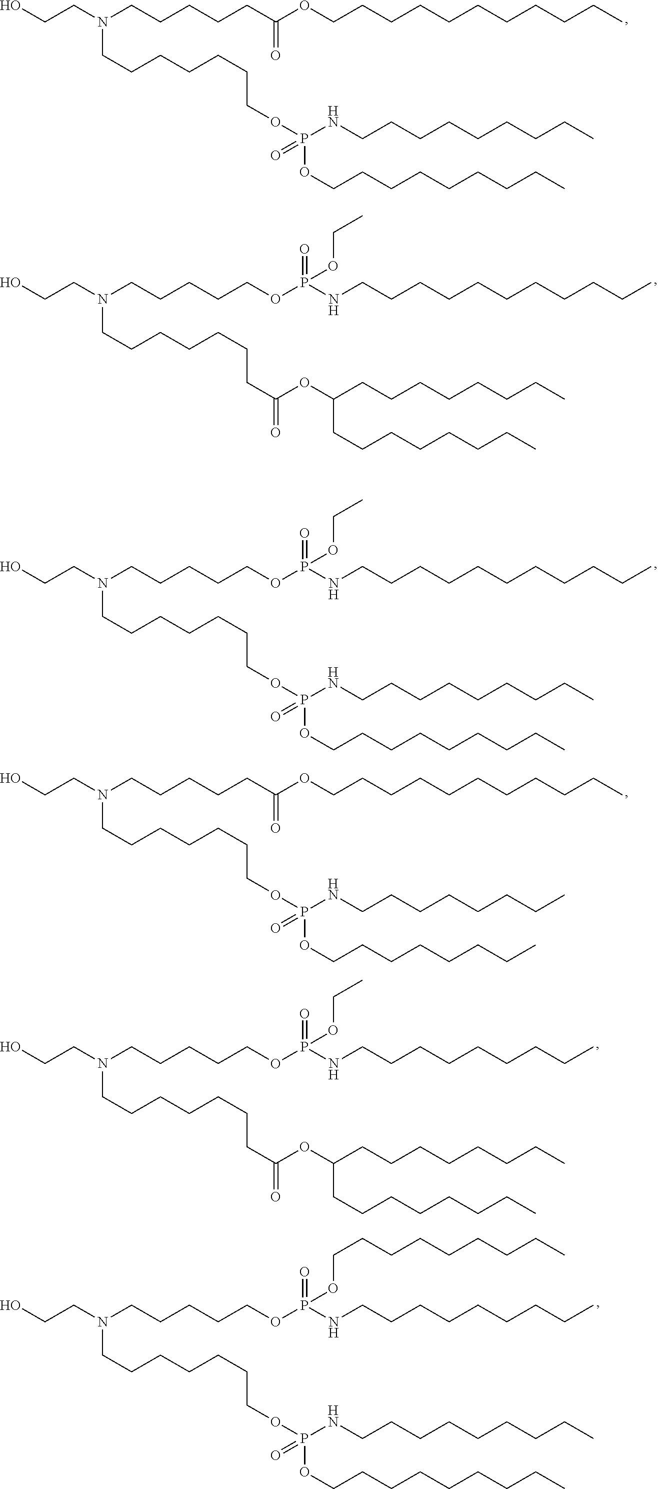

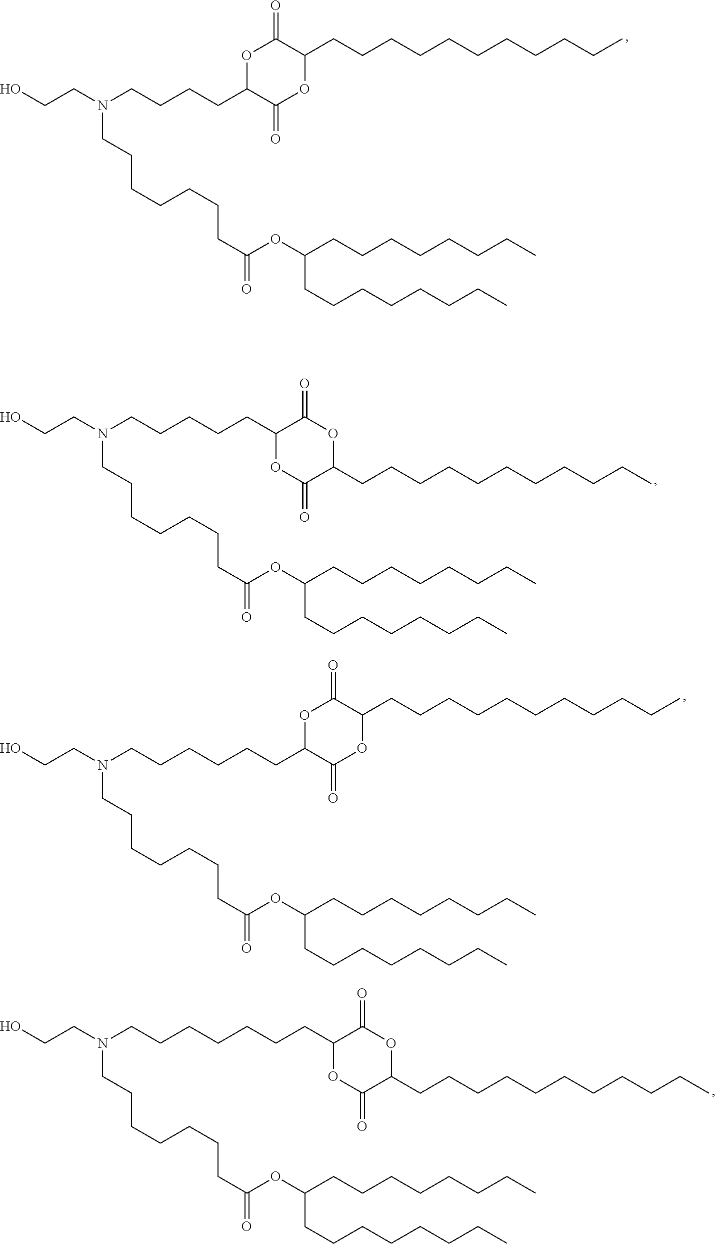

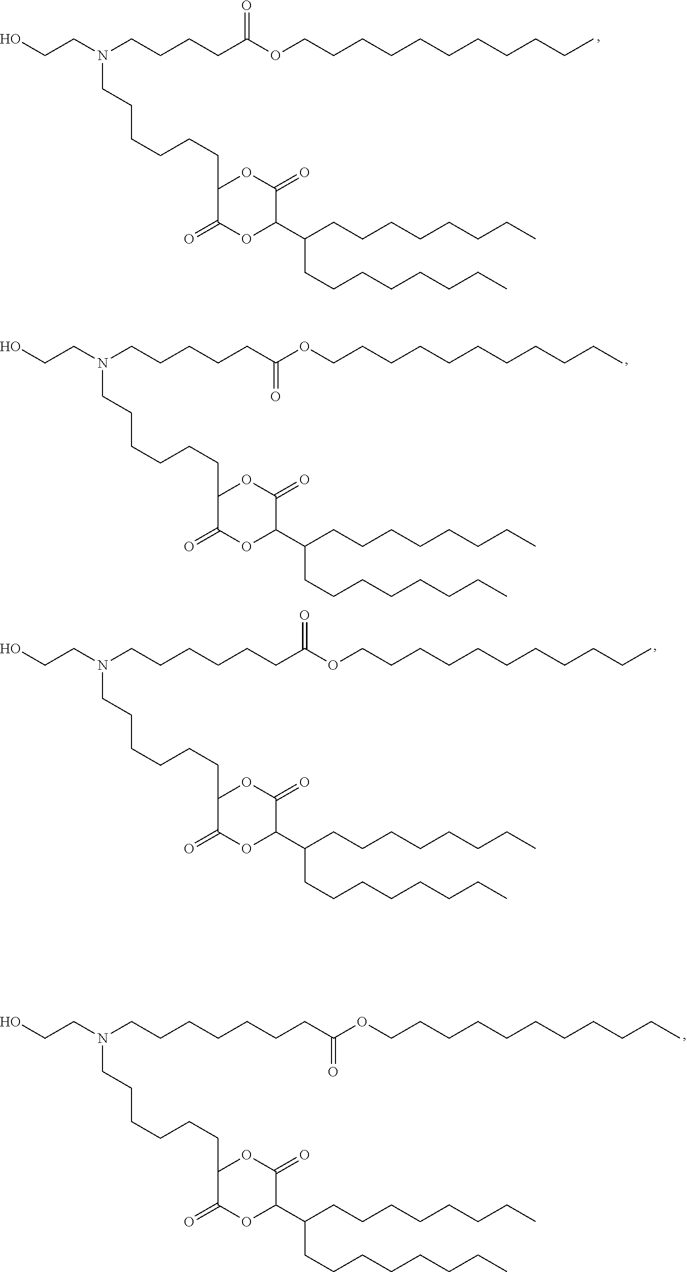

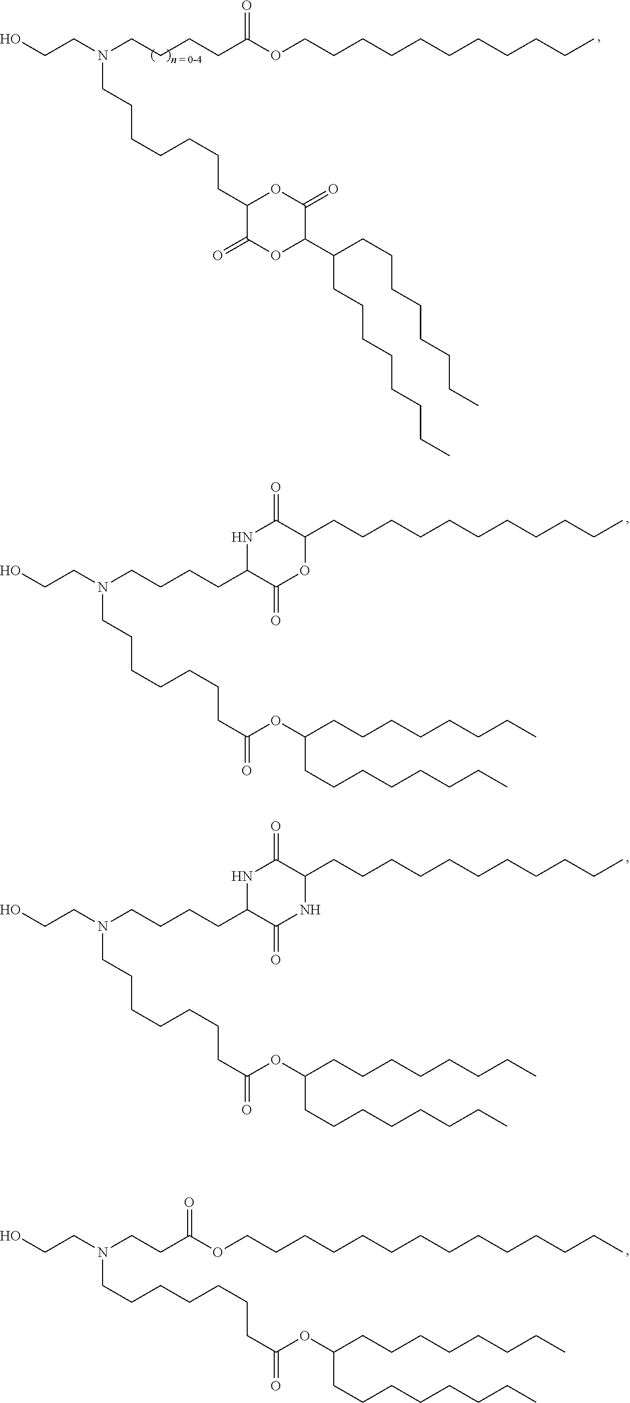

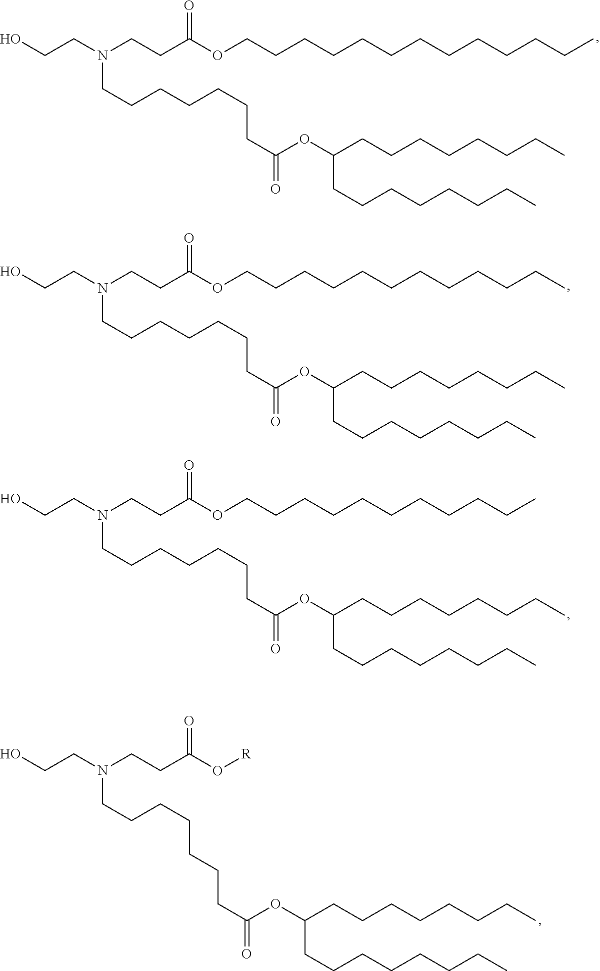

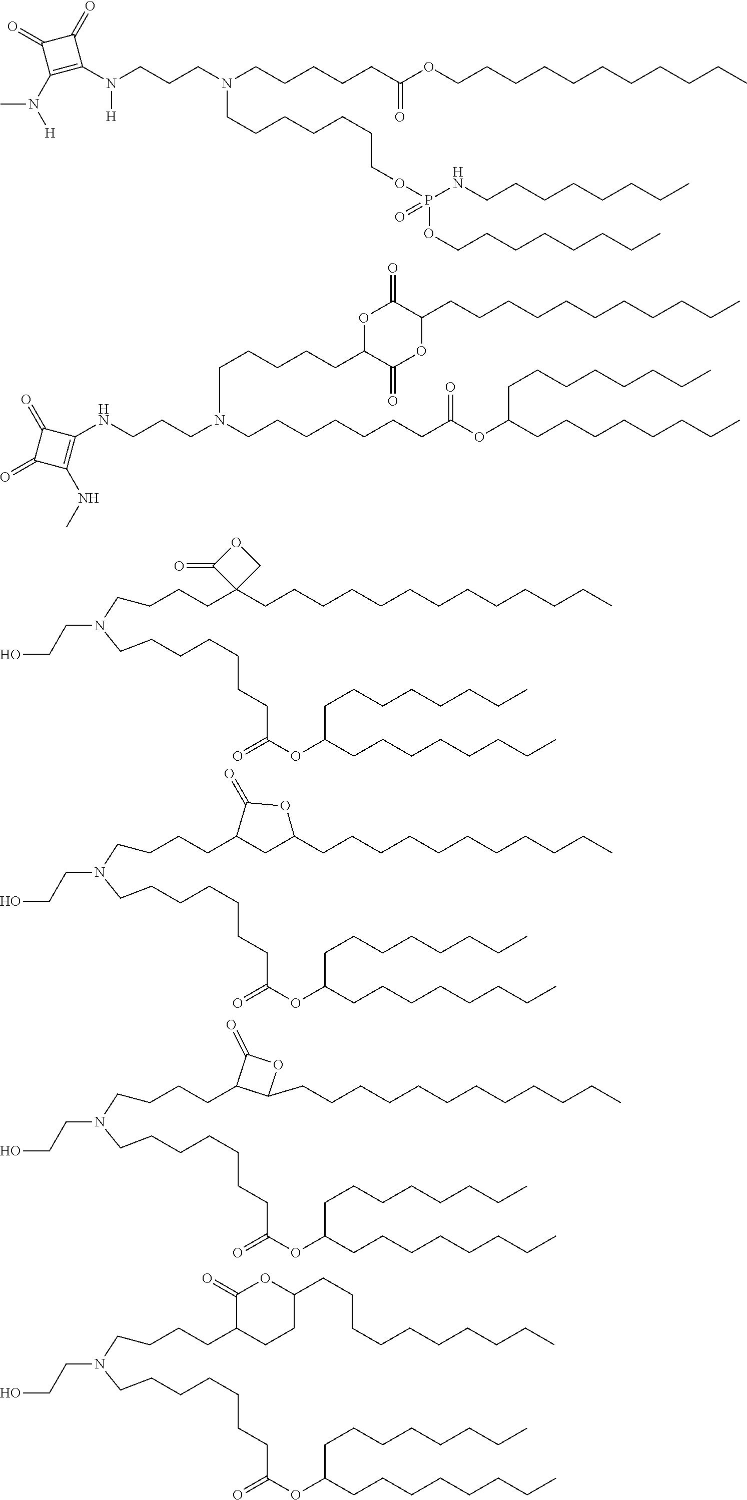

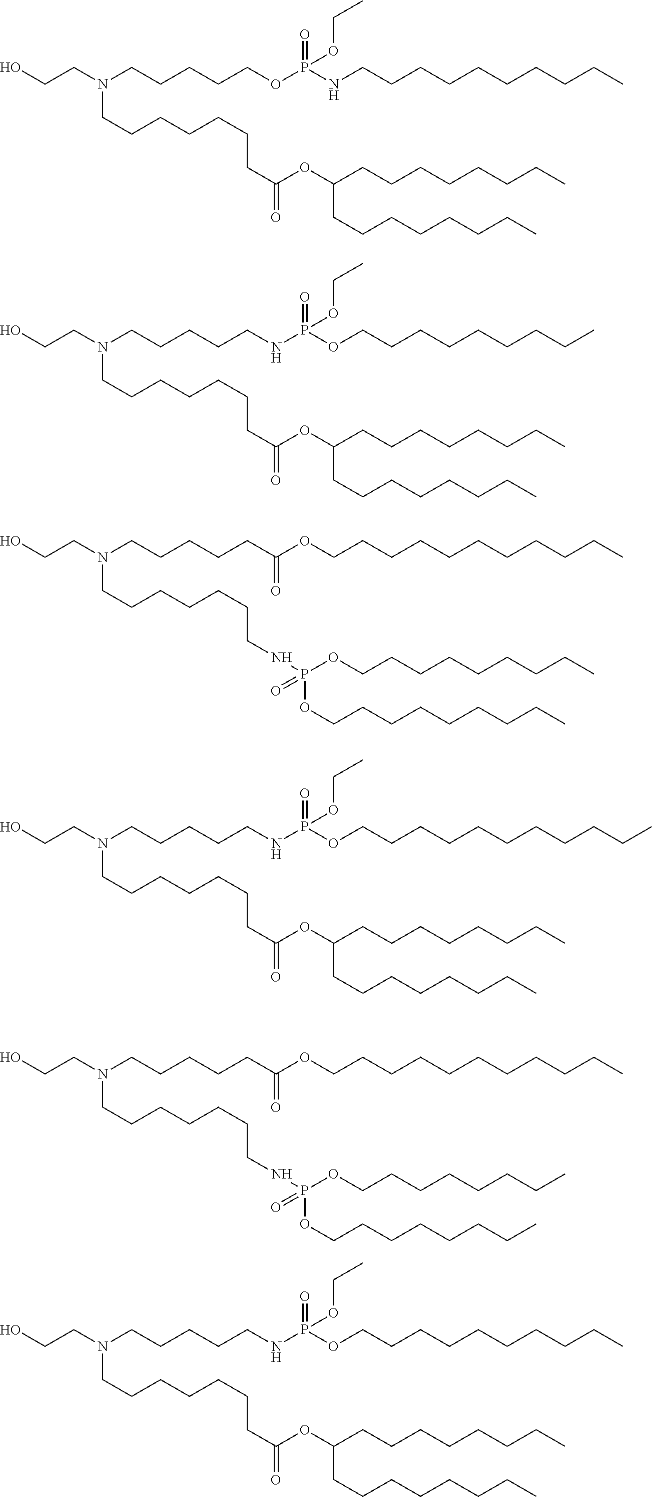

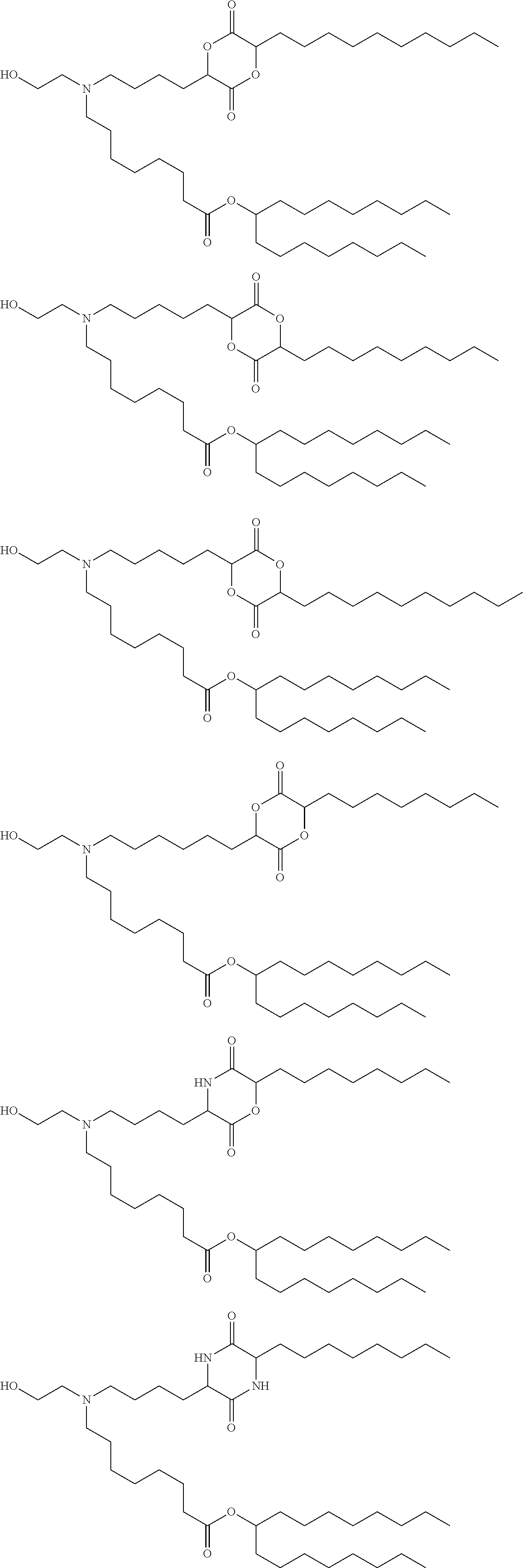

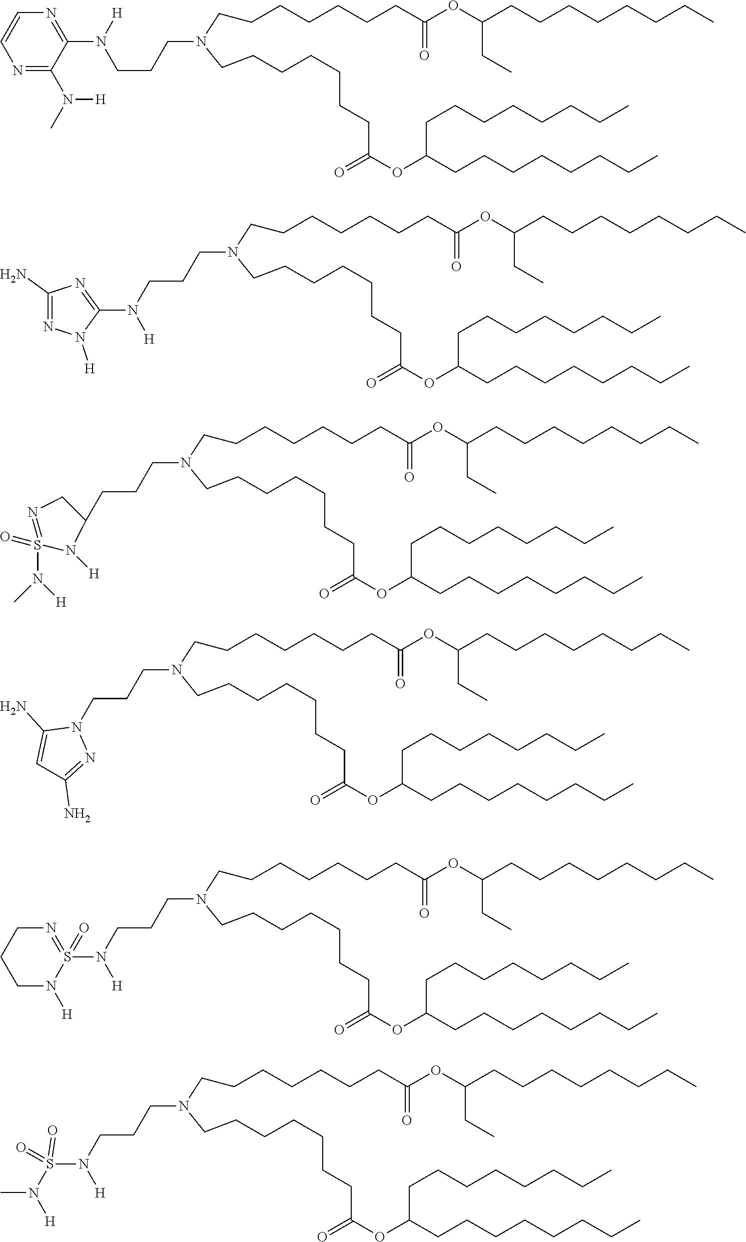

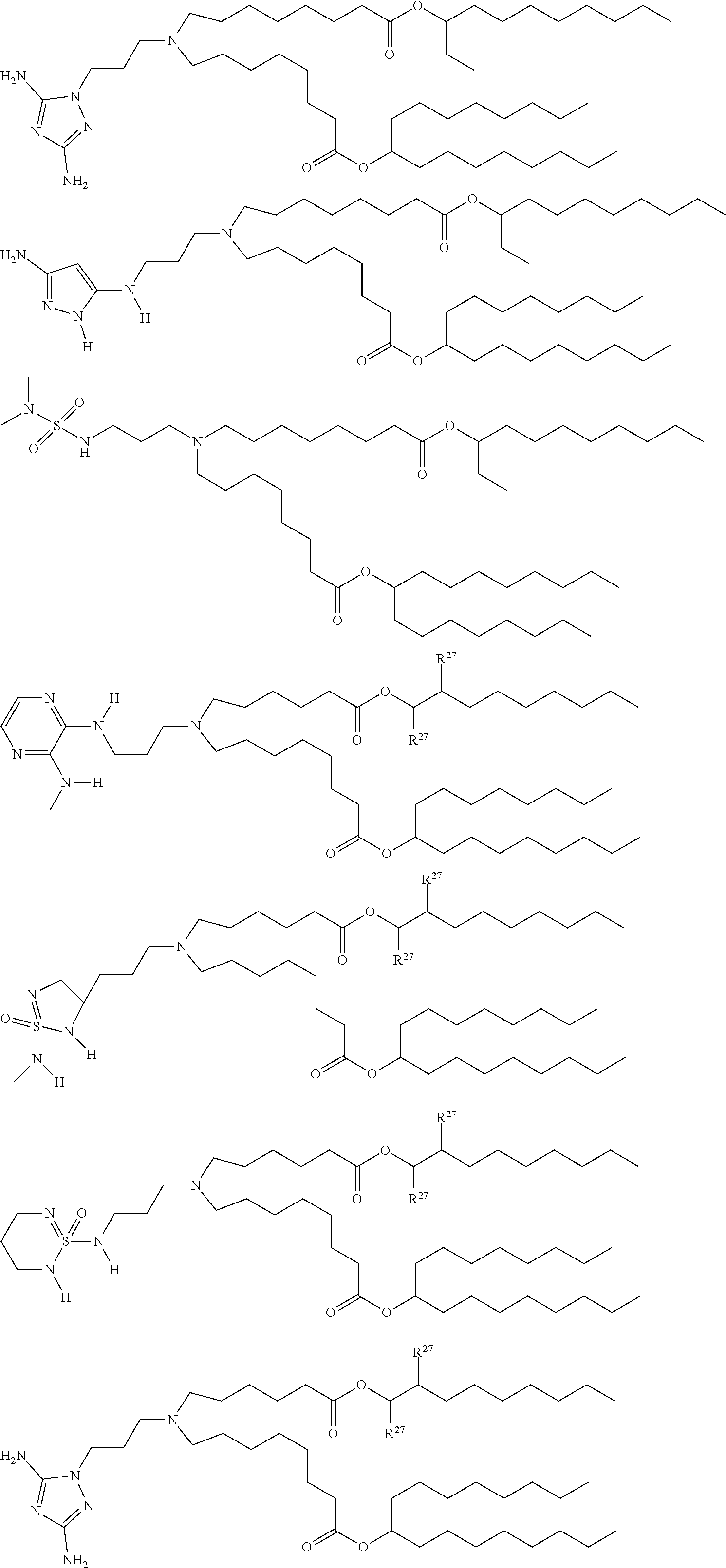

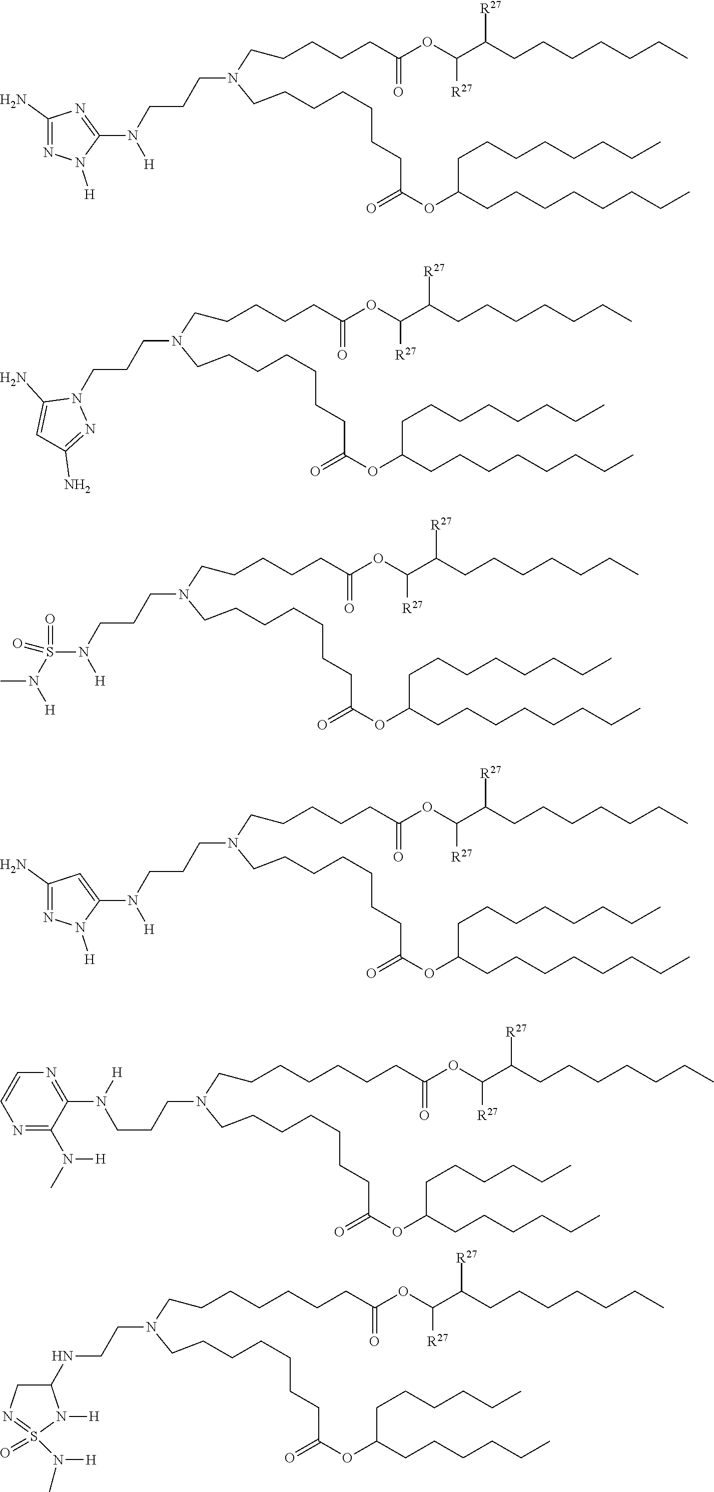

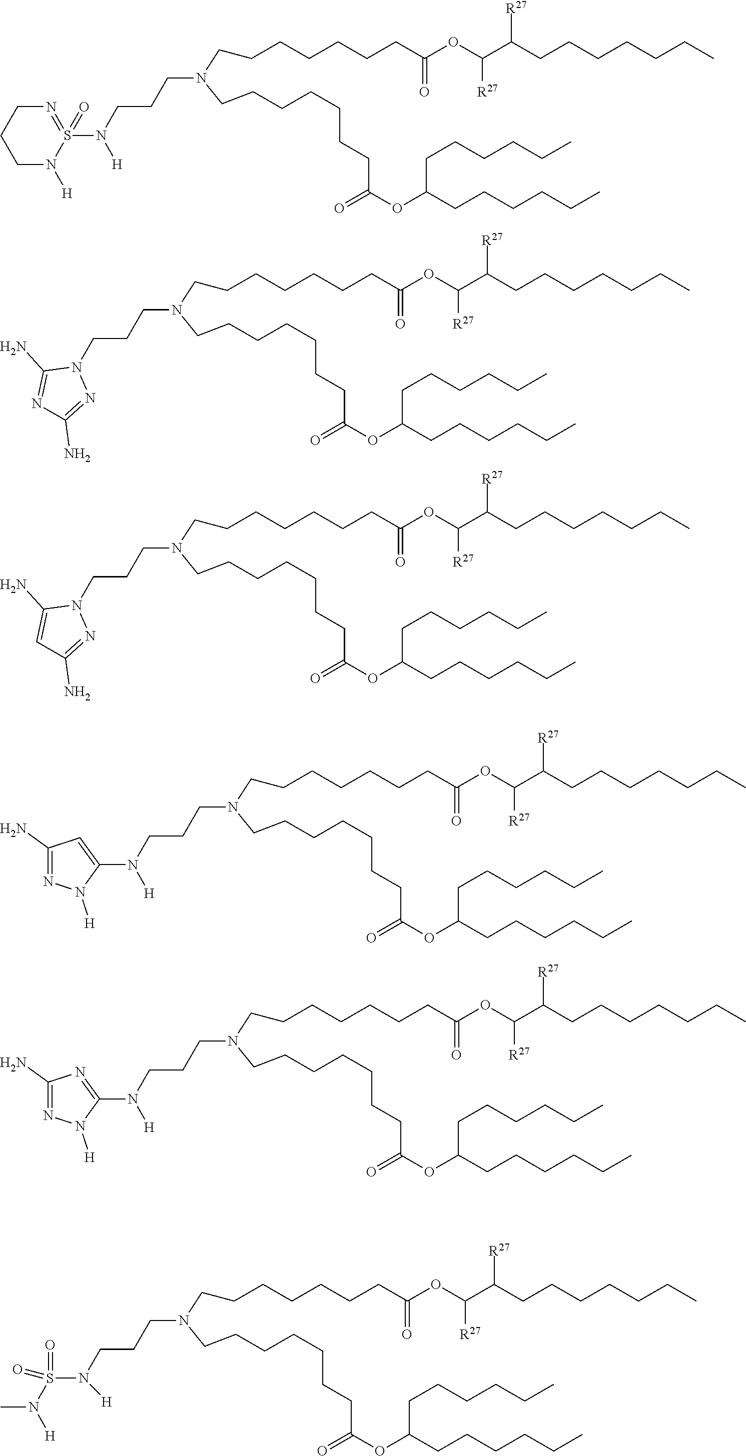

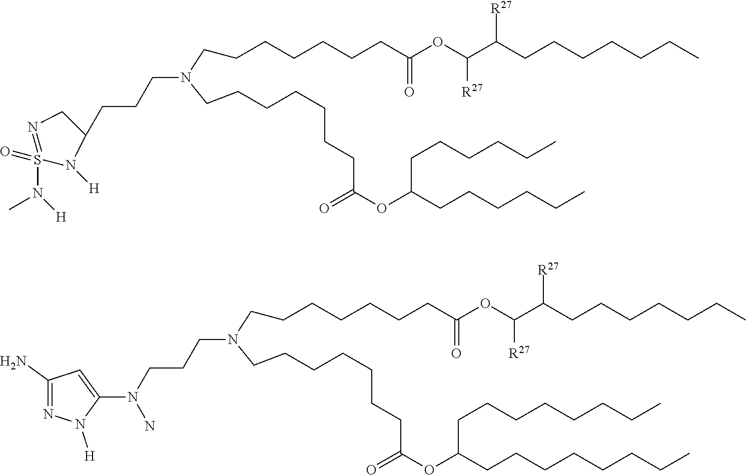

wherein R=

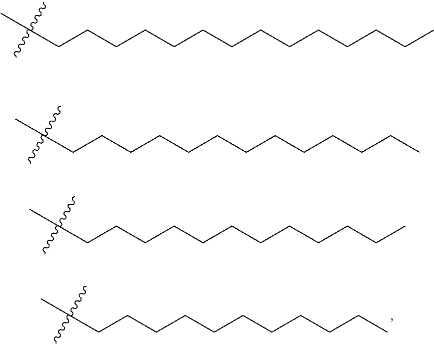 [SMILES: *CCCCCCCCCCCCCC ; *CCCCCCCCCCCCC ; *CCCCCCCCCCCC ; *CCCCCCCCCCC]

and each R²⁷ is independent H, C₁-C₁₅ alkyl, C₂-C₈ alkenyl, or C₂-C₈ alkynyl.

In some embodiments, the present disclosure relates to a lipid-based carrier comprising a compound of the present disclosure, e.g., a compound of formula (Ia) or (AL-GI), wherein the lipid-based carrier is a lipid nanoparticle.

In some embodiments, the present disclosure relates to a method of delivering an effector, e.g., a therapeutic agent to a subject, the method comprising administering to the subject the lipid-based carrier of the present disclosure, which lipid-based carrier comprises the effector.

In some embodiments, the present disclosure relates to a pharmaceutical composition comprising the lipid-based carrier of the present disclosure, and a pharmaceutically acceptable excipient.

### BRIEF DESCRIPTION OF THE DRAWINGS

FIG. 1 is a bar graph showing in vivo hEPO expression in Lipids 1-22 versus a control.

### DETAILED DESCRIPTION OF THE INVENTION

Disclosed herein are amine-containing novel ionizable lipids having various cleavable linkers useful for formation of lipid carriers or lipid nanoformulations (such as a lipid nanoparticle (LNP) or a liposome), that can be used in pharmaceutical composition or methods as described herein. These lipid carriers or lipid nanoformulations employing the novel ionizable lipids may have properties advantageous for delivering a therapeutic agent (such as a nucleic acid molecule) to cells. The disclosure thus provides the lipid carriers or lipid nanoformulations comprising these novel lipids. The disclosure also provides pharmaceutical compositions comprising these lipid-based carriers or lipid nanoformulations. Additionally, the disclosure provides methods of delivering an effector, e.g., a therapeutic agent to a cell or subject by administering to the cell or subject the pharmaceutical compositions, or the lipid carriers or lipid nanoformulations, containing the effector.

In some embodiments, the present disclosure relates to a compound having the structure of formula (Ia):

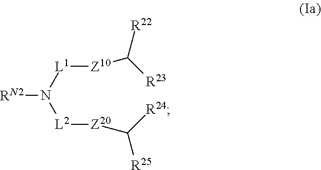

or a salt thereof;
wherein:
R^{N2} is —(CH₂)ₘ(NH)ₙQ²;
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3;
n is an integer from 0-1;
L¹ and L² are each independently (C₁-C₁₅)alkylene;
Z¹⁰ and Z²⁰ are each independently

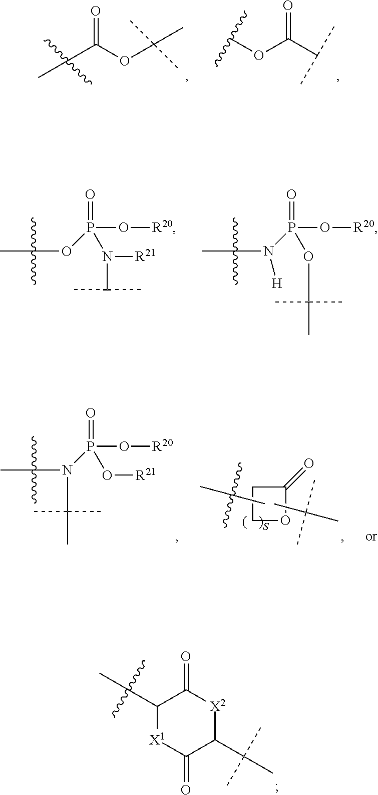

each of X¹ and X² is independently O, S, or N(R²¹);
R²⁰ is branched (C₁-C₁₅)alkyl or unbranched (C₁-C₁₅)alkyl;
R²¹ is H, (C₁-C₁₅)alkyl, or (C₃-C₈)cycloalkyl;
s is an integer from 1 to 4; [symbol]
   in Z¹⁰, [symbol] indicates the point of attachment to L¹;
   in Z²⁰, [symbol] a indicates the point of attachment to L²;
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H;
wherein when Q² is —OH, then at least one of (i) or (ii) applies:
   (i) at least one of Z¹⁰ and Z²⁰ is

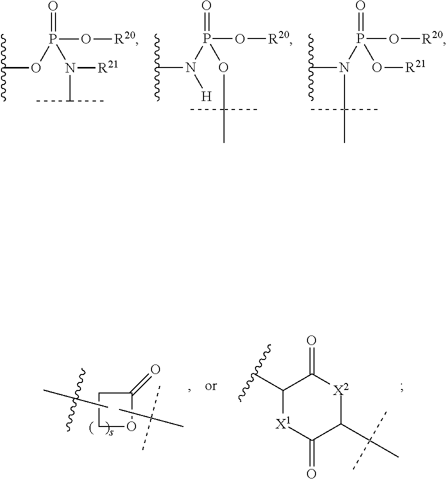

(ii) L₁ and L² are not identical.

In some embodiments, R^{N2} is —(CH₂)ₘQ².

In some embodiments, Q² is —OH.

In some embodiments, R^{N2} is —CH₂CH₂OH.

In some embodiments, R^{N2} is —(CH₂)ₘ(NH)Q².

In some embodiments, Q² is —SO₂NH(alkyl) or —SO₂N(alkyl)₂.

In some embodiments, Q² is optionally substituted cycloalkyl, optionally substituted aryl, optionally substituted heteroaryl, optionally substituted heterocycloalkyl, or optionally substituted heterocycloalkenyl; wherein, valence permitting, the optional substituents are selected from oxo, amino, alkylamino, and dialkylamino.

In some embodiments, Q² is

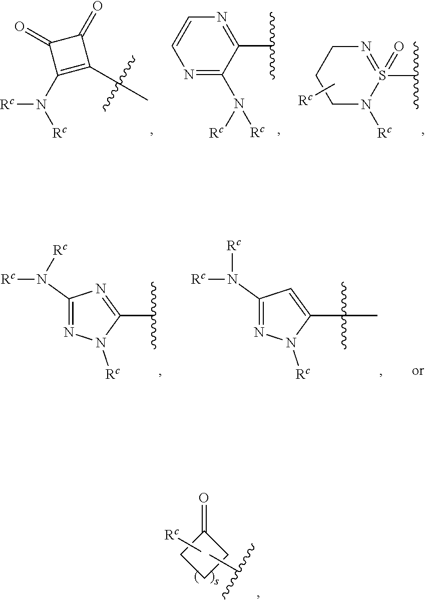

wherein each R^{c} is independently H or C₁-C₃ alkyl, and s is an integer from 1 to 4.

In some embodiments, R^{N} is

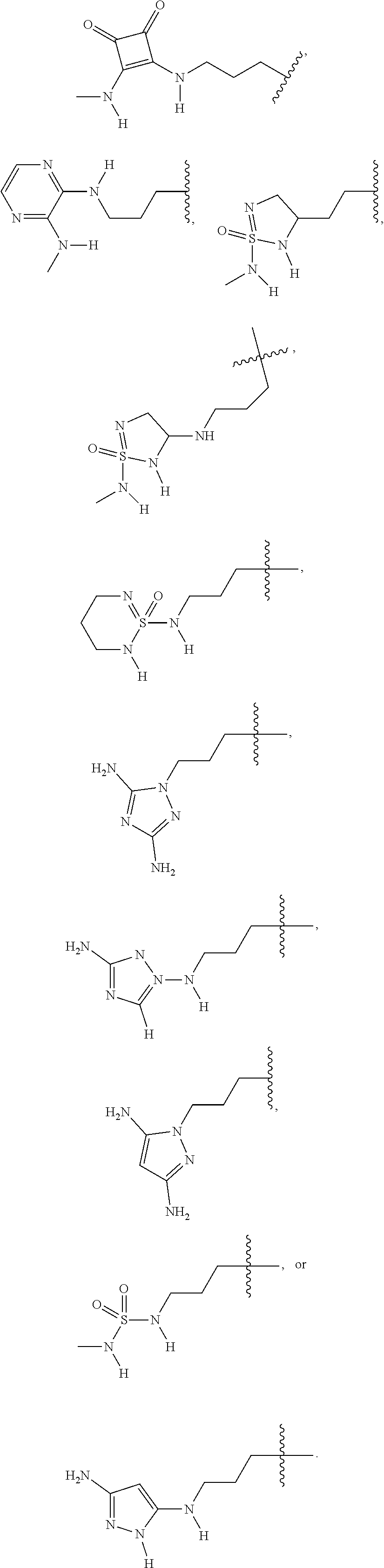

In some embodiments, L¹ and L² are each independently (C₂-C₁₀)alkylene.

In some embodiments, L¹ and L² are each independently (C₂-C₈)alkylene.

In some embodiments, L¹ and L² are each independently (C₄-C₈)alkylene.

In some embodiments, L¹ and L² are not identical.

In some embodiments, Z¹⁰ and Z²⁰ are each independently

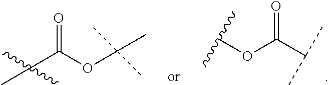

In some embodiments, R²² is not H; R²⁴ is not H; and at least one of R²³ and R²⁵ is not H.

In some embodiments, R²², R²³, R²⁴, and R²⁵ are independently branched (C₁-C₁₅)alkyl, or unbranched (C₁-C₁₅)alkyl.

In some embodiments, R²² and R²³ are identical.

In some embodiments, R²² and R²³ are not identical.

In some embodiments, R²⁴ and R²⁵ are identical.

In some embodiments, R²⁴ and R²⁵ are not identical.

In some embodiments, one of Z¹⁰ and Z²⁰ is

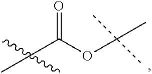

and the other of Z¹⁰ and Z²⁰

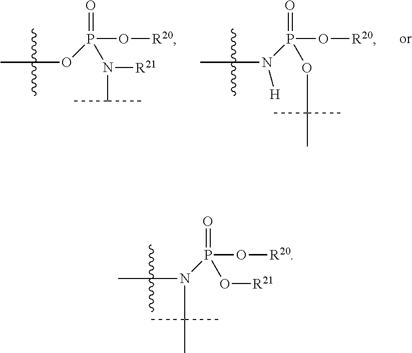

In some embodiments, each of Z¹⁰ and Z²⁰ is independently

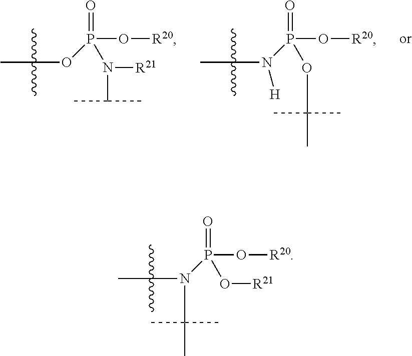

In some embodiments, when Z¹⁰ is

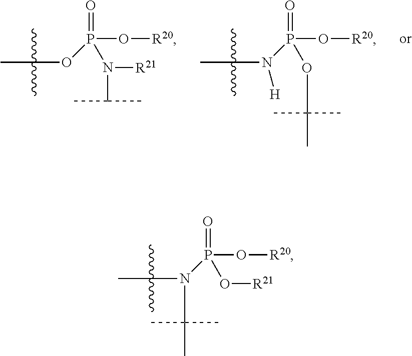

then R²² is H; and
when Z²⁰ is

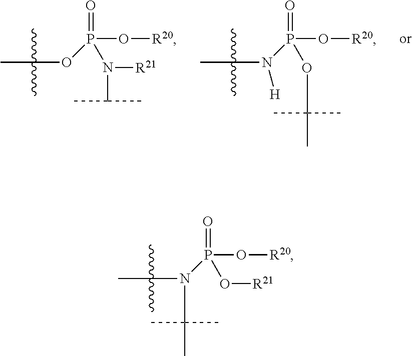

then R²⁴ is H.

In some embodiments, one of Z¹⁰ and Z²⁰ is

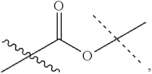

and the other of Z¹⁰ and Z²⁰ is

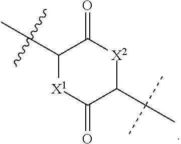

In some embodiments, X¹ and X² are each O.

In some embodiments, R²² is not H; R²⁴ is not H; and at least one of R²³ and R²⁵ is not H.

In some embodiments, one of Z¹⁰ and Z²⁰ is

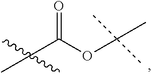

and the other of Z¹⁰ and Z²⁰ is

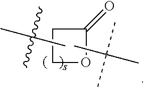

In some embodiments, s is an integer from 1-3.

In some embodiments the compound has the formula:

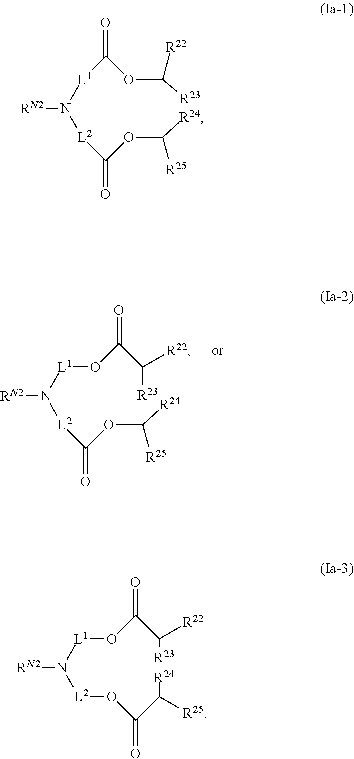

In some embodiments, R²² and R²³ are each independently unsubstituted C₅-C₈alkyl; or R²³ is H, and R²² is unsubstituted C₁₀-C₁₄ alkyl; and
R²⁴ and R²⁵ are each independently unsubstituted C₅-C₈alkyl; or R² is H, and R²⁴ is unsubstituted C₁₀-C₁₄ alkyl;
wherein R²³ and R²⁵ are not both H.

In some embodiments, the compound has the formula:

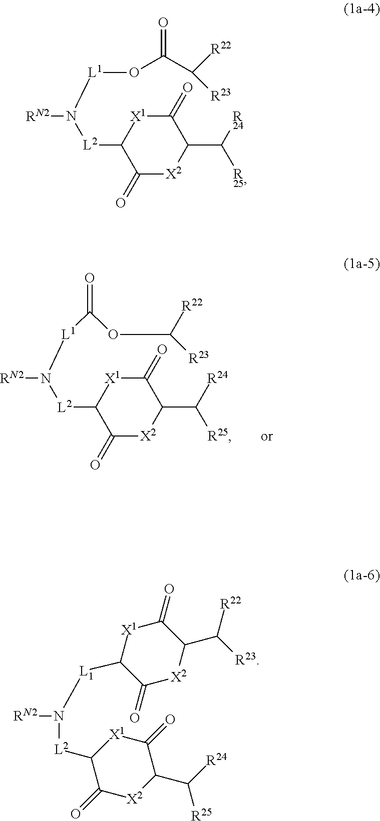

In some embodiments, X¹ and X² are each O.

In some embodiments, R²² is not H; R²⁴ is not H; and at least one of R²³ and R²¹ is not H.

In some embodiments, the compound the formula

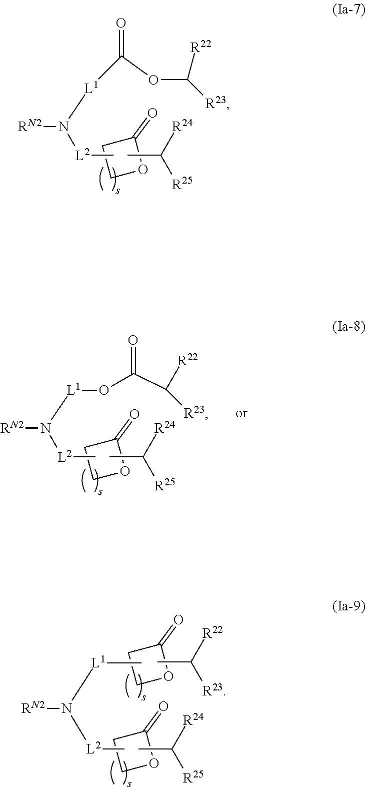

In some embodiments, the compound has the formula:

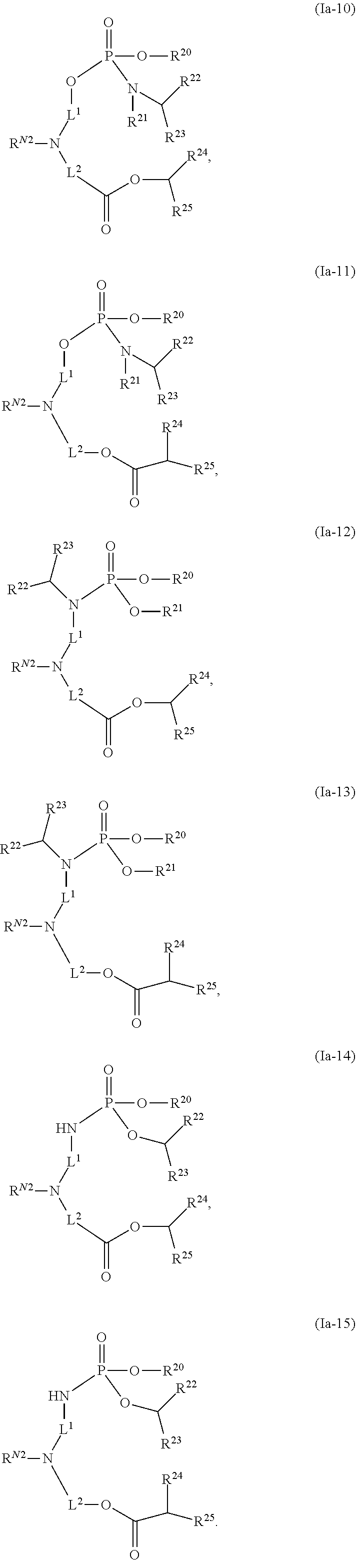

In some embodiments, R²³ is H.

In some embodiments, the compound has one of the following structures:

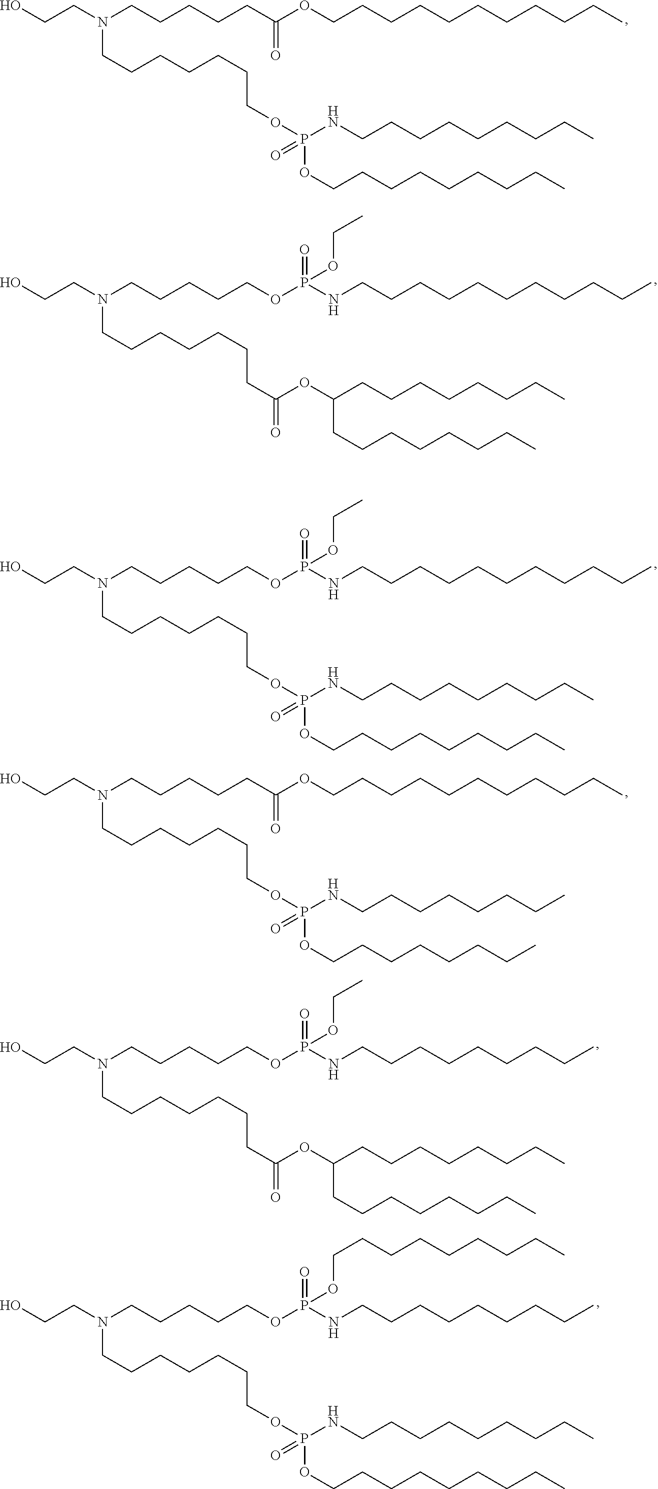

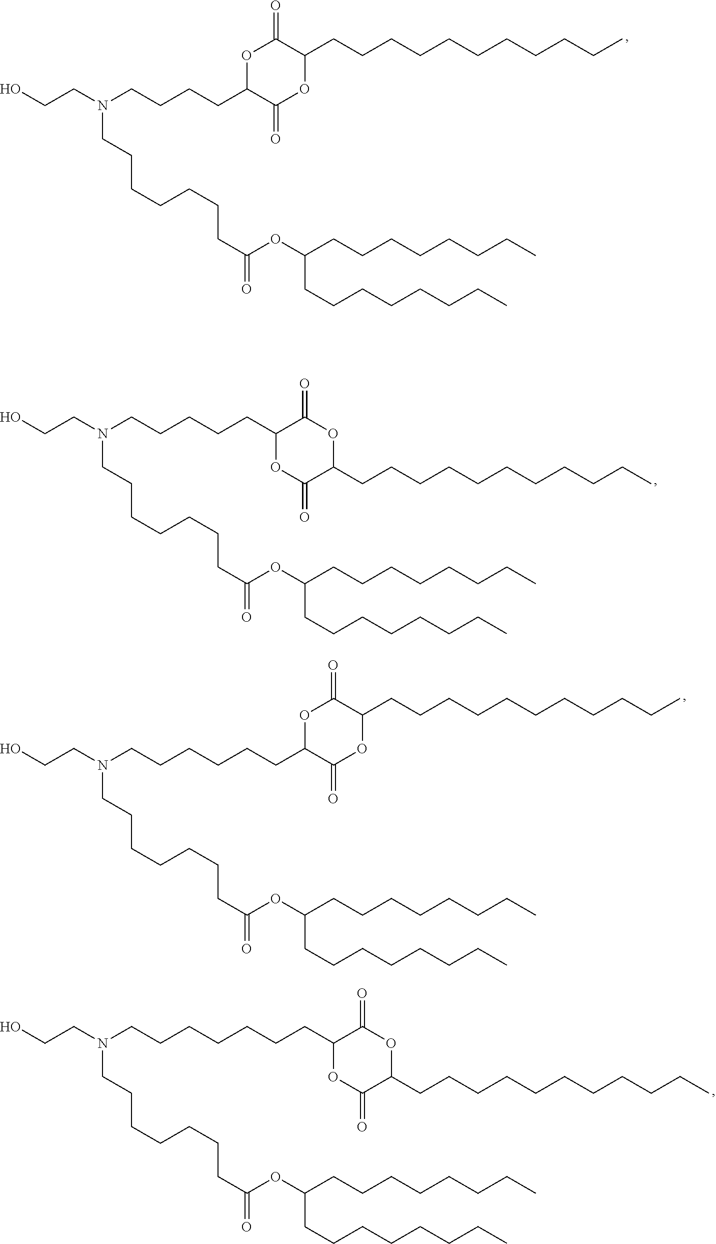

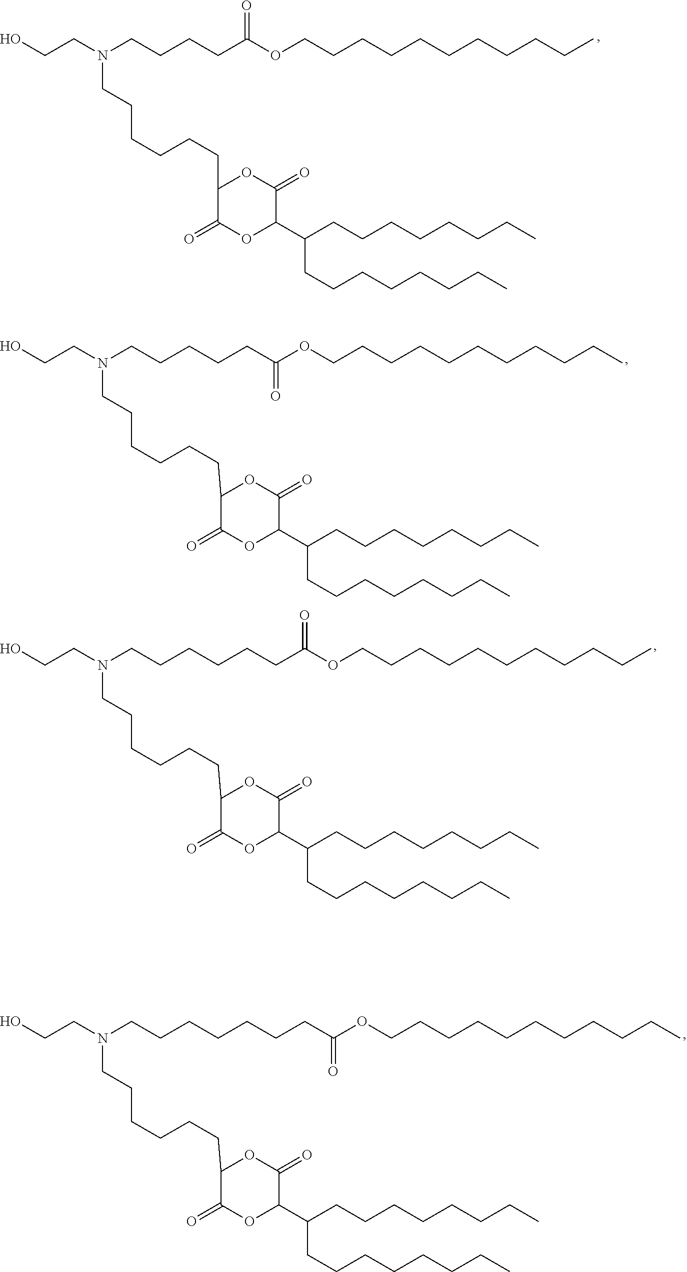

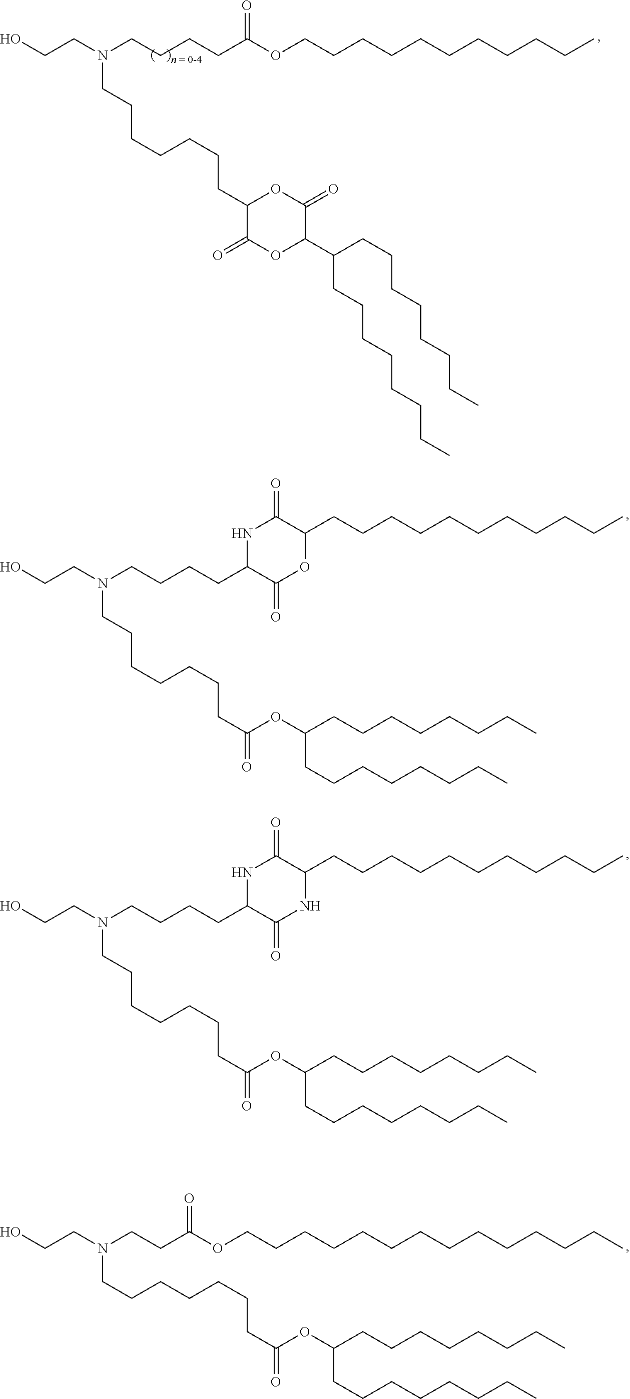

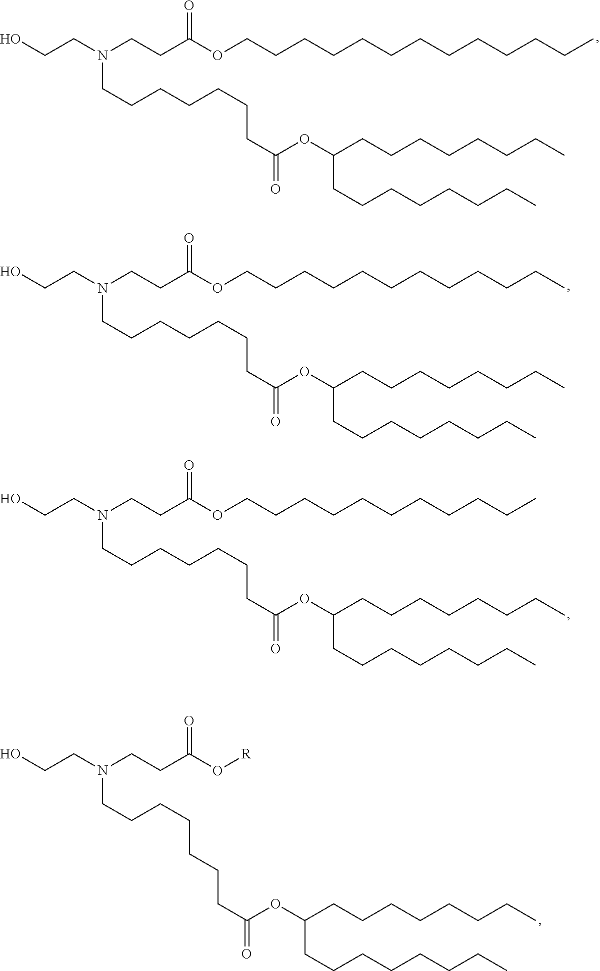

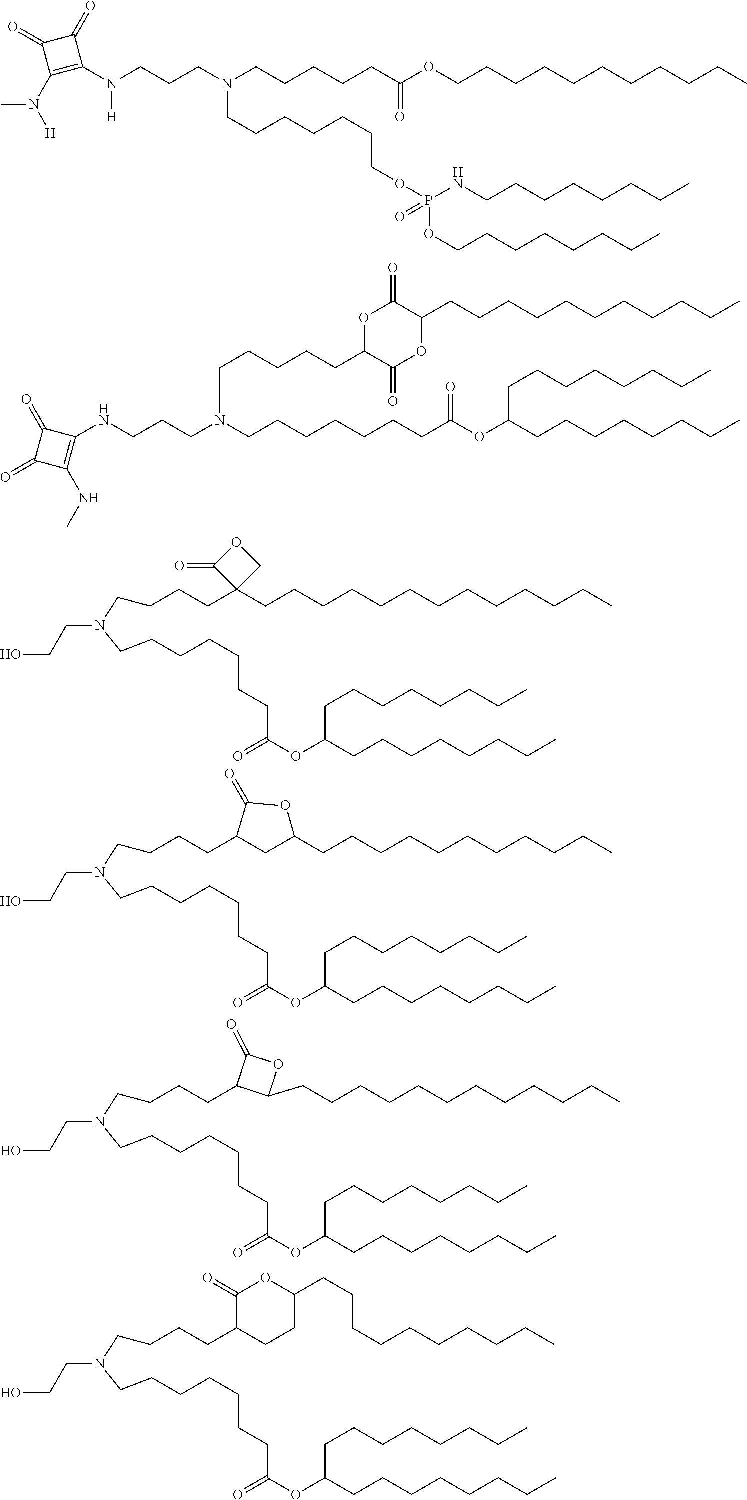

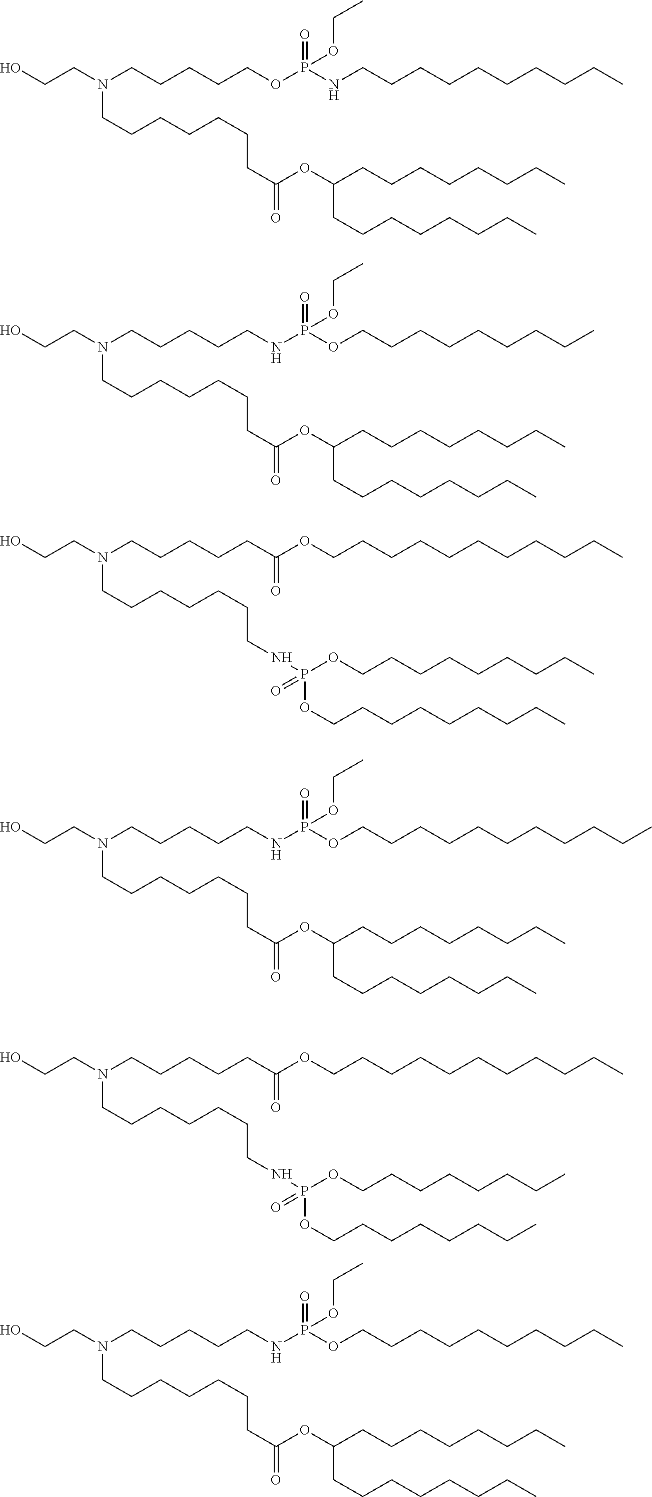

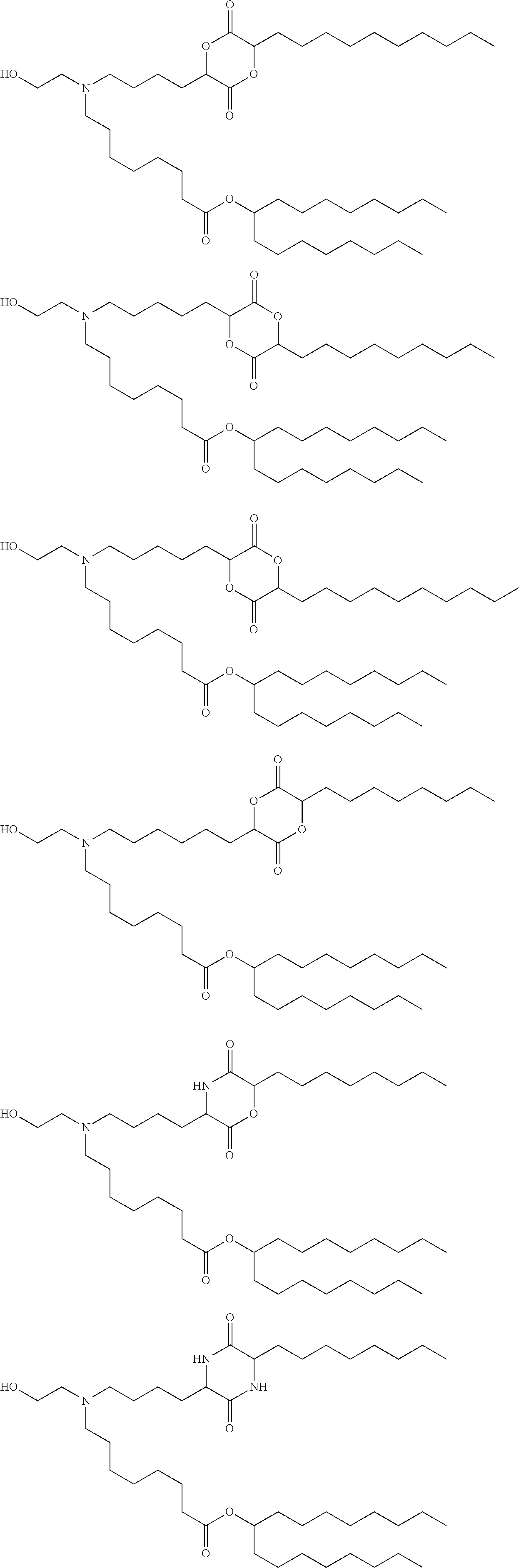

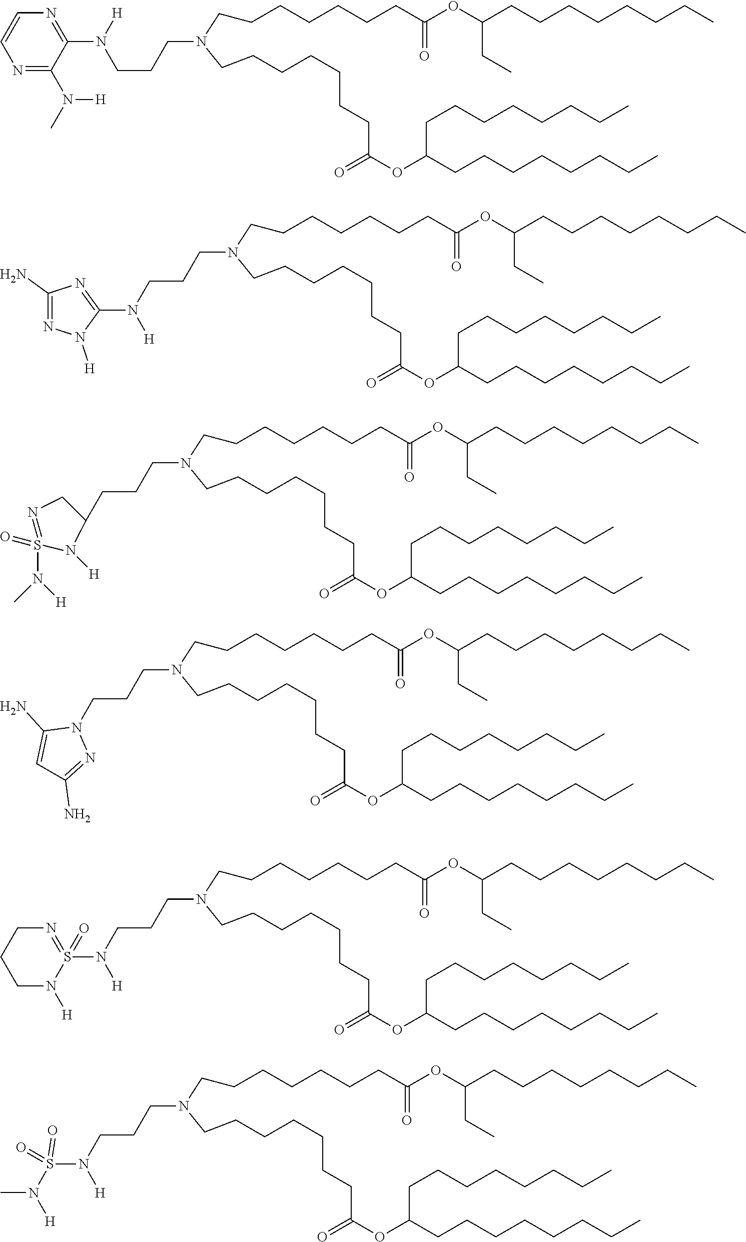

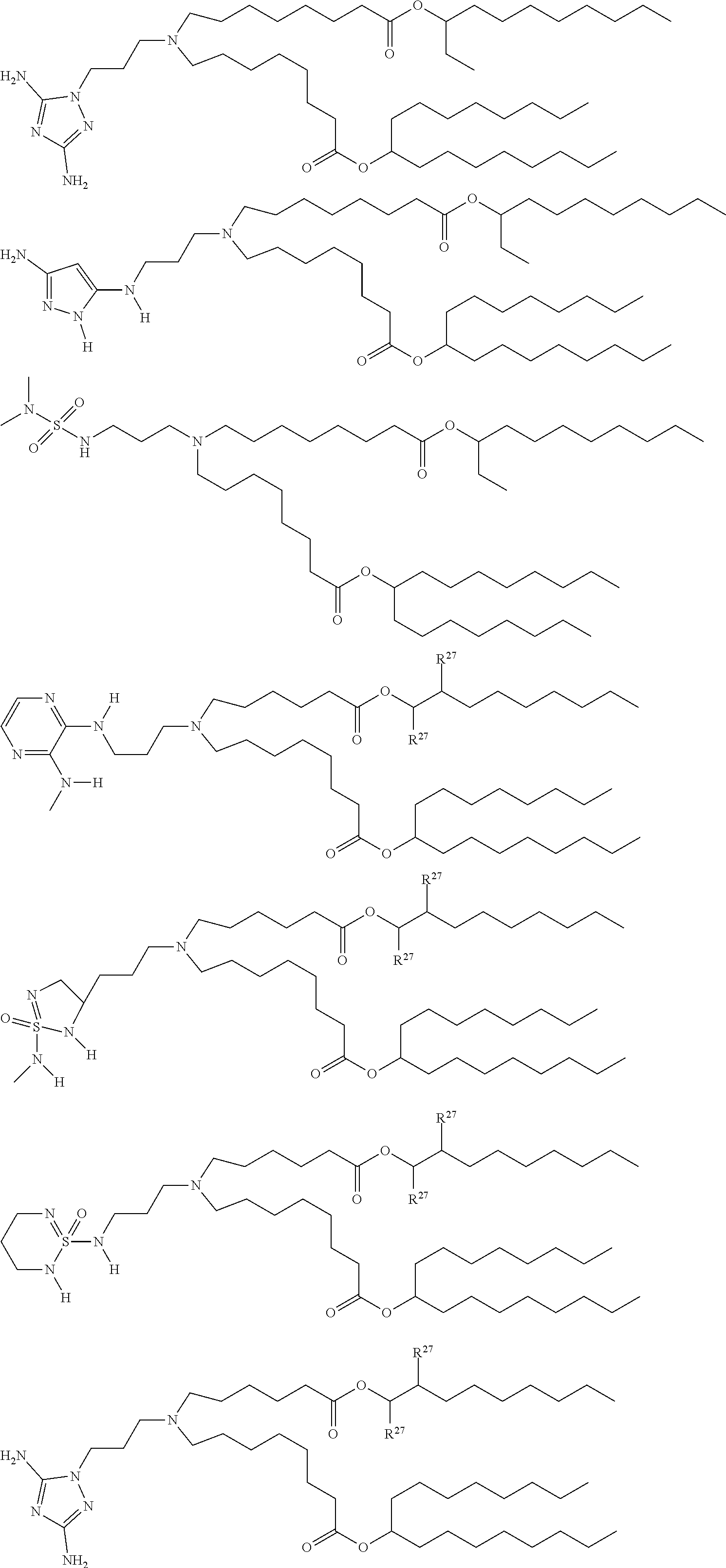

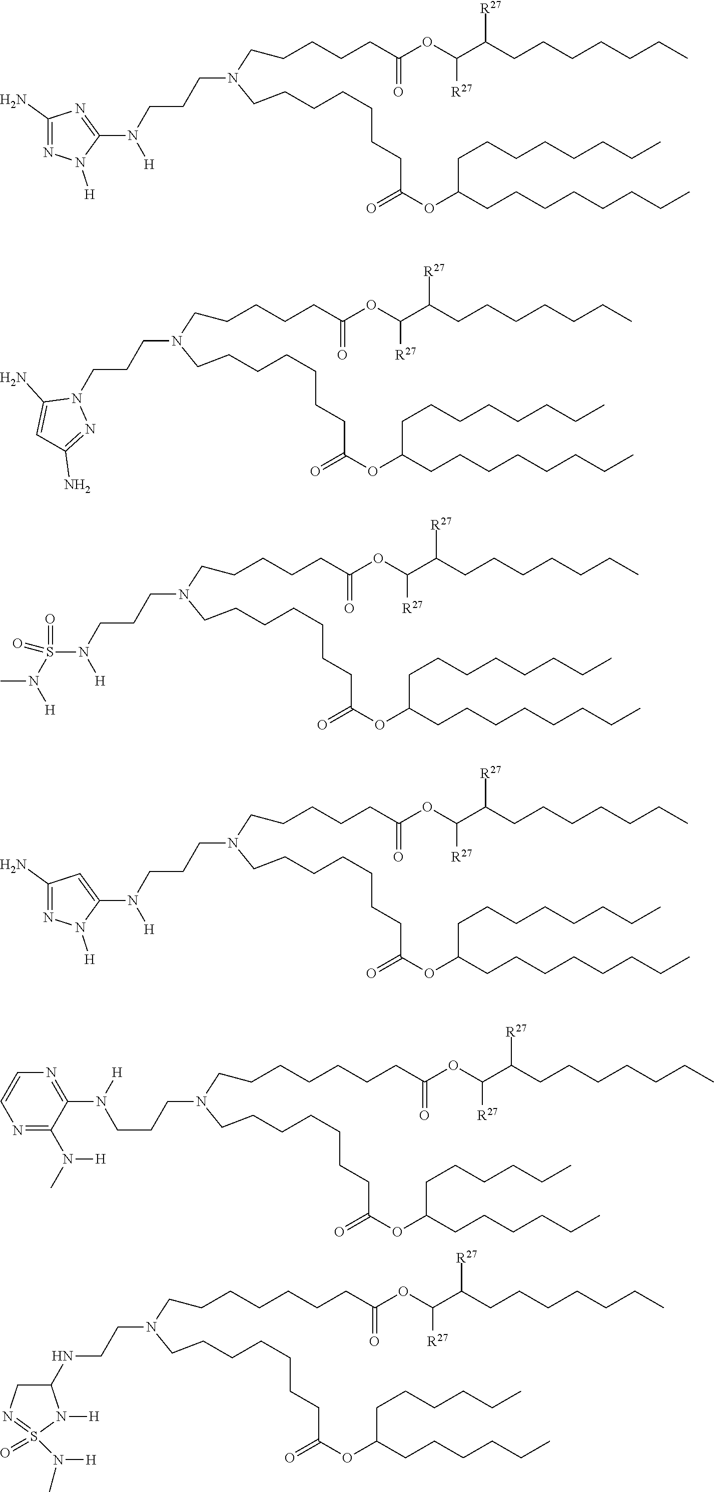

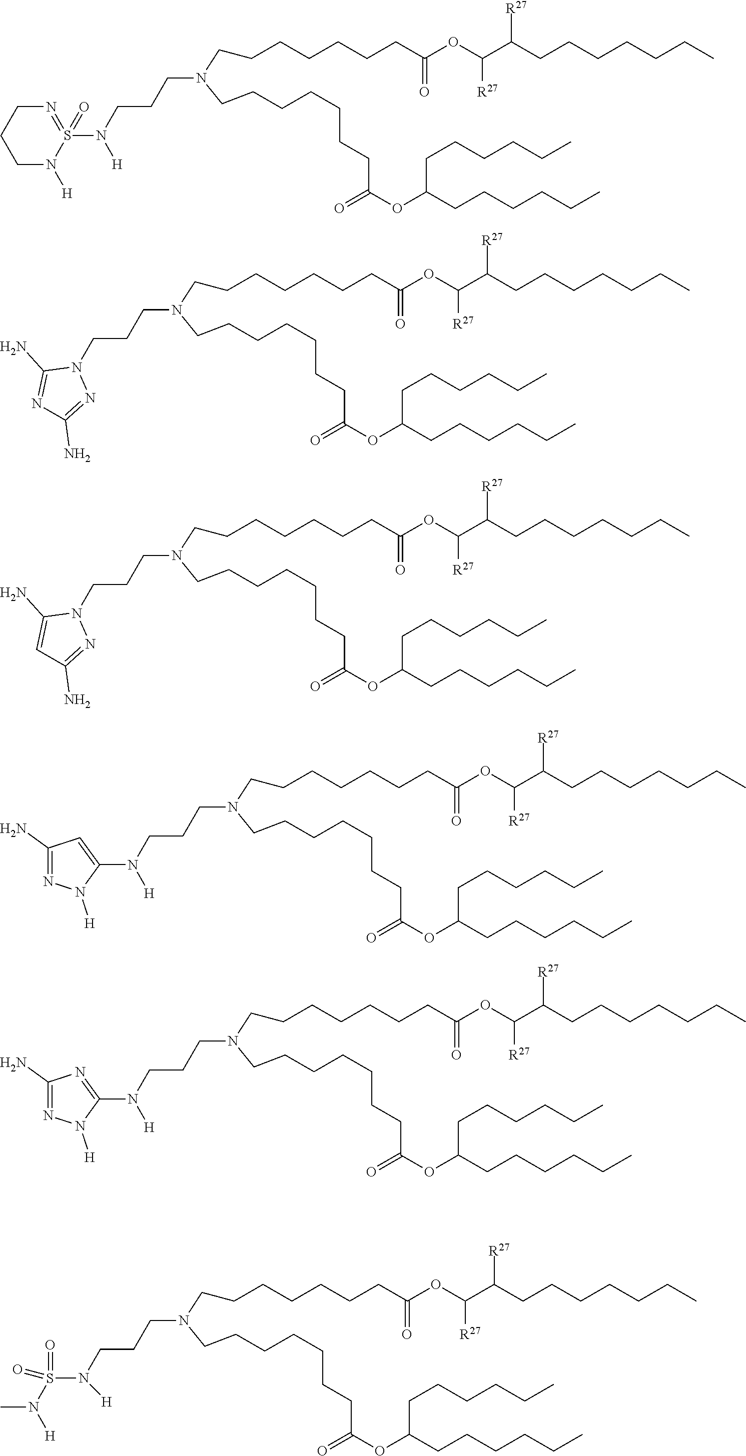

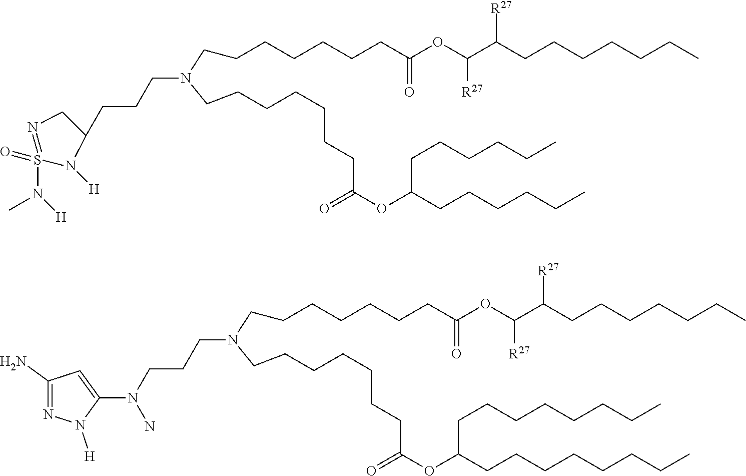

wherein R=

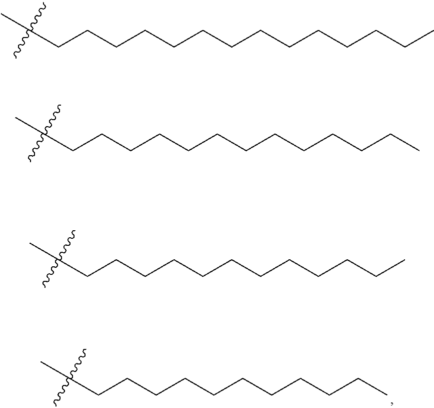 [SMILES: *CCCCCCCCCCCCCC ; *CCCCCCCCCCCCC ; *CCCCCCCCCCCC ; *CCCCCCCCCCC]

and each R²⁷ is independent HK C₁-C₁₅ alkyl, C₂-C₈ alkenyl, or C₂-C₈ alkynyl.

In some embodiments, the present disclosure relates to a compound of formula (AL-GI):

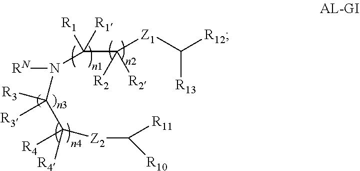

or a salt thereof;
wherein:
R^{N} is substituted or unsubstituted C₁-C₆ alkyl or C₃-C₈ cycloalkyl;
each of R₁, R_{1′}, R₂, R_{2′}, R₃, R_{3′}, R₄, and R_{4′}, independently for each occurrence, is H, branched C₁-C₃ alkyl, unbranched C₁-C₃ alkyl, branched C₂-C₃ alkenyl, or unbranched C₂-C₃ alkenyl;
each of R₁₀, R₁₁, R₁₂, and R₁₃ is independently H or a substituted or unsubstituted branched C₁-C₁₅ alkyl or unbranched C₁-C₁₅ alkyl; provided that at least one of R₁₀ and R₁₁ is not H, and at least one of R₁₂ and R₁₃ is not H;
each of Z₁ and Z₂ is independently

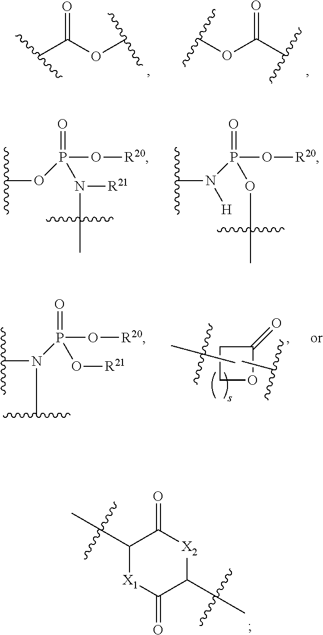

each of X¹ and X² is independently O, S, or N(R₂₁);
R₂₀ is a substituted or unsubstituted branched C₁-C₁₅ alkyl or unbranched C₁-C₁₅ alkyl;
R₂₁ is H, substituted or unsubstituted C₁-C₅ alkyl, or substituted or unsubstituted C₃-C₈ cycloalkyl;
s is an integer from 1 to 4; and
each of n₁, n₂, n₃, and n₄ is independently an integer from 0 to 15, wherein n₁, n₂ ranges from 1 to 15 and n₃ n₄ ranges from 1 to 15;
wherein when R^{N} is C₁-C₆ alkyl substituted with hydroxy, then at least one of (i) or (ii) applies:
   (i) at least one of Z₁ and Z₂ is

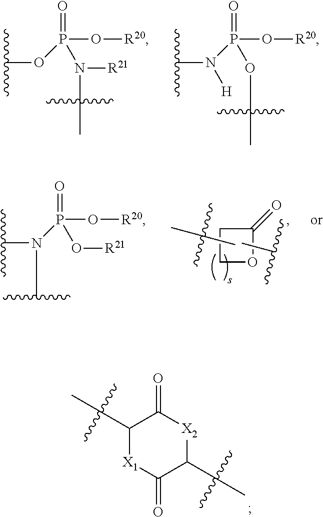

or
(ii) the sum of n₁ and n₂ does not equal the sum of n₃ and n₄.

In some embodiments, R^{N} is C₁-C₆ alkyl, C₃-C₈ cycloalkyl, —(CH₂)ᵥQ, —(CH₂)ᵥN(R″)Q, —C(Q)(R)₂, or —(CH₂)ᵥC(Q)(R)₂;
each Q is independently —OR″, —SR″, C₃-C₈ cycloalkyl, C₃-C₈ cycloalkenyl, heterocyclyl, heterocycloalkenyl, aryl, heteroaryl, —O(CH₂)ᵥN(R″)₂, —C(O)OR″, —OC(O)R, —C(R′)₃, —CN, —C(O)N(R″)₂, —N(R″)C(O)R, —N(R″)S(O)₂R, —N(R″)C(O)N(R″)₂, —N(R″)C(S)N(R″)₂, —N(R″)R^{a}, —O(CH₂)ᵥOR″, —N(R″)C(═NR^{b})N(R″)₂, —N(R″)C(═CHR^{b})N(R″)₂, —OC(O)N(R″)₂, —N(R″)C(O)OR″, —N(OR″)C(O)R, —N(OR″)S(O)₂R, —N(OR″)C(O)OR″, —N(OR″)C(O)N(R″)₂, —N(OR″)C(S)N(R″)₂, —N(OR″)C(═NR″)N(R″)₂, —N(OR″)C(═CHR^{b})N(R″)₂, —C(═Na)N(R″)₂, —C(═NR)R, or —C(O)N(R″)OR″,
each R is independently H, C₁-C₃ alkyl, C₂-C₃ alkenyl, amino, monoalkylamino, or dialkylamino;
each R′ is independently H, F, Cl, Br, or I;
each R″ is independently H, C₁-C₃ alkyl, or C₂-C₃ alkenyl;
each R^{a} is independently H or C₃-C₈ cycloalkyl;
each R^{b} is independently H, CN, NO₂, C₁-C₆ alkyl, —OR, —S(O)₂R, —S(O)₂N(R″)₂, C₂-C₆ alkenyl, C₃-C₈ cycloalkyl, or heterocyclyl; and
v is i an integer from 1 to 6;
wherein each of the alkyl, cycloalkyl, cycloalkenyl, heterocyclyl, heterocycloalkenyl, aryl, and heteroaryl groups are optionally substituted by one or more substituents selected from the group consisting of oxo (═O), OH, amino, monoalkylamino, dialkylamino, and C₁-C₃ alkyl.

In some embodiments, R^{N} is unsubstituted C₁₋₄ alkyl, —(CH₂) N(R″)Q, or —(CH₂)ᵥQ;
Q is —OH, —SH, —NHC(S)N(R″)₂, —NHC(O)N(R″)₂, —N(R″)C(O)R, —N(R″)S(O)₂R, —N(R″)R^{a}, —NHC(═NR^{b})N(R″)₂, —NHC(═CHR^{b})N(R″)₂, —OC(O)N(R″)₂, —N(R″)C(O)OR″, heterocyclyl, or heteroaryl; and

In some embodiments, R^{N} is —(CH₂)ᵥOH, and v is 2, 3, or 4.

In some embodiments, R^{N} is —CH₂CH₂OH.

In some embodiments, R^{N} is —(CH₂)ᵥQ or —(CH₂)ᵥN(R″)Q, Q is heterocyclyl, heterocycloalkenyl, aryl, or heteroaryl, optionally substituted with one or more substituents.

In some embodiments, R^{N} is —(CH₂)ᵥN(R″)S(O)₂R.

In some embodiments, R^{N} is

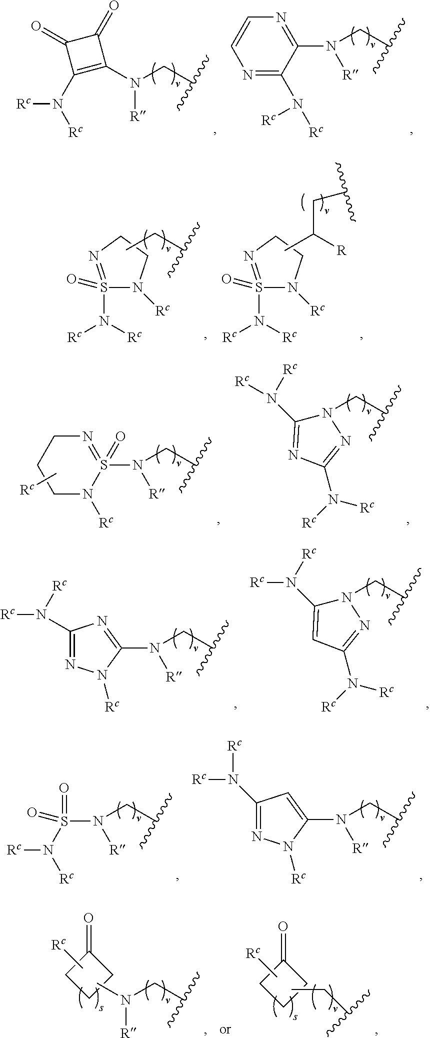

wherein each R is independently H or C₁-C₃ alkyl, and s is an integer from 1 to 4.

In some embodiments, R^{N} is

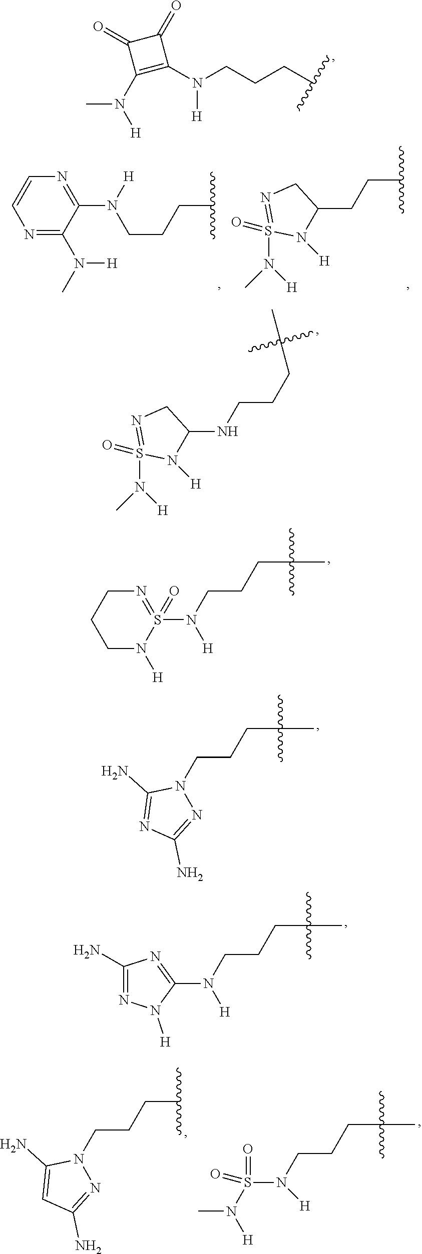

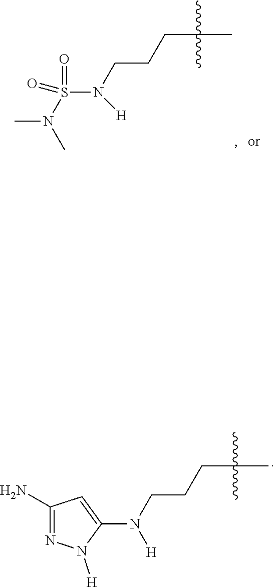 [SMILES: *CCCNS(=O)(=O)N(C)C ; *CCCNc1cc(N)n[nH]1]

In some embodiments, n₁+n₂ ranges from 1 to 10 and n₃+n₄ ranges from 1 to 10.

In some embodiments, n₁+n₂ ranges from 2 to 7 and n₃+n₄ ranges from 2 to 7, and n₁+n₂ and n₃+n₄ are the same.

In some embodiments, n₁+n₂ ranges from 2 to 7 and n₃+n₄ ranges from 2 to 7, and n₁+n₂ and n₃+n₄ are different.

In some embodiments, each of Z₁ and Z₂ is independently

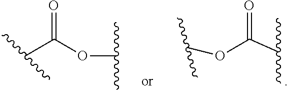 [SMILES: *OC(*)=O ; *OC(*)=O]

In some embodiments, one of Z₁ and Z₂ is

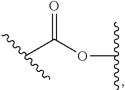 [SMILES: *OC(*)=O]

and the other of Z₁ and Z₂ is

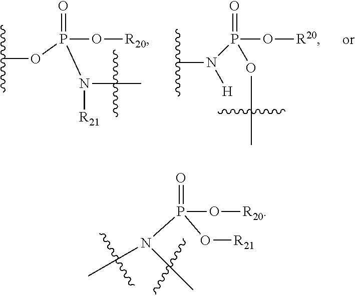

In some embodiments, each of Z₁ and Z₂ is independently

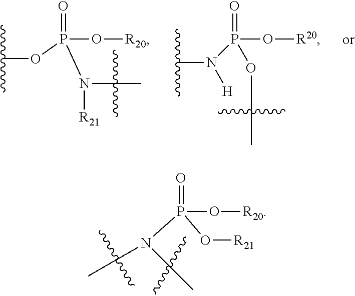

In some embodiments:
when Z₂ is

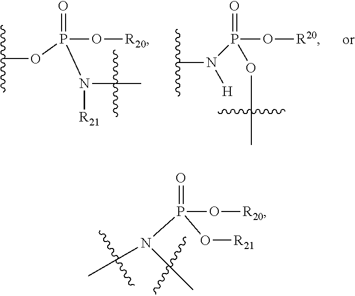

### R¹¹ is H; and

when Z₁ is

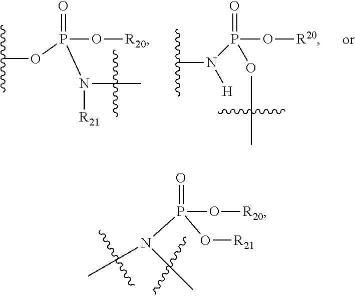

### R¹³ is H.

In some embodiments, each of Z₁ and Z₂ is

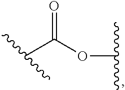 [SMILES: *OC(*)=O]

and the other of Z₁ and Z₂ is

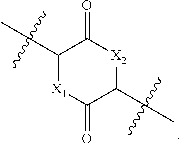

In some embodiments, each of Z₁ and Z₂ is independently a same or different

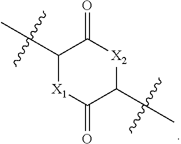

In some embodiments, one of Z₁ and Z₂ is

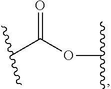 [SMILES: *OC(*)=O]

and the other of Z₁ and Z₂ is

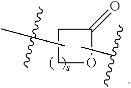

In some embodiments, each of Z₁ and Z₂ is independently a same or different

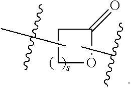

In some embodiments, the compound has the formula:

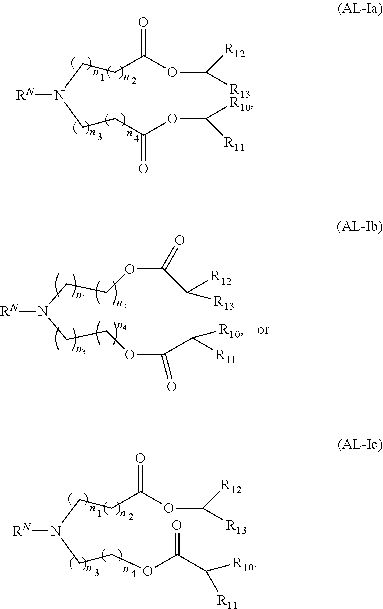

In some embodiments, R₁₀ and R₁₁ are each independently unsubstituted C₅-C₈ alkyl; or R₁₁ is H, and R₁₀ is unsubstituted C₁₀-C₁₄ alkyl; and
R₁₂ and R₁₃ are each independently unsubstituted C₅-C₈alkyl; or R₁₃ is H, and R₁₂ is unsubstituted C₁₀-C₁₄ alkyl;
wherein R₁₁ and R₁₃ are not both H.

In some embodiments, n₁+n₂ is an integer from 2 to 4, and n₃+n₄ is an integer from 5 to 7; or n₁+n₂ is an integer from 5 to 7, and n₃+n₄ is an integer from 2 to 4.

In some embodiments, the compound has the formula:

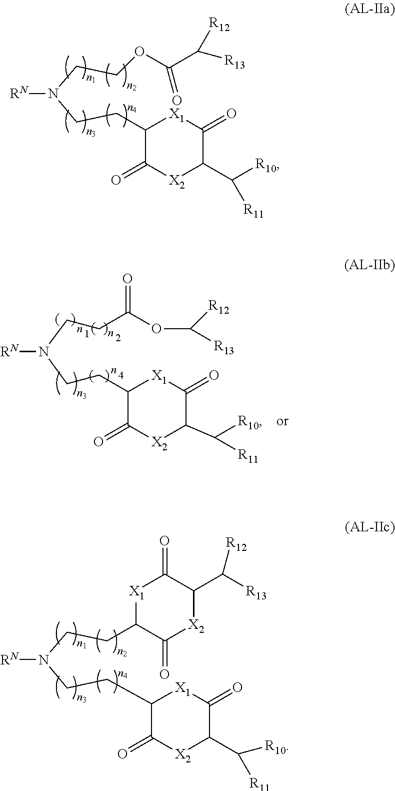

In some embodiments, the compound the formula:

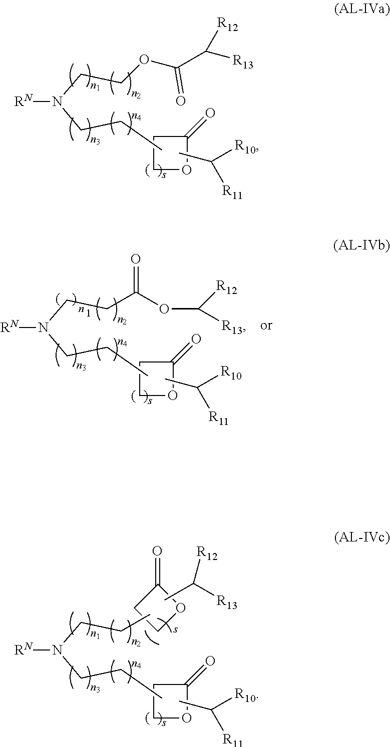

In some embodiments, R₁₀ and R₁₁ are each independently unsubstituted C₅-C₈ alkyl; or R₁₁ is H, and R₁₀ is unsubstituted C₇-C₁₁ alkyl; and
R₁₂ and R¹³ are each independently unsubstituted C₅-C₈ alkyl; or R₃ is H, and R₁₂ is unsubstituted C₇-C₁₁ alkyl,
wherein R₁₁ and R₁₃ are not both H.

In some embodiments, each of X₁ and X₂ is independently O or N(R₂₁), and R₂₁ is H or C₁-C₃ alkyl.

In some embodiments, n₁+n₂ is an integer from 4 to 7, and n₃+n₄ is an integer from 6 to 7; or n₁+n₂ is an integer from 6 to 7, and n₃+n₄ is an integer from 4 to 7.

In some embodiments, the compound has the formula:

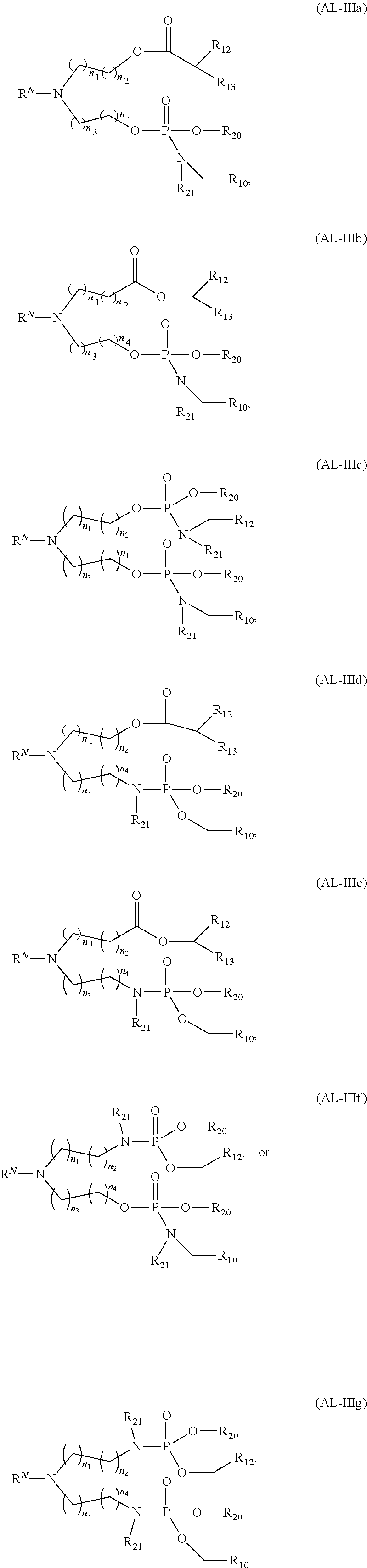

In some embodiments, R₁₀ is unsubstituted C₇-C₁₁ alkyl; R₁₂ and R₁₃ are each independently unsubstituted C₅-C₈alkyl; or R₁₃ is H, and R₁₂ is unsubstituted C₇-C₁₁ alkyl;
each R₂₁ is H; and
each R₂₀ is independently unsubstituted C₂-C₉ alkyl.

In some embodiments, each of n₁+n₂ and n₃+n₄ is independently an integer from 5 to 7.

In some embodiments, the present disclosure relates to a lipid-based carrier comprising a compound of the present disclosure, e.g., a compound of formula (Ia) or (AL-GI), wherein the lipid-based carrier is a lipid nanoparticle.

In some embodiments, the lipid-based carrier further comprises a second lipid.

In some embodiments, the second lipid is a cationic, anionic, ionizable, or zwitterionic lipid.

In some embodiments, the lipid-based carrier further comprises a PEGylated lipid, a sterol, a phospholipid, and/or a neutral lipid.

In some embodiments, the lipid component of the lipid-based carrier comprises:
about 25-100 mol % of the compound,
about 0-50 mol % phospholipid,
about 0-50 mol % sterol, and
about 0-10 mol % PEGylated lipid.

In some embodiments, the lipid component of the lipid-based carrier comprises:
about 30-60 mol % of the compound,
about 0-30 mol % phospholipid,
about 15-50 mol % sterol, and
about 0-10 mol % PEGylated lipid.

In some embodiments, the lipid nanoparticle further comprises an effector, such as a therapeutic agent.

In some embodiments, the therapeutic agent is a nucleic acid molecule.

In some embodiments, the nucleic acid molecule is a nucleic acid selected from the group consisting of a plasmid, an immunostimulatory oligonucleotide, an antisense oligonucleotide, an antagomir, an aptamer, a deoxyribozyme (DNAzyme), and a ribozyme.

In some embodiments, the nucleic acid molecule is DNA or RNA.

In some embodiments, the DNA is a linear DNA, circular DNA, single stranded DNA, or double stranded DNA.

In some embodiments, the RNA is selected from the group consisting of an mRNA, miRNA, siRNA or siRNA precursor, RNA aptamer, linear RNA, circular RNA, single stranded RNA, double stranded RNA, tRNA, microRNA (miRNA) or miRNA precursor, a Dicer substrate small interfering RNA (dsiRNA), a short hairpin RNA (shRNA), an asymmetric interfering RNA (aiRNA), a guide RNA (gRNA), lncRNA, ncRNA, sncRNA, rRNA, snRNA, piRNA, snoRNA, snRNA, scaRNA, exRNA, scaRNA, Y RNA, and hnRNA.

In some embodiments, the RNA is mRNA.

In some embodiments, the nucleic acid molecule comprises one or more nucleic acid analogs selected from the group consisting of a phosphoramide, a phosphorothioate, a phosphorodithioate, an O-methylphosphoroamidate, a morpholino, a locked nucleic acid (LNA), a glycerol nucleic acid (GNA), a threose nucleic acid (TNA), and a peptide nucleic acid (PNA).

In some embodiments, the therapeutic agent is a protein or small molecule drug.

In some embodiments, the lipid nanoparticle comprises an antigen.

In some embodiments, the antigen is a protein or a nucleic acid.

In some embodiments, the antigen is a protein.

In some embodiments, the antigen is a nucleic acid.

In some embodiments, the lipid nanoparticle comprises an mRNA molecule comprising a nucleotide sequence encoding an antigen.

In some embodiments, the present disclosure relates to a method of delivering an effector, such as a therapeutic agent to a subject, the method comprising administering to the subject a lipid-based carrier of the present disclosure, wherein the lipid-based carrier comprises the effector.

In some embodiments, the present disclosure relates to a method of vaccinating a subject in need thereof, comprising administering to the subject an effective amount of the lipid-based carrier of the present disclosure, wherein the lipid-based carrier comprises an antigen.

In some embodiments, the present disclosure relates to a pharmaceutical composition comprising the lipid-based carrier of the present disclosure, and a pharmaceutically acceptable excipient.

### The Novel Ionizable Lipids

One aspect of the invention relates to a compound of formula (AL-GI):

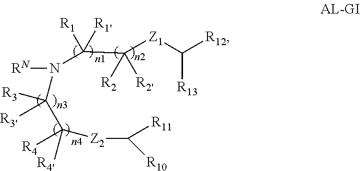

wherein:
R^{N} is substituted or unsubstituted C₁-C₆ alkyl or C₃-C₈ cycloalkyl;
each of R₁, R_{1′}, R₂, R_{2′}, R₃, R_{3′}, R₄, and R_{4′}, is, for each occurrence, independently H, branched or unbranched C₁-C₃ alkyl, or branched or unbranched C₂-C₃ alkenyl;
each of R₁₀, R₁₁, R₁₂, and R₁₃ is independently H or a branched or unbranched, substituted or unsubstituted C₁-C₁₅ alkyl; provided that at least one of R₁₀ and R₁₁ is not H, and at least one of R₁₂ and R₁₃ is not H;
each of Z₁ and Z₂ is independently

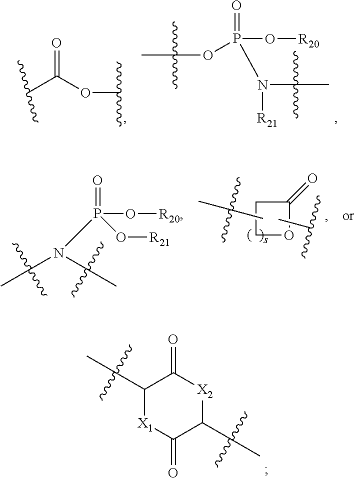

each of X₁ and X₂ is independently O, S, or N(R₂₁);
R₂₀ is a branched or unbranched, substituted or unsubstituted C₁-C₁₅ alkyl;
R²¹ is H, substituted or unsubstituted C₁-C₅ alkyl, or substituted or unsubstituted C₃-C₈ cycloalkyl;
s is an integer from 1 to 4; and
each of n₁, n₂, n₃, and n₄ is independently an integer from 0 to 15, wherein n₁+n₂ ranges from 1 to 15 and n₃, n₄ ranges from 1 to 15.

In any of the formulas described herein, R^{N} may be C₁-C₆ alkyl or C₃-C₈ cycloalkyl. Each of the alkyl and cycloalkyl groups may be unsubstituted, or substituted with one or more substituents.

In some embodiments, in any of the formulas described herein, R^{N} is C₁-C₆ alkyl, C₃-C₈ cycloalkyl, —(CH₂)ᵥQ, —(CH₂)ᵥN(R)Q, —C(Q)(R)₂, or —(CH₂)ᵥC(Q)(R)₂.

Each Q is independently —OR, —SR, C₃-C₈ cycloalkyl, heterocyclyl, heteroaryl, —O(CH₂)ᵥN(R)₂, —C(O)OR, —OC(O)R, —C(R′)₃, —CN, —C(O)N(R)₂, —N(R)C(O)R, —N(R)S(O)₂R, —N(R)C(O)N(R)₂, —N(R)C(S)N(R)₂, —N(R)R^{a}, —O(CH₂)ᵥOR, —N(R)C(═NR^{b})N(R)₂, —N(R)C(═CHR^{b})N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR^{b})N(R)₂, —N(OR)C(═CHR^{b})N(R)₂, —C(═NR^{b})N(R)₂, —C(═NR^{b})R, or —C(O)N(R)OR.

Each of the alkyl, cycloalkyl, heterocyclyl, and heteroaryl groups may be optionally substituted by one or more substituents selected from the group consisting of oxo (═O), OH, amino, mono- or di-alkylamino, and C₁-C₃ alkyl. Each R is independently H, C₁-C₃ alkyl, C₂-C₃ alkenyl, amino, or mono- or di-alkylamino. Each R′ is independently H, F, Cl, Br, or I. Each R^{a} is independently H or C₃-C₈ cycloalkyl. Each R^{b} is independently H, CN, NO₂, C₁-C₆ alkyl, —OR, —S(O)₂R, —S(O)₂N(R)₂, C₂-C₆ alkenyl, C₃-C₈ cycloalkyl, or heterocyclyl. Each v is independently an integer from 1 to 6.

In some embodiments, in any of the formulas described herein, R^{N} is unsubstituted C₁-C₆ alkyl, such as unsubstituted C₁-C₄alkyl.

In some embodiments, in any of the formulas described herein, R^{N} is C₁-C₆ alkyl, such as C₁-C₄ alkyl, substituted with one or more substituents. In some embodiments, the substituents are OH, oxo (═O), amino, mono- or di-alkylamino, or C₁-C₃ alkyl.

In some embodiments, in any of the formulas described herein, R^{N} is unsubstituted C₃-C₈ cycloalkyl, such as C₃-C₆ cycloalkyl (e.g., C₅-C₆ cycloalkyl).

In some embodiments, in any of the formulas described herein, R^{N} is C₃-C₈ cycloalkyl, such as C₃-C₆ cycloalkyl (e.g., C₅-C₆ cycloalkyl), substituted with one or more substituents. In some embodiments, the substituents are OH, oxo (═O), amino, mono- or di-alkylamino, or C₁-C₃alkyl.

In some embodiments, in any of the formulas described herein, R^{N} is —(CH₂)ᵥQ or
—(CH₂)—N(R)Q, Q is OH, SH, —NHC(S)N(R)₂, —NHC(O)N(R)₂, —N(R)C(O)R, —N(R)S(O)₂R,
—N(R)R^{a}, —NHC(═NR″)N(R)₂, —NHC(═CHR^{b})N(R)₂, —OC(O)N(R)₂, —N(R)C(O)OR, heterocyclyl, or heteroaryl. Each heterocyclyl and heteroaryl group may be substituted with one or more substituents. Each v is independently 2, 3, 4, or 5. In some embodiments, Q is OH,
—NHC(S)N(R)₂, —NHC(O)N(R)₂, —N(R)C(O)R, or —N(R)S(O)₂R.

In some embodiments, in any of the formulas described herein, R^{N} is —(CH₂)ᵥOH, and v is an integer from 1 to 6, e.g., v is 2, 3, or 4.

In some embodiments, in any of the formulas described herein, R^{N} is —(CH₂)ᵥQ or
—(CH₂)ᵥN(R)Q. Q is —N(R)S(O)₂R, heterocyclyl, or heteroaryl. Each heterocyclyl and heteroaryl group may be substituted with one or more substituents. Each v is independently 2, 3, 4, or 5.

In some embodiments, in any of the formulas described herein, R^{N} is —(CH₂)ᵥQ. Q is heterocyclyl, which may be substituted with one or more substituents. In some embodiments, the substituents may be oxo (═O), OH, amino, mono- or di-alkylamino, or C₁-C₃ alkyl.

In some embodiments, in any of the formulas described herein, R^{N} is —(CH₂)ᵥQ. Q is heteroaryl, which may be substituted with one or more substituents. In some embodiments, the substituents may be oxo (═O), OH, amino, mono- or di-alkylamino, or C₁-C₃ alkyl.

In some embodiments, in any of the formulas described herein, R^{N} is —(CH₂)ᵥN(R)Q. Q is heterocyclyl, which may be substituted with one or more substituents. In some embodiments, the substituents may be oxo (═O), OH, amino, mono- or di-alkylamino, or C₁-C₃ alkyl. Each R is independently H, C₁-C₃ alkyl, C₂-C₃ alkenyl, amino, or mono- or di-alkylamino.

In some embodiments, in any of the formulas described herein, R^{N} is —(CH₂)ᵥN(R)Q. Q is heteroaryl, which may be substituted with one or more substituents. In some embodiments, the substituents may be oxo (═O), OH, amino, mono- or di-alkylamino, or C₁-C₃ alkyl. Each R is independently H, C₁-C₃ alkyl, C₂-C₃ alkenyl, amino, or mono- or di-alkylamino.

In some embodiments, in any of the formulas described herein, R^{N} is —(CH₂), N(R)S(O)₂R. Each R is independently H, C₁-C₃ alkyl, C₂-C₃ alkenyl, amino, or mono- or di-alkylamino.

In some embodiments, in any of the formulas described herein, R^{N} has the following structures:

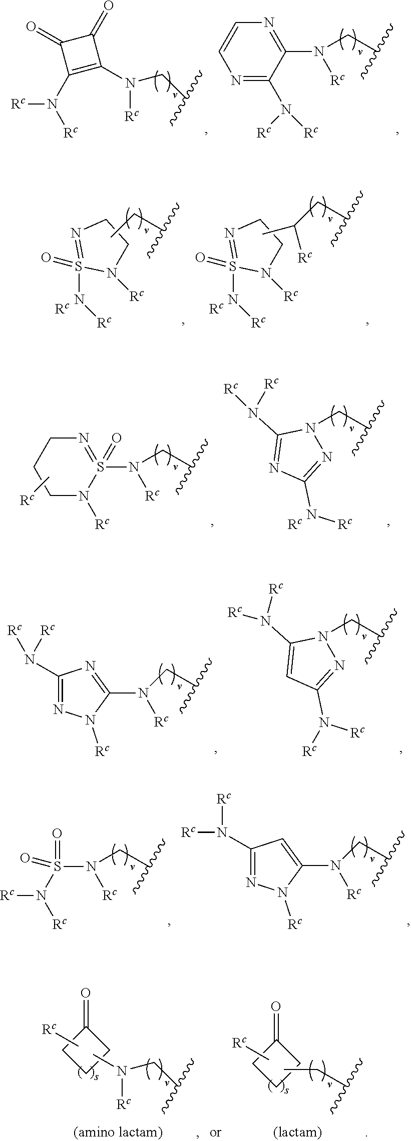

Each R^{c} is independently H or C₁-C₃ alkyl. v is an integer from 1 to 6, e.g., v is 2, 3, or 4. s is an integer from 1 to 4.

In some embodiments, in any of the formulas described herein, R^{N} has the structure of:

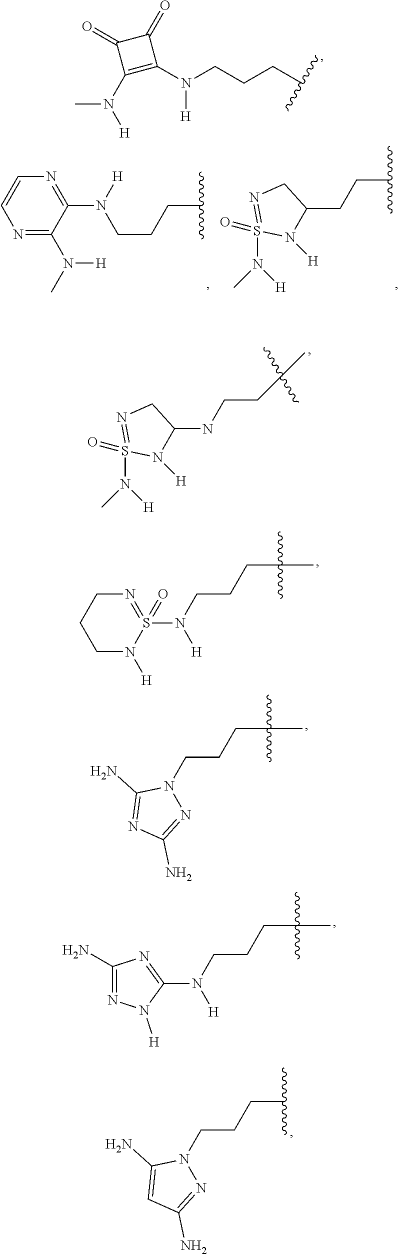

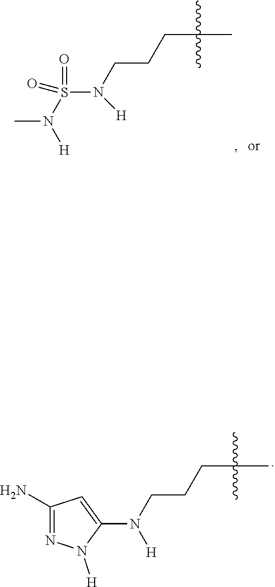

In any of the formulas described herein, each of n₁, n₂, n₃, and n₄ is independently an integer from 0 to 15, for instance, each of n₁, n₂, n₃, and n₄ is independently an integer from 0 to 10, from 0 to 7, or from 0 to 4. In some embodiments, n₁+n₂ ranges from 1 to 15 and n₃+n₄ ranges from 1 to 15. For instance, n₁+n₂ may range from 1 to 10 and n₃+n₄ may range from 1 to 10.

In some embodiments, in any of the formulas described herein, n₁+n₂ ranges from 2 to 7 and n₃+n₄ ranges from 2 to 7, and n₁+n₂ and n₃+n₄ are the same.

In some embodiments, in any of the formulas described herein, n₁+n₂ ranges from 2 to 7 and n₃+n₄ ranges from 2 to 7, and n₁+n₂ and n₃+n₄ are different.

In any of the formulas described herein, each of R₁, R_{1′}, R₂, R_{2′}, R₃, R_{3′}, R₄, and R_{4′} is independently H, branched or unbranched C₁-C₃ alkyl, or branched or unbranched C₂-C₃ alkenyl.

Each of the alkyl and C₂-C₃ alkenyl groups may be unsubstituted or substituted with one or more substituents. In some embodiments, each of the alkyl and C₂-C₃ alkenyl groups are unsubstituted. In some embodiments, the alkyl or C₂-C₃ alkenyl groups are substituted with one or more substituents. In some embodiments, each of R₁, R_{1′}, R₂, R_{2′}, R₃, R_{3′}, R₄, and R_{4′} is independently H or unsubstituted C₁-C₃ alkyl. In some embodiments, each of R₁, R_{1′}, R₂, R_{2′}, R₃, R_{3′}, R₄, and R_{4′} is independently H or methyl. In one embodiment, each of R₁, R_{1′}, R₂, R_{2′}, R₃, R_{3′}, R₄, and R_{4′} is H.

In some embodiment, n₁ is at least 1, and R₁ and R_{1′} are different, for at least one occurrence. In some embodiment, n₁ is at least 1, and R₁ and R_{1′} are the same, for at least one occurrence. In some embodiment, n₁ is at least 2, and R₁ and R_{1′} are different, for at least two occurrences. In some embodiment, n₁ is at least 2, and R₁ and R_{1′} are the same, for at least two occurrences. In some embodiment, n₁ is at least 3, and R₁ and R_{1′} are different, for at least three occurrences. In some embodiment, n₁ is at least 3, and R₁ and R_{1′} are the same, for at least three occurrences. In some embodiment, n₁ is at least 4, and R₁ and R_{1′} are different, for at least four occurrences. In some embodiment, n₁ is at least 4, and R₁ and R_{1′} are the same, for at least four occurrences. In some embodiment, n₁ is at least 5, and R₁ and R_{1′} are different, for at least five occurrences. In some embodiment, n₁ is at least 5, and R₁ and R_{1′} are the same, for at least five occurrences. In some embodiment, R₁ and R_{1′} are different, for each occurrence. In some embodiment, R₁ and R_{1′} are the same, for each occurrence.

In some embodiment, n₂ is at least 1, and R₂ and R_{2′} are different, for at least one occurrence. In some embodiment, n₂ is at least 1, and R₂ and R_{2′} are the same, for at least one occurrence. In some embodiment, n₂ is at least 2, and R₂ and R_{2′} are different, for at least two occurrences. In some embodiment, n₂ is at least 2, and R₂ and R_{2′} are the same, for at least two occurrences. In some embodiment, n₂ is at least 3, and R₂ and R_{2′} are different, for at least three occurrences. In some embodiment, n₂ is at least 3, and R₂ and R_{2′} are the same, for at least three occurrences. In some embodiment, n₂ is at least 4, and R₂ and R_{2′} are different, for at least four occurrences. In some embodiment, n₂ is at least 4, and R₂ and R_{2′} are the same, for at least four occurrences. In some embodiment, n₂ is at least 5, and R₂ and R_{2′} are different, for at least five occurrences. In some embodiment, n₂ is at least 5, and R² and R_{2′} are the same, for at least five occurrences. In some embodiment, R₂ and R_{2′} are different, for each occurrence. In some embodiment, R₂ and R_{2′} are the same, for each occurrence.

In some embodiment, n₃ is at least 1, and R₃ and R_{3′} are different, for at least one occurrence. In some embodiment, n₃ is at least 1, and R₃ and R_{3′} are the same, for at least one occurrence. In some embodiment, n₃ is at least 2, and R₃ and R_{3′} are different, for at least two occurrences. In some embodiment, n₃ is at least 2, and R₃ and R_{3′} are the same, for at least two occurrences. In some embodiment, n₃ is at least 3, and R₃ and R_{3′} are different, for at least three occurrences. In some embodiment, n₃ is at least 3, and R₃ and R_{3′} are the same, for at least three occurrences. In some embodiment, n₃ is at least 4, and R₃ and R_{3′} are different, for at least four occurrences. In some embodiment, n₃ is at least 4, and R₃ and R_{3′} are the same, for at least four occurrences. In some embodiment, n₃ is at least 5, and R₃ and R_{3′} are different, for at least five occurrences. In some embodiment, n₃ is at least 5, and R₃ and R_{3′} are the same, for at least five occurrences. In some embodiment, R₃ and R_{3′} are different, for each occurrence. In some embodiment, R₃ and R_{3′} are the same, for each occurrence.

In some embodiment, n₄ is at least 1, and R₄ and R_{4′} are different, for at least one occurrence. In some embodiment, n₄ is at least 1, and R₄ and R_{4′} are the same, for at least one occurrence. In some embodiment, n₄ is at least 2, and R₄ and R_{4′} are different, for at least two occurrences. In some embodiment, n₄ is at least 2, and R₄ and R_{4′} are the same, for at least two occurrences. In some embodiment, n₄ is at least 3, and R₄ and R_{4′} are different, for at least three occurrences. In some embodiment, n₄ is at least 3, and R₄ and R_{4′} are the same, for at least three occurrences. In some embodiment, n₄ is at least 4, and R₄ and R_{4′} are different, for at least four occurrences. In some embodiment, n₄ is at least 4, and R₄ and R_{4′} are the same, for at least four occurrences. In some embodiment, n₄ is at least 5, and R₄ and R_{4′} are different, for at least five occurrences. In some embodiment, n₄ is at least 5, and R₄ and R_{4′} are the same, for at least five occurrences. In some embodiment, R₄ and R_{4′} are different, for each occurrence. In some embodiment, R₄ and R_{4′} are the same, for each occurrence.

In any of the formulas described herein, each of R₁₀, R₁₁, R₁₂, and R₁₃ is independently H or a branched or unbranched, substituted or unsubstituted C₁-C₁₅ alkyl. In some embodiments, each of R₁₀, R₁₁, R₁₂, and R₁₁ is independently H or a C₁-C₁₅ alkyl, substituted with one or more substituents. In some embodiments, each of R₁₀, R₁₁, R₁₂, and R₁₃ is independently H or a branched C₁-C₁₅ alkyl. In some embodiments, each of R₁₀, R₁₁, R₁₂, and R₁₃ is independently H or a C₁-C₁₅ alkyl optionally substituted by one or more substituents selected from the group consisting of oxo (═O), OH, amino, mono- or di-alkylamino, C₁-C₁₅ alkyl, C₂-C₈ alkenyl, and C₂-C₈ alkynyl. In some embodiments, each of R₁₀, R₁₁, R₁₂, and R₁₃ is independently H or a unbranched, unsubstituted C₁-C₁₅ alkyl.

In some embodiments, at least one of R₁₀ and R₁₁ is not H, and at least one of R₁₂ and R₁₃ is not H. In some embodiments, R₁₁ is H, and R₁₀, R₁₂, and R₁₃ are not H. In some embodiments, R₁ is H, and R₁₀, R₁₁, and R₁₂ are not H. In some embodiments, R₁₁ and R₁₃ are H, and R₁₀ and R₁₂ are not H. In some embodiments, R₁₀ and R₁₁ are the same and are not H. In some embodiments, R₁₂ and R₁₃ are the same and are not H.

In some embodiments, R₁₀ and R₁₁ are each independently C₅-C₈ alkyl (e.g., unsubstituted and/or unbranched); or R₁₁ is H, and R₁₀ is C₁₀-C₁₄ alkyl (e.g., unsubstituted and/or unbranched); and R₁₂ and R₁₃ are each independently C₅-C₈alkyl (e.g., unsubstituted and/or unbranched); or R₁₃ is H, and R₁₂ is C₁₀-C₁₄ alkyl (e.g., unsubstituted and/or unbranched); wherein R₁₁ and R₁₁ are not both H.

In some embodiments, R₁₀ and R₁₁ are each independently C₅-C₈ alkyl (e.g., unsubstituted and/or unbranched); or R₁₁ is H, and R₁₀ is C₇-C₁₁ alkyl (e.g., unsubstituted and/or unbranched); and R₁₂ and R₁₃ are each independently C₅-C₈alkyl (e.g., unsubstituted and/or unbranched); or R₁₃ is H, and R₁₂ is C₇-C₁₁ alkyl (e.g., unsubstituted and/or unbranched), wherein R₁₁ and R₁₃ are not both H.

In any of the formulas described herein, each of Z₁ and Z₂ is independently a cleavable linker,

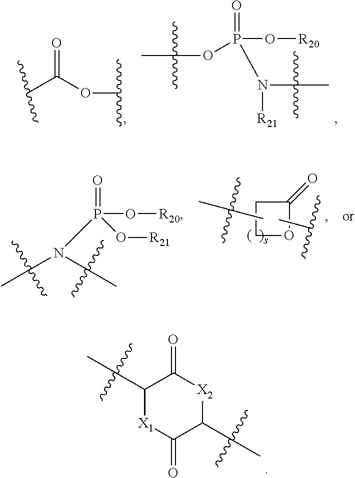

[symbol]

indicates the attachment of Z₁ or Z₂ to the formula, and is not meant to be directional. For instance,

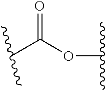 [SMILES: *OC(*)=O]

represents that both orientations,

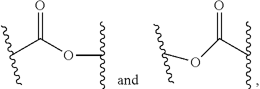 [SMILES: *OC(*)=O ; *OC(*)=O]

are possible. Similarly,

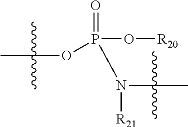

represents that both orientations,

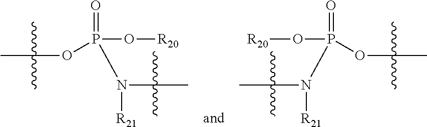

are possible.

In some embodiments, in any of the formulas described herein, each of Z₁ and Z₂ is independently

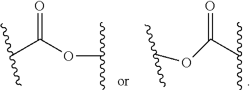 [SMILES: *OC(*)=O ; *OC(*)=O]

The lipid compounds thus contain at least two ester linker groups.

For instance, the lipid compounds may have the formula of:

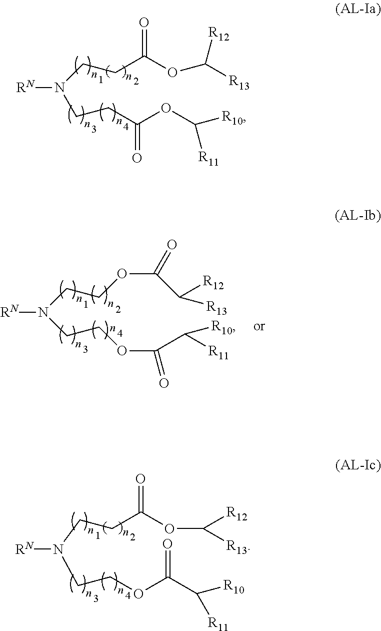

The variables R^{N}, R₁₀, R₁₁, R₁₂, R₁₃, n₁, n₂, n₃, and n₄ have been defined in various embodiments above.

In formulas (AL-Ia), (AL-Ib), or (AL-Ic), in some embodiments, R₁₀ and R₁₁ are each independently C₅-C₈ alkyl (e.g., unsubstituted and/or unbranched); or R₁₁ is H, and R₁₀ is C₁₀-C₁₄ alkyl (e.g., unsubstituted and/or unbranched); and R₁₂ and R₁₃ are each independently C₅-C₈ alkyl (e.g., unsubstituted and/or unbranched); or R₁₃ is H, and R₁₂ is C₁₀-C₁₄ alkyl (e.g., unsubstituted and/or unbranched); wherein R₁₁ and R₁₃ are not both H. In some embodiment, each alkyl group may be optionally substituted by one or more substituents selected from the group consisting of oxo (═O), OH, amino, mono- or di-alkylamino, C₁-C₁₅ alkyl, C₂-C₈ alkenyl, and C₂-C₈ alkynyl. In some embodiments, n₁+n₂ is an integer from 2 to 4, and n₃+n₄ is an integer from 5 to 7; or n₁+n₂ is an integer from 5 to 7, and n₃+n₄ is an integer from 2 to 4.

In some embodiments, in any of the formulas described herein, at least one of Z₁ and Z₂ is

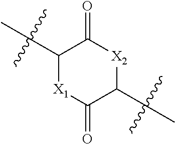

(lactide or derivative). Each of X₁ and X₂ is independently O, S, or N(R₂₁). For instance, the lactide or derivative may have the formula of

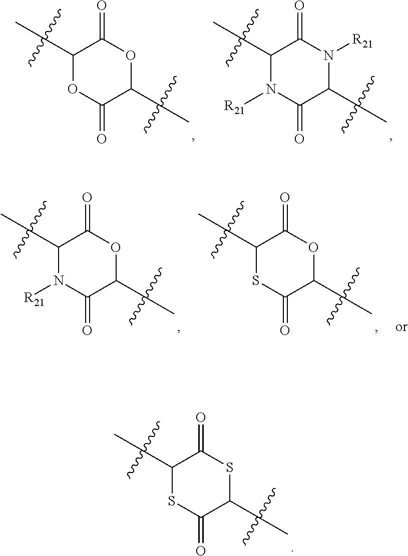

R₂₁ is H, substituted or unsubstituted C₁-C₅ alkyl, or substituted or unsubstituted C₃-C₈ cycloalkyl. In some embodiments, R₂₁ is H. In some embodiments, R₂₁ is C₁-C₅ alkyl such as C₁-C₃ alkyl (e.g., unsubstituted and/or unbranched).

In some embodiments, one of Z₁ and Z₂ is

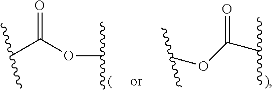 [SMILES: *OC(*)=O ; *OC(*)=O]

and the other of Z₁ and Z₂ is

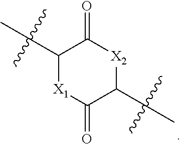

The lipids thus contain at least one ester linker group and one lactide (or its derivative) linker group.

In some embodiments, each of Z₁ and Z₂ is independently

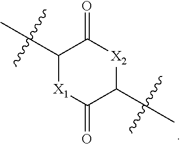

The lipids thus contain at least two lactide (or its derivative) linker groups. In some embodiments, Z₁ and Z₂ each contain a same

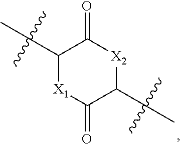

wherein the X₁ and X₂ variables for Z₁ are the same as the X₁ and X₂ variables for Z₂. In some embodiments, Z₁ and Z₂ each contain a different

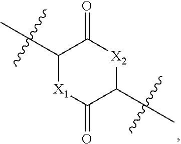

wherein the X₁ and X₂ variables for Z₁ are different than the X₁ and X₂ variables for Z₂.

For instance, the lipid compounds may have the formula of:

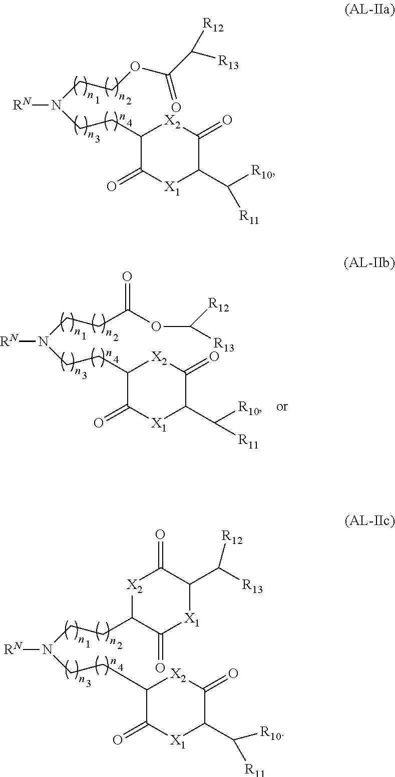

The variables R^{N}, R₁₀, R₁₁, R₁₂, R₁₃, X₁, X₂, n₁, n₂, n₃, and n₄ have been defined in various embodiments above.

In formulas (AL-IIa), (AL-IIb), or (AL-Ic), in some embodiments, R₁₀ and R₁₁ are each independently C₅-C₈ alkyl (e.g., unsubstituted and/or unbranched); or R₁, is H, and R₁₀ is C₇-C₁₁ alkyl (e.g., unsubstituted and/or unbranched); and R₁₂ and R₁₃ are each independently C₅-C₈alkyl (e.g., unsubstituted and/or unbranched); or R₁₃ is H, and R₁₂ is C₇-C₁₁ alkyl (e.g., unsubstituted and/or unbranched); wherein R₁₁ and R₁₃ are not both H. In some embodiments, each alkyl group may be optionally substituted by one or more substituents selected from the group consisting of oxo (═O), OH, amino, mono- or di-alkylamino, C₁-C₁₁ alkyl, C₂-C₈ alkenyl, and C₂-C₈ alkynyl. In some embodiments, n₁+n₂ is an integer from 4 to 7, and n₃+n₄ is an integer from 6 to 7; or n₁+n₂ is an integer from 6 to 7, and n₃+n₄ is an integer from 4 to 7.

In some embodiments, each of X₁ and X₂ is independently O or N(R₂₁). In some embodiments, R₂₁ is H or C₁-C₃ alkyl.

In some embodiments, in any of the formulas described herein, at least one of Z₁ and Z₂ is

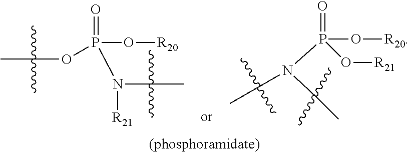

R₂₀ is a branched or unbranched, substituted or unsubstituted C₁-C₁₅ alkyl. In some embodiments, R₂₀ is C₂-C₉ alkyl (e.g., unsubstituted and/or unbranched). R₂₁ is H, substituted or unsubstituted C₁-C₅ alkyl, or substituted or unsubstituted C₃-C₈ cycloalkyl. In some embodiments, R₂₁ is H. In some embodiments, R₂₁ is C₁-C₅ alkyl such as C₁-C₃ alkyl (e.g., unsubstituted and/or unbranched).

In some embodiments, one of Z₁ and Z₂ is

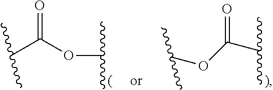 [SMILES: *OC(*)=O ; *OC(*)=O]

and the other of Z₁ and Z₂ is

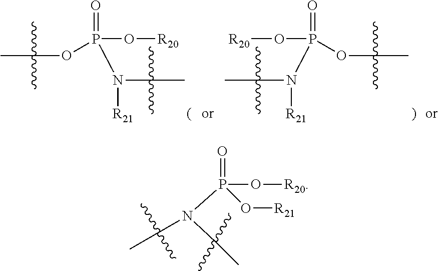

The lipid compounds thus contain one ester linker group and one phosphoramidate linker group.

In some embodiments, each of Z₁ and Z₂ is independently

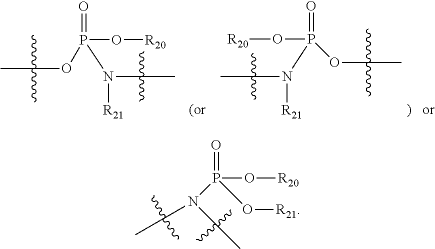

The lipid compounds thus contain at least two phosphoramidate linker groups. In some embodiments, Z₁ and Z₂ each contain a same

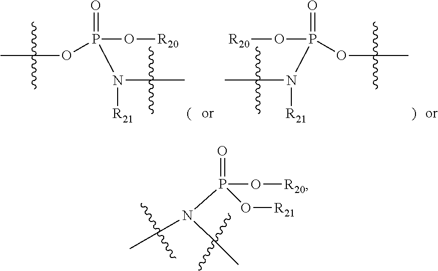

wherein the R₂₀ and R₂₁ variables for Z₁ are the same as the R₂₀ and R₂₁ variables for Z₂.

In some embodiments, Z₁ and Z₂ each contain a different

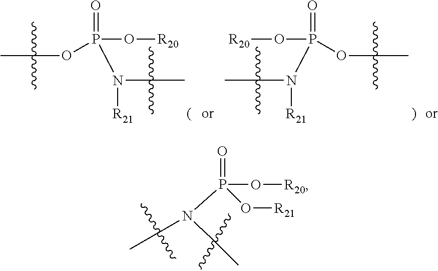

wherein the R₂₀ and R₂₁ variables for Z₁ are different than the R₂₀ and R₂₁ variables for Z₂. In some embodiments, Z₁ and Z₂ each contain a different

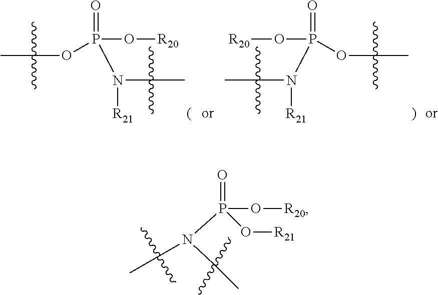

wherein one of Z₁ and Z₂ represents

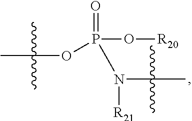

and the other of Z₁ and Z₂ represents

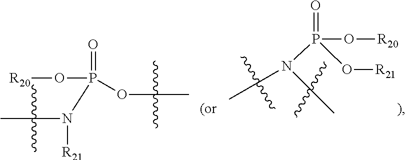

wherein the R₂₀ and R₂₁ variables for Z₁ can be the same as or different than the R₂₀ and R₂₁ variables for Z₂.

In some embodiments, when Z₂ is

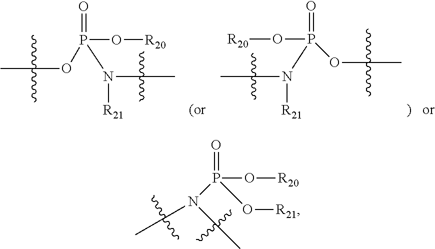

R₁₁ is H, and when Z₁ is

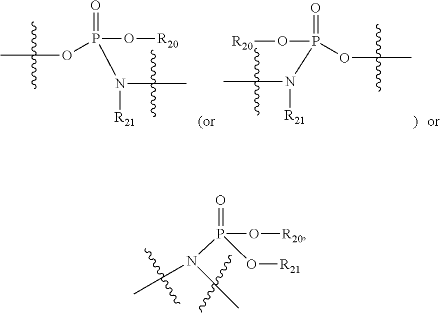

### R₁₃ is H.

For instance, the lipid compounds may have the formula of:

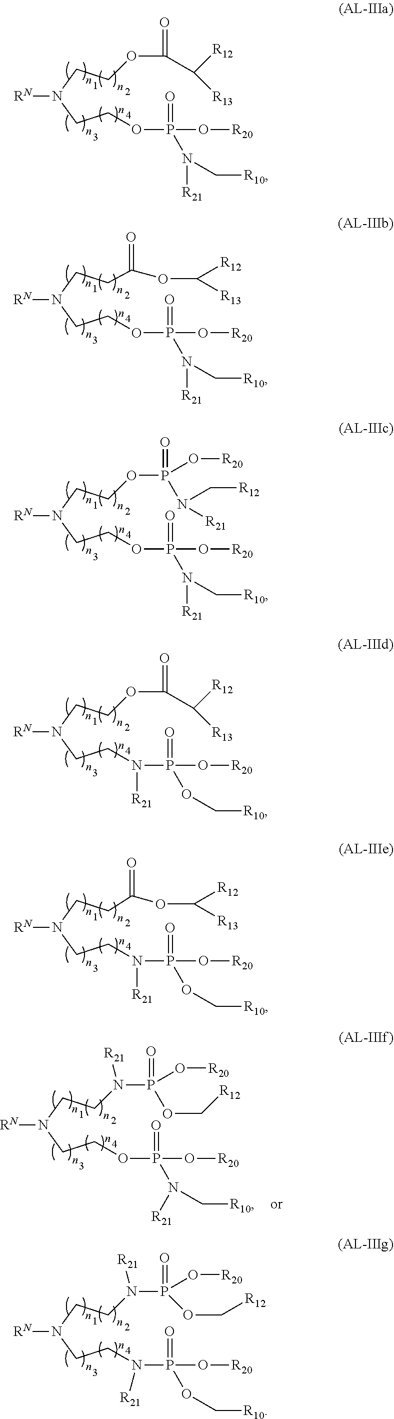

The variables R^{N}, R₁₀, R₁₁, R₁₂, R₁₃, R₂₀, R₂₁, n₁, n₂, n₃, and n₄ have been defined in various embodiments above.

In formulas (AL-IIIa), (AL-IIIb), or (AL-IIIc), (AL-IIId), (AL-IIIe), (AL-IIIf), and (AL-IIIg), in some embodiments, R₁₀ is C₇-C₁₁ alkyl (e.g., unsubstituted and/or unbranched); R₁₂ and R₁₃ are each independently C₅-C₈ alkyl (e.g., unsubstituted and/or unbranched); or R₁ is H, and R₁₂ is C₇-C₁₁ alkyl (e.g., unsubstituted and/or unbranched); each R₂₁ is H; and each R₂₀ is independently C₂-C₉ alkyl (e.g., unsubstituted and/or unbranched). In some embodiments, each alkyl group may be optionally substituted by one or more substituents selected from the group consisting of oxo (═O), OH, amino, mono- or di-alkylamino, C₁-C₁₅ alkyl, C₂-C₈ alkenyl, and C₂-C₈ alkynyl. In some embodiments, each of n₁+n₂ and n₃+n₄ is independently an integer from 5 to 7.

In some embodiments, in any of the formulas described herein, at least one of Z₁ and Z₂ is

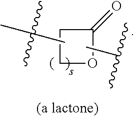

s is an integer from 1 to 4. For instance, the lactone linker group may have the formula of

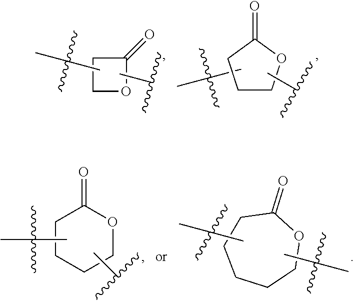

In some embodiments, one of Z₁ and Z₂ is

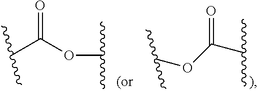 [SMILES: *OC(*)=O ; *OC(*)=O]

and the other of Z₁ and Z₂ is

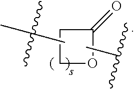

The lipids thus contain at least one ester linker group and one lactone linker group.

In some embodiments, each of Z₁ and Z₂ is independently a same or different

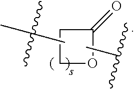

The lipids thus contain at least two lactone linker groups. In some embodiments, Z₁ and Z₂ each contain a same

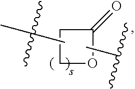

wherein the s variable and the positions on the lactone ring connecting to the formula for Z₁ are the same as the s variable and the positions on the lactone ring connecting to the formula for Z₂.

In some embodiments, Z₁ and Z₂ each contain a different

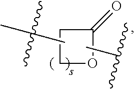

wherein the s variable for Z₁ is different than the s variable for Z₂. In some embodiments, Z₁ and Z₂ each contain a different

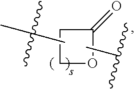

wherein the s variable for Z₁ is the same as the s variable for Z₂, but the positions on the lactone ring connecting to the formula for Z₁ is different than the positions on the lactone ring connecting to the formula for Z₂.

For instance, the lipid compounds may have the formula of:

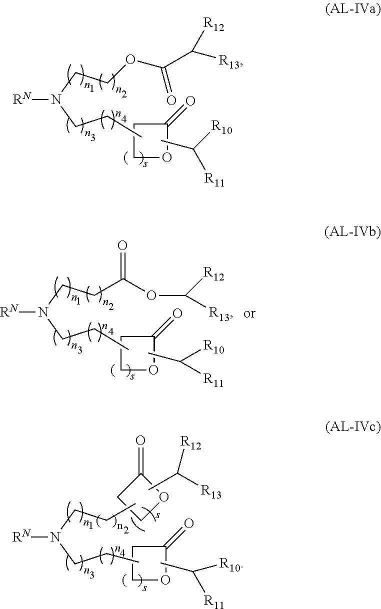

The variables R^{N}, R₁₀, R₁₁, R₁₂, R₁₃, X₁, X₂, n₁, n₂, n₃, and n₄ have been defined in various embodiments above.

In formulas (AL-IVa), (AL-IVb), or (AL-IVc), in some embodiments, R₁₀ and R₁₁ are each independently C₅-C₈ alkyl (e.g., unsubstituted and/or unbranched); or R₁₁ is H, and R₁₀ is C₇-C₁₁ alkyl (e.g., unsubstituted and/or unbranched); and R₁₂ and R₁₃ are each independently C₅-C₈ alkyl (e.g., unsubstituted and/or unbranched); or R³ is H, and R₁₂ is C₇-C₁₁ alkyl (e.g., unsubstituted and/or unbranched); wherein R₁₁ and R₁₃ are not both H. In some embodiments, each alkyl group may be optionally substituted by one or more substituents selected from the group consisting of oxo (═O), OH, amino, mono- or di-alkylamino, C₁-C₁₅ alkyl, C₂-C₈ alkenyl, and C₂-C₈ alkynyl. In some embodiments, n₁+n₂ is an integer from 4 to 7, and n₃+ₙ₄ is an integer from 6 to 7; or n₁+n₂ is an integer from 6 to 7, and n₃+n₄ is an integer from 4 to 7.

Additional exemplary formulas for the lipids compounds include but are not limited to the following.

a. Lipids Having at Least Two Ester Groups:

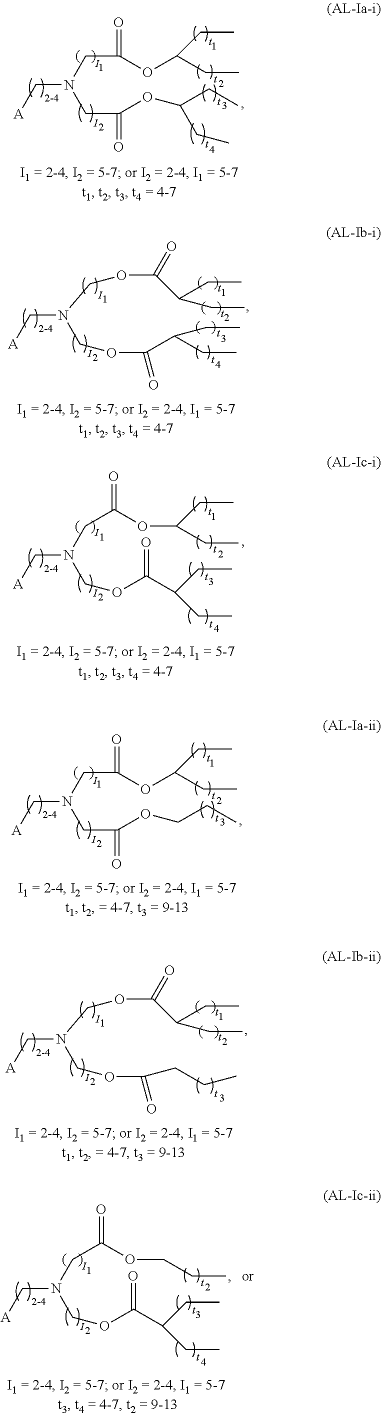

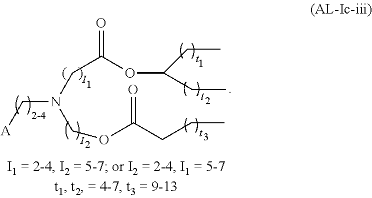

In the above formulas:
A=H, OH, Q, or NHQ
Q=heterocycle, heteroaryl, or S(O)₂NR₂₁;
R₂₁═H or methyl.
   b. Lipids Containing a Lactide (or its Derivative) Group:

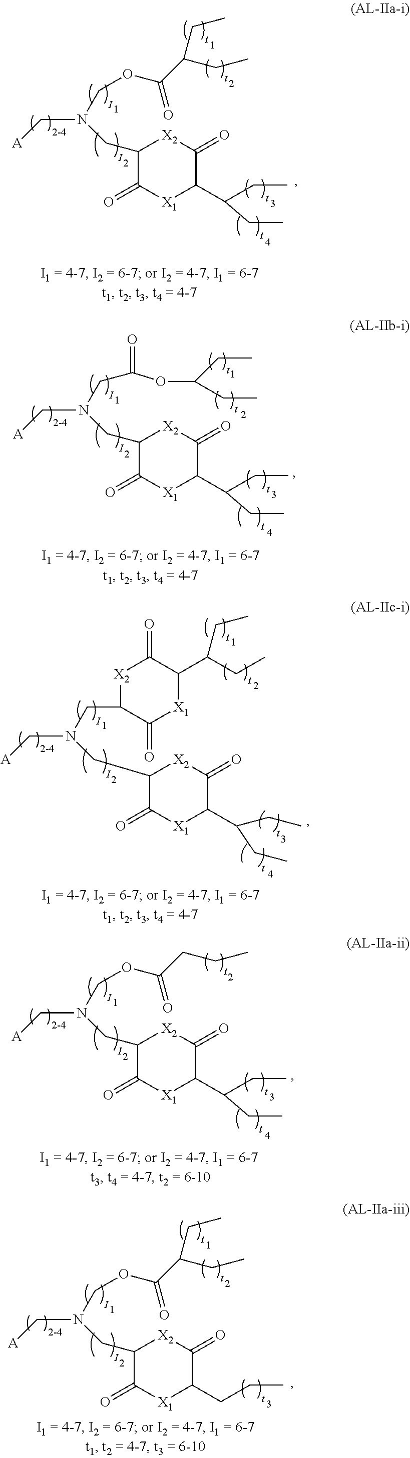

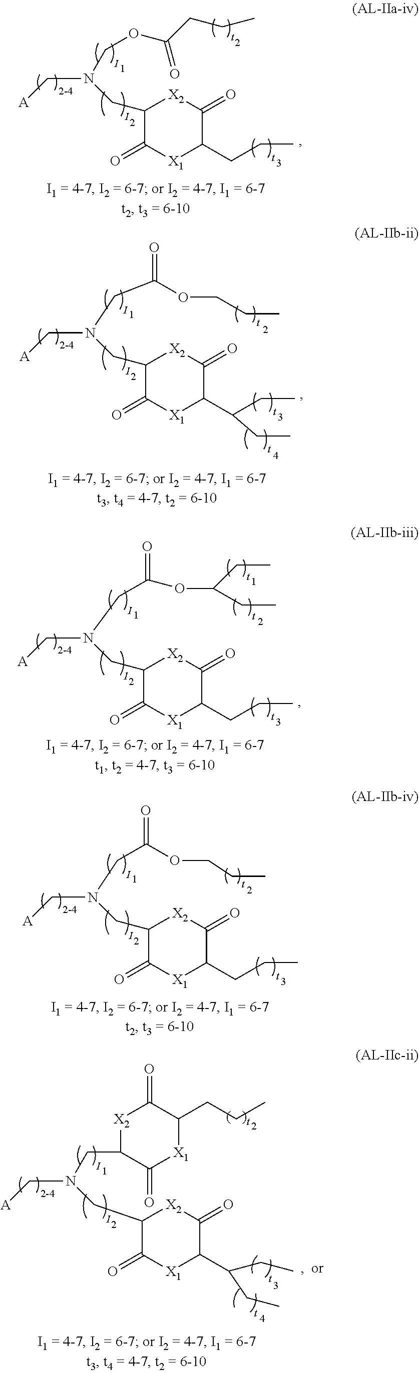

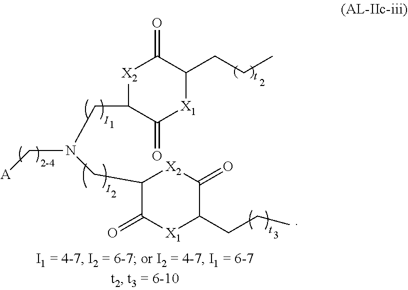

In all the above formulas:
A=H, OH, Q, or NHQ;
Q=heterocycle, heteroaryl, or S(O)₂NR₂₁;
R₂₁═H or methyl;
X₁, X₂═O or NH.
   c. Lipids Containing a Lactone Group:

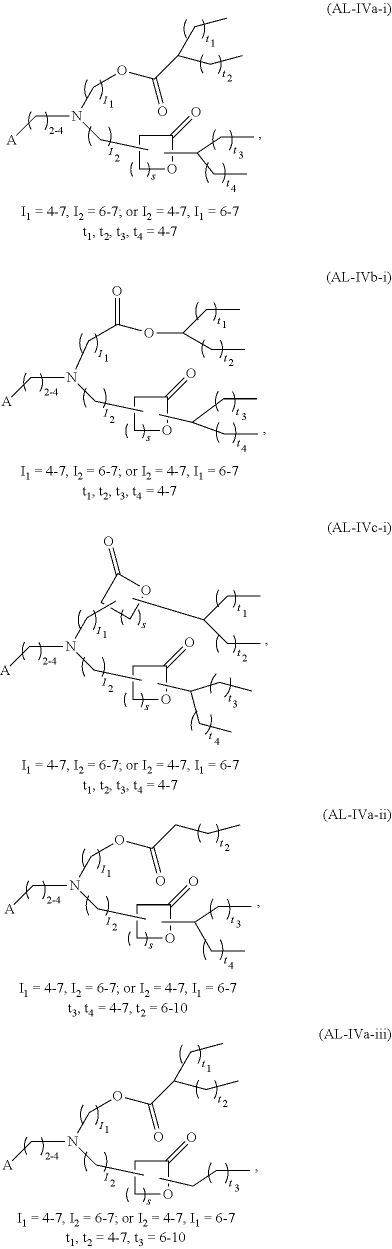

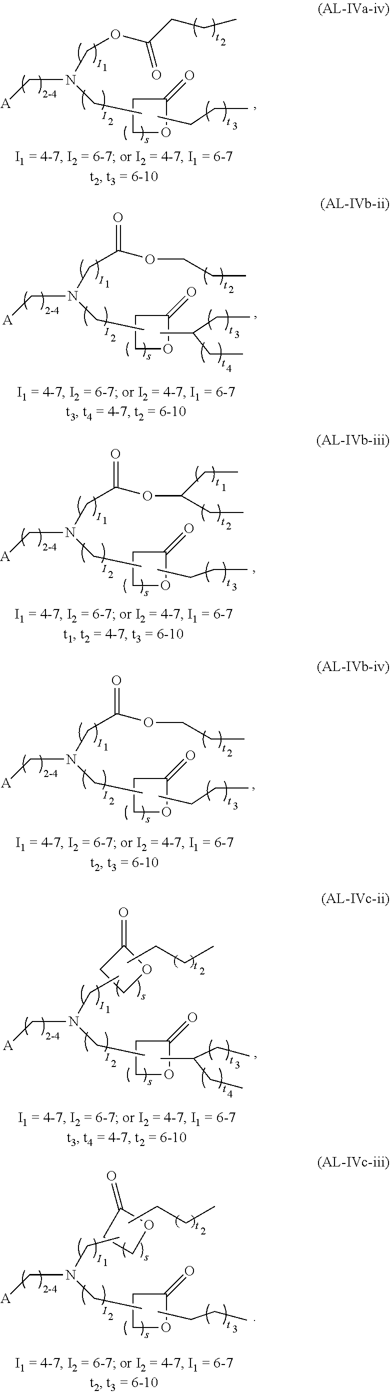

In all the above formulas:
A=H, OH, Q, or NHQ;
Q=heterocycle, heteroaryl, or S(O)₂NR₂₁;
R₂₁═H or methyl.
   d. Lipids Containing a Phosphoramidate Group:

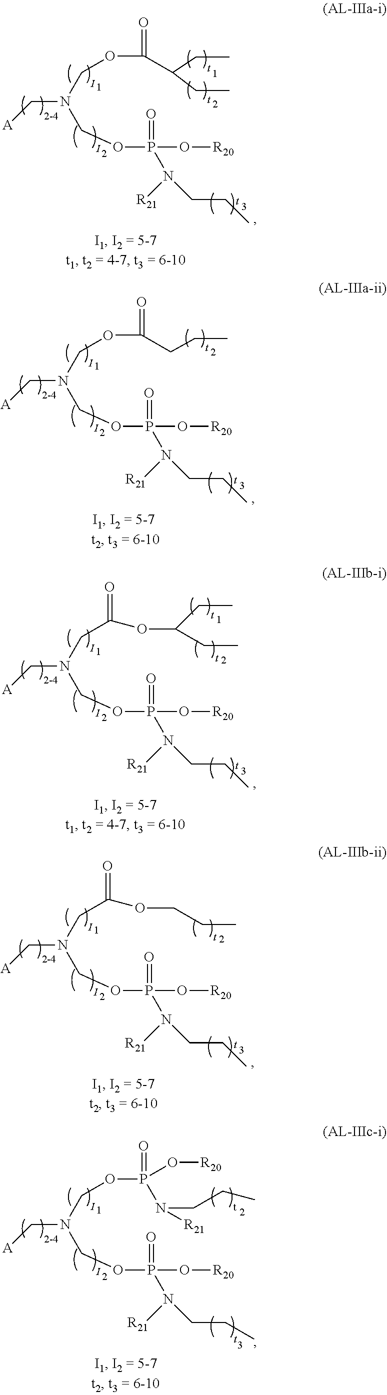

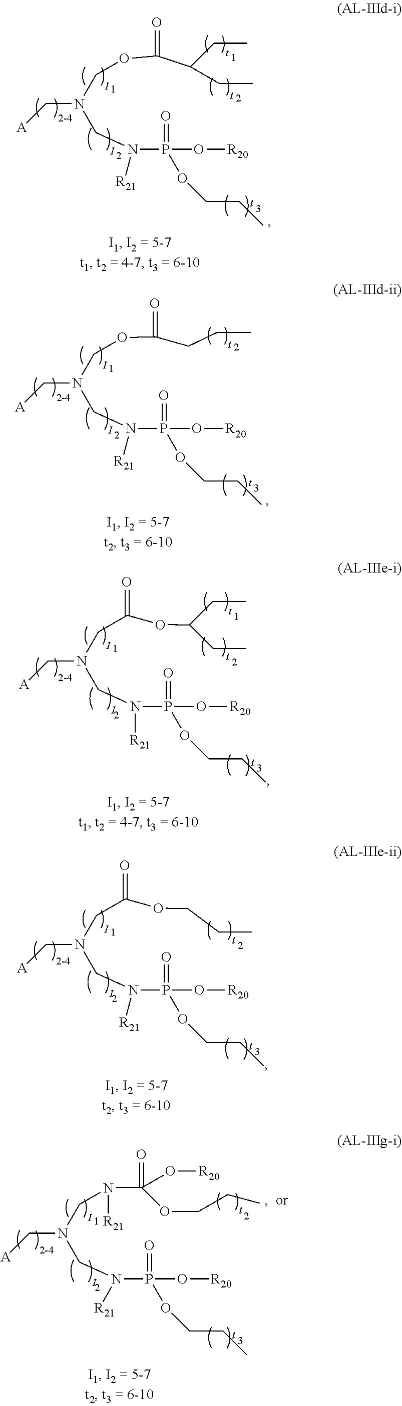

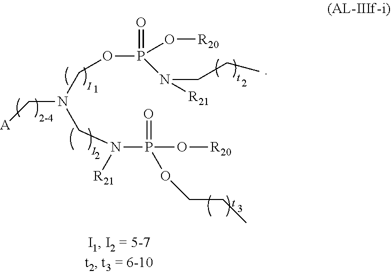

In all the above formulas;
A=H, OH, Q, or NHQ;
Q=heterocycle, heteroaryl, or S(O)₂NR₂₁,
R₂₀═C₂-C₉ alkyl; R₂₁═H or methyl.

Non-limiting examples of the lipid compounds disclosed herein are set forth below.

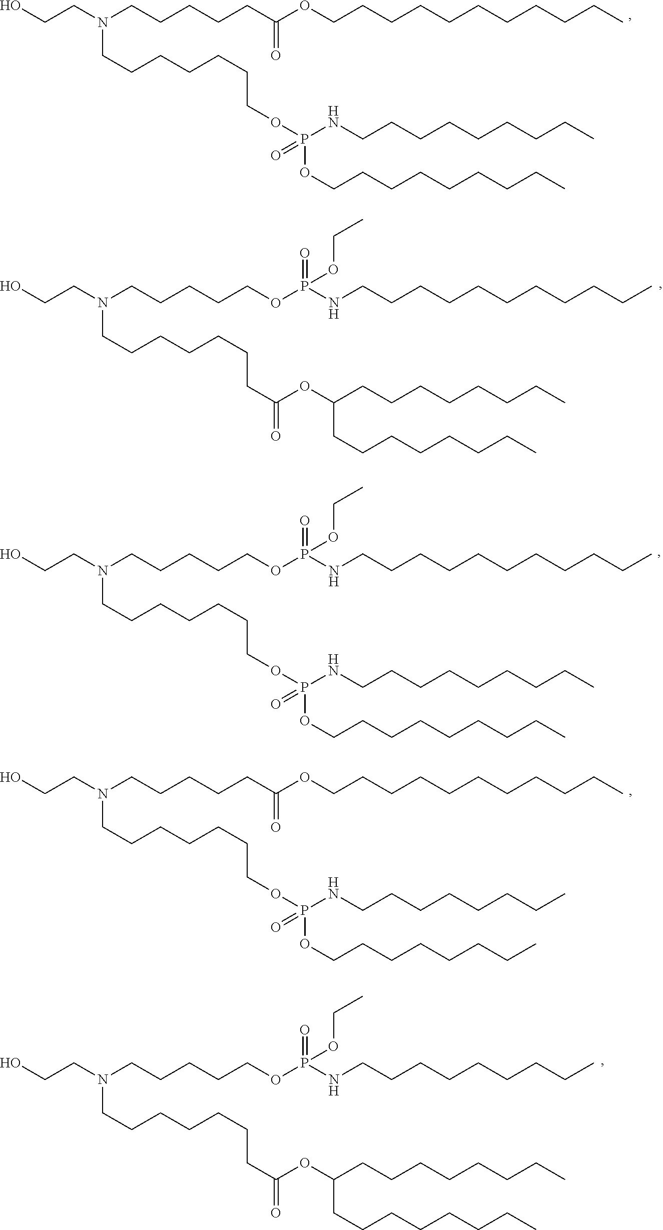

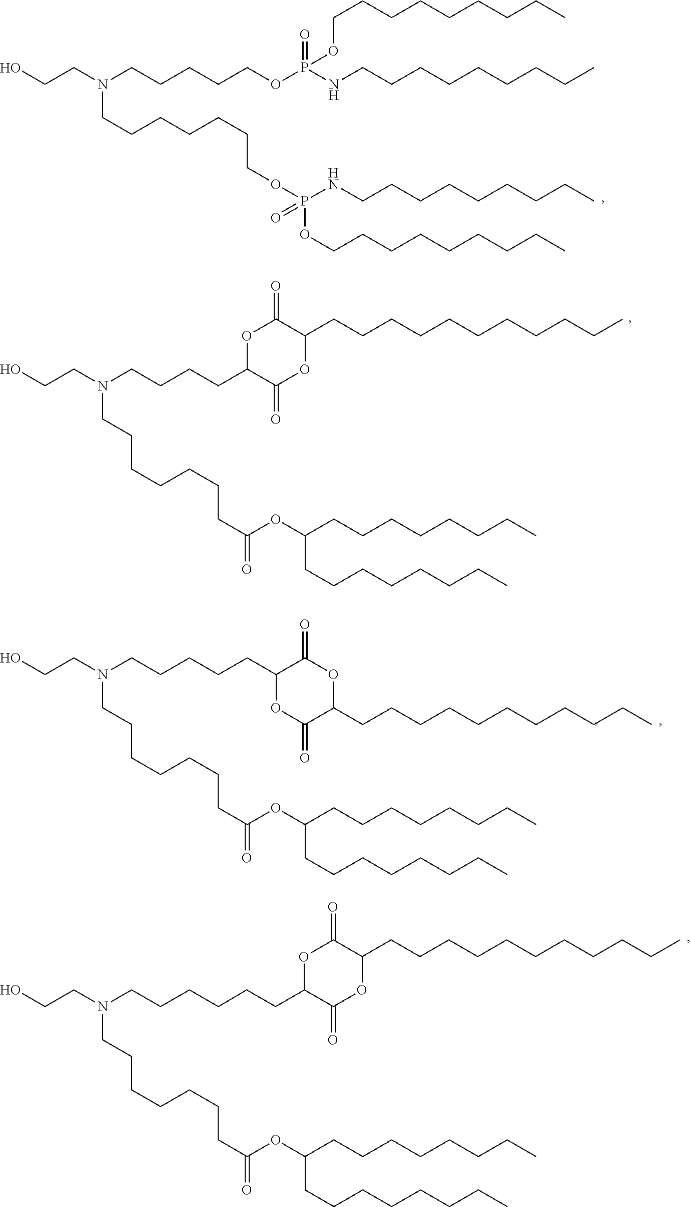

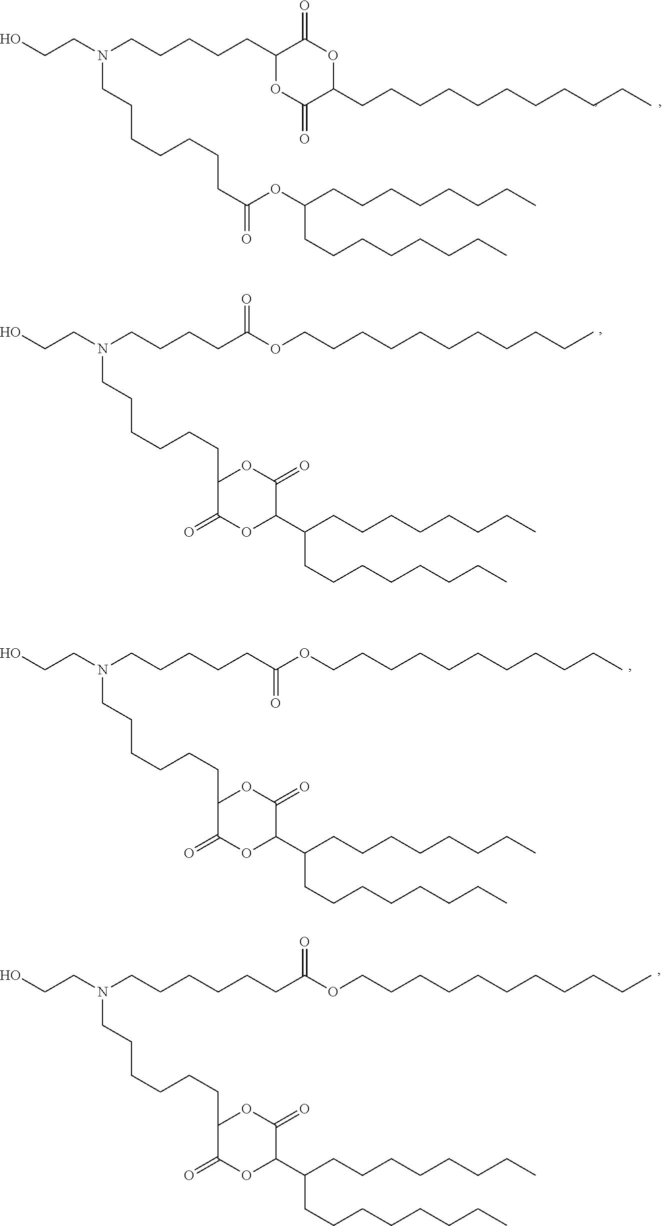 [SMILES: CCCCCCCCCCCC1OC(=O)C(CCCCCN(CCO)CCCCCCCC(=O)OC(CCCCCCCC)CCCCCCCC)OC1=O ; CCCCCCCCCCCOC(=O)CCCCN(CCO)CCCCCCC1OC(=O)C(C(CCCCCCCC)CCCCCCCC)OC1=O ; CCCCCCCCCCCOC(=O)CCCCCN(CCO)CCCCCCC1OC(=O)C(C(CCCCCCCC)CCCCCCCC)OC1=O ; CCCCCCCCCCCOC(=O)CCCCCCN(CCO)CCCCCCC1OC(=O)C(C(CCCCCCCC)CCCCCCCC)OC1=O]

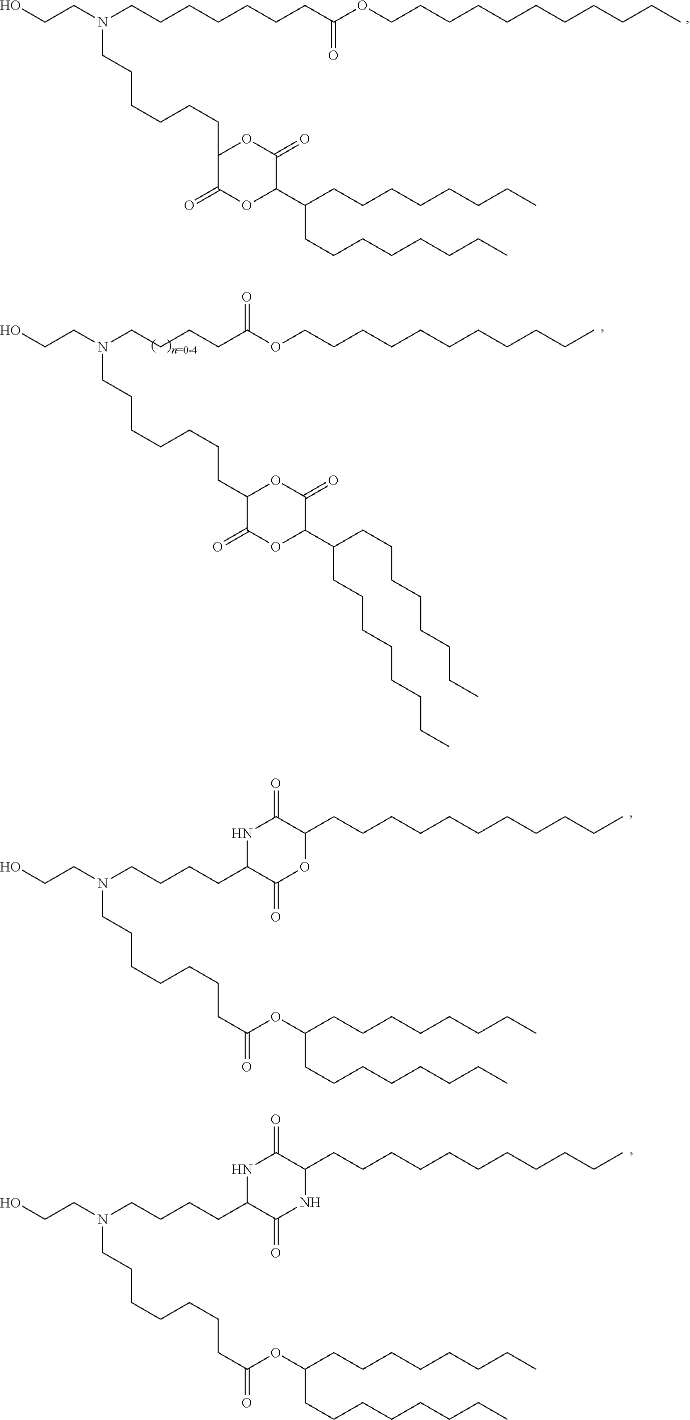

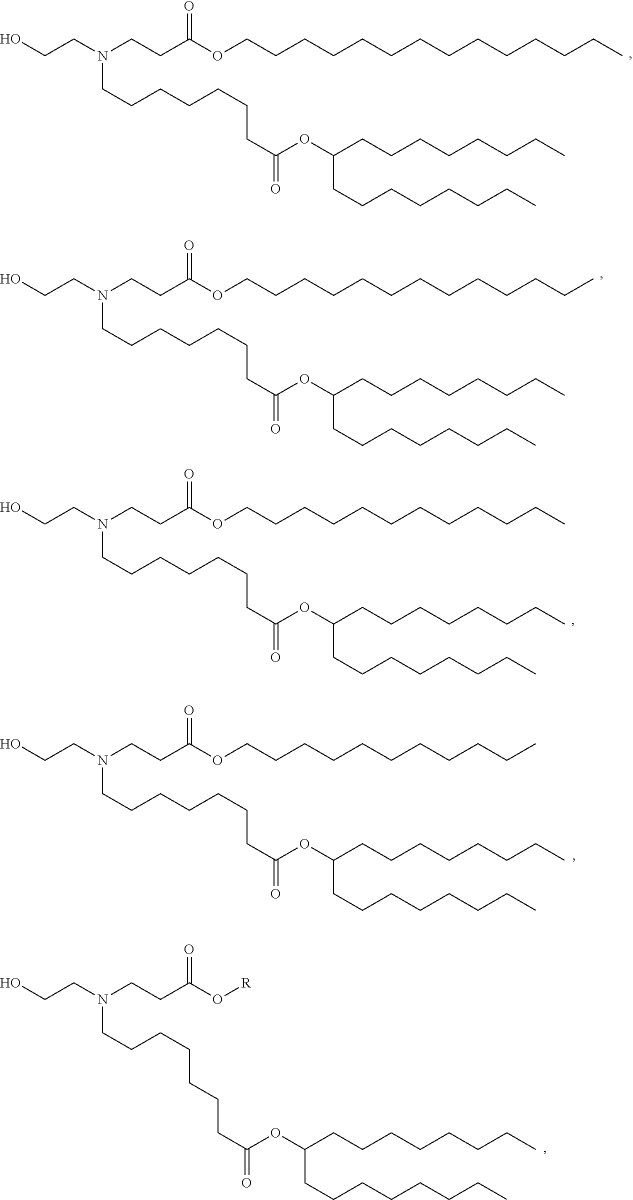

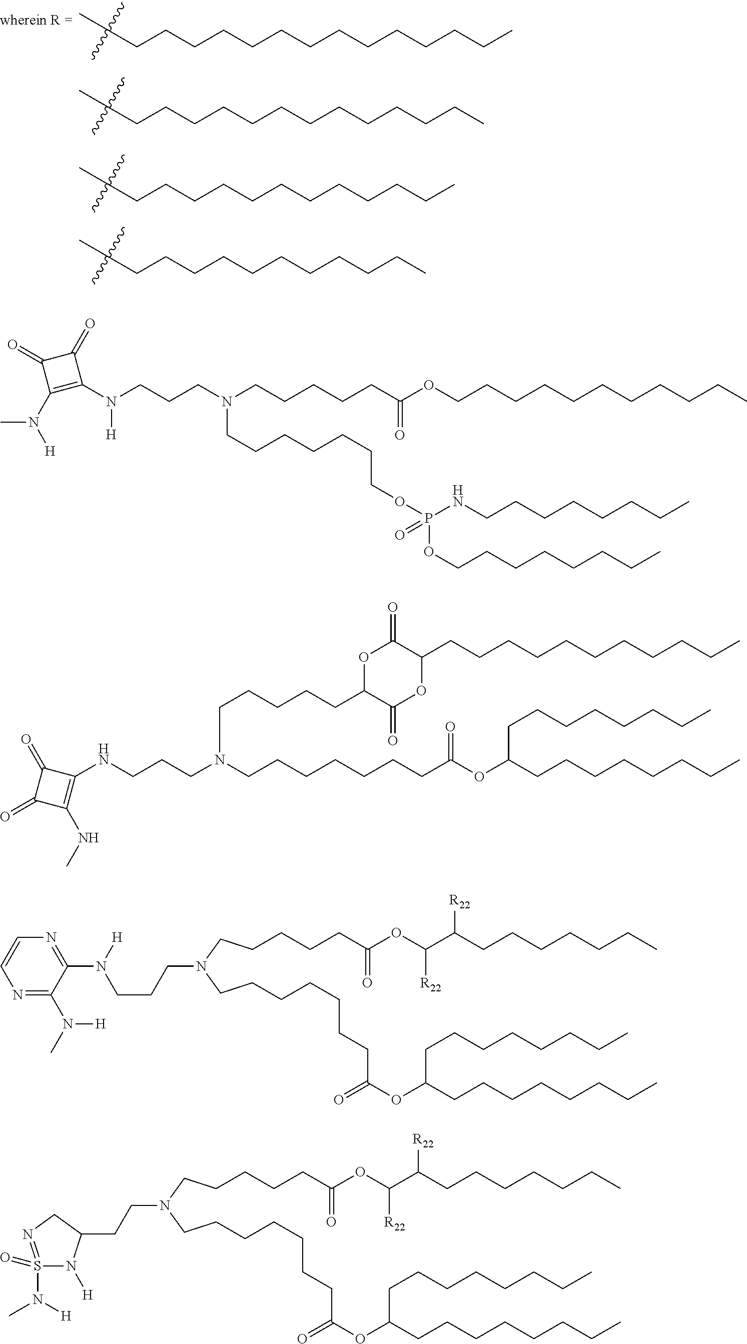

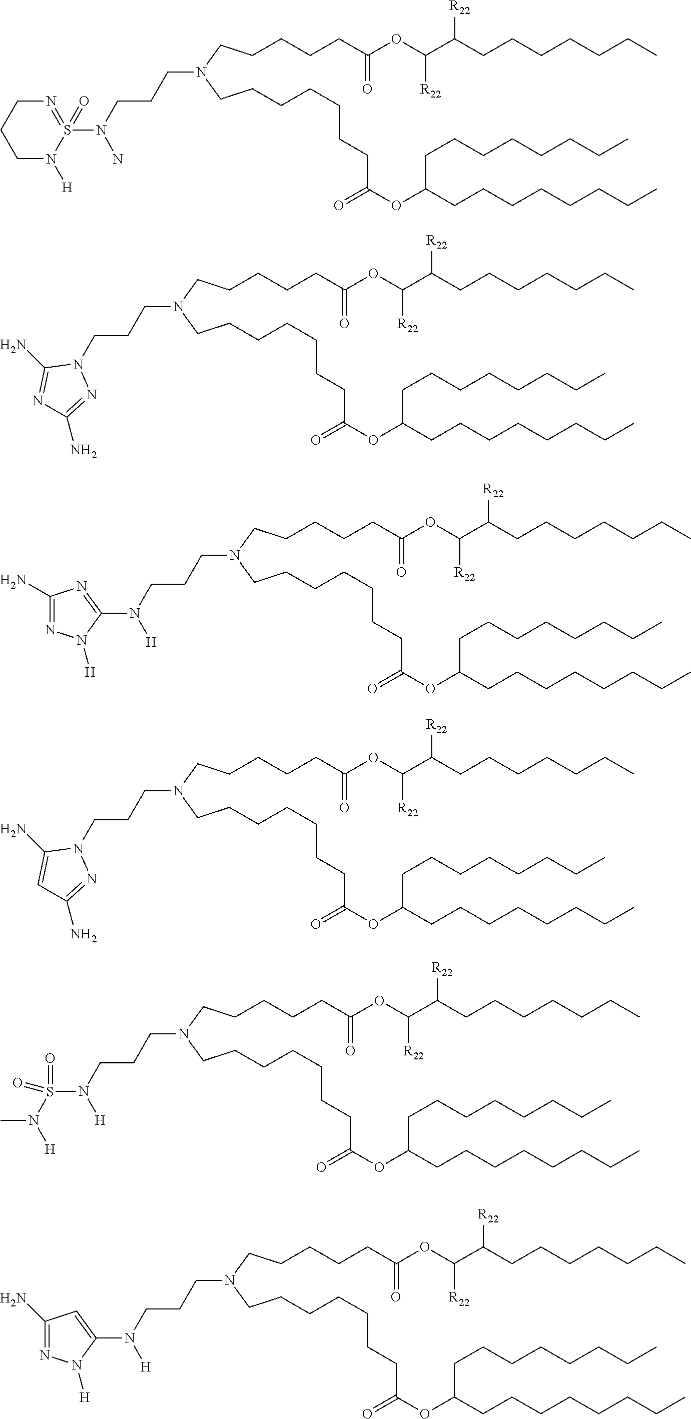

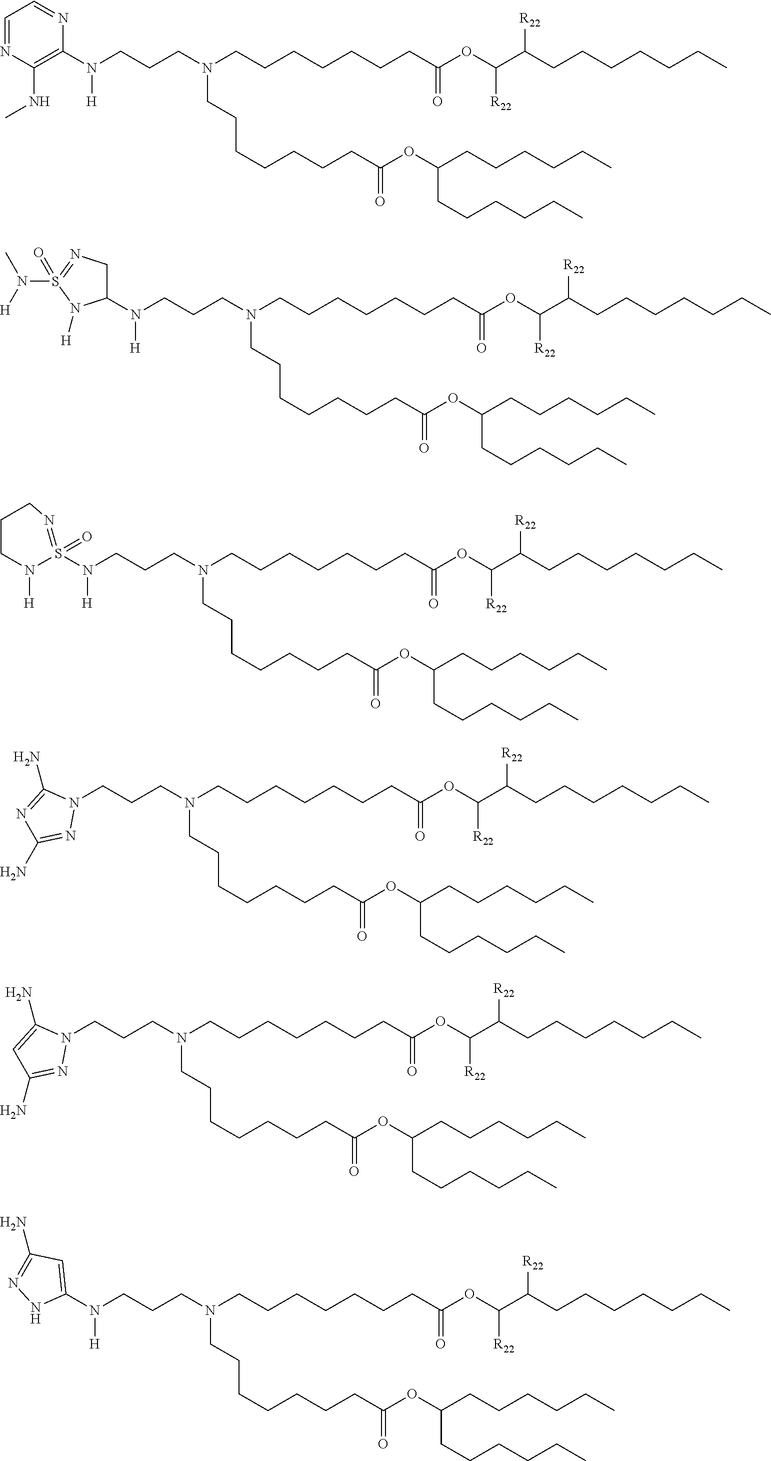

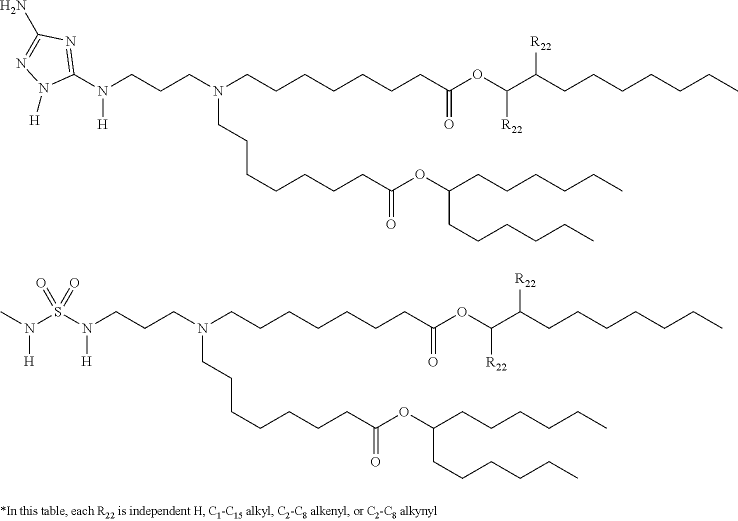

In a first embodiment the invention relates to a compound of formula (AL-GI):

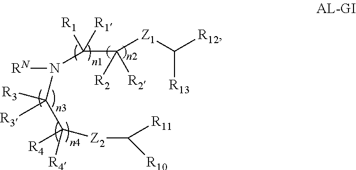

wherein:
R^{N} is substituted or unsubstituted C₁-C₆ alkyl or C₃-C₈ cycloalkyl;
each of R₁, R_{1′}, R₂, R_{2′}, R₃, R_{3′}, R₄, and R_{4′} is, for each occurrence, independently H, branched or unbranched C₁-C₃ alkyl, or branched or unbranched C₂-C₃ alkenyl;
each of R₁₀, R₁₁, R₁₂, and R₁₃ is independently H or a branched or unbranched, substituted or unsubstituted C₁-C₁₅ alkyl; provided that at least one of R₁₀ and R₁₁ is not H, and at least one of R₁₂ and R₁₃ is not H;
each of Z₁ and Z₂ is independently

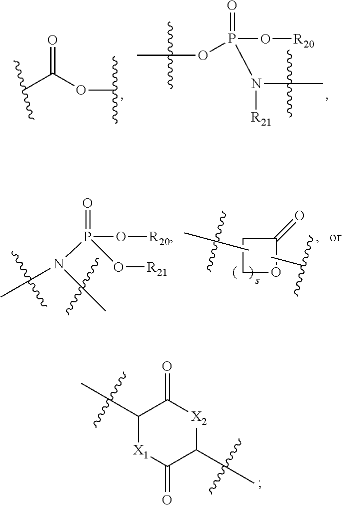

each of X₁ and X₂ is independently O, S, or N(R₂₁);
R₂₀ is a branched or unbranched, substituted or unsubstituted C₁-C₁₅ alkyl;
R₂₁ is H, substituted or unsubstituted C₁-C₅ alkyl, or substituted or unsubstituted C₃-C₈ cycloalkyl;
s is an integer from 1; and
each of n₁, n₂, n₃, and n₄ is independently an integer from 0 to 15, wherein n₁+n₂ ranges from 1 to 15 and n₃. n₄ ranges from 1 to 15.

In a first aspect of the first embodiment, R^{N} is C₁-C₆ alkyl, C₃-C₈ cycloalkyl, —(CH₂)ᵥQ, —(CH₂)ᵥN(R)Q, —C(Q)(R)₂, or —(CH₂)ᵥC(Q)(R)₂; each Q is independently —OR, —SR, C₃-C₈ cycloalkyl, heterocyclyl, heteroaryl, —O(CH₂)ᵥN(R)₂, —C(O)OR, —OC(O)R, —C(R′)₃, —CN, —C(O)N(R)₂, —N(R)C(O)R, —N(R)S(O)₂R, —N(R)Cᵥ(O)N(R)₂, —N(R)C(S)N(R)₂, —N(R)R^{a}, —O(CH₂)ᵥOR, —N(R)C(═NR^{b})N(R)₂, —N(R)C(═CHR^{b})N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR^{b})N(R)₂, —N(OR)C(═CHR^{b})N(R)₂, —C(═NR)N(R)₂, —C(═NR)R, or —C(O)N(R)OR, wherein each of the alkyl, cycloalkyl, heterocyclyl, and heteroaryl groups are optionally substituted by one or more substituents selected from the group consisting of oxo (═O), OH, amino, mono- or di-alkylamino, and C₁-C₃ alkyl; each R is independently H, C₁-C₃ alkyl, C₂-C₃ alkenyl, amino, or mono- or di-alkylamino; each R′ is independently H, F, Cl, Br, or I, each R′ is independently H or C₃-C₈ cycloalkyl, each R^{b} is independently H, CN, NO₂, C₁-C₆ alkyl, —OR, —S(O)₂R, —S(O)₂N(R)₂, C₂-C₆ alkenyl, C₃-C₈ cycloalkyl, or heterocyclyl; and each v is independently an integer from 1 to 6.

In a second aspect of the first embodiment, R^{N} is unsubstituted C₁-4 alkyl, —(CH₂)ᵥN(R)Q, or —(CH₂)ᵥQ. Q is OH, SH, —NHC(S)N(R)₂, —NHC(O)N(R)₂, —N(R)C(O)R, —N(R)S(O)₂R, —N(R)Ra, —NHC(═NR″)N(R)₂, —NHC(═CHR^{b})N(R)₂, —OC(O)N(R)₂, —N(R)C(O)OR, heterocyclyl, or heteroaryl; and each v is independently 2, 3, 4, or 5. The remainder of features and example features of the second aspect is as described above with respect to the first aspect of the first embodiment.

In a third aspect of the first embodiment, R^{N} is —(CH₂)ᵥOH, and v is independently 2, 3, or 4. The remainder of features and example features of the third aspect is as described above with respect to the first and second aspects of the first embodiment.

In a fourth aspect of the first embodiment, R^{N} is —(CH₂)ᵥQ or —(CH₂)ᵥN(R)Q and Q is heterocyclyl or heteroaryl, optionally substituted with one or more substituents. The remainder of features and example features of the fourth aspect is as described above with respect to the first through the third aspects of the first embodiment.

In a fifth aspect of the first embodiment, R^{N} is —(CH₂)ᵥQ or —(CH₂)ᵥN(R)Q and Q is heterocyclyl or heteroaryl, optionally substituted with one or more substituents. The remainder of features and example features of the fifth aspect is as described above with respect to the first through the fourth aspects of the first embodiment.

In a sixth aspect of the first embodiment, R^{N} is —(CH₂)ᵥN(R)S(O)₂R. The remainder of features and example features of the sixth aspect is as described above with respect to the first through the fifth aspects of the first embodiment.

In a seventh aspect of the first embodiment, R^{N} has the following structures:

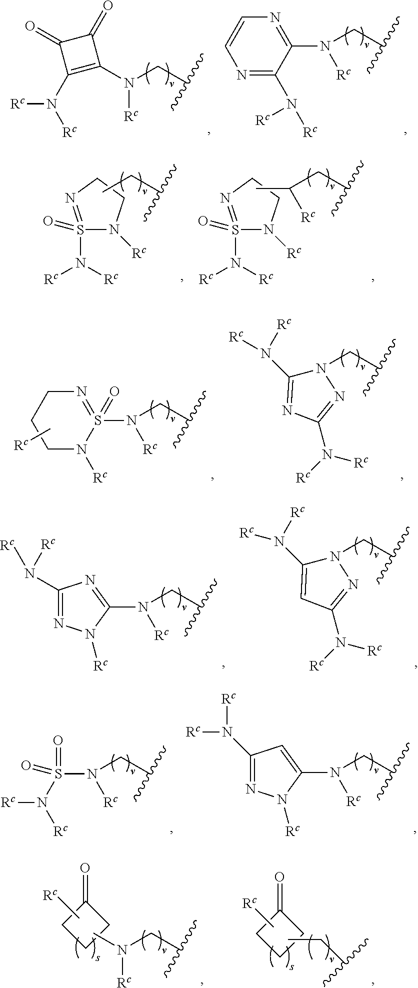

wherein each R^{c} is independently H or C₁-C₃ alkyl, and s is an integer from 1 to 4. The remainder of features and example features of the seventh aspect is as described above with respect to the first through the sixth aspects of the first embodiment.

In an eighth aspect of the first embodiment, R^{N} has the structure of:

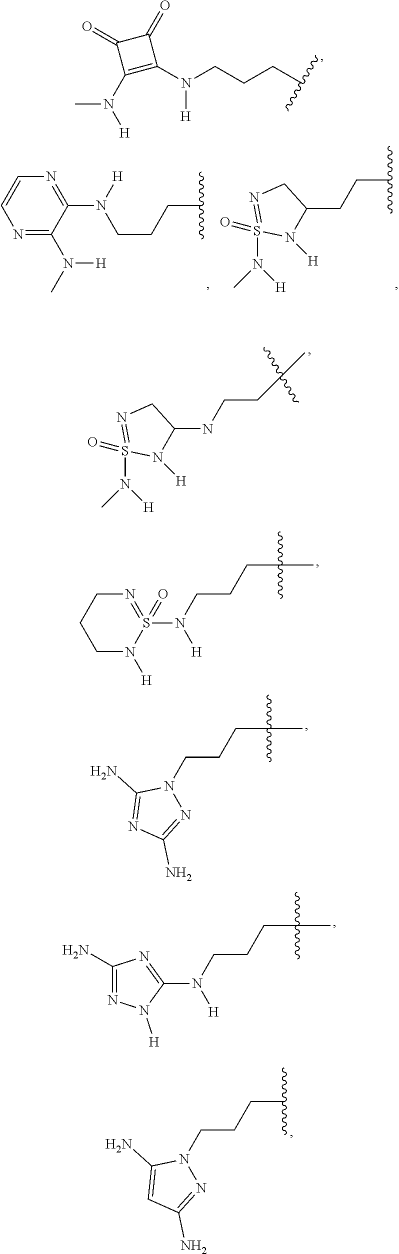

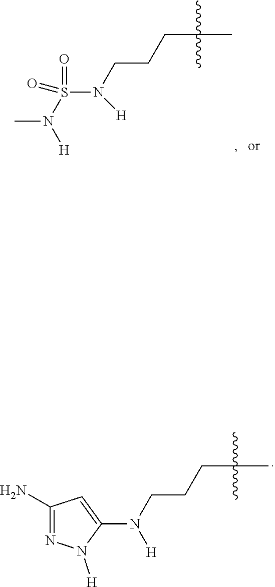

The remainder of features and example features of the eighth aspect is as described above with respect to the first through the seventh aspects of the first embodiment.

In a ninth aspect of the first embodiment, n₁+n₂ ranges from 1 to 10 and n₃+n₄ ranges from 1 to 10. For instance, n₁+n₂ ranges from 2 to 7 and n₃+n₄ ranges from 2 to 7, and n₁+n₂ and n₃+n₄ are the same. In certain embodiments, n₁+n₂ ranges from 2 to 7 and n₃+n₄ ranges from 2 to 7, and n₁+n₂ and n₃+n₄ are different. The remainder of features and example features of the ninth aspect is as described above with respect to the first through the eighth aspects of the first embodiment.

In a tenth aspect of the first embodiment, each of Z₁ and Z₂ is independently

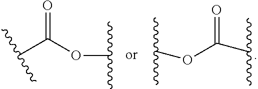

The remainder of features and example features of the tenth aspect is as described above with respect to the first through the ninth aspects of the first embodiment.

In an eleventh aspect of the first embodiment, one of Z₁ and Z₂ is

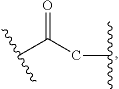

and the other of Z₁ and Z₂ is

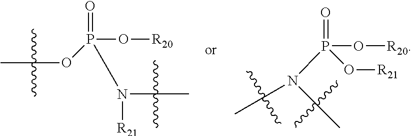

The remainder of features and example features of the eleventh aspect is as described above with respect to the first through the tenth aspects of the first embodiment.

In a twelfth aspect of the first embodiment, each of Z₁ and Z₂ is a same or different

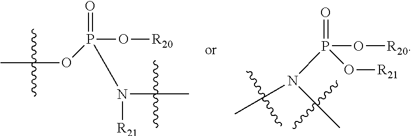

The remainder of features and example features of the twelfth aspect is as described above with respect to the first through the eleventh aspects of the first embodiment.

In a thirteenth aspect of the first embodiment, when Z² is

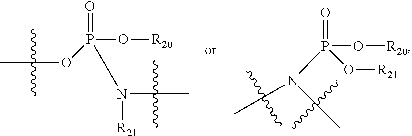

R₁₁ is H, and when Z₁ is

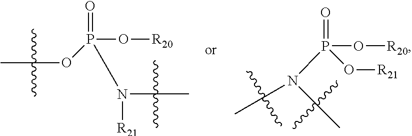

R₁₃ is H. The remainder of features and example features of the thirteenth aspect is as described above with respect to the first through the twelfth aspects of the first embodiment.

In a fourteenth aspect of the first embodiment, one of Z₁ and Z² is

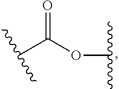 [SMILES: *OC(*)=O]

and the other of Z₁ and Z₂ is

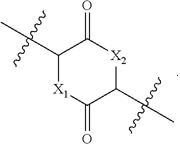

The remainder of features and example features of the fourteenth aspect is as described above with respect to the first through the thirteenth aspects of the first embodiment.

In a fifteenth aspect of the first embodiment, each of Z₁ and Z₂ is independently a same or different

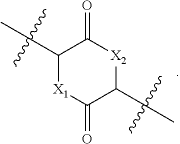

The remainder of features and example features of the fifteenth aspect is as described above with respect to the first through the fourteenth aspects of the first embodiment.

In a sixteenth aspect of the first embodiment, one of Z₁ and Z₂ is

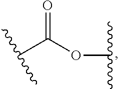 [SMILES: *OC(*)=O]

and the other of Z¹ and Z₂ is

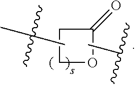

The remainder of features and example features of the sixteenth aspect is as described above with respect to the first through the fifteenth aspects of the first embodiment.

In a seventeenth aspect of the first embodiment, each of Z₁ and Z₂ is independently a same or different

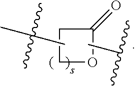

The remainder of features and example features of the seventeenth aspect is as described above with respect to the first through the sixteenth aspects of the first embodiment.

In an eighteenth aspect of the first embodiment, the compound has the formula:

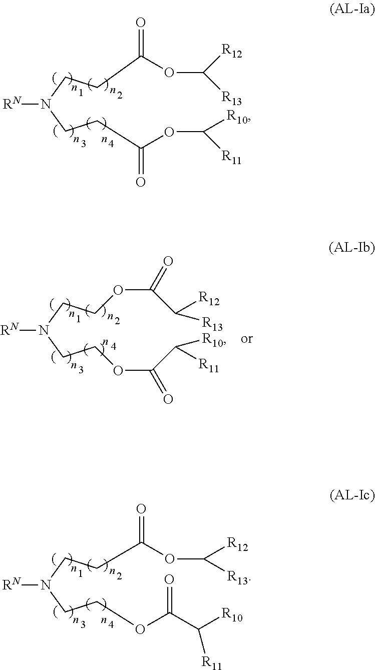

The remainder of features and example features of the eighteenth aspect is as described above with respect to the first through the seventeenth aspects of the first embodiment. The remainder of features and example features of the nineteenth aspect is as described above with respect to the first through the eighteenth aspects of the first embodiment.

In a nineteenth aspect of the first embodiment, R₁₀ and R₁₁ are each independently unsubstituted C₅-C₈alkyl; or R₁₁ is H, and R₁₀ is unsubstituted C₁₀-C₁₄ alkyl; and R₁₂ and R₁₃ are each independently unsubstituted C₅-C₈alkyl; or R₁₃ is H, and R₁₂ is unsubstituted C₁₀-C₁₄ alkyl; wherein R₁₁ and R₁₃ are not both H. The remainder of features and example features of the nineteenth aspect is as described above with respect to the first through the eighteenth aspects of the first embodiment.

In a twenty-first aspect of the first embodiment, n₁+n₂ is an integer from 2 to 4, and n₃+n₄ is an integer from 5 to 7; or n₁+n₂ is an integer from 5 to 7, and n₃+n₄ is an integer from 2 to 4. The remainder of features and example features of the twenty-first aspect is as described above with respect to the first through the twentieth aspects of the first embodiment.

In a twenty-second aspect of the first embodiment, the compound has the formula:

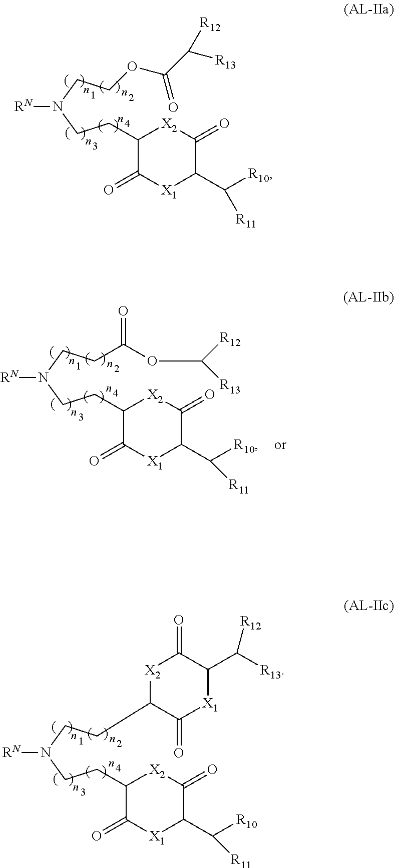

The remainder of features and example features of the twenty-second aspect is as described above with respect to the first through the twenty-first aspects of the first embodiment.

In a twenty-third aspect of the first embodiment, R₁₀ and R₁₁ are each independently unsubstituted C₅-C₈alkyl; or R₁, is H, and R₁₀ is unsubstituted C₇-C₁₁ alkyl; and R¹² and R₁₃ are each independently unsubstituted C₅-C₈alkyl; or R₁₃ is H, and R₁₁ is unsubstituted C₇-C₁₁ alkyl, wherein R₁₁ and R₁₃ are not both H. The remainder of features and example features of the twenty-third aspect is as described above with respect to the first through the twenty-second aspects of the first embodiment.

In a twenty-fourth aspect of the first embodiment, each of X₁ and X₂ is independently O or N(R²¹), and R₂₁ is H or C₁-C₃ alkyl. In some embodiments, n₁+n₂ is an integer from 4 to 7, and n₃+n₄ is an integer from 6 to 7; or n₁+n₂ is an integer from 6 to 7, and n₃+n₄ is an integer from 4 to 7. The remainder of features and example features of the twenty-fourth aspect is as described above with respect to the first through the twenty-third aspects of the first embodiment.

In a twenty-fifth aspect of the first embodiment, the compound has the formula:

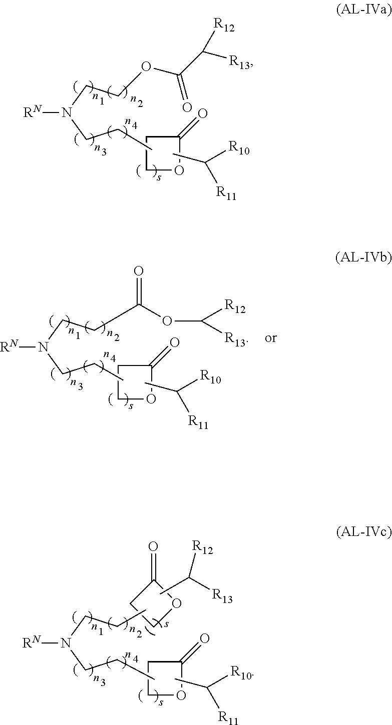

In some embodiments, R₁₀ and R₁₁ are each independently unsubstituted C₅-C₈alkyl; or R₁₁ is H, and R₁₀ is unsubstituted C₇-C₁₁ alkyl; and R₁₂ and R₁₃ are each independently unsubstituted C₅-C₈ alkyl; or R₁₃ is H, and R₁₂ is unsubstituted C₇-C₁₁ alkyl, wherein R₁₁ and R₁₃ are not both H. The remainder of features and example features of the twenty-fifth aspect is as described above with respect to the first through the twenty-fourth aspects of the first embodiment.

In a twenty-sixth aspect of the first embodiment, n₁+n₂ is an integer from 4 to 7, and n₃+n₄ is an integer from 6 to 7; or n₁+n₂ is an integer from 6 to 7, and n₃+n₄ is an integer from 4 to 7. The remainder of features and example features of the twenty-sixth aspect is as described above with respect to the first through the twenty-fifth aspects of the first embodiment.

In a twenty-seventh aspect of the first embodiment, the compound has the formula:

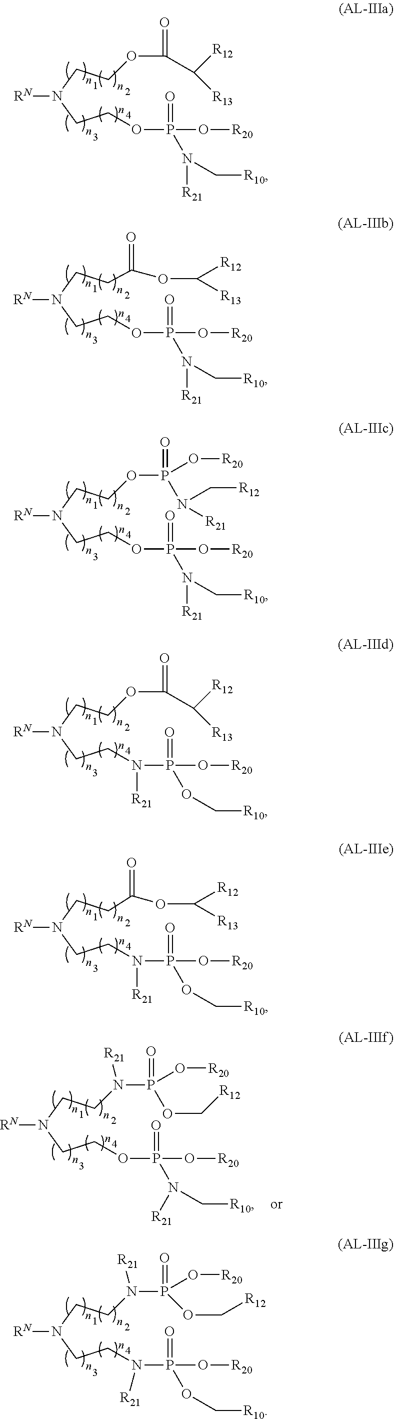

The remainder of features and example features of the twenty-seventh aspect is as described above with respect to the first through the twenty-sixth aspects of the first embodiment.

In a twenty-eighth aspect of the first embodiment, R₁₀ is unsubstituted C₇-C₁₁ alkyl; R₁₂ and R₁₃ are each independently unsubstituted C₅-C₈ alkyl; or R₁₃ is H, and R₁₂ is unsubstituted C₇-C₁₁ alkyl; each R₂₁ is H; and each R₂₀ is independently unsubstituted C₂-C₉ alkyl. The remainder of features and example features of the twenty-eighth aspect is as described above with respect to the first through the twenty-seventh aspects of the first embodiment.

In a twenty-ninth aspect of the first embodiment, each of n₁+n₂ and n₃+n₄ is independently an integer from 5 to 7. The remainder of features and example features of the twenty-ninth aspect is as described above with respect to the first through the twenty-eighth aspects of the first embodiment.

In a thirtieth aspect of the first embodiment, the compound has one of the following structures:

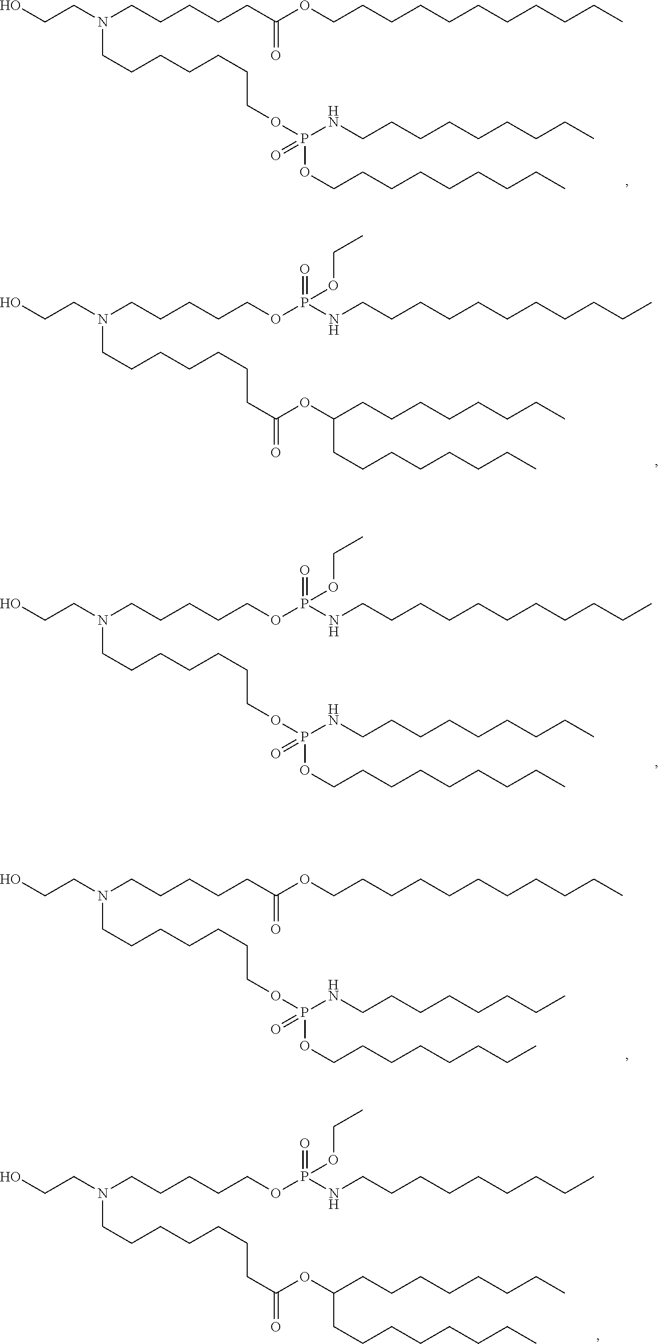

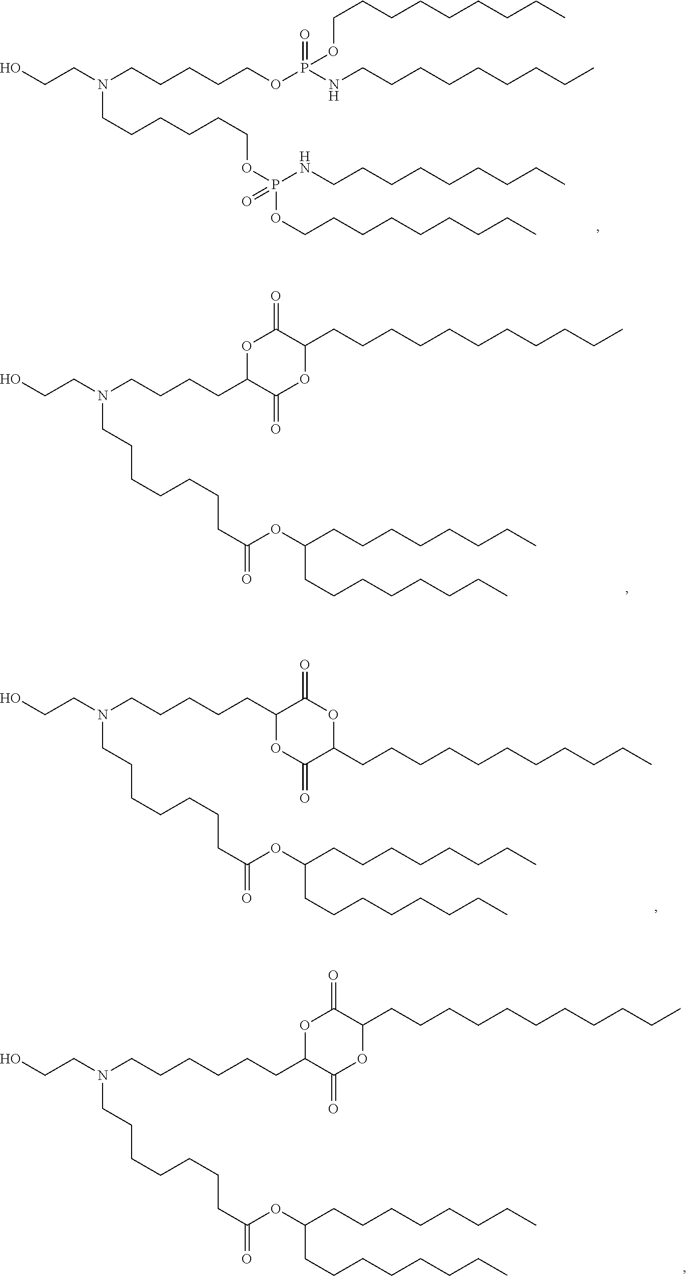

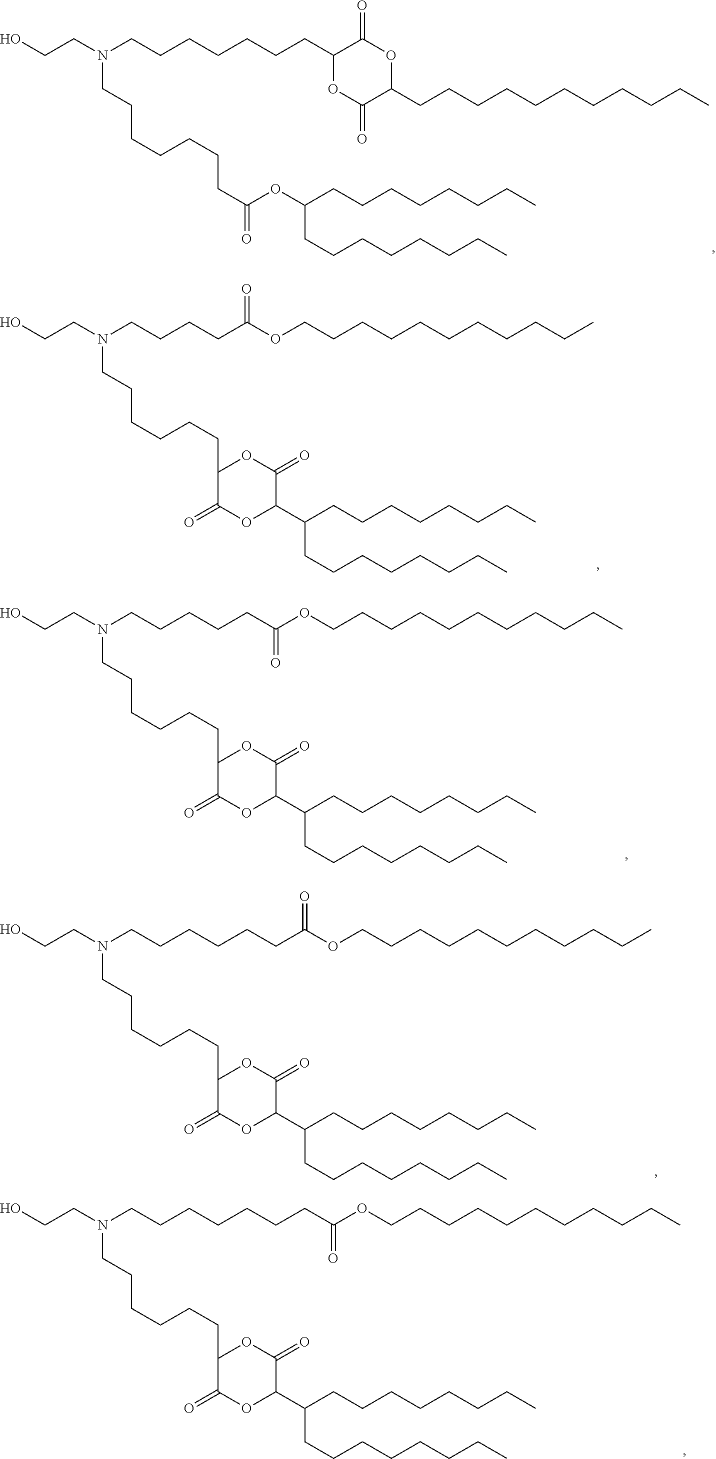

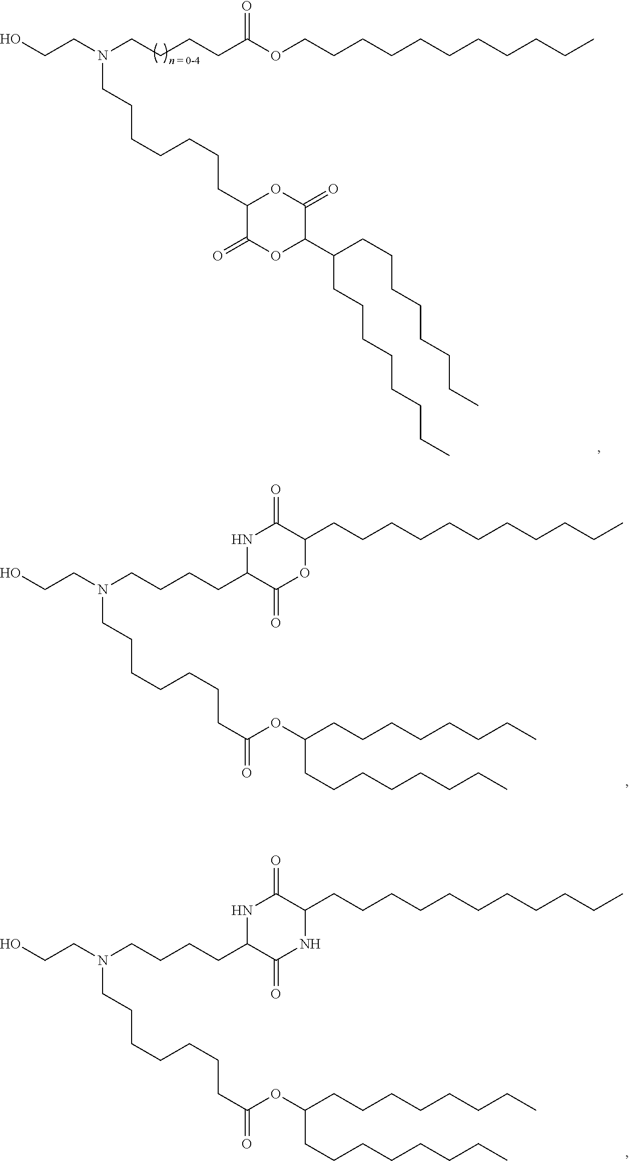

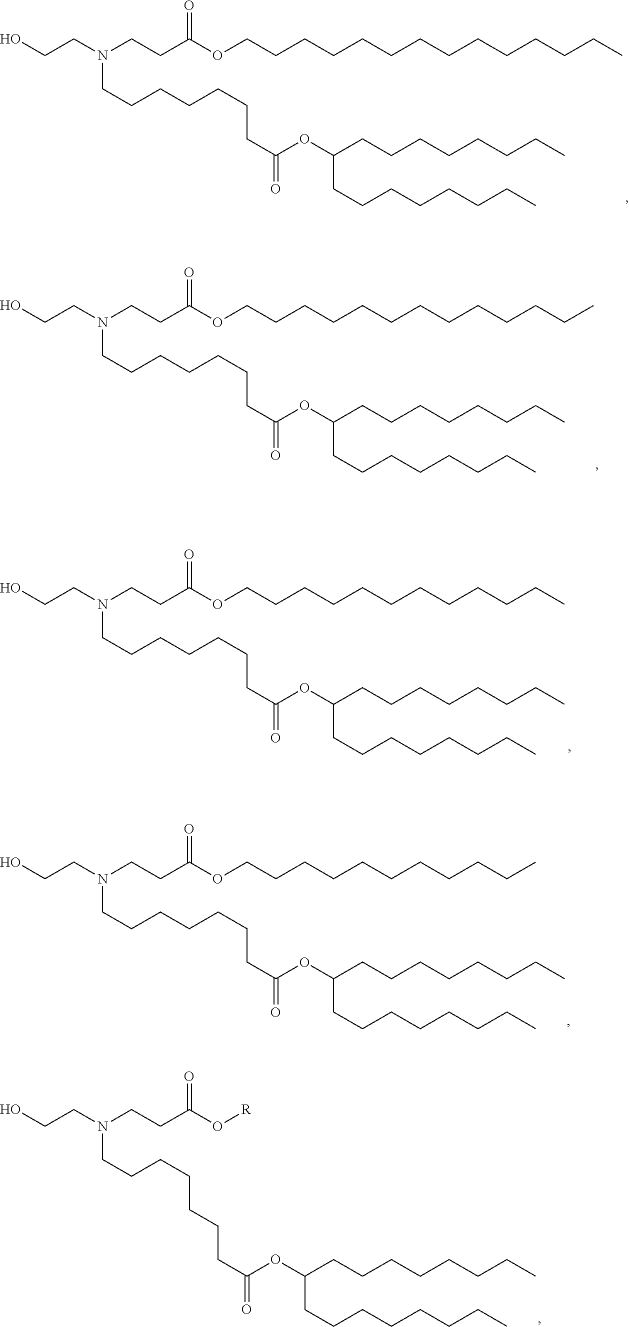

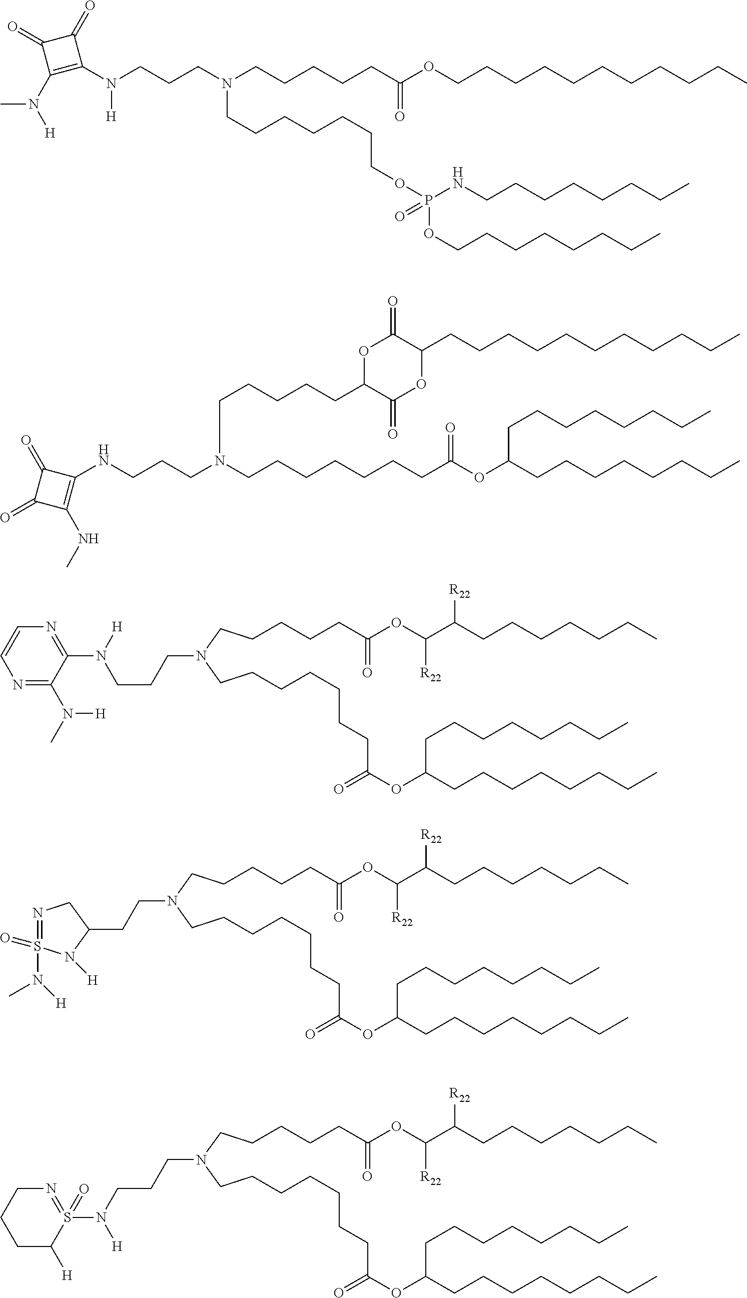

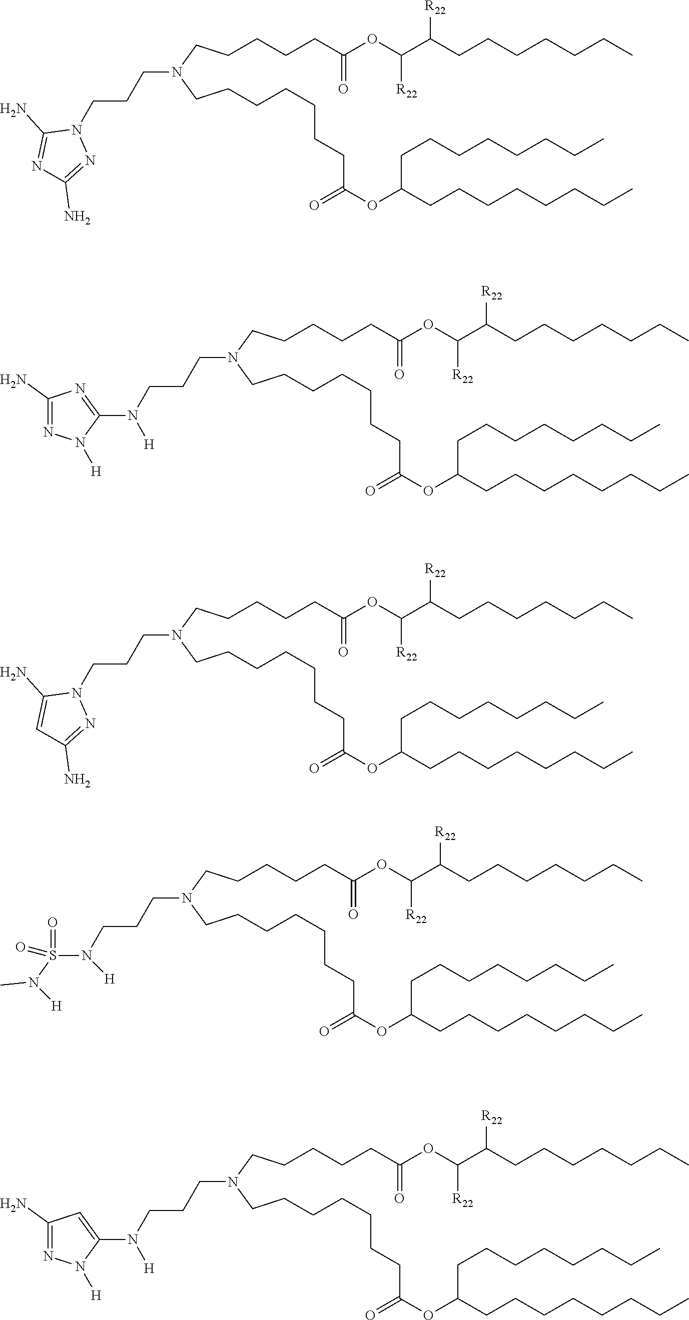

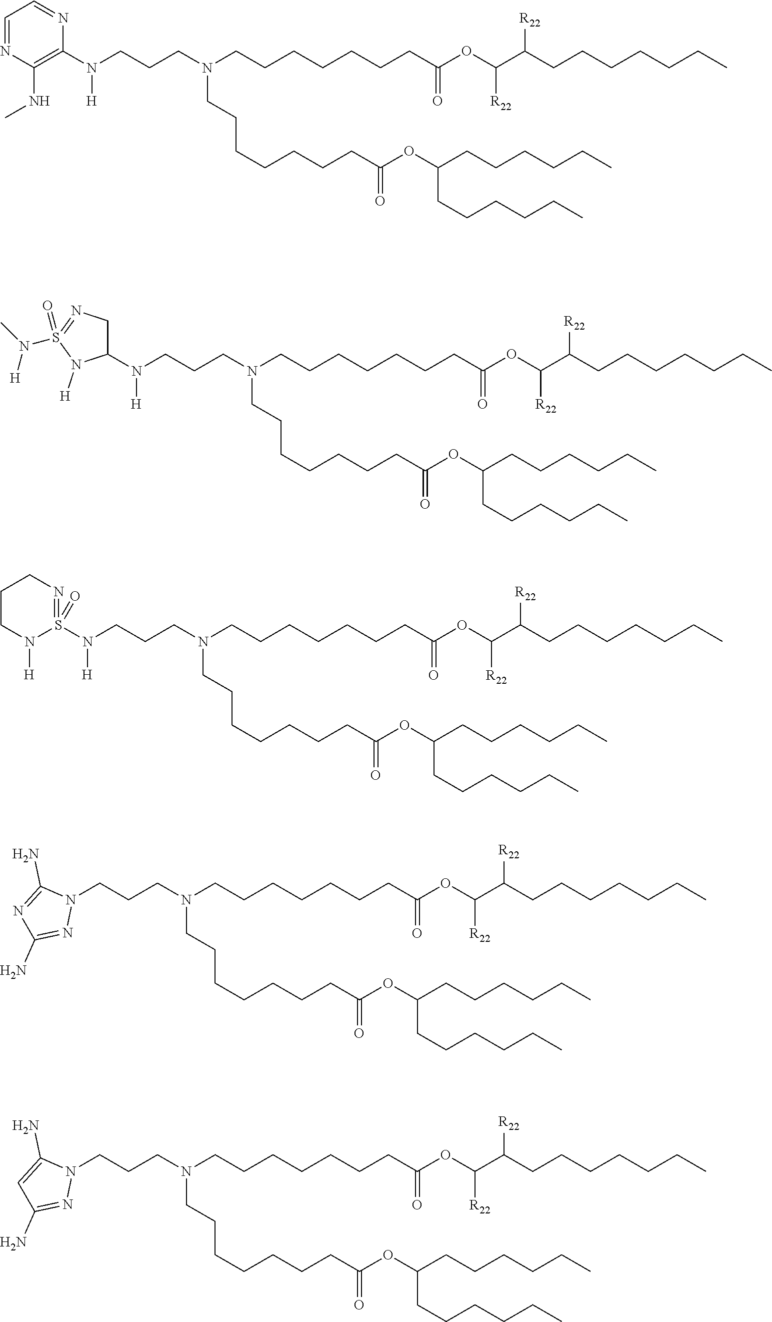

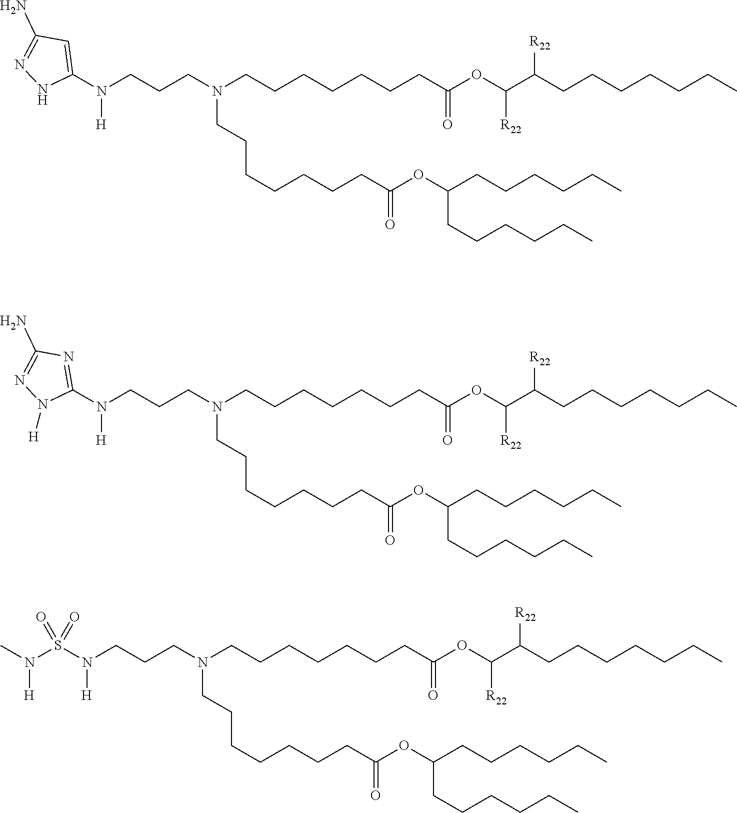

wherein R=

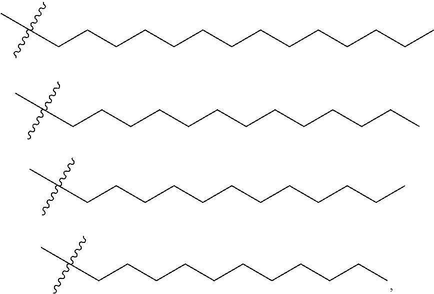 [SMILES: *CCCCCCCCCCCCCC ; *CCCCCCCCCCCCC ; *CCCCCCCCCCCC ; *CCCCCCCCCCC]

and each R₂₂ is independent H, C₁-C₁₅ alkyl, C₂-C₈ alkenyl, or C₂-C₈ alkynyl.

In a second embodiment, the invention relates to a lipid-based carrier comprising a compound of formula (AL-GI) as described herein, wherein the lipid-based carrier is a lipid nanoparticle.

In a first aspect of the second embodiment, the lipid-based carrier further comprises a second lipid.

In a second aspect of the second embodiment, the second lipid is cationic, anionic, ionizable, or zwitterionic lipid. The remainder of features and example features of the second aspect is as described above with respect to the first aspect of the second embodiment.

In a third aspect of the second embodiment, the lipid-based carrier further comprises a PEGylated lipid, a sterol, a phospholipid, and/or a neutral lipid. The remainder of features and example features of the third aspect is as described above with respect to the first through the second aspects of the second embodiment.

In a fourth aspect of the second embodiment, the lipid component of the lipid-based carrier comprises:
about 25-100 mol % of the compound,
about 0-50 mol % phospholipid,
about 0-50 mol % sterol, and
about 0-10 mol % PEGylated lipid.
   The remainder of features and example features of the fourth aspect is as described above with respect to the first through the third aspects of the second embodiment.

In a fifth aspect of the second embodiment, the lipid component of the lipid-based carrier comprises:
about 30-60 mol % of the compound,
about 0-30 mol % phospholipid,
about 15-50 mol % sterol, and
about 0-10 mol % PEGylated lipid.
   The remainder of features and example features of the fifth aspect is as described above with respect to the first through the fourth aspects of the second embodiment.

In a third embodiment the invention relates to a pharmaceutical composition comprising the lipid-based carrier as described herein, and a pharmaceutically acceptable excipient. The pharmaceutical composition may further comprise a therapeutic agent.

In a first aspect of the third embodiment, the pharmaceutical composition further comprises a therapeutic agent.

In a second aspect of the third embodiment, the therapeutic agent is a nucleic acid molecule. For instance, the nucleic acid molecule is a nucleic acid selected from the group consisting of a plasmid, an immunostimulatory oligonucleotide, an antisense oligonucleotide, an antagomir, an aptamer, a deoxyribozyme (DNAzyme), and a ribozyme. The remainder of features and example features of the second aspect is as described above with respect to the first aspect of the third embodiment.

In a third aspect of the third embodiment, the nucleic acid molecule is DNA or RNA. The remainder of features and example features of the third aspect is as described above with respect to the first and second aspects of the third embodiment.

In a fourth aspect of the third embodiment, the nucleic acid molecule is DNA. In some embodiments, the DNA is a linear DNA, circular DNA, single stranded DNA, or double stranded DNA. The remainder of features and example features of the fourth aspect is as described above with respect to the first through the third aspects of the third embodiment.

In a fifth aspect of the third embodiment, the nucleic acid molecule is RNA. In some embodiments, the RNA is selected from the group consisting of an mRNA, miRNA, siRNA or siRNA precursor, RNA aptamer, linear RNA, circular RNA, single stranded RNA, double stranded RNA, tRNA, microRNA (miRNA) or miRNA precursor, a Dicer substrate small interfering RNA (dsiRNA), a short hairpin RNA (shRNA), an asymmetric interfering RNA (aiRNA), a guide RNA (gRNA), lncRNA, ncRNA, sncRNA, rRNA, snRNA, piRNA, snoRNA, snRNA, scaRNA, exRNA, scaRNA, Y RNA, and hnRNA. In one embodiment, the RNA is mRNA. The remainder of features and example features of the fifth aspect is as described above with respect to the first through the fourth aspects of the third embodiment.

In a sixth aspect of the third embodiment, the nucleic acid molecule comprises one or more nucleic acid analogs selected from the group consisting of a phosphoramide, a phosphorothioate, a phosphorodithioate, an O-methylphosphoroamidate, a morpholino, a locked nucleic acid (LNA), a glycerol nucleic acid (GNA), a threose nucleic acid (TNA), and a peptide nucleic acid (PNA). The remainder of features and example features of the sixth aspect is as described above with respect to the first through the fifth aspects of the third embodiment.

In a seventh aspect of the third embodiment, the therapeutic agent is a protein or small molecule drug. The remainder of features and example features of the seventh aspect is as described above with respect to the first through the sixth aspects of the third embodiment.

In an eighth aspect of the third embodiment, the pharmaceutical composition is a vaccine. The remainder of features and example features of the eighth aspect is as described above with respect to the first through the seventh aspects of the third embodiment.

In a fourth embodiment the invention relates to a method of delivering a therapeutic agent to a subject.

In a first aspect of the fourth embodiment, the method comprises administering to the subject the pharmaceutical composition as described herein.

Additional aspects, advantages and features of the invention are set forth in this specification, and in part will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### Definitions

As used in the specification and claims, the singular forms “a”, “an” and “the” include the plural reference, and reference to a particular numerical value includes at least that particular value, unless the context indicates otherwise. For example, reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element and every combination is to be interpreted as separate embodiments. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself.

It is noted that the claims may be drafted to exclude an optional element. As such, this statement is intended to serve as antecedent basis for use of such exclusive terminology as “solely,” “only” and the like in connection with the recitation of claim elements, or use of a “negative” limitation.

As used herein, the term “compound,” is meant to include all the isomers and isotopes of the structure depicted, all the pharmaceutically acceptable salts, solvates, or hydrates thereof, and all crystal forms (e.g., crystal polymorphs), crystal form mixtures, or anhydrides or hydrates thereof.

“Isotopes” refers to atoms having the same atomic number but different mass numbers resulting from a different number of neutrons in the nuclei. For example, isotopes of hydrogen include tritium (³H) and deuterium (²H).

“Isomers.” The compounds described herein or their pharmaceutically acceptable salts may include all isomers, such as geometrical isomers, optical isomers based on an asymmetrical carbon, stereoisomers, tautomers, and the like. For instance, the compounds can contain one or more stereocenters and may thus give rise to geometic isomers (e.g., double bond causing geometric E/Z isomers), enantiomers, diastereomers (e.g., enantiomers (i.e., (+) or (−)) or cis/trans isomers), and other stereoisomeric configurations that may be defined, in terms of absolute stereochemistry, as (R)- or (S)- such as for sugar anomers, or as (D)- or (L)- such as for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Enantiomeric and stereometric mixtures of compounds and means of resolving them into their component enantiomers or stereoisomers are well-known. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Crystallization of the compounds disclosed herein may produce a solvate. As used herein, the term “solvate” refers to an aggregate that comprises one or more molecules of an ionizable lipid of the disclosure with one or more molecules of solvent. The solvent may be water, in which case the solvate may be a hydrate, including a monohydrate, dihydrate, hemihydrate, sesquihydrate, trihydrate, tetrahydrate and the like. Alternatively, the solvent may be an organic solvent.

As used herein, “pharmaceutically acceptable salts” refers to derivatives of the disclosed compounds wherein the parent compound is altered by converting an existing acid or base moiety to its salt form (e.g., by reacting a free base group with a suitable organic acid). The salts retain the biological effectiveness and properties of the free acids, which are not biologically or otherwise undesirable. Examples of pharmaceutically acceptable salts include, but are not limited to, mineral or organic acid salts of basic residues such as amines; alkali or organic salts of acidic residues such as carboxylic acids, and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. Non-limiting examples of inorganic salts are ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Non-limiting examples of organic bases are isopropylamine, diethylamine, ethanolamine, trimethylamine, dicyclohexylamine, choline and caffeine. The pharmaceutically acceptable salts of the compounds can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences (17th ed., Mack Publishing Company, Easton, Pa., 1985, p. 1418); Pharmaceutical Salts: Properties, Selection, and Use (P. H. Stahl and C. G. Wermuth (eds.), Wiley-VCH, 2008); and Berge et al., Journal of Pharmaceutical Science, 66:1-19 (1977), each of which is incorporated herein by reference in its entirety.

“Pharmaceutically acceptable excipient,” as used herein, refers to any ingredient other than the compounds described herein and having the properties of being substantially nontoxic and non-inflammatory in a patient. Excipients may include, for example: adjuvants, anti-adherents, antioxidants, binders, coatings, compression aids, disintegrants, dyes (colorants), emollients, emulsifiers, fillers, solvents, diluents, film formers or coatings, flavors, flavor enhancers, fragrances, glidants (flow enhancers), surfactants, wetting agents, lubricants, preservatives, stabilizers, printing inks, sorbents, suspending or dispersing agents, sweeteners, isotonic agents, and waters of hydration, which has been approved by the United States Food and Drug Administration as being acceptable for use in humans or domestic animals. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. Unless otherwise indicated, “alkyl” generally refers to C₁-C₂₄ alkyl (e.g., C₁-C₁₅ alkyl, C₁-C₁₂ alkyl, C₁-C₈ alkyl, C₁-C₆ alkyl, C₁-C₄ alkyl, or C₁-C₃ alkyl). Unless stated otherwise specifically in the specification, an alkyl group is optionally substituted.

The term “alkylene” refers to a divalent alkyl group. An “alkylene chain” is a polymethylene group, i.e., —(CH₂)ₙ—, wherein n is a positive integer, e.g., from 1 to 6, from 1 to 4, from 1 to 3, from 1 to 2, or from 2 to 3.

The term “haloalkyl” refers to an alkyl in which one or more hydrogen atoms are replaced by halo, and includes alkyl moieties in which all hydrogens have been replaced by halo (e.g., perfluoroalkyl). Alkyl and haloalkyl groups may be optionally inserted with O, N, or S.

The terms “aralkyl” refers to an alkyl moiety in which an alkyl hydrogen atom is replaced by an aryl group. Aralkyl includes groups in which more than one hydrogen atom has been replaced by an aryl group. Examples of “aralkyl” include benzyl, 9-fluorenyl, benzhydryl, and trityl groups.

The term “alkenyl” refers to a straight or branched hydrocarbon chain and characterized in having one or more double bonds. Unless otherwise indicated, “alkenyl” generally refers to C₂-C₈ alkenyl (e.g., C₂-C₆ alkenyl, C₂-C₄ alkenyl, or C₂-C₃ alkenyl). Examples of typical alkenyl groups are allyl, propenyl, 2-butenyl, 3-hexenyl and 3-octenyl groups.

The term “alkynyl” refers to a straight or branched hydrocarbon chain and characterized in having one or more triple bonds. Unless otherwise indicated, “alkynyl” generally refers to C₂-C₈ alkynyl (e.g., C₂-C₆ alkynyl, C₂-C₄ alkynyl, or C₂-C₃ alkynyl). Some examples of typical alkynyl groups are ethynyl, 2-propynyl, and 3-methylbutynyl, and propargyl. The sp² and sp³ carbons may optionally serve as the point of attachment of the alkenyl and alkynyl groups, respectively.

The term “cycloalkyl” includes saturated and partially unsaturated, but not aromatic, cyclic hydrocarbon groups having 3 to 12 carbons, for example, 3 to 8 carbons, 3 to 7 carbons, 3 to 6 carbons, or 3 to 5 carbons, wherein the cycloalkyl group additionally may be optionally substituted. Some examples of typical cycloalkyl groups include, without limitation, cyclopropyl, cyclobutyl, cyclopentyl, cyclopentenyl, cyclohexyl, cyclohexenyl, cycloheptyl, and cyclooctyl.

The term “heterocyclyl,” “heterocycle,” or “heterocyclic ring” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic. The heteroatoms may be selected from O, N, or S (e.g., carbon atoms and 1-3, 1-6, or 1-9 heteroatoms of N, O, or S, if monocyclic, bicyclic, or tricyclic, respectively). For each ring of the heterocycle, 0, 1, 2 or 3 atoms may be substituted by a substituent. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl). Examples of heterocyclyl groups include trizolyl, tetrazolyl, piperazinyl, pyrrolidinyl, dioxanyl, dioxolanyl, diazepinyl, oxazepinyl, thiazepinyl, morpholinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, quinuclidinyl, and the like.

The term “heterocyclylalkyl” refers to an alkyl group substituted by a heterocyclyl, wherein the alkyl and heterocyclyl portions independently are optionally substituted.

The term “aryl” refers to a 6-carbon monocyclic or 10-carbon bicyclic aromatic ring system wherein 0, 1, 2, 3, or 4 atoms of each ring may be substituted by a substituent. The term “aryl” may be used interchangeably with the term “aryl ring.” Examples of aryl groups include phenyl, biphenyl, naphthyl, anthracyl, and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “arylalkyl” or the term “aralkyl” refers to alkyl substituted with an aryl. The term “arylalkoxy” refers to an alkoxy substituted with aryl.

The term “heteroaryl” or “heteroar-” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. The term also include groups in which a heteroaromatic ring is fused to one or more aryl, cycloalkyl, or heterocyclyl rings, where the radical or point of attachment is on the heteroaromatic ring. Examples of heteroaryl groups include pyrrolyl, pyridyl, pyridazinyl, pyrazolyl, triazolyl, tetrazolyl, oxazolyl, isoxazolyl, oxadiazolyl, furanyl, imidazolyl, benzimidazolyl, pyrimidinyl, pyrazinyl, indolizinyl, thiophenyl or thienyl, quinolinyl, indolyl, thiazolyl, isothiazolyl, thiadiazolyl, purinyl, naphthyridinyl, pteridinyl, isoindolyl, benzothienyl, benzofuranyl, dibenzofuranyl, indazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one and the like.

The term “heteroarylalkyl” or the term “heteroaralkyl” refers to an alkyl substituted with a heteroaryl. The term “heteroarylalkoxy” refers to an alkoxy substituted with heteroaryl.

A divalent radical of an alkyl, alkenyl, aryl, heteroaryl, cycloalkyl, heterocyclyl is formed by removal of a hydrogen atom from an alkyl, alkenyl, aryl, heteroaryl, cycloalkyl, and heterocyclyl radical, respectively (or by removal of two hydrogen atoms from an alkane, alkene, arene, heteroarene, cycloalkane, or heterocycle, respectively).

The term “alkoxy” refers to an —O-alkyl radical. The term “aminoalkyl” refers to an alkyl substituted with an amino. The term “mercapto” refers to an —SH radical. The term “thioalkoxy” refers to an —S-alkyl radical.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” means that for any of the above groups (e.g., alkyl, cycloalkyl, heterocyclyl, alkenyl, alkynyl, aryl, heteroaryl, etc.), one or more hydrogen radicals in that group is replaced with the radical of a specified substituent including, but not limited to: halo (e.g., F, Cl, Br, or I), oxo (═O), hydroxyl (—OH), alkoxy, alkoxyalkyl, aralkoxy, alkyl, cycloalkyl, alkenyl, alkynyl, aryl, aralkyl heterocyclyl, heterocyclyl, heteroaryl, thiol, alkylthio, arylthio, alkylthioalkyl, arylthioalkyl, alkylsulfonyl, alkylsulfonylalkyl, arylsulfonylalkyl, aryloxy, carboxyalkyl, alkoxycarbonylalkyl, aminocarbonylalkyl, acyl, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, aralkoxycarbonyl, sulfonyl alkylaminolactams, alkylaminoheteroaryls, alkylaminoheterocycyls, and aminosulfonamides. Exemplary substituents also include: —(C═O)OR^{s}, —O(C═O)R^{s}, —C(═O)R^{s}, —OW, —S(O)ₖR^{s}, —S—SR^{s}, —C(═O)SR^{s}, —SC(═O)R^{s}, —NR^{s}R^{s′}, —R¹C(═O)R^{s}, —C(═O)R^{s}R^{s′}, —R¹C(═O)R^{s}R^{s′}; OC(═O)R^{s}R^{s′}, —R¹C(═O)OR^{s}, —R¹S(O)ₖ R^{s}R^{s′}, —R¹S(O)ₖR^{s}, and —S(O)ₖR^{s}R^{s′}, wherein: R^{s} and R^{s′} is each independently H, C₁-C₁₅ alkyl or cycloalkyl, each R′ is C₁-C₁₅ alkylene, and k is 0, 1 or 2. In some embodiments, the substituent is a C₁-C₁₂ alkyl, C₁-C₆ alkyl, or C₁-C₃ alkyl. In some embodiments, the substituent is a C₃-C₈ cycloalkyl group. In some embodiments, the substituent is a C₂-C₃ alkenyl group. In some embodiments, the substituent is a halo group, such as F or Br. In some embodiments, the substituent is an oxo group. In some embodiments, the substituent is a hydroxyl group. In some embodiments, the substituent is a hydroxyalkylene group (—R¹—OH). In some embodiments, the substituent is an alkoxy group (—OR^{s}). In some embodiments, the substituent is a carboxyl group.

In some embodiments, the substituent is an amino group (—NR^{s}R^{s′}). Suitable substituents also include divalent substituents on a saturated carbon atom, including but are not limited to: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, substituted or unsubstituted C₁₋₆ alkyl, or an unsubstituted 5-6-membered saturated or partially unsaturated ring, or an aryl ring having 0-4 heteroatoms independently selected from nitrogen, oxygen, or sulfur.

As used herein, “expression” of a nucleic acid sequence refers to translation of an mRNA into a polypeptide or protein and/or post-translational modification of a polypeptide or protein.

As used herein, “encapsulation efficiency” refers to the percentage of an encapsulated cargo (e.g., a therapeutic and/or prophylactic agent) that is successfully incorporated into (e.g., encapsulated or otherwise associated with) the lipid-based carrier or lipid nanoformulation, relative to the initial total amount of therapeutic and/or prophylactic agent provided. For example, if 97 mg of therapeutic and/or prophylactic agent are encapsulated in a lipid-based carrier or lipid nanoformulation out of a total 100 mg of therapeutic and/or prophylactic agent initially provided, the encapsulation efficiency may be given as 97%. Encapsulation efficiency can be used to indicate the efficiency of an encapsulated cargo (e.g., a nucleic acid molecule) loading into the lipid-based carrier or lipid nanoformulation using a particular formulation method and formulation recipe.

As used herein, the term “lipid component” refers to a component in the lipid carrier or lipid nanoformulation that includes one or more lipids. For example, the lipid component may include one or more of a cationic/anionic/ionizable/zwitterionic lipid, a neutral lipid, a PEGylated lipid, or other lipid, such as a phospholipid.

The term “lipid nanoformulation” refers generally to a lipid vesicle that is able to carry a cargo (e.g., an encapsulated therapeutic agent such as a nucleic acid) at least partially within its protective layer of lipids, and deliver the cargo to a desirable target site. Typical lipid nanoformulation described herein include liposome and lipid nanoparticle (LNP).

As used herein, the term “lipid carrier” and “lipid nanoformulation” can be used interchangeably to refer a composition comprising one or more lipids, and encompasses lipid nanoparticles (LNPs), liposomes (e.g., lipid vesicles), and lipoplexes. These compositions are typically sized on the order of micrometers or smaller and may include a lipid bilayer.

The term “formulation recipes” as used herein is meant to define the molar ratios of the components in the lipid nanoformulation to be mixed with the encapsulated molecule (e.g., nucleic acid molecule), formulated together to generate a lipid nanoformulation (e.g., LNP composition).

The term “liposome” as used herein refers to a composition comprising an outer lipid layer membrane (e.g., a single lipid bi-layer known as unilamellar liposomes or multiple lipid bi-layers known as multilamellar liposomes) surrounding an internal aqueous space which may contain a cargo. See, e.g., Cullis et ah, Biochim. Biophys Acta, 559: 399-420 (1987), which is incorporated herein by reference in its entirety. A unilamellar liposome generally has a diameter in the range of about 20 to about 400 nanometers (nm), about 50 to about 300 nm, about 100 to about 200 nm, or about 300 to about 400 nm. A multilamellar liposome usually has a diameter in the range of about 1 to about 10 μm and may comprise anywhere from 2 to hundreds of concentric lipid bilayers alternating with layers of an aqueous phase.

The term “lipid nanoparticle” or “LNP” refers to a composition comprising a lipid (e.g., ionic (e.g., cationic or anionic), zwitterionic, or ionizable lipid) for encapsulation of a cargo. LNPs may also include neutral lipids such as phospholipid molecules belonging to the phosphatidylcholine (PC) class; sterols, such as cholesterol; and polyethylene glycol (PEG). LNPs may be taken up by cells via endocytosis and the ionizability of the lipids at low pH enables endosomal escape, which can allow release of cargo into the cytoplasm. LNPs are liposome-like structures. However, LNPs may not have a contiguous bilayer; some LNPs may have a single phospholipid outer layer encapsulating the interior assuming a micelle-like structure (e.g., [symbol] ), which can have a non-aqueous core. Exemplary lipid nanoparticle composition are formulations of ionizable lipids, sterols (or hydrophobic molecules), structural lipids such as phospholipids, polyethyleneglycol (PEG) lipids, and potentially additional components (see Nature Nanotechnology 15:313-320 (2020), which is incorporated herein by reference in its entirety), or single molecules containing combinations of ionizable lipid, sterol, structural phospholipid, and shielding groups (see Nature Materials 20:701-710 (2021), which is incorporated herein by reference in its entirety). These components may be mixed with a cargo molecule (e.g., nucleic acid molecules such as mRNA) to be formulated into LNP composition.

The term “ionizable lipid” refers to a molecule having both an ionizable and lipophilic component. “Ionizable” means a group contained in the lipid (e.g., a head group) can be ionized, e.g., dissociated to produce one or more electrically charged species, under a given condition (e.g., pH). For instance, an ionizable lipid may carry a net positive charge at a selected pH, such as physiological pH (e.g., pH of about 7.0). In some embodiments, the hydrophilic component contains an ionizable amine. In some embodiments, the hydrophobic component contains one or more linear or branched lipids.

As used herein, the terms “PEG-lipid” and “PEGylated lipid” are interchangeable and refer to a lipid comprising a polyethylene glycol component.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the term “size” refers to the hydrodynamic diameter of a lipid nanoparticle population. The measurement of the size of a lipid nanoformulation may be used to indicate the size and population distribution (polydispersity index, PDI) of the composition.

As used herein, the “polydispersity index” is a ratio between weight-average molar mass and Mn is the number-average molar mass that describes the homogeneity of the particle size distribution of a system. A small value, e.g., less than 0.3, indicates a narrow particle size distribution.

As used herein, the term “apparent pKa” refers to the pH at which 50% of the lipid nanoformulation (e.g., LNP) is protonated. This can be used as an indicator of the pH range that the lipid nanoformulation (e.g., LNP) will be protonated, and thus initiate the endosomal escape process in a nucleotide delivery.

As used herein, the term “zeta potential” refers to the electrokinetic potential of lipid, e.g., in a lipid nanoformulation (e.g., a LNP composition). The zeta potential may describe the surface charge of a LNP composition. Zeta potential is useful in predicting organ tropism and potential interaction with serum proteins.

As used herein, “methods of administration” may include both systemic delivery and local delivery. “Systemic delivery” means that a useful, such as a therapeutic, amount of an agent is delivered to most parts of the body. Systemic delivery of a liposome or LNP can be carried out by any means known in the art including, for example, intravenous, intraarterial, intramuscular, intradermal, subcutaneous, and intraperitoneal delivery. In some embodiments, systemic delivery of lipid nanoparticles is by intravenous delivery. “Local delivery,” as used herein, refers to delivery of an agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect.

As used herein, the term “polypeptide” or “polypeptide of interest” refers to a polymer of amino acid residues typically joined by peptide bonds that can be produced naturally (e.g., isolated or purified) or synthetically.

“Nucleic acid” is meant to define an oligonucleotide or polynucleotide sequence. Non-limiting examples of oligonucleotide or polynucleotides are DNA, plasmid DNA, self-amplifying RNA, mRNA, siRNA and tRNA. The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

As used herein, the term “subject” or “patient” refers to any organism to which a composition in accordance with this disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, the subject is a mammal, such as a human. In some embodiments, the subject is a veterinary or farm animal, a domestic animal or pet, or animal used for clinical research. In some embodiments, the subject is an adult, i.e., >18 years of age. In some embodiments, the subject is a pediatric subject, i.e., <18 years of age.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “effective amount” or “therapeutically effective amount” means an amount of an active agent or therapeutic agent to be delivered (e.g., nucleic acid, small molecule drug, therapeutic peptide or protein composition, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. Effective amounts vary, as recognized by those skilled in the art, depending on route of administration, excipient usage, and co-usage with other active agents. Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

### Lipid Nanoformulations/Lipid-Based Carriers

In some embodiments, compounds described herein are formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more compounds described herein, or a pharmaceutically acceptable salt thereof.

All above descriptions and all embodiments discussed in the above aspects relating to the aspects of the lipid compounds, including the compounds covered by formulas (Ia) or (AL-GI), are all applicable to these aspects of the invention relating to the lipid-based carriers (or a lipid nanoformulation).

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more compounds described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

In some embodiments, the lipid nanoparticle of the invention may be conjugated to a targeting moiety (e.g., an antibody or antigen-binding fragment thereof) through a linking group. Various linking groups known in the art may be used in the lipid nanoparticles of the invention, and can comprise one or more of optionally substituted alkylene, optionally substituted heteroalkylene, optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted arylene, optionally substituted heteroarylene, a peptide moiety, a dipeptide moiety, —(C═O)—, a disulfide, a hydrazone, thioester, sulfone, sulfoxide, thiosulfinate, thiosulfonate, sulfate, sulfonate, sulfonylurea, ether, thioether, ester, amide, carbonate, carbamate, urea, sulfamide, succinimide, maleimide, phosphate, diphosphate, triazole, or a saccharide, or a combination thereof. Suitable linking groups are described, e.g., in WO 2024/015229, WO 2024/006272, and WO 2023/225359.

### Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

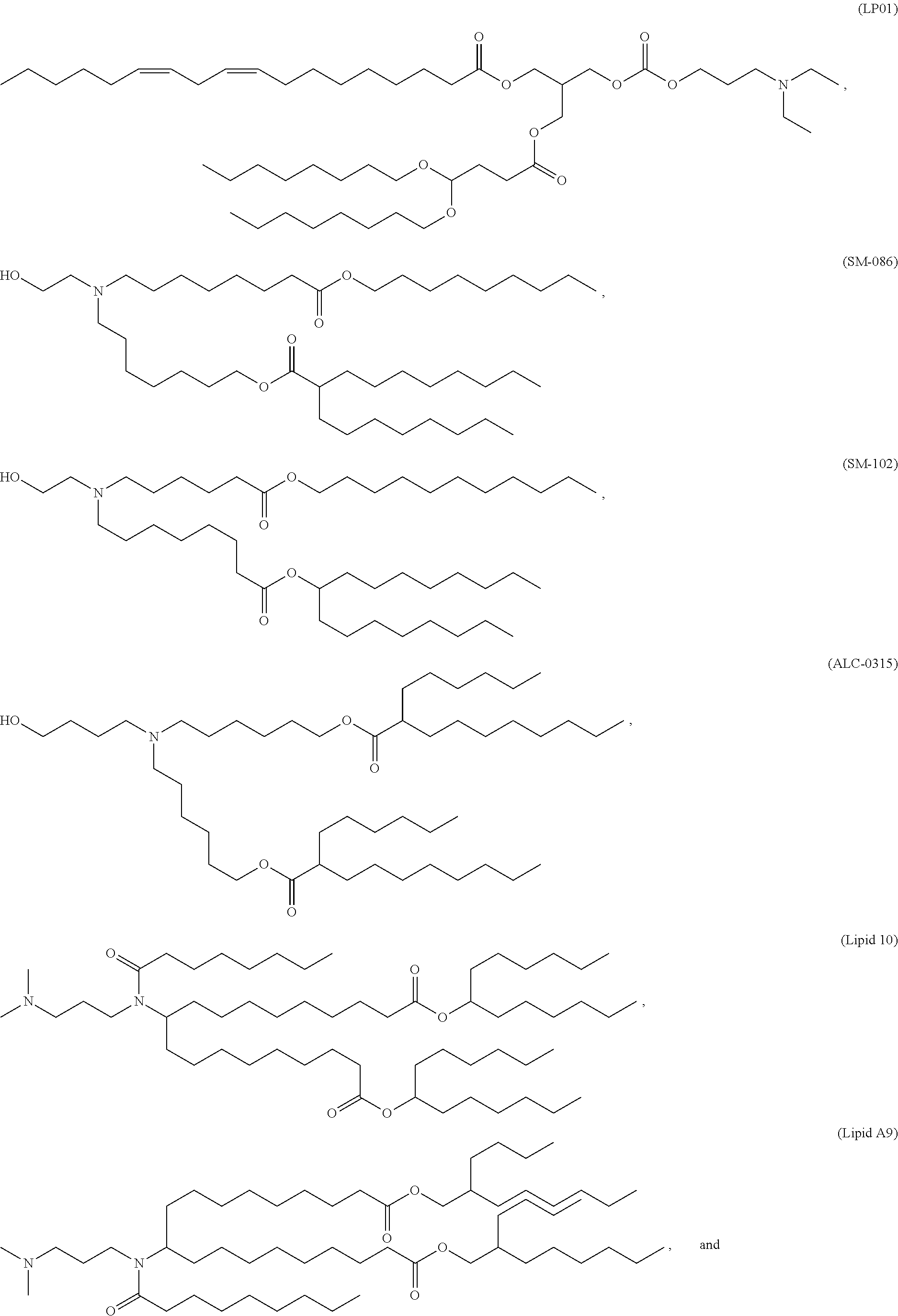

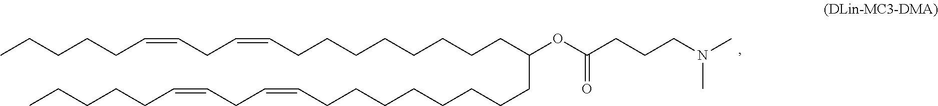 [SMILES: CCCCC/C=C\C/C=C\CCCCCCCCC(CCCCCCCC/C=C\C/C=C\CCCCC)OC(=O)CCCN(C)C]

(see WO 2017/004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et at. Nat Rev Mater 6, 1078-1094 (2021). https://doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, IL, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; 1 of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, I, III, IV, V, VI, VH, VIII, IX, X, XI, XII of US 2012/0202871; I, IL, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; I of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

Non-Cationic Lipids (e.g., Phospholipids) In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, which is incorporated herein by reference. Such lipids include, in some embodiments, plant lipids found to improve liver transfection with mRNA (e.g., DGTS). In some embodiments, saturated long-chain phosphatidylcholines are less permeable and more stable in vivo than their unsaturated counterparts.

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20(6):4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as Sa-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### Polymers and Polyethylene Glycol (PEG)—Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350] (mPEG 350 PE); 1,2-Diacyl-sn-Glycero-3-Phosphoethanolamine-N-[Methoxy(Polyethylene glycol)-550] (mPEG 550 PE); 1,2-Diacyl-sn-Glycero-3-Phosphoethanolamine-N-[Methoxy(Polyethylene glycol)-750] (mPEG 750 PE); 1,2-Diacyl-sn-Glycero-3-Phosphoethanolamine-N-[Methoxy(Polyethylene glycol)-1000] (mPEG 1000 PE); 1,2-Diacyl-sn-Glycero-3-Phosphoethanolamine-N-[Methoxy(Polyethylene glycol)-2000] (mPEG 2000 PE); 1,2-Diacyl-sn-Glycero-3-Phosphoethanolamine-N-[Methoxy(Polyethylene glycol)-3000] (mPEG 3000 PE); 1,2-Diacyl-sn-Glycero-3-Phosphoethanolamine-N-[Methoxy(Polyethylene glycol)-5000] (mPEG 5000 PE); N-Acyl-Sphingosine-1-[Succinyl(Methoxy Polyethylene Glycol) 750] (mPEG 750 Ceramide); N-Acyl-Sphingosine-1-[Succinyl(Methoxy Polyethylene Glycol) 2000] (mPEG 2000 Ceramide); and N-Acyl-Sphingosine-1-[Succinyl(Methoxy Polyethylene Glycol) 5000] (mPEG 5000 Ceramide). In some embodiments, the PEG lipid is a polyethyleneglycol-diacylglycerol (i.e, polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol). In some embodiments, the conjugated lipid molecule may be used to inhibit aggregation of lipid nanoparticles and/or provide steric stabilization.

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18). The PEG conjugate can also comprise PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000].

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, HII-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

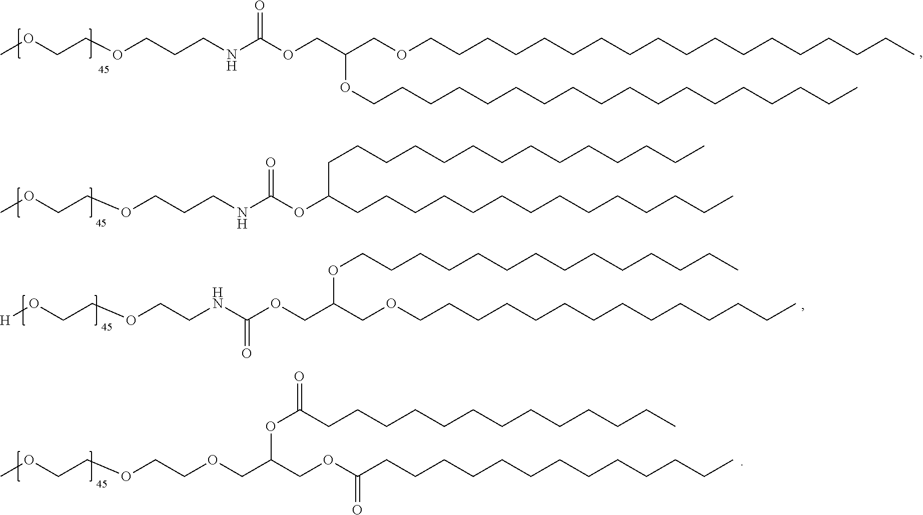

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), which are incorporated herein by reference in their entirety.

### Percentages of Lipid Nanoformulation Components

In some embodiments, the lipid-based carrier (or lipid nanoformulation) comprises one of more of the compounds described herein, optionally a non-cationic lipid (e.g., a phospholipid), a sterol, a neutral lipid, and optionally conjugated lipid (e.g., a PEGylated lipid) that inhibits aggregation of particles. The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

Molar ratios of the ionizable lipid, non-cationic-lipid, sterol, and conjugated lipid (e.g., the PEGylated lipid) can be varied as needed. For example, the lipid-based carrier (or lipid nanoformulation) can include, by mol % or wt % of the total lipid components, 30-70% ionizable lipid (including the lipid compound as described herein), 0-60% sterol (e.g., cholesterol or derivative), 0-30% non-cationic-lipid, and 1-10% conjugated lipid (e.g., the PEGylated lipid). For instance, the lipid-based carrier (or lipid nanoformulation) can include, by mol % or wt % of the total lipid components, 30-40% ionizable lipid (including the lipid compound as described herein), 40-50% sterol (e.g., cholesterol or derivative), and 10-20% non-cationic-lipid. In some embodiments, the lipid-based carrier (or lipid nanoformulation) can include, by mol % or wt % of the total lipid components, 50-75% ionizable lipid (including the lipid compound as described herein), 20-40% sterol (e.g., cholesterol or derivative), and 5 to 10% non-cationic-lipid, and 1-10% conjugated lipid (e.g., the PEGylated lipid). The composition may contain 60-70% ionizable lipid by mole or by total weight of the composition, 25-35% cholesterol by mole or by total weight of the composition, and 5-10% non-cationic-lipid by mole or by total weight of the composition. In some embodiments, the lipid-based carrier (or lipid nanoformulation) can include, by mol % or wt % of the total lipid components, up to 90% ionizable lipid (including the lipid compound as described herein), and 2 to 15% non-cationic lipid.

In some embodiments, the lipid-based carrier (or lipid nanoformulation) can include, by mol % or wt % of the total lipid components, 8-30% ionizable lipid (including the lipid compound as described herein), 5-30% non-cationic lipid, and 0-20% sterol (e.g., cholesterol or derivative).

In some embodiments, the lipid-based carrier (or lipid nanoformulation) can include, by mol % or wt % of the total lipid components, 4-25% ionizable lipid (including the lipid compound as described herein), 4-25% non-cationic lipid, 2 to 25% sterol (e.g., cholesterol or derivative), and 10 to 35% conjugated lipid (e.g., the PEGylated lipid).

In some embodiments, the lipid-based carrier (or lipid nanoformulation) can include, by mol % or wt % of the total lipid components, 2-30% ionizable lipid (including the lipid compound as described herein), 2-30% non-cationic lipid, 1 to 15% sterol (e.g., cholesterol or derivative), and 2 to 35% conjugated lipid (e.g., the PEGylated lipid).

In some embodiments, the lipid-based carrier (or lipid nanoformulation) can include, by mol % or wt % of the total lipid components, up to 90% ionizable lipid (including the lipid compound as described herein) and 2-10% non-cationic lipids.

In some embodiments, the lipid compound described herein is a component of the lipid-based carrier (or lipid nanoformulation) and comprises from 10 mol % to 95 mol %, from 10 mol % to 90 mol %, from 10 mol % to 80 mol %, from 10 mol % to 70 mol %, from 10 mol % to 60 mol %, from 20 mol % to 55 mol %, from 20 mol % to 45 mol %, 20 mol % to 40 mol %, from 25 mol % to 50 mol %, from 25 mol % to 45 mol %, from 30 mol % to 50 mol %, from 30 mol % to 45 mol %, from 30 mol % to 40 mol %, from 35 mol % to 45 mol %, or from 37 mol % to 42 mol % (or any fraction of these ranges) of the total lipid components.

In some embodiments, where the lipid-based carrier (or lipid nanoformulation) contains a mixture of phospholipid and sterol (e.g. cholesterol or derivative), the mixture may be present up to 40 mol %, 45 mol %, 50 mot %, 55 mol %, or 60 mol % of the total lipid components.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, where the lipid-based carrier (or lipid nanoformulation) is phospholipid-free, the sterol component (e.g. cholesterol or derivative) may be present up to 25 mol %, 30 mol %, 35 mol %, 40 mol %, 45 mol %, 50 mol %, 55 mol %, or 60 mol % of the total lipid components. For instance, the sterol component (e.g. cholesterol or derivative) may be present from 25 mol % to 65 mol %, from 25 mol % to 60 mol %, from 25 mol % to 55 mol %, from 25 mol % to 50 mol %, from 25 mol % to 45 mol %, from 25 mol % to 40 mol %, from 30 mol % to 45 mol %, from 30 mol % to 40 mol %, from 35 mol % to 45 mol %, from 30 mol % to 35 mol %, or from 35 mol % to 40 mol % (or any fraction thereof or range therein) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the cargo (e.g., an encapsulated therapeutic agent such as a nucleic acid) can be varied as desired. For example, the total lipid components to the cargo (mass or weight) ratio can be from about 10:1 to about 30:1. In some embodiments, the total lipid components to the cargo ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the cargo can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation)'s overall lipid content can range from about 5 mg/ml to about 30 mg/mL. Nitrogen:phosphate ratios (N:P ratio) is evaluated at values between 0.1 and 100.

In some embodiments, the lipid-based carrier (or lipid nanoformulation) includes the ionizable lipid compound as described herein, phospholipid, cholesterol, and a PEG-ylated lipid in a molar ratio of 50:10:38.5:1.5. In some embodiments, the lipid-based carrier (or lipid nanoformulation) includes the ionizable lipid compound as described herein, cholesterol and a PEG-ylated lipid in a molar ratio of 60:38.5:1.5.

In some embodiments of any of the aspects or embodiments herein, the lipid-based carrier (or lipid nanoformulation) further comprises a tissue targeting moiety. The tissue targeting moiety can be a peptide, oligosaccharide or the like, which can be used for the delivery of the lipid-based carrier (or lipid nanoformulation) to one or more specific tissues such as the liver. In some embodiments, the tissue targeting moiety is a ligand for liver specific receptors. In one embodiment, the ligand of liver specific receptors used for liver targeting is an oligosaccharide such as N-Acetylgalactosamine (GalNAc) which is covalently attached to a component of a lipid-based carrier (or lipid nanoformulation), e.g., PEG-lipid conjugates or the like. In some embodiments, the GalNAc is covalently attached to, for example, PEG-lipid conjugate. In some embodiments, the GalNAc is conjugated to DSPE-PEG2000. In some embodiments, the GalNAc-PEG-lipid conjugate is present in the lipid-based carrier (or lipid nanoformulation) at a molar percentage of 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total lipid. In some embodiments, the GalNAc-PEG-lipid conjugate is present in the lipid-based carrier (or lipid nanoformulation) at a molar percentage of 0.2% of the total lipid. In some embodiments, the GalNAc-PEG-lipid conjugate is present in the lipid-based carrier (or lipid nanoformulation) at a molar percentage of 0.3% of the total lipid. In some embodiments, the GalNAc-PEG-lipid conjugate is present in the lipid-based carrier (or lipid nanoformulation) at a molar percentage of 0.4% of the total lipid. In some embodiments, the GalNAc-PEG-lipid conjugate is present in the lipid-based carrier (or lipid nanoformulation) at a molar percentage of 0.5% of the total lipid. In some embodiments, the GalNAc-PEG-lipid conjugate is present in the lipid-based carrier (or lipid nanoformulation) at a molar percentage of 0.6% of the total lipid. In some embodiments, the GalNAc-PEG-lipid conjugate is present in the lipid-based carrier (or lipid nanoformulation) at a molar percentage of 0.7% of the total lipid. In some embodiments, the GalNAc-PEG-lipid conjugate is present in the lipid-based carrier (or lipid nanoformulation) at a molar percentage of 0.8% of the total lipid. In some embodiments, the GalNAc-PEG-lipid conjugate is present in the lipid-based carrier (or lipid nanoformulation) at a molar percentage of 0.9% of the total lipid. In some embodiments, the GalNAc-PEG-lipid conjugate is present in the lipid-based carrier (or lipid nanoformulation) at a molar percentage of 1.0% of the total lipid. In some embodiments, the GalNAc-PEG-lipid conjugate is present in the lipid-based carrier (or lipid nanoformulation) at a molar percentage of about 1.5% of the total lipid. In some embodiments, the GalNAc-PEG-lipid conjugate is present in the lipid-based carrier (or lipid nanoformulation) at a molar percentage of 2.0% of the total lipid.

### Properties of Lipid Nanoformulations

In some embodiments, the average particle diameter of the lipid-based carrier (or lipid nanoformulation) may be between 10s of nm and 100s of nm, e.g., measured by dynamic light scattering (DLS). In some embodiments, the average particle diameter of the lipid-based carrier (or lipid nanoformulation) ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, from about 38 mm to about 42 mm, from about 40 nm to about 150 nm (such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm), from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm.

The lipid-based carrier or lipid nanoformulation (e.g., liposome or LNP) may, in some instances, be relatively homogenous. A polydispersity index may be used to indicate the homogeneity of a lipid nanoformulation (e.g., liposome or LNP), e.g., the particle size distribution of the liposome or LNP. A small (e.g., less than 0.3) polydispersity index generally indicates a narrow particle size distribution. A lipid-based carrier or lipid nanoformulation (e.g., liposome or LNP) may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of the lipid-based carrier or lipid nanoformulation (e.g., liposome or LNP) may be from about 0.10 to about 0.20.

The zeta potential of a lipid-based carrier or a lipid nanoformulation (e.g., liposome or LNP) may be used to indicate the electrokinetic potential of the composition. In some embodiments, the zeta potential may describe the surface charge of a liposome or LNP. Lipid nanoformulations (e.g., liposomes or LNP) with relatively low charges, positive or negative, are generally desirable, as more highly charged species may interact undesirably with cells, tissues, and other elements in the body. In some embodiments, the zeta potential of a liposome or LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a cargo such as a protein and/or nucleic acid, describes the amount of protein and/or nucleic acid that is encapsulated or otherwise associated with a lipid nanoformulation (e.g., liposome or LNP) after preparation, relative to the initial amount provided. The encapsulation efficiency is desirably high (e.g., at least 70%, 80%, 90%, 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the liposome or LNP described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

The lipid carrier or lipid nanoformulation may optionally include one or more coatings. In some embodiments, the lipid carrier or lipid nanoformulation (e.g., liposome or LNP) may be formulated in a capsule, film, or tablet having a coating. A capsule, film, or tablet including a composition described herein may have any useful size, tensile strength, hardness, or density.

Additional exemplary lipids, formulations, methods, and characterization of a lipid carrier or lipid nanoformulation (e.g., liposome or LNP) are taught by WO 2020/061457 and WO 2021/113777, which are incorporated herein by reference in their entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TranslT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

Lipid nanoformulations (e.g., liposome or LNP) optimized for the delivery of CRISPR-Cas systems, e.g., Cas9-gRNA RNP, gRNA, Cas9 mRNA, are described in WO 2019067992 and WO 2019067910, which are incorporated by reference in their entirety.

Additional specific lipid nanoformulations (e.g., liposome or LNP) useful for delivery of nucleic acid effector molecules are described in U.S. Pat. Nos. 8,158,601 and 8,168,775, which are incorporated by reference in their entirety.

A variety of methods can be used for preparing the lipid carrier or lipid nanoformulation (e.g., liposomes or LNPs) described herein. Such methods are known in the art or disclosed herein, for example, the methods described in Lichtenberg and Barenholz in Methods of Biochemical Analysis, 33:337-462 (1988), which is incorporated herein by reference in its entirety. See also Szoka et al., Ann. Rev. Biophys. Bioeng. 9.467 (1980); U.S. Pat. Nos. 4,235,871; 4,501,728; and 4,837,028; Liposomes, Marc J. Ostro, ed., Marcel Dekker, Inc., New York, 1983, Chapter 1; and Hope, et al., Chem. Phys. Lip. 40:89 (1986), which are incorporated herein by reference in their entirety. Small unilamellar vesicles (SUV, size <100 nm) can be prepared by a combination of standard methods of thin-film hydration and repeated extrusion.

Techniques for sizing the lipid carrier or lipid nanoformulations (e.g., liposomes or LNPs) to a desired size are well-known to one skilled in the art. See, e.g., U.S. Pat. No. 4,737,323, and Hope et al., Biochim. Biophys. Acta, 812: 55-65, which are incorporated by reference in their entirety. Sonicating a lipid nanoformulation (e.g., liposome or LNP) suspension either by bath or probe sonication produces a progressive size reduction down to small unilamellar vesicles less than about 50 nm in size. Homogenization or microfluidization are other methods which rely on shearing energy to fragment large lipid nanoformulations (e.g., liposomes or LNPs) into smaller ones. In a typical homogenization procedure, multilamellar vesicles are recirculated through a standard emulsion homogenizer until selected lipid nanoformulation (e.g., liposome or LNP) sizes, typically between about 100 and 500 nm, are observed. In both methods, the particle size distribution can be monitored by conventional laser-beam particle size discrimination.

Extrusion of lipid nanoformulations (e.g., liposomes or LNPs) through a small-pore polycarbonate membrane or an asymmetric ceramic membrane is a very effective method for reducing liposome or LNP sizes to a relatively well-defined size distribution. Typically, the suspension is cycled through the membrane one or more times until the desired liposome or LNP size distribution is achieved. The lipid-based carrier or lipid nanoformulations may be extruded through successively smaller-pore membranes, to achieve a gradual reduction in liposome or LNP size.

Any of the lipid-based carrier or lipid nanoformulations described herein can be analyzed by methods well-known to one skilled in the art to determine its physical and/or chemical features. For example, a phosphate assay can be used to determine the concentration of the lipid nanoformulations. One phosphate assay is based on the interaction between molybdate and malachite green dye. The main principle involves the reaction of inorganic phosphate with molybdate to form a colorless unreduced phosphomolybdate complex which is converted to a blue colored complex when reduced under acidic conditions. Phosphomolybdate gives 20 or 30 times more color when complexed with malachite green. The final product, reduced green soluble complex is measured by its absorbance at 620 nm and is a direct measure of inorganic phosphate in solution.

In some embodiments, the lipid-based carrier or lipid nanoformulations disclosed herein are tested for particle size, lipid concentration, and active agent encapsulation.

### Further Ionizable Lipids

Some non-limiting examples of additional lipid compounds that may be used (e.g., in combination with the lipid compound described herein and other lipid components) to form the lipid-based carrier (or lipid nanoformulation) include:

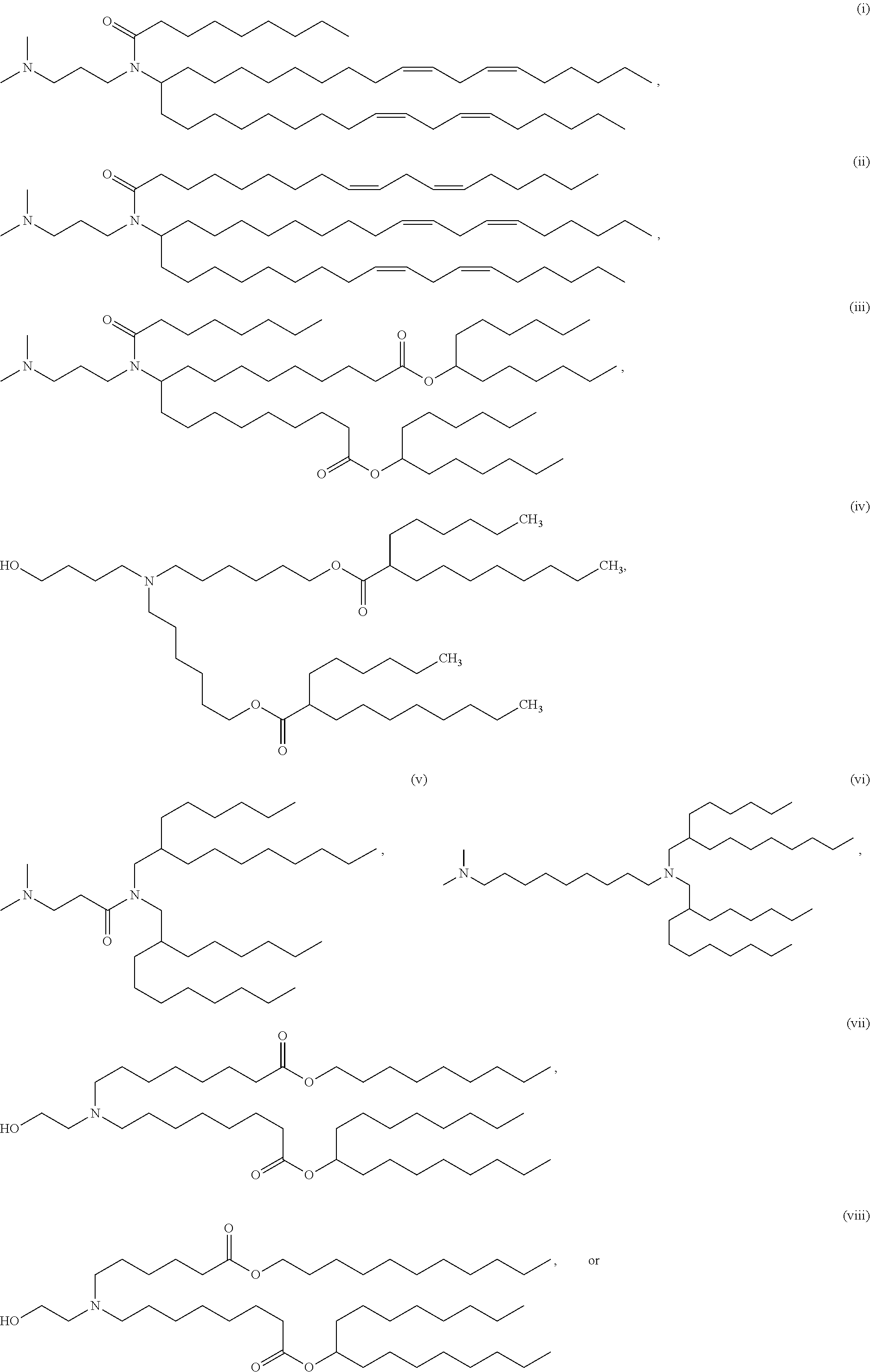

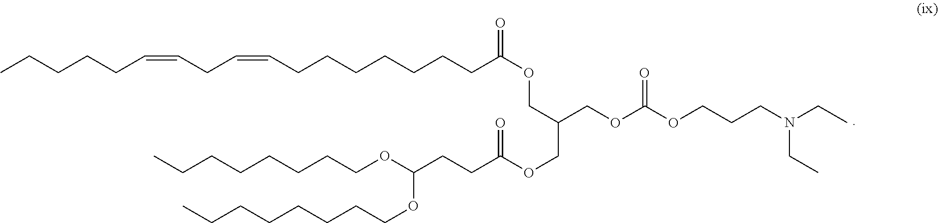 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments, the lipid-based carrier (or lipid nanoformulation) further comprises the lipids in formula (i), (ii), (iii), (iv), (v), (vi), (vii), (viii), or (ix).

In some embodiments, the lipid-based carrier (or lipid nanoformulation) further comprises the following compounds having the structure of:

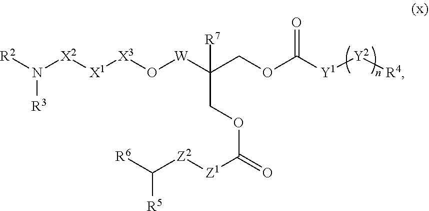

wherein:
   X¹ is O, NR¹, or a direct bond, X² is C₂₋₅ alkylene, and X³ is C(═O) or a direct bond;
   R′ is H or Me, R³ is C₁₋₃ alkyl, R² is C₁₋₃ alkyl, or R² taken together with the nitrogen atom to which it is attached and 1-3 carbon atoms of X² form a 4-, 5-, or 6-membered ring; or
   X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring;
   Y¹ is C₂₋₁₂ alkylene, and Y² is selected from

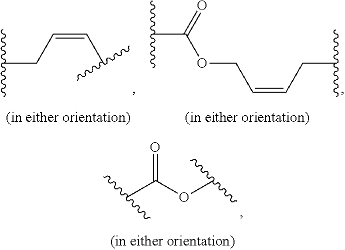 [SMILES: */C=C\C* ; *C/C=C\COC(*)=O ; *OC(*)=O]

n is 0 to 3:
R⁴ is C₁₋₁₅ alkyl;
Z¹ is C₁₋₆ alkylene or a direct bond, and Z² is

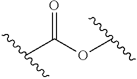 [SMILES: *OC(*)=O]

(in either orientation) or absent, provided that if Z¹ is a direct bond, Z² is absent;
R⁵ is C₅₋₉ alkyl or C₆₋₁₀ alkoxy, R⁶ is C₅₋₉ alkyl or C₆₋₁₀ alkoxy;
W is methylene or a direct bond; and
R⁷ is H or Me, or a salt thereof;
provided that if R³ and R² are C₂ alkyls, X¹ is O, X² is linear C₃ alkylene, X³ is C(═O),
Y¹ is linear C₅ alkylene, (Y²)n-R⁴ is

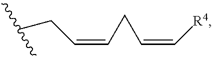

R⁴ is linear C₅ alkyl, Z¹ is C₂ alkylene, Z² is absent, W is methylene, and R⁷ is H, then R⁵ and R⁶ are not C₂ alkoxy.

In some embodiments, the lipid-based carrier (or lipid nanoformulation) further comprises one or more compounds of formula (x).

Additional non-limiting examples of lipid compounds that may be further included in the lipid-based carrier (or lipid nanoformulation) further comprises (e.g., in combination with the lipid compounds described herein and other lipid components):

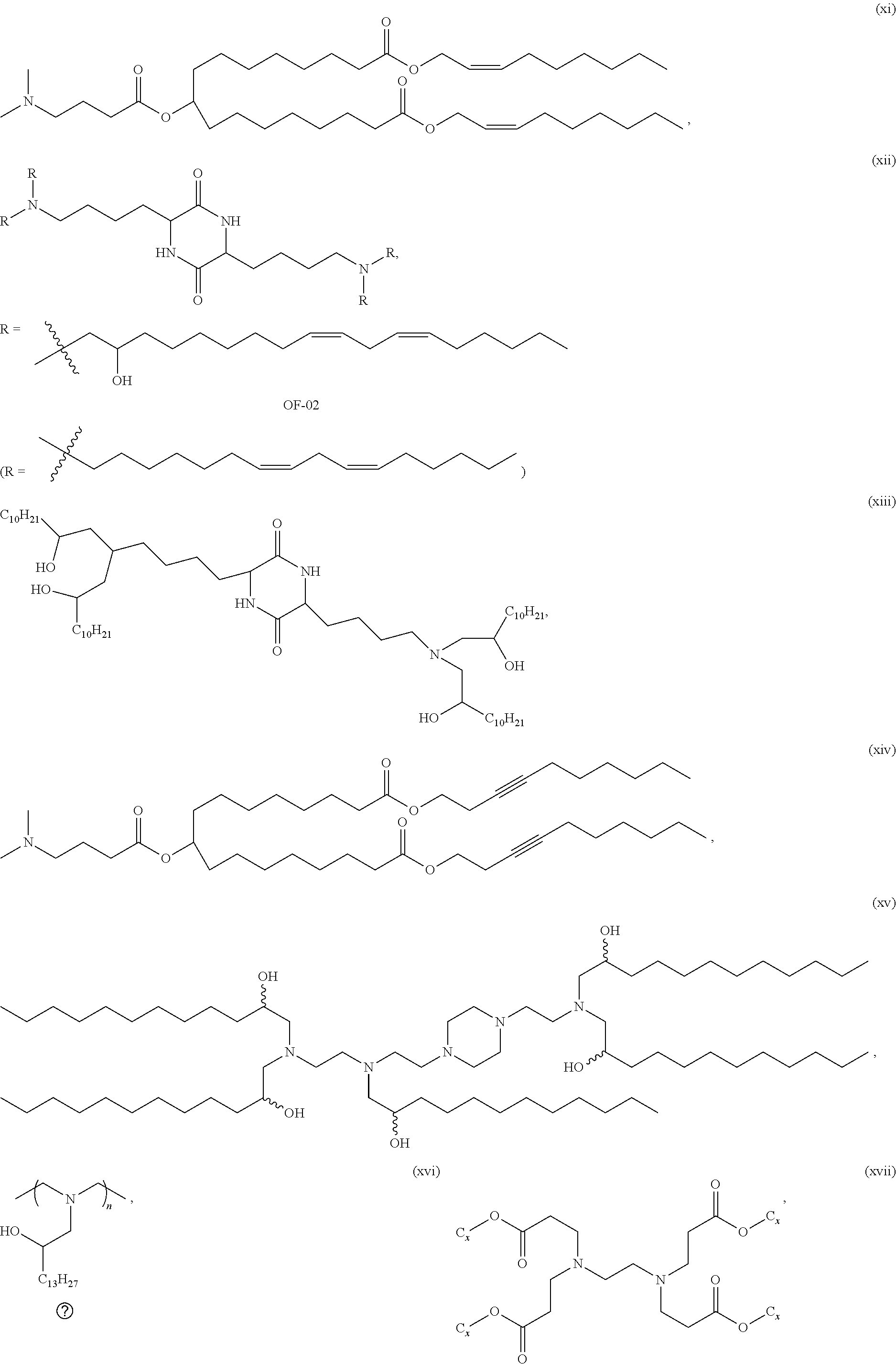

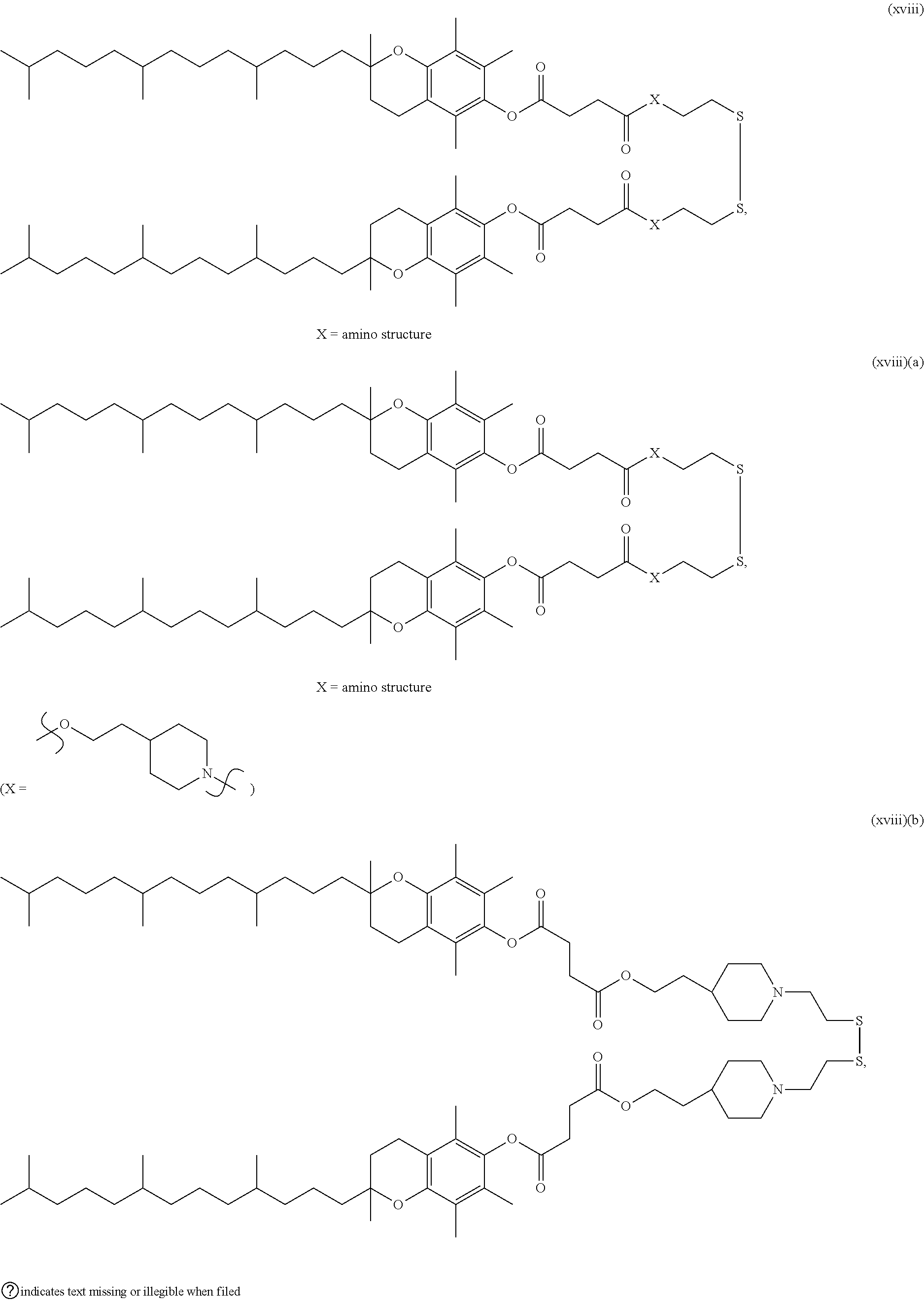

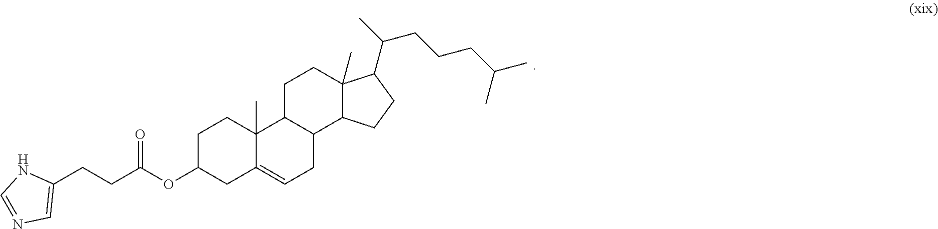 [SMILES: CC(C)CCCC(C)C1CCC2C3CC=C4CC(OC(=O)CCc5cnc[nH]5)CCC4(C)C3CCC12C]

In some embodiments, the lipid-based carrier (or lipid nanoformulation) further comprises one or more compounds of formula (xi), (xii), (xiii), (xiv), (xv), (xvi), (xvii), (xviii) (e.g., (xviii)a, (xviii)b), or (xix).

In some embodiments, the lipid-based carrier (or lipid nanoformulation) further comprises lipids formed by one of the following reactions:

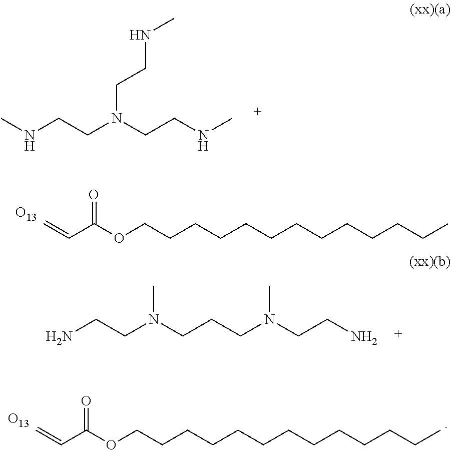

In some embodiments, the lipid-based carrier (or lipid nanoformulation) further comprises the lipid (e.g., in combination with the lipid compounds described herein and other lipid components) having the formula (xxi):

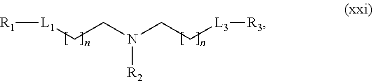

wherein:
   each n is independently an integer from 2-15;
   L₁ and L₃ are each independently —OC(O)—* or —C(O)O—*, wherein “*” indicates the attachment point to R₁ or R₃;
   R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkylxalkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
   R₂ is selected from a group consisting of

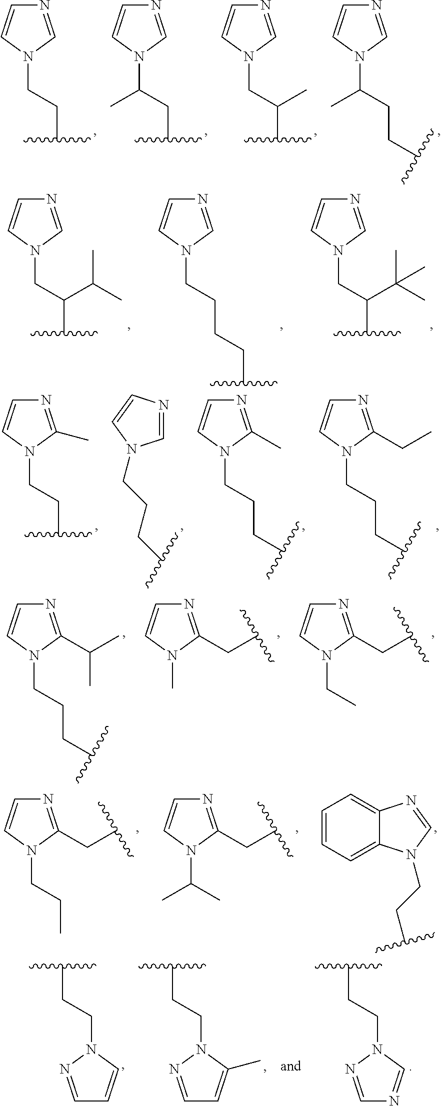

In some embodiments, the lipid-based carrier (or lipid nanoformulation) further comprises one or more compounds of formula (xxi). In some embodiments, the compounds of formula (xxi) include those described by WO 2021/113777 (e.g., a lipid of Formula (1) such as a lipid of Table 1 of WO 2021/113777), which is incorporated herein by reference in its entirety.

In some embodiments, the lipid-based carrier (or lipid nanoformulation) further comprises lipids (e.g., in combination with the lipid compound described herein and other lipid components) having the formula (xxii):

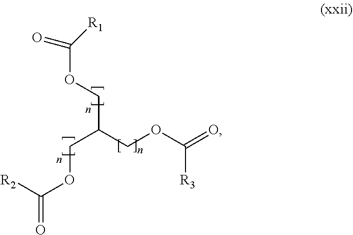

wherein:
   each n is independently an integer from 1-15;
   R₁ and R₂ are each independently selected from a group consisting of:

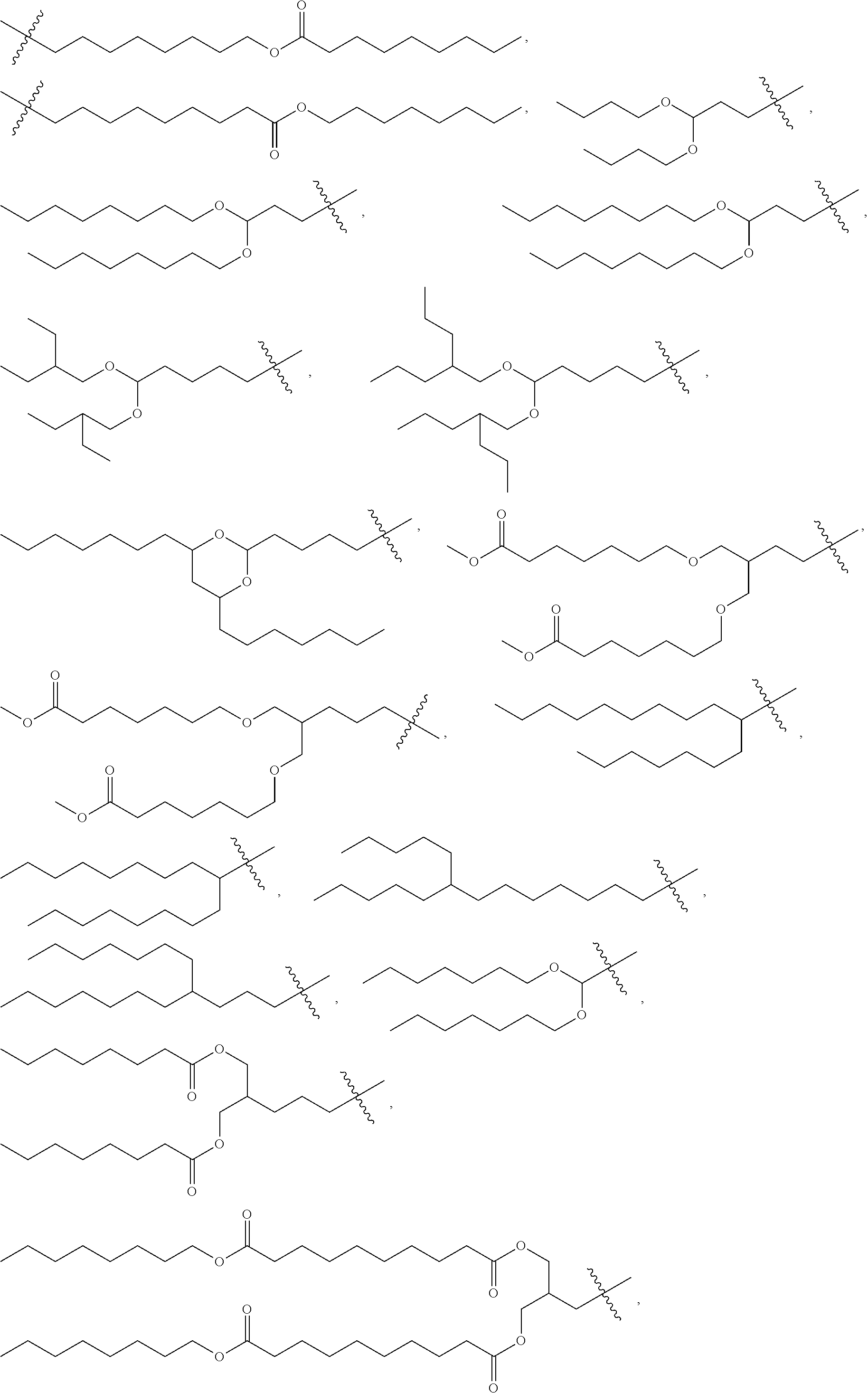

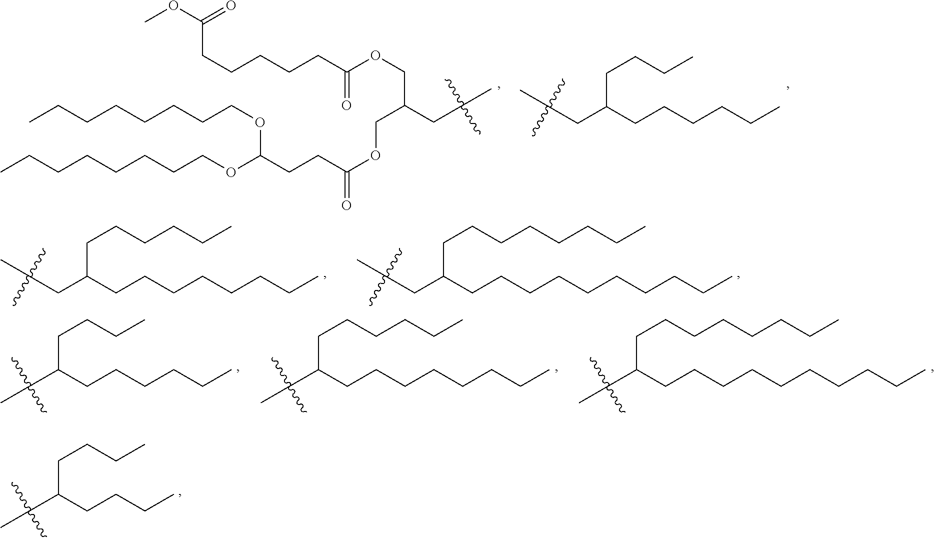

R₃ is selected from a group consisting of:

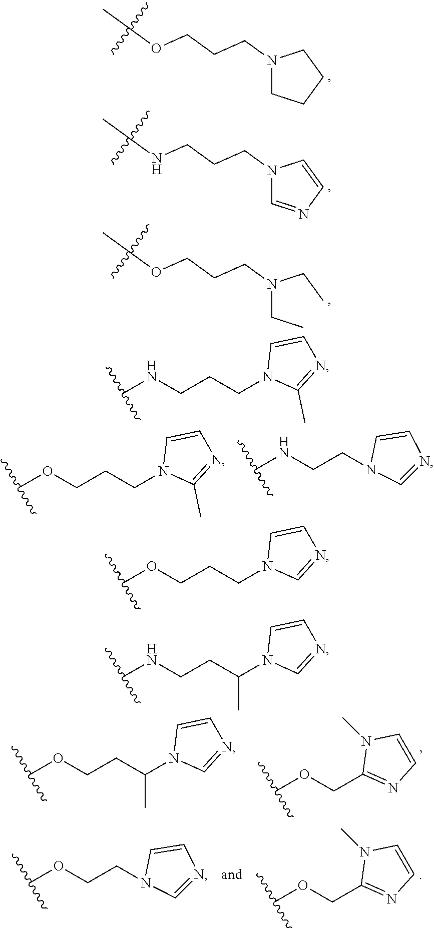

In some embodiments, the lipid-based carrier (or lipid nanoformulation) further comprises one or more compounds of formula (xxii). In some embodiments, the compounds of formula (xxii) include those described by WO 2021/113777 (e.g., a lipid of Formula (2) such as a lipid of Table 2 of WO 2021/113777), which is incorporated herein by reference in its entirety.

In some embodiments, the lipid-based carrier (or lipid nanoformulation) further comprises lipids (e.g., in combination with the lipid compound described herein and other lipid components) having the formula (xxiii):

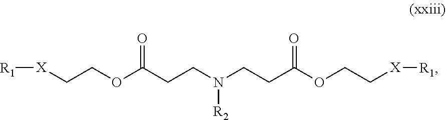

wherein
X is selected from —O—, —S—, or —OC(O)—*, wherein * indicates the attachment point to R₁,
R₁ is selected from a group consisting of

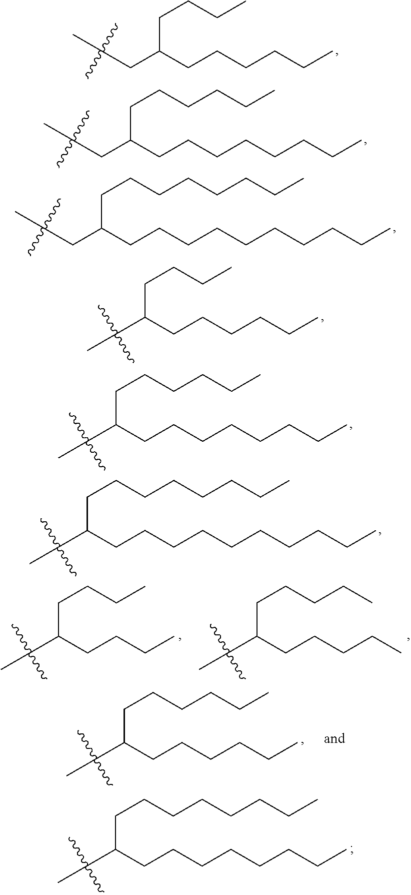

and
R₂ is selected from a group consisting of:

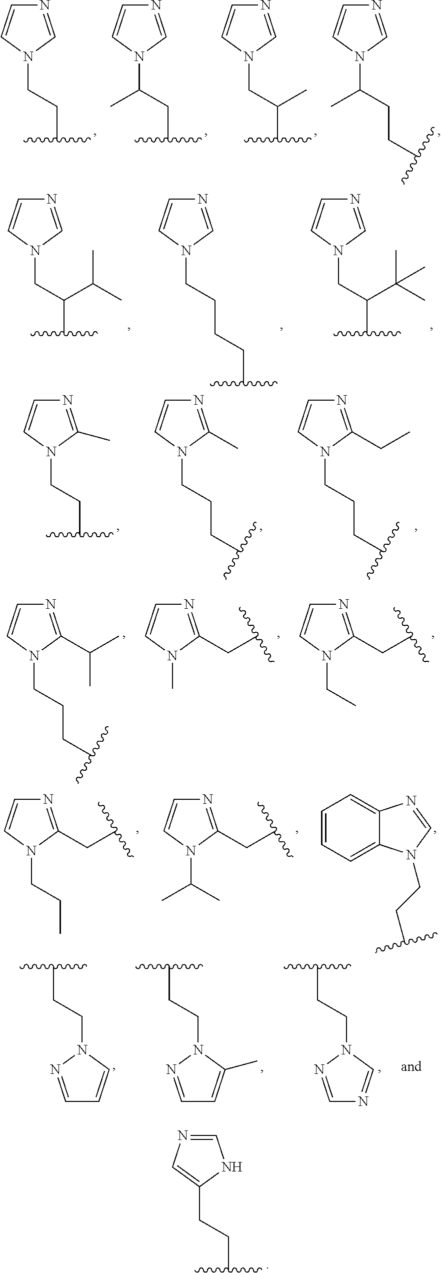

In some embodiments, the lipid-based carrier (or lipid nanoformulation) further comprises one or more compounds of formula (xxiii). In some embodiments, the compounds of formula (xxiii) include those described by WO 2021/113777 (e.g., a lipid of Formula (3) such as a lipid of Table 3 of WO 2021/113777), which is incorporated herein by reference in its entirety.

In some embodiments, the lipid-based carrier (or lipid nanoformulation) further comprises one or more additional ionizable lipids.

In one embodiment, the additional ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (which is incorporated by reference herein in its entirety).

In one embodiment, the additional ionizable lipid is 9Z,12Z)-3-((4,4-bi s(octyloxy)butanoyl)oxy)-2-((((3-(diethylamino)propoxy)carbonyl)oxy)methyl)propyl octadeca-9,12-dienoate (LP01), e.g., as synthesized in Example 13 of WO 2015/095340 (which is incorporated by reference herein in its entirety).

In one embodiment, the additional ionizable lipid is Di((Z)-non-2-en-1-yl) 9-((4-dimethylamino)butanoyl)oxy)heptadecanedioate (L319), e.g., as synthesized in Example 7, 8, or 9 of US 2012/0027803 (which is incorporated by reference herein in its entirety).

In one embodiment, the additional ionizable lipid is 1,1′-((2-(4-(2-((2-(Bis(2-hydroxydodecyl)amino)ethyl)(2-hydroxydodecyl)amino)ethyl)piperazin-1-yl)ethyl)azanediyl)bis(dodecan-2-ol) (C12-200), e.g., as synthesized in Examples 14 and 16 of WO 2010/053572 (which is incorporated by reference herein in its entirety).

In one embodiment, the additional ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO 2020/106946 (which is incorporated by reference herein in its entirety).

In one embodiment, the additional ionizable lipid is MC3 (6Z,9Z,28Z,3 lZ)-heptatriaconta-6,9,28,31-tetraen-19-yl-4-(dimethylamino) butanoate (DLin-MC3-DMA or MC3), e.g., as described in Example 9 of WO 2019/051289A9, which is incorporated by reference herein in its entirety.

In one embodiment, the additional ionizable lipid is lipid ATX-002, e.g., as described in Example 10 of WO 2019/051289A9, which incorporated by reference herein in its entirety.

In one embodiment, the additional ionizable lipid is (13Z,16Z)-A,A-dimethyl-3-nonyldocosa-13, 16-dien-1-amine (Compound 32), e.g., as described in Example 11 of WO 2019/051289A9 (which is incorporated by reference herein in its entirety).

In one embodiment, the additional ionizable lipid is Compound 6 or Compound 22, e.g., as described in Example 12 of WO 2019/051289A9, which is incorporated by reference herein in its entirety.

Examples of additional ionizable lipids that can be used in lipid-based carrier (or lipid nanoformulation) include, without limitation, those listed in Table 1 of WO 2019/051289, which is incorporated herein by reference.

### Pharmaceutical Compositions

The disclosure also provides pharmaceutical compositions comprising the lipid-based carrier as described herein, and a pharmaceutically acceptable excipient. The pharmaceutical composition may further comprise a therapeutic agent.

All above descriptions and all embodiments discussed in the above aspects relating to the aspects of the lipid compounds, including the compounds covered by formulas (AL-GI), (AL-Ia)-(AL-Ic), (AL-IIa)-(AL-IIc), (AL-IIIa)-(AL-IIIg), (AL-IVa)-(AL-IVc), and the exemplary formulas for lipids having at least two ester group, lipids containing a lactide (or its derivative) group, and lipids containing a phosphoramidate group are all applicable to these aspects of the invention relating to the pharmaceutical composition.

All above descriptions and all embodiments discussed in the above aspects relating to the aspects of the lipid-based carrier (or lipid nanoformulation), including various other lipid components, are applicable to these aspects of the invention relating to the pharmaceutical composition.

### Therapeutic Agents

In some embodiments, the invention provides a method of delivering an effector, the method comprising administering, e.g., to a patient, a lipid nanoparticle of the invention, wherein the lipid nanoparticle comprises an effector. In certain embodiments, the effector comprises a therapeutic agent.

### Nucleic Acid Molecule

In some embodiments, the therapeutic agent is a nucleic acid molecule. The nucleic acid molecule may be any nucleic acid molecule that can function as a therapeutic or diagnostic agent. For instance, the nucleic acid molecule may be a DNA or RNA.

In some embodiments, the nucleic acid molecule is a nucleic acid selected from the group consisting of a plasmid, an immunostimulatory oligonucleotide, an antisense oligonucleotide, an antagomir, an aptamer, a deoxyribozyme (DNAzyme), and a ribozyme.

In some embodiments, the therapeutic agent is DNA. The DNA may be selected by one skilled in the art. In some embodiments, the DNA is linear DNA, circular DNA, single stranded DNA, or double stranded DNA.

In one embodiment, the therapeutic agent is linear DNA.

In one embodiment, the therapeutic agent is circular DNA.

In one embodiment, the therapeutic agent is single stranded DNA.

In one embodiment, the therapeutic agent is double stranded DNA.

In some embodiments, the therapeutic agent is RNA. The RNA may be selected by one skilled in the art. In certain embodiments, the RNA is mRNA, miRNA, siRNA or siRNA precursor, RNA aptamer, linear RNA, circular RNA, single stranded RNA, double stranded RNA, tRNA, microRNA (miRNA) or miRNA precursor, a Dicer substrate small interfering RNA (dsiRNA), a short hairpin RNA (shRNA), an asymmetric interfering RNA (aiRNA), a guide RNA (gRNA), lncRNA, ncRNA, sncRNA, rRNA, snRNA, piRNA, snoRNA, snRNA, scaRNA, exRNA, scaRNA, Y RNA, or hnRNA.

In some embodiments, the nucleic acid molecule comprises one or more nucleic acid analogs selected from the group consisting of a phosphoramide, a phosphorothioate, a phosphorodithioate, an O-methylphosphoroamidate, a morpholino, a locked nucleic acid (LNA), a glycerol nucleic acid (GNA), a threose nucleic acid (TNA), and a peptide nucleic acid (PNA).

In one embodiment, the therapeutic agent is an mRNA (messenger RNA).

In one embodiment, the therapeutic agent is a miRNA (microRNA) or miRNA precursor.

In one embodiment, the therapeutic agent is a siRNA (small interfering RNA) or siRNA precursor.

In one embodiment, the therapeutic agent is a Dicer substrate small interfering RNA (dsiRNA).

In one embodiment, the therapeutic agent is a short hairpin RNA (shRNA).

In one embodiment, the therapeutic agent is an asymmetric interfering RNA (aiRNA).

In one embodiment, the therapeutic agent is a guide RNA (gRNA).

In one embodiment, the therapeutic agent is an RNA aptamer.

In one embodiment, the therapeutic agent is a circular RNA, e.g., a circular RNA encoding a therapeutic polypeptide, or a non-coding circular RNA.

In one embodiment, the therapeutic agent is a tRNA (transfer RNA).

In one embodiment, the therapeutic agent is a rRNA (ribosomal RNA).

In one embodiment, the therapeutic agent is a lncRNA (long non-coding RNA).

In one embodiment, the therapeutic agent is a snRNA (small nuclear RNA).

In one embodiment, the therapeutic agent is a ncRNA (non-coding RNA).

In one embodiment, the therapeutic agent is a sncRNA (small noncoding RNA).

In one embodiment, the therapeutic agent is a snoRNA (small nucleolar RNA).

In one embodiment, the therapeutic agent is a piRNA (piwi-interacting RNA).

In one embodiment, the therapeutic agent is a scaRNA (small cajal body-specific RNA).

In one embodiment, the therapeutic agent is an exRNA (extracellular RNA).

In one embodiment, the therapeutic agent is a Y RNA (small non-coding RNAs that are components of the Ro60 ribonucleoprotein particle).

In one embodiment, the therapeutic agent is a hnRNA (heterogeneous nuclear RNA).

In one embodiment, the therapeutic agent is a shRNA (small hairpin RNA).

In some embodiments, the therapeutic agent is an enzymatic nucleic acid molecule. The term “enzymatic nucleic acid molecule” refers to a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner et al., Nucleic Acids Research 23:2092-2096 (1995); Hammann et al., Antisense and Nucleic Acid Drug Dev. 9:25-31 (1999), which are incorporated herein by reference in their entirety).

The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity.

In some embodiments, the therapeutic agent is an antisense nucleic acid. The term “antisense nucleic acid” refers to a non-enzymatic nucleic acid molecule that binds to target RNA by means of RNA-RNA or RNA-DNA or RNA-PNA (protein nucleic acid) interactions and alters the activity of the target RNA. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. In addition, antisense DNA can be used to target RNA by means of DNA-RNA interactions, thereby activating RNase H, which digests the target RNA in the duplex.

In some embodiments, the nucleic acid molecule may be a 2-5A antisense chimera. The term “2-5A antisense chimera” refers to an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA.

In some embodiments, the nucleic acid molecule may be a triplex forming oligonucleotide. The term “triplex forming oligonucleotide” refers to an oligonucleotide that can bind to a double-stranded DNA in a sequence-specific manner to form a triple-strand helix.

In some embodiments, the nucleic acid molecule may be a decoy RNA. The term “decoy RNA” refers to a RNA molecule or aptamer that is designed to preferentially bind to a predetermined ligand. Such binding can result in the inhibition or activation of a target molecule.

In some embodiments, the nucleic acid molecule (e.g., RNA or DNA) encodes a therapeutic peptide or polypeptide. In the case of a DNA, the nucleic acid comprises a promoter operably linked to the sequence encoding the therapeutic peptide or polypeptide. The therapeutic peptide or polypeptide may be, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a Gene Writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the nucleic acid molecule (e.g., a DNA or RNA) encodes (if DNA) or is (if RNA) a non-coding RNA, e.g., one or more of a siRNA, a miRNA, long non-coding RNA, a piRNA, a snoRNA, a scaRNA, a tRNA, a rRNA, a therapeutic RNA aptamer, and a snRNA.

In some embodiments, the therapeutic nucleic acid molecule targets a host gene, e.g., the nucleic acid effector hybridizes to an endogenous gene.

In some embodiments, the nucleic acid molecule is an antisense RNA; a guide RNA; a nucleic acid that hybridizes to an exogenous nucleic acid such as a viral DNA or RNA, nucleic acid that hybridizes to an RNA; a nucleic acid that interferes with gene transcription; a nucleic acid that interferes with RNA translation; a nucleic acid that stabilizes RNA or destabilizes RNA such as through targeting for degradation; or a nucleic acid that modulates a DNA or RNA binding factor.

In some embodiments, the nucleic acid molecule targets a sense strand of a host gene. In some embodiments, the nucleic acid molecule targets an antisense strand of a host gene.

In some embodiments, the nucleic acid molecule is or encodes a guide RNA. Guide RNA sequences are generally designed to have a length of between 15-30 nucleotides (e.g., 17, 19, 20, 21, 24 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991. The gRNA may recognize specific DNA sequences (e.g., sequences adjacent to or within a promoter, enhancer, silencer, or repressor of a gene). In one embodiment, the gRNA is used as part of a CRISPR system for gene editing. For the purposes of gene editing, the ssDNA construct or sequence disclosed herein may be designed to include one or multiple sequences encoding guide RNA sequences corresponding to a desired target DNA sequence; see, for example, Cong et al. (2013) Science, 339:819-823; Ran et al. (2013) Nature Protocols, 8:2281-2308.

In some embodiments, the nucleic acid molecule can include a plurality of sequences. The plurality may be the same or different types. The plurality of sequences may be the same or different sequences of the same type.

All the nucleic acid molecules described herein can be chemically modified. The various modification strategy to the nucleic acid molecules are well known to one skilled in the art. In some embodiments, the nucleic acid molecule comprises one or more modifications selected from the group consisting of pseudouridine, 5-bromouracil, 5-methylcytosine, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. In some embodiments, the antisense oligonucleotide may be a locked nucleic acid oligonucleotide (LNA). The term “locked nucleic acid (LNA)” refers to oligonucleotides that contain one or more nucleotide building blocks in which an extra methylene bridge fixes the ribose moiety either in the C3′-endo (beta-D-LNA) or C2′-endo (alpha-L-LNA) conformation (Grunweller A, Hartmann R K, BioDrugs, 21(4): 235-243 (2007)).

Additional examples of the nucleic acid molecules (including tumor suppressor genes, antisense oligonucleotides, siRNA, miRNA, or shRNA) may be found in U.S. Published Patent Application No. 2007/0065499 and U.S. Pat. No. 7,780,882, which are incorporated by reference herein in their entireties.

In some embodiments, the pharmaceutical composition can include a plurality of nucleic acid molecules, which may be the same or different types.

In some embodiments, the N:P ratio of the nucleic acid molecule-encapsulated lipid-based carrier or lipid nanoformulation ranges from 1:1 to 30:1, for instance from 3:1 to 20:1, from 3:1 to 15:1, from 3:1 to 10:1, or from 3:1 to 6:1. An N:P ratio refers to the molar ratio of the amines present in the lipid-based carrier or lipid nanoformulation (e.g., the amines in the ionizable lipids) to the phosphates present in the nucleic acid molecule. It is a factor for efficient packaging and potency. In one embodiment, the N:P ratio of the nucleic acid molecule-encapsulated lipid-based carrier or lipid nanoformulation ranges from 3:1 to 15:1.

### Other Therapeutic Agents

The therapeutic agent can be a nucleic acid, a peptide or protein, or a small molecule drug, encapsulated in the lipid-based carrier or lipid nanoformulation. The pharmaceutical composition can contain two or more different therapeutic agents from the nucleic acid molecule, peptide or protein, and small molecule drug.

In some embodiments, the protein effector may be any peptide or protein molecule that can function as a therapeutic or diagnostic agent.

In some embodiments, the protein may be a peptide or polypeptide, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase, a deubiquitinase, an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In one embodiment, the protein is a ribonucleoprotein (RNP) that a complex of ribonucleic acid and RNA-binding protein.

In one embodiment, the protein is a recombinant cytokine.

In some embodiments, the pharmaceutical composition can include a plurality of protein molecules, which may be the same or different types.

In some embodiments, the therapeutic agent is a small molecule drug, for instance, a small molecule drug approved for use in humans by an appropriate regulatory authority.

In some embodiments, the small molecule drug is an HDAC inhibitor, a kinase inhibitor, a cytotoxic molecule, a chromatin modulator, an RNAi modulator, transcription factor, an adjuvant, or a combination of two or more.

In some embodiments, the small molecule drug can be a small molecule that lacks cell permeability properties.

In some embodiments, the pharmaceutical composition can include a plurality of small molecule drugs, which may be the same or different types.

In some embodiments, the therapeutic agent is a vaccine. In some embodiments, the vaccine is a RNA vaccine, such as a RNA cancer vaccine or RNA vaccine for infectious disease (e.g., a virus, such as an influenza virus vaccine or a corona virus vaccine (e.g., COVID-19 vaccine).

### Other Ingredients

The pharmaceutical compositions may contain one or more pharmaceutically acceptable excipients. The pharmaceutically acceptable excipient is selected on the basis of the mode and route of administration. Suitable pharmaceutical carriers or excipients for use in pharmaceutical formulations are described in Remington: The Science and Practice of Pharmacy, 21^{st} Ed., Gennaro, Ed., Lippencott Williams & Wilkins (2005); Handbook of Pharmaceutical Excipients, 6^{th} Edition, Rowe et al., Eds., Pharmaceutical Press (2009); and the USP/NF (United States Pharmacopeia and the National Formulary), which are herein incorporated by reference in their entirety.

In some embodiments, the pharmaceutically acceptable excipient includes one or more of an antioxidant, binder, antiadherent, buffer, coloring agent, diluent (e.g., solid or liquid), disintegrant (e.g., coatings disintegrate), dispersing agent, dyestuff, filler, emulsifier, flavoring agent, lubricant, pH adjuster, pigment, preservative, stabilizer, solubilizing agent, solvent, suspending agent, sweetener, or wetting agent, or combination thereof.

Examples of suitable excipients include, without limitation, acacia, alginate, calcium phosphate, calcium carbonate, calcium silicate, carbopol gel, carboxymethyl cellulose, carnauba wax, cellulose, crospovidone, dextrose, diacetylated monoglycerides, ethylcellulose, gelatin, glyceryl monostearate 40-50, gum acacia, gum arabic, hydroxyethyl cellulose, hydroxypropyl cellulose, hydroxypropylmethyl cellulose, hypromellose phthalate, hypromellose, lactose, lecithin, magnesium stearate, kaolin, methacrylic acid copolymer type C, mannitol, methyl cellulose, methylhydroxybenzoate, microcrystalline cellulose, povidone, polyethylene glycol, polysorbate 80, polyvinylpyrrolidone, propylhydroxybenzoate, sodium carboxymethyl cellulose sodium hydroxide, sodium stearyl fumarate, sodium starch glycolate, starch, sorbitan monooleate sorbitol, sorbic acid, sucrose, talc, tragacanth, talc, triethyl citrate, titanium dioxide, yellow ferric oxide, talc, oil medium (e.g., peanut oil, liquid paraffin, mineral oil, olive oil, almond oil, glycerin, propylene glycol), or water,

When the excipient serves as a diluent, it can be a solid, semisolid, or liquid material (e.g., normal saline), which acts as a vehicle, carrier or medium for the active ingredient. As is known in the art, the type of diluent can vary depending upon the intended route of administration.

The pharmaceutical compositions can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl chloride; hexamethonium chloride; benzalkonium chloride, benzethonium chloride; phenol, butyl or benzil alcohol; alkyl parabens such as methyl or propyl paraben; catechol, resorcinol; cyclohexanol; 3-pentanol, and m-cresol); low molecular weight (less than about 10 residues) polypeptides; proteins, such as serum albumin, gelatin, or immunoglobulins; hydrophilic polymers such as polyvinylpyrrolidone, amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Suitable carriers or excipients for the pharmaceutical compositions may also include a substance that enhances the ability of the body of an individual to absorb the LNP or liposome. Suitable carriers and/or excipients also include any substance that can be used to bulk up formulations with a LNP or liposome, to allow for convenient and accurate dosage. In addition, carriers and/or excipients may be used in the manufacturing process to aid in the handling of a LNP or liposome. Depending on the route of administration, and form of medication, different carriers and/or excipients may be used.

Carriers and/or excipients may also include vehicles and/or diluents. “Vehicles” indicates any of various media acting usually as solvents or carriers; “diluent” indicates a diluting agent which is issued to dilute an active ingredient of a composition; suitable diluent include any substance that can decrease the viscosity of a medicine. The type and amounts of carriers and/or excipients are chosen in function of the chosen pharmaceutical form; suitable pharmaceutical forms are liquid systems like solutions, infusions, suspensions; semisolid systems like colloids, gels, pastes or creams; solid systems like powders, granulates, tablets, capsules, pellets, microgranulates, minitablets, microcapsules, micropellets, suppositories; etc.

Each of the above systems can be suitably formulated for normal, delayed or accelerated release, using techniques well-known in the art.

### Formulations, Dosages, and Routes of Administration

The pharmaceutical compositions described herein can be prepared according to standard techniques, as well as those techniques described herein. For instance, the pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are known in the art. See, e.g., Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York.

The therapeutic agent may be encapsulated in the lipid-based carrier (or lipid nanoformulation), for instance, the therapeutic agent may be completely or partially located in the interior space of the LNPs, within the lipid layer/membrane, or associated with the exterior surface of the lipid layer/membrane. One purpose of incorporating therapeutic agents into LNPs is to protect the therapeutic agents from environments which may contain enzymes or chemicals or conditions that degrade the therapeutic agents and/or systems or receptors that cause the rapid excretion of the therapeutic agents. Moreover, incorporating therapeutic agents into LNPs may promote uptake of the therapeutic agent, and hence, may enhance the therapeutic effect.

In some embodiments, in the pharmaceutical composition, the lipid components to therapeutic agent ratio (mass/mass ratio:w/w ratio) can range from about 1:1 to about 25:1, 10:1 to about 14:1, about 3:1 to about 15:1, about 4:1 to about 10:1, about 5:1 to about 9:1, or about 6:1 to about 9:1.

The lipid-based carrier or pharmaceutical composition may contain about 5 to about 95% by weight the therapeutic agent, based on the weight of the lipid-based carrier or pharmaceutical composition. In some embodiments, the lipid-based carrier or pharmaceutical composition contains about 5%, about 10%, about 20, about 30, about 40, about 50, about 60, about 70, about 80, about 90, or about 95% by weight, based on the weight of the LNP or pharmaceutical composition, of the therapeutic agent. In some embodiments, the lipid-based carrier or pharmaceutical composition contains the therapeutic agent in an amount about 5-95%, about 5-90%, about 5-80%, about 5-70%, about 5-60%, about 5-50/o, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30-95%, about 30-90%, about 30-80%, about 30-70%, about 30-60%, about 30-50%, about 30-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-80%, about 80-95%, about 80-90/o, or about 90-95%, based on the weight of the lipid-based carrier or pharmaceutical composition.

The lipid-based carrier (or lipid nanoformulation) or pharmaceutical compositions can contain total lipids at an amount of about 5 to about 95% by weight, based on the weight of the lipid-based carrier (or lipid nanoformulation) or pharmaceutical composition. In some embodiments, the lipid-based carrier (or lipid nanoformulation) or pharmaceutical compositions contain total lipids at an amount of about 5-95%, about 5-90%, about 5-80%, about 5-70%, about 5-60%, about 5-50%, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30-95%, about 30-90%, about 30-80%, about 30-70%, about 30-60%, about 30-50%, about 30-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-80%, about 80-95%, about 80-90%, or about 90-95%, based on the weight of the lipid-based carrier or pharmaceutical composition.

In some embodiments, the pharmaceutical compositions can be formulated for parenteral administration, including intracanalicular administration, intravenous administration, subcutaneous administration, or intramuscular administration.

As used herein, the term “parenteral” refers to routes of administration aside from enteral administration. Examples of parenteral administration include, without limitation, buccal, epicutaneous, epidural, extra-amniotic, intra-arterial, intra-articular, intracardiac, intracavernous, intracerebral, intracerebroventricular, intradermal, intralesional, intramuscular, intraocular, intraosseous infusion, intraperitoneal, intrapulmonary, intrathecal, intrauterine, intravaginal, intravenous, intravesical, intravitreal, nasal, perivascular, subcutaneous, sublingual, transdermal, topical, transepithelial, or transmucosal. Parenteral administration may be by continuous infusion over a selected period of time.

In some embodiments, the pharmaceutical compositions are administered intravenously by a bolus injection or infusion. Suitable formulations for use may are found in Remington's Pharmaceutical Sciences, Mack Publishing Company, Philadelphia, Pa., 17th ed. (1985), which is incorporated herein by reference in its entirety.

In some embodiments, the pharmaceutical composition is formulated for injection, such as intravenous infusion A sterile injectable composition, e.g., a sterile injectable aqueous or oleaginous suspension, can be formulated according to techniques known in the art using suitable dispersing or wetting agents (such as Tween 80) or suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically.

Any of the pharmaceutical compositions described herein can be used for delivering an active molecule or therapeutic agent (such as a nucleic acid molecule) encapsulated in the lipid-based carrier (or lipid nanoformulation) to a desired target. To practice this use, an effective amount of a pharmaceutical composition as described herein can be administered to a subject in need of the treatment (e.g., a human subject) via a suitable route, such as those described herein

The disclosure also provides dosage units containing the lipid-based carriers or pharmaceutical compositions disclosed herein. One skilled in the art would be able to select a dosage form for use herein. For example, the dosage unit may be a solid dosage form, liquid dosage form, or solid/liquid dosage form. In some embodiments, the dosage unit is a solid dosage form. In some embodiments, the dosage form is a liquid dosage form.

The dosage unit may be formulated for the delivery that is most useful to the subject. In some embodiments, the dosage unit is for enteral or parenteral administration. Examples of enteral administration include, without limitation, oral, rectal, sublingual, or buccal.

In some embodiments, the dosage unit is administered intravenously, intraperitoneally, intramuscularly, or subcutaneously. In some embodiments, the dosage unit is administered orally, intravenously, intraperitoneally, intramuscularly, or subcutaneously. In some embodiments, the dosage unit is administered orally.

In some embodiments, the dosage unit is for parenteral administration, i.e., a parenteral dosage unit. Parenteral dosage units are known in the art and include, without limitation, injectable solutions, inhalants, infusions, patches, and suppositories. In certain aspects, the parenteral dosage unit is an injectable solution. In other embodiments, the dosage unit is formulated for oral delivery, i.e., an oral dosage unit. In certain aspects, the oral dosage unit is a pill (e.g., tablet, caplet, capsule (e.g., soft gelatin, hard gelatin, gel capsule)), effervescent dosage form, elixir, film, liquid/solution (e.g., suspension, emulsion), lollipop, lozenge, paste, powder, sachet, or syrup. In some embodiments, the oral dosage unit is a pill, tablet, capsule, syrup, liquid solution, powder, paste, patch, pump, or film. In some embodiments, the oral dosage unit is a dry product for reconstitution with water or other suitable vehicle before use.

When the dosage form is a solid dosage form, an enteric coating can be applied or the solid dosage form may be scored. An enteric coating can be stable at low pH (e.g., in the stomach) and can dissolve at higher pH (e.g., in the small intestine).

Regardless of the type of dosage unit, it contains a therapeutically effective amount of one or more lipid compounds or the lipid nanoformulations described herein. One skilled in the art can determine a suitable amount of the compound to incorporate into the lipid-based carriers or lipid nanoformulation, or pharmaceutical composition, in the dosage units.

In some embodiments, the lipid-based carriers, pharmaceutical compositions, or dosage units contain about 0.01 to about 1000 mg of one or more lipid compounds described herein. In some embodiments, the lipid-based carriers, pharmaceutical compositions, or dosage units contain about 0.01, about 0.1, about 0.5, about 1, about 5, about 10, about 25, about 50, about 75, about 100, about 125, about 150, about 175, about 200, about 225, 250, about 275, about 300, about 350, about 400, about 450, about 500, about 550, about 600, about 650, about 700, about 750, about 800, about 850, about 900, about 950, or about 1000 mg of one or more lipid compounds described herein. In some embodiments, the lipid-based carriers, pharmaceutical compositions, or dosage units contain about 0.01 to about 750 mg, about 0.01 to about 500 mg, about 0.01 to about 250 mg, about 0.01 to about 100 mg, about 0.01 to about 50 mg, about 0.01 to about 25 mg, about 0.01 to about 10 mg, about 0.01 to about 5 mg, about 0.01 to about 0.1 mg, about 0.1 to about 1000 mg, about 0.1 to about 750 mg, about 0.1 to about 500 mg, about 0.1 to about 250 mg, about 0.1 to about 100 mg, about 0.1 to about 50 mg, about 0.1 to about 25, about 0.1 to about 10 mg, about 0.1 to about 5 mg, about 0.1 to about 1 mg, about 1 to about 1000 mg, about 1 to about 750 mg, about I to about 500 mg, about 1 to about 250 mg, about 1 to about 100 mg, about 1 to about 50 mg, about 1 to about 25 mg, about 1 to about 10 mg, about 1 to about 5 mg, about 5 to about 1000 mg, about 5 to about 750 mg, about 5 to about 500 mg, about 5 to about 250 mg, about 5 to about 100 mg, about 5 to about 50 mg, about 5 to about 25 mg, about 5 to about 10 mg, about 10 to about 1000 mg, about 10 to about 750 mg, about 10 to about 500, about 10 to about 250 mg, about 10 to about 100 mg, about 10 to about 50 mg, about 10 to about 25 mg, about 25 to about 1000 mg, about 25 to about 750 mg, about 25 to about 500 mg, about 25 to about 250 mg, about 25 to about 100 mg, about 25 to about 50 mg, about 50 to about 1000, mg about 50 to about 750 mg, about 50 to about 500 mg, about 50 to about 250 mg, about 50 to about 100 mg, about 100 to about 1000 mg, about 100 to about 750 mg, about 100 to about 500 mg, about 100 to about 250 mg, about 250 to about 1000 mg, about 250 to about 750 mg, about 250 to about 500 mg, about 500 to about 1000 mg, about 500 to about 750 mg, or about 750 to about 1000 mg of one or more lipid compounds described herein.

### Methods of Using the Pharmaceutical Composition

Certain aspects of the invention also relates to various methods of using the pharmaceutical composition described herein

All above descriptions and all embodiments discussed in the above aspects relating to the aspects of the lipid compounds, including the compounds covered by formulas (la), (AL-GI), (AL-Ia)-(AL-Ic), (AL-IIa)-(AL-IIc), (AL-IIIa)-(AL-IIIg), (AL-IVa)-(AL-IVc), and the exemplary formulas for lipids having at least two ester group, lipids containing a lactide (or its derivative) group, and lipids containing a phosphoramidate group are all applicable to these aspects of the invention relating to various methods of using the pharmaceutical composition described herein.

All above descriptions and all embodiments discussed in the above aspects relating to the aspects of the lipid-based carrier (or lipid nanoformulation), including various other lipid components, are applicable to these aspects of the invention relating to various methods of using the pharmaceutical composition described herein.

All above descriptions and all embodiments discussed in the above aspects relating to the aspects of the pharmaceutical composition, including various aspects of the therapeutic agents and other ingredients, are applicable to these aspects of the invention relating to various methods of using the pharmaceutical composition described herein.

Some embodiments relate to a method for delivering a therapeutic agent (encapsulated in a lipid-based carrier, or a pharmaceutical composition described herein) to a cell or cells in a subject or organism, the method comprising administering the pharmaceutical composition described herein (containing the lipid-based carrier which contains the lipid compound described herein), under conditions suitable for delivery of the pharmaceutical composition described herein to the cell or cells of the subject or organism.

Some embodiments relate to a method for modulating the expression of a target gene within a cell comprising, introducing the pharmaceutical composition described herein (containing the lipid-based carrier which contains the lipid compound described herein and a therapeutic agent such as a nucleic acid, e.g., mRNA) into a cell under conditions suitable to modulate the expression of the target gene in the cell. In one embodiment, the cell is a liver cell (e.g., hepatocyte).

Some embodiments relate to a method for modulating the expression of more than one target gene within a cell comprising, introducing the pharmaceutical composition described herein (containing the lipid-based carrier which contains the lipid compound described herein and a therapeutic agent such as a nucleic acid, e.g., mRNA) into the cell under conditions suitable to modulate the expression of the target genes in the cell. In one embodiment, the cell is a liver cell (e.g., hepatocyte).

Some embodiments relate to a method for expressing an RNA or polypeptide in a subject or organism in need thereof, comprising contacting the subject or organism with the pharmaceutical composition described herein (containing the lipid-based carrier which contains the lipid compound described herein and a therapeutic agent such as a nucleic acid, e.g., the RNA) under conditions suitable to express the RNA or polypeptide in the subject or organism.

Some embodiments relate to a method for preventing or treating a disease, disorder, and/or condition in a subject in need thereof, wherein the disease, disorder, and/or condition may be characterized by missing or aberrant protein or polypeptide activity. The method comprises administering the pharmaceutical composition described herein (containing the lipid-based carrier which contains the lipid compound described herein and a therapeutic agent such as a nucleic acid, e.g., the RNA), wherein the RNA may be an mRNA encoding a poly peptide that antagonizes or otherwise overcomes the aberrant protein or polypeptide activity present in the cell of the subject, thereby preventing or treating the disease, disorder, and/or condition. In one embodiment, the cell is a liver cell (e.g., hepatocyte).

“Treating” or variations thereof refers ameliorating or reducing the development of a disease or disorder, i.e., delaying the onset of the disease. In certain embodiments, “treating” refers to ameliorating or reducing at least one physical parameter of the disease or disorder. In other embodiments, “treating” is directed to improving the disease or disorder. In further embodiments, “treating” is directed to the cause of the disease or disorder. In yet other embodiments, “treating” is directed to relieving symptoms of the disease or disorder. In still further embodiments, “treating” is directed to treating the disease or disorder as a supplement another therapy.

In one embodiment, in any of the above methods, the method comprises contacting the subject or organism with the pharmaceutical composition described herein via local administration to relevant tissues or cells.

In one embodiment, in any of the above methods, the method comprises contacting the subject or organism with the pharmaceutical composition described herein via systemic administration (such as via intravenous or subcutaneous administration of the formulation or composition) to relevant tissues or cells. The formulation or composition of the invention can be formulated or conjugated as described herein or otherwise known in the art to target appropriate tissues or cells in the subject or organism.

In any of the above methods, the pharmaceutical composition described herein can be administered at various time intervals, such as once per day, once every two days, once every three days, once every four days, once every five days, once every six days, once per week, once every other week, once per month, etc. In one embodiment, the administration is once every 1, 2, 3, 4, 5, 6, 7, 8, 9, or 10 weeks.

In any of the above methods, the pharmaceutical composition described herein can be administered to the subject systemically as described herein or otherwise known in the art Systemic administration can include, for example, intravenous, subcutaneous, intramuscular, catheterization, nasopharyngeal, transdermal, or gastrointestinal administration as is generally known in the art.

In one embodiment, in any of the above methods of treatment or prevention, the pharmaceutical composition described herein can be administered to the subject locally or to local tissues as described herein or otherwise known in the art. Local administration can include, for example, catheterization, implantation, osmotic pumping, direct injection, intrathecal, ventricular, dermal/transdermal application, stenting, ear/eye drops, or portal vein administration to relevant tissues, or any other local administration technique, method or procedure, as is generally known in the art.

### Kits

The disclosure also provide kits for use in delivering the pharmaceutical composition to a target site or for use in diagnostic or therapeutic purpose. Such kits can include one or more containers comprising any of the pharmaceutical compositions described herein, and a pharmaceutically acceptable carrier/excipient.

In some embodiments, the kit can comprise instructions for use in accordance with any of the methods described herein. The included instructions can comprise a description of administration of the pharmaceutical composition in according to any of the methods described herein. The kit may further comprise a description of selecting an individual suitable for diagnosis or treatment.

The instructions relating to the use of the pharmaceutical composition described herein, generally include information as to dosage, dosing schedule, and route of administration for the intended treatment. The containers may be unit doses, bulk packages (e.g., multi-dose packages) or sub unit doses. Instructions supplied in the kits are typically written instructions on a label or package insert (e.g., a paper sheet included in the kit), but machine-readable instructions (e.g., instructions carried on a magnetic or optical storage disk) are also acceptable.

The kits as described herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle) The container may also have a sterile access port (for example the container may be an intravenous solution bag or a vial having a stopper pierceable by a hypodermic injection needle) The kits described herein may optionally provide additional components such as buffers and interpretive information Normally, the kit comprises a container and a label or package insert(s) on or associated with the container. In some embodiments, the present disclosure provides articles of manufacture comprising contents of the kits described above.

### EXAMPLES

The following examples are for illustrative purposes only and are not intended to limit, in any way, the scope of the present invention. To the extent that specific materials are mentioned, it is merely for illustrative purpose and is not intended to limit the invention. One skilled in the art may develop equivalent methods or reactants without the exercise of inventive capacity and without departing from the scope of the invention.

### Example 1—General Reactions for the Synthesis of Ionizable Lipid Compounds

General reaction schemes for synthesis of exemplary ionizable lipid compounds, containing various cleavable linkers are shown in Schemes 1-3.

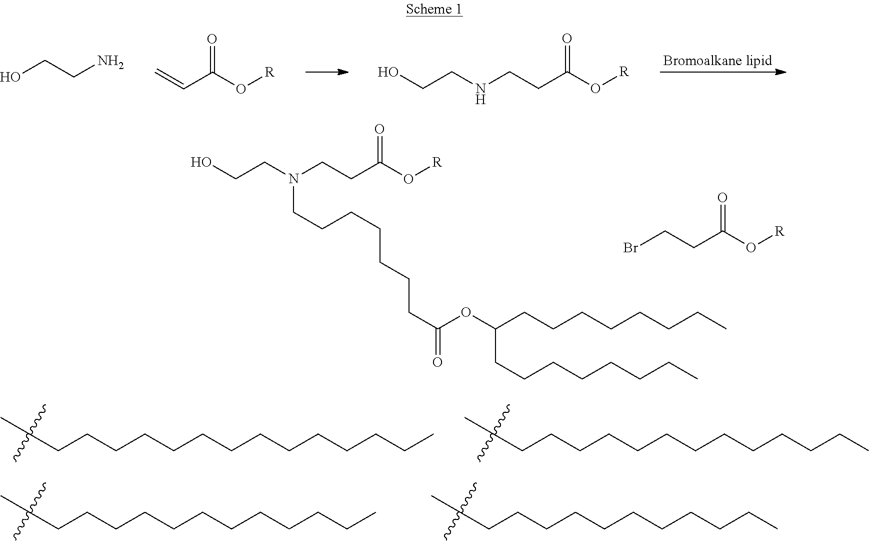

As shown in Scheme 1, the synthesis starts with coupling an acrylated lipid with ethanolamine, which is then alkylated with a brominated lipid to produce the final product, containing an ester cleavable linker at the lipid tails. Both cis and trans stereoisomers can be synthesized.

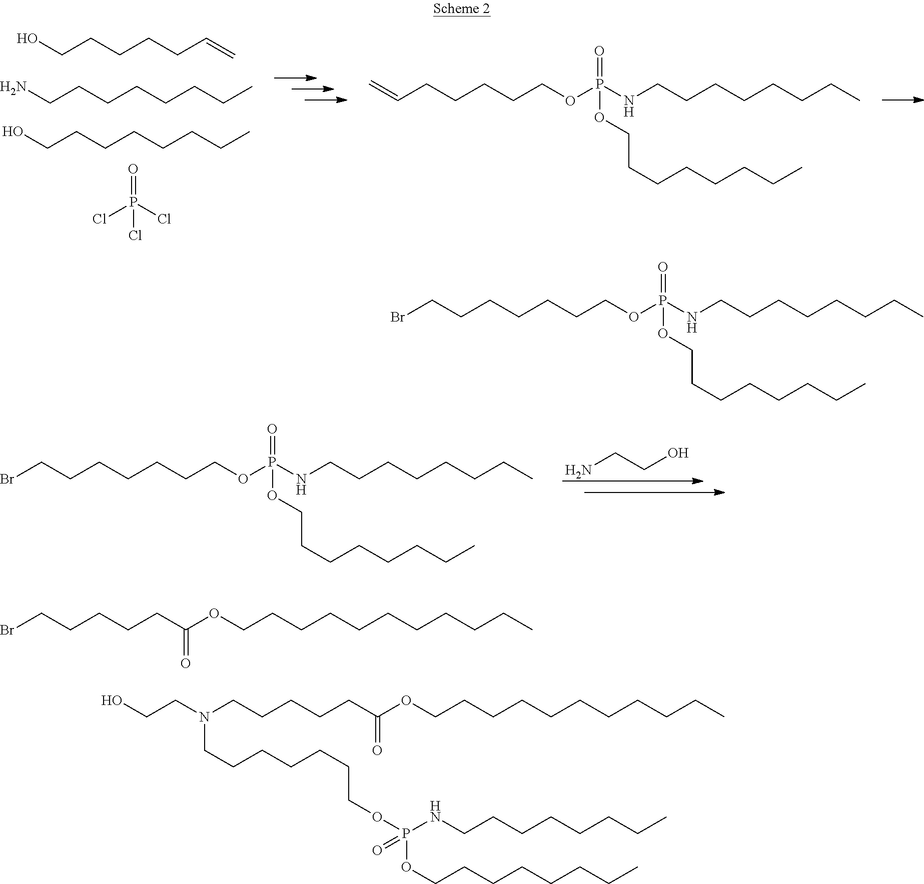

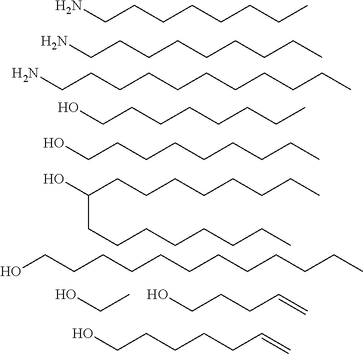

As shown in Scheme 2, the synthesis starts with functionalization of POCl₃ with an alkenyl alcohol, an alkyl alcohol, and an amine to generate an alkenyl phosphoramidate. The intermediate compound is then hydrobrominated to form a brominated phosphoramidate lipid, which is then reacted with ethanolamine and another brominated lipid to form the final product. Both cis and trans stereoisomers can be synthesized.

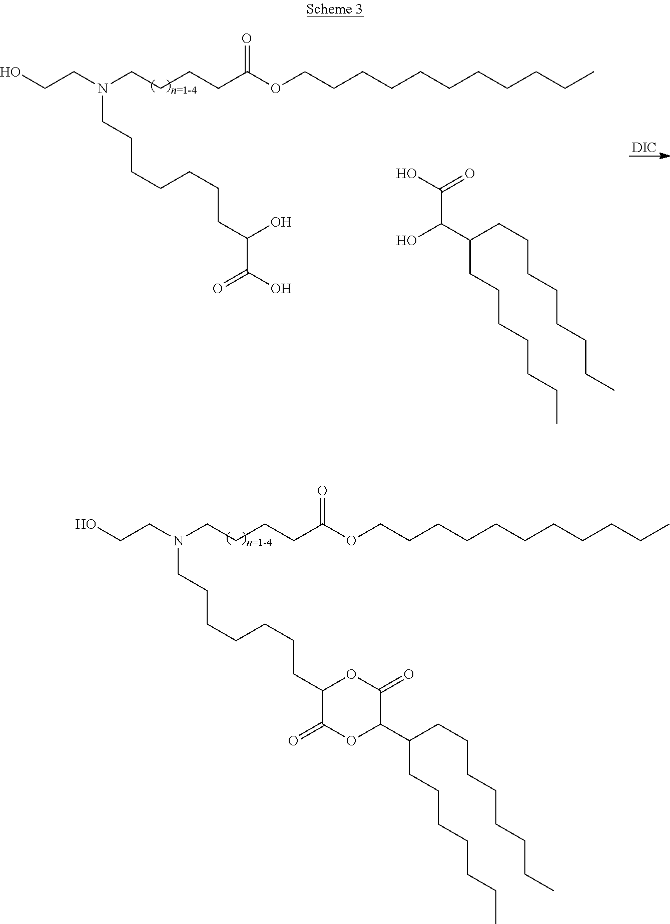

As shown in Scheme 3, the synthesis is based on coupling two lipid building blocks, each having a lactate-like unit, via a coupling agent (such as N,N′-diisopropylcarbodiimide) to cyclize the two lipid building blocks to form the final product. Both cis and trans stereoisomers can be synthesized.

### Example 2—Preparation of Exemplary Lipid Nanoparticle Compositions

Lipid nanoparticle compositions including a therapeutic composition can be prepared by selection of a lipid compound described herein (e.g., those covered by formulas (AL-GI), (AL-Ia)-(AL-Ic), (AL-IIa)-(AL-IIc), (AL-IIIa)-(AL-IIIg), (AL-IVa)-(AL-IVc), and the exemplary formulas for lipids having at least two ester group, lipids containing a lactide (or its derivative) group, and lipids containing a phosphoramidate group), the selection of additional lipids, the amount of each lipid in the lipid component, and the wt:wt ratio of the lipid component, as described herein.

### Preparation of Lipid Nanoparticles:

To obtain various characteristics of a lipid nanoparticle, the particular elements and ratios of the lipid nanoparticle (LNP) are selected. The lipid nanoparticle formulation recipe for a LNP compositions includes various molar ratios of ionizable lipid, cholesterol, phospholipid (or another structural lipid), PEG-lipid, and/or additional components (e.g., RNA payload). The LNP composition formulations are evaluated to determine characteristics such as encapsulation, size, zeta potential, apparent pKa, and in vitro/in vivo protein expression levels.

In one example, molar ratios of ionizable lipid/sterol/phospholipid (or another structural lipid)/PEG-lipid/additional components is varied in the following ranges: ionizable lipid (25-100%); sterol (0-50%): phospholipid (DSPC) (0-40%); and PEG lipid (0-5%). Nitrogen:phosphate ratios (N:P ratio) is evaluated at values between 0.1 and 100.

### Lipid Nanoparticle Formulation Procedure:

In one example, an ionizable lipid, structural lipid (e.g. phospholipids such as DSPC), sterol (e.g. cholesterol) and PEG lipid (e.g. DSPE PEG 2k) is individually dissolved in ethanol. The separate lipid solutions is combined by pipette mixing at molar ratios of ionizable lipid/cholesterol/DSPC/DSPE PEG 2k of 50/38.5/10/1.5, producing a lipid stock solution. The nucleic acid (e.g., mRNA) is diluted in a buffer (e.g., 10 mM citrate) and mixed with the ethanol lipid solution via syringe pump or microfluidic mixing. The resulting LNP composition is collected for further processing.

In another example, the NanoAssembir® Ignite™ (Precision Nanosystems) system is utilized. For instance, the lipid solution containing molar ratios of the lipids such as that described above, is loaded into a syringe. The mRNA solution (0.25 mg/mL) is prepared using citrate buffer (10 mM, pH 4) and loaded into a syringe. The lipid and RNA solutions are mixed at 2.5 and 7.5 mL/minute using the NanoAssembler® microfluidics chip. The resulting nanoparticles are in a buffer to ethanol ratio of 3:1 and ready for further downstream processing and purifications.

### Processing/Purification

The processing of LNP compositions is important for maintaining sizes and physical properties of the formulated lipid nanoparticles. Storage buffers and residual ethanol can affect lipid nanoparticle stability. Processing can remove the ethanol present upon formulation and allow for buffer exchange.

After formulation, the LNP composition is allowed to sit for 30 minutes and is then diluted 1:1 with deionized water. To purify and concentrate the lipid nanoparticle composition, the solution will be loaded onto a desalting column (e.g., a PD-10 Sepharose desalting column) to exchange the buffer into 1×PBS, and subsequently concentrated to approximately 0.5 mg/ml (nucleotide cargo) on an Amicon centrifugal spin filter. Finally, the LNP composition is filtered through a 0.2 μm filter.

### Example 3—Characterization of Lipid Nanoparticle Compositions

To investigate the safety and efficacy of the lipid nanoparticle compositions for use in the delivery of a therapeutic molecule to cells, the various lipid nanoparticle formulations are tested and characterized.

Various lipid nanoparticle compositions comprising a nucleic acid payload are characterized by a variety of methods. For example, dynamic light scattering (DLS) is used to determine particle sizes (Wyatt Dynapro Platereader 111). In addition, instruments such as the Zetasizer Nano ZS (Malvern Instruments Ltd) are used to measure multiple physical properties of a lipid nanoparticle composition, such as particle size, polydispersity index (PDI), and zeta potential. Fluorescence-based assays are used to determine the efficiencies of nucleic acid encapsulation in the lipid nanoparticles. Fluorescence-based assays are used to determine the “apparent pKa” of the lipid nanoparticles. Both fluorescence assays are evaluated on a Varioskanlux plate reader (ThermoScientific).

### Nanoparticle Size

After formulation and processing of the lipid nanoparticle compositions, the Z-average size of lipid nanoparticle composition are assayed by dynamic light scattering (DLS) (e.g., using a Wyatt Dynapro Platereader III or Malvern Zetasizer ZS). Briefly, an aliquot of 2 uL of the processed nanoparticle composition mixture (at an RNA concentration of approximately 0.5 mg/mL) is diluted into 50 uL phosphate buffered saline (0.1×PBS) and loaded into a 384 well plate. The sample is then analyzed to determine nanoparticle size.

### Encapsulation Efficiency Assay/Ribogreen Assay

For lipid nanoparticle formulations containing an RNA, a QUANTIT™ RIBOGREEN® RNA assay (Invitrogen Corporation) is used to evaluate the encapsulation of RNA within the lipid nanoparticle formulation.

The samples are diluted to ˜10 ug/ml in TE buffer. In a 96 well plate, 50 ul of the diluted sample is added to 50 uL TE buffer in duplicates and 20 uL of diluted sample is added to 80 uL Triton X buffer (2% triton in TE buffer) in duplicates. The plate is incubated for 10 minutes at room temperature. Next, RIBOGREEN® is diluted 1:200 in TE BUFFER, and 100 uL is added to each sample well. The fluorescence intensity is measured using a fluorescence plate reader (e.g., Varioskanlux, ThermoScientific) at an excitation wavelength of about 480 nm and an emission wavelength of about 520 nm. The encapsulation efficiency can be determined by using the percentage ratio of non-encapsulated RNA (TE sample) over the total RNA (TX sample), then subtracting from 100 to find the percentage of encapsulated RNA amount.

TNS Assay/Protocol to Determine “Apparent pKa”

The apparent pKa of lipid nanoparticle composition is measured using a 6-(p-Toluidino)-2-naphthalenesulfonic acid (TNS) assay. Briefly, lipid nanoparticles (10 uL of approximately 0.5 mg mRNA/mL) and TNS probe (40 uL of 1 mM stock) are added to 750 uL of TNS buffer (25 mM citric acid, 20 mM sodium phosphate, 150 mM NaCl, and 20 mM ammonium acetate), at a pH ranging from 2 to 10. 20 uL of each sample are added to 80 uL of buffer at each pH in a 96-well plate. The plate is incubated for 10 minutes, and the fluorescence signal is measured by means of a spectrofluorometer (Varioskanlux, ThermoScientific) with excitation/emission settings of λₑₓ=321 nm, and λₑₘ=445 nm. The apparent pKa of the nanoparticle composition is determined by deriving the pH at which 50% of the pH 2 fluorescent signal is present. All datapoints are normalized to the fluorescent signal at pH 2.

### Zeta Potential Assay

A Zetasizer Nano ZS (Malvern Instruments Ltd) is used to determine the zeta potential of the nanoparticle compositions. Briefly, a 5 uL aliquot of an approximately 0.5 mg mRNA/mL sample is diluted into 800 uL 0.1×PBS. This sample is then transferred to a disposable conducting cuvette and the zeta potential is measured.

### Example 4—In Vitro and In Vivo Testing of Lipid Nanoparticle Compositions

For both in vitro and in vivo settings, lipid nanoparticle compositions including polynucleotides such as mRNA are useful in the evaluation of the efficacy and biological activity of various lipid nanoparticle formulations. Higher levels of protein expression generated by administration of a formulation including an mRNA will be indicative of higher mRNA translation and/or lipid nanoparticle mRNA delivery efficiencies.

Following administration of lipid nanoparticle compositions to mice or cells, dose delivery profiles, dose responses, and toxicity of particular formulations and the doses used are measured by enzyme-linked immunosorbent assays (ELISA), bioluminescent imaging, fluorescence assisted cell sorting (FACS), or other methods. For lipid nanoparticle compositions including mRNA, time courses of protein expression can also be assessed. Samples used for analysis may include cell suspension, supernatant, or adhered cells. For in vivo work, samples collected from the rodents for evaluation may include blood, sera, and tissue (e.g. muscle tissue from the site of an intramuscular injection or harvested organs); sample collection may involve sacrifice of the animals.

In Vitro hEPO Assay

Briefly, 10,000 cells from selected cell lines (e.g. immortalized cells such as HeLas and primary cells such as PBMCs) are plated in 96 well plates and kept in an incubator overnight. Media is replaced with Opti-MEM and cells are treated with hEPO-mRNA-containing lipid nanoparticles at doses ranging from 500 ng/well to 50 pg/well in triplicates for dose response evaluations. After a 6 hour incubation, 20 uL of supernatant is collected from each well for quantitation using EPO Human ProQuantum Immunoassay Kit (ThermoFisher Scientific) following the manufacturer's protocol. After 24 hours, cellular toxicities are evaluated via fluorescence-based assays (such as the CellTiter-Fluor™ Cell Viability Assay, Promega) using a spectrofluorometer (Varioskanlux, ThermoScientific). The expression levels of EPO are indicative of the protein expression generated by administration of a particular lipid nanoparticle formulation. The cellular toxicity is also informative of the therapeutic potential of the particular lipid nanoparticle formulation.

### In Vivo EPO Assay

Mice are intravenously, intramuscularly, intraarterially, or intratumorally administered a single dose including a lipid nanoparticle composition with a formulation such as those provided in Example 2. Doses may range from 0.001 mg/kg to 10 mg/kg, where 10 mg/kg describes a dose including 10 mg of a polynucleotide in a lipid nanoparticle composition for each 1 kg of body mass of the mouse. A control composition including PBS is used.

In one example, groups of mice (e.g., n=5) are injected via tail vein (IV), or intramuscularly (IM), and imaged via IVIS (luciferase mRNA) at 6, 24 and 48 hours post injection. In another example, secreted protein levels of Human erythropoietin (EPO mRNA) will be measured from blood draws at 6, 24 and 48 hours post injection, and assayed for concentration of EPO by ELISA. Blood/serum samples will allow assessment of liver enzyme levels such as ALT/AST and will be measured by ELISA. Blood/serum samples will also be used to assay cytokine production, which will be measured by Luminex (TRONSITE-LX200, Millipore sigma).

### Example 5: Synthesis of Common Intermediates

Compounds A-G were synthesized in bulk then used as intermediates throughout these experimental procedures. For the synthesis of each Lipid (Example 6) compound numbering starts with Compound 1.

### Compound A:

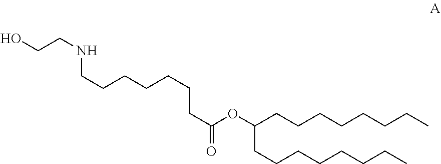 [SMILES: CCCCCCCCC(CCCCCCCC)OC(=O)CCCCCCCNCCO]

### Synthesis of Compound A:

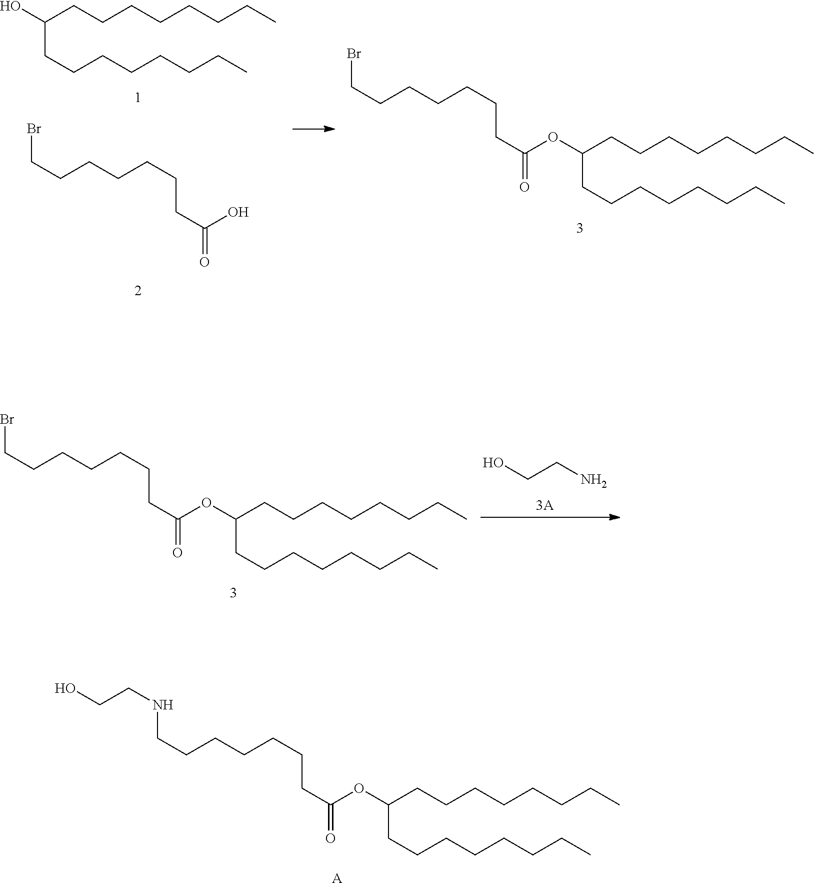

### Preparation of Compound 3:

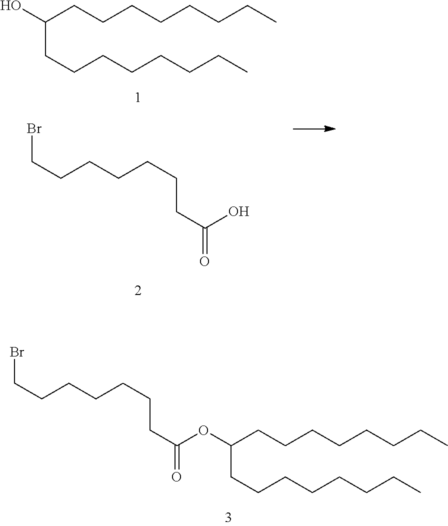 [SMILES: CCCCCCCCC(O)CCCCCCCC ; O=C(O)CCCCCCCBr ; CCCCCCCCC(CCCCCCCC)OC(=O)CCCCCCCBr]

To a solution of Compound 1 (9 g, 35.09 mmol, 1 eq.) in DCM (90 mL) was added Compound 2 (11.74 g, 52.64 mmol, 1.5 eq.), DMAP (857.43 mg, 7.02 mmol, 0.2 eq.), EDCI (8.07 g, 42.11 mmol, 1.2 eq.) and DIEA (9.07 g, 70.18 mmol, 12.22 mL, 2 eq.). The resulting mixture was stirred at 20° C. for 16 hours. TLC indicated Compound 1 was consumed and one major new spot was detected. The reaction mixture was diluted with DCM (100 mL) and washed with NH₄Cl (30 mL*2), dried over Na₂SO₄, filtered and concentrated under reduced pressure to give a residue. The residue was purified by column chromatography (SiO₂, Petroleum ether) to give Compound 3 (6.25 g, 13.54 mmol, 38.59% yield, 100% purity) as white solid.

¹H NMR (400 MHz, CHLOROFORM-d) δ=4.92-4.83 (m, 1H), 3.56-3.50 (m, 1H), 3.43-3.36 (m, 1H), 2.31-2.26 (m, 2H), 1.90-1.72 (m, 2H), 1.70-1.59 (m, 2H), 1.55-1.39 (m, 6H), 1.39-1.05 (m, 28H), 0.91-0.85 (m, 6H)

### Preparation of Compound A:

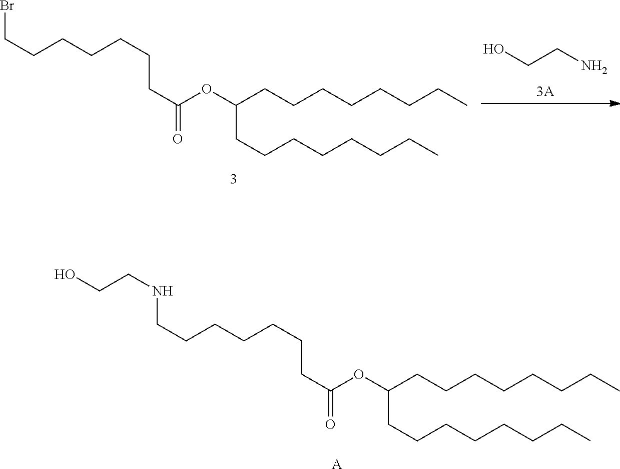

To a solution of Compound 3 (3 g, 6.50 mmol, 1 eq.) in ACN (30 mL) was added Compound 3A (2.78 g, 45.50 mmol, 2.75 mL, 7 eq.), K₂CO₃ (1.80 g, 13.00 mmol, 2 eq.) and KI (1.19 g, 7.15 mmol, 1.1 eq.). The resulting mixture was heated to 80° C. for 16 hours. LCMS showed Compound 3 was consumed and desired MS was detected. The reaction mixture was filtered and the filtrate was concentrated to give a residue. The residue was purified by column chromatography (SiO₂, Dichloromethane:Methanol=40:1 to 4:1) to give Compound A (1.7 g, 3.85 mmol, 59.21% yield, 100% purity) as yellow oil. LCMS [M+1]⁺=442.5

¹H NMR (400 MHz, CHLOROFORM-d) δ=4.91-4.83 (m, 1H), 3.71-3.65 (m, 2H), 3.03-2.91 (m, 2H), 2.85-2.80 (m, 2H), 2.70-2.63 (m, 2H), 2.32-2.25 (m, 2H), 1.68-1.58 (m, 2H), 1.55-1.50 (m, 4H), 1.37-1.22 (m, 32H), 0.92-0.85 (m, 6H)

### Compound B:

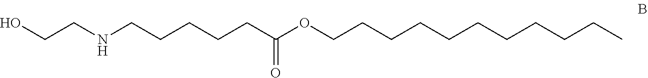 [SMILES: CCCCCCCCCCCOC(=O)CCCCCNCCO]

### Synthesis of Compound B:

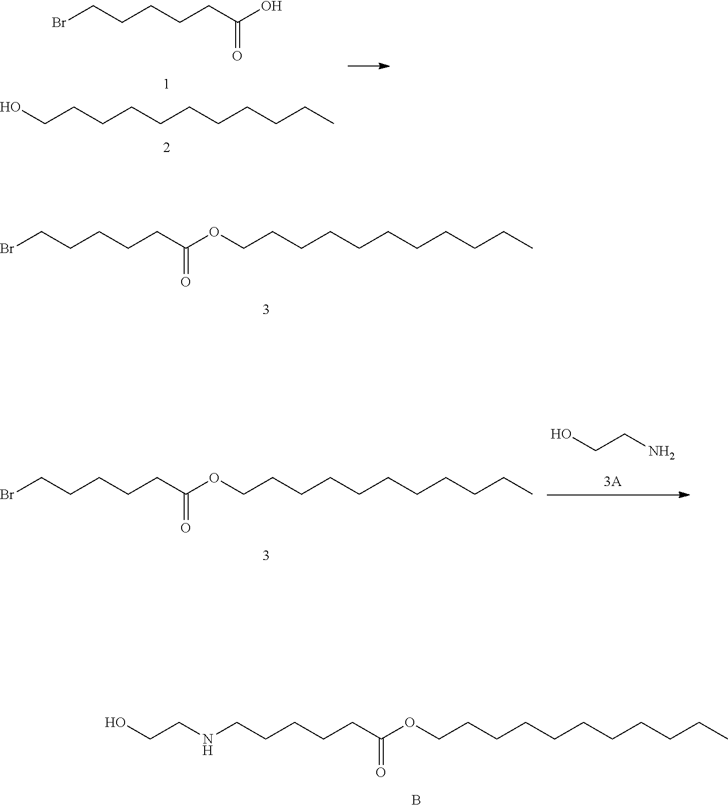

### Preparation of Compound 3:

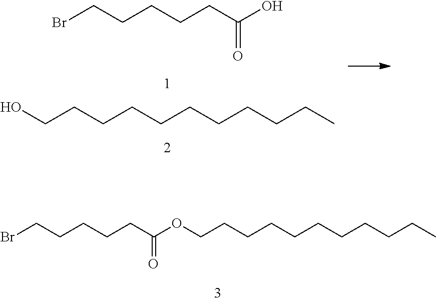 [SMILES: O=C(O)CCCCCBr ; CCCCCCCCCCCO ; CCCCCCCCCCCOC(=O)CCCCCBr]

To a solution of Compound 2 (2 g, 11.61 mmol, 1 eq.) in toluene (20 mL) was added Compound 1 (2.72 g, 13.93 mmol, 1.2 eq.) and 4-methylbenzene sulfonic acid (999.39 mg, 5.80 mmol, 0.5 eq.), and the mixture was heated to 50° C. for 16 hours under N₂ atmosphere. LCMS indicated Compound 2 was consumed completely and one main peak with desired MS was detected. The residue was diluted with H₂O (50 mL) and extracted with EA (50 mL*3). The combined organic layers were dried over Na₂SO₄, filtered and concentrated under reduced pressure to give a residue. The residue was purified by column chromatography (SiO₂, Petroleum ether: Ethyl acetate=99:1 to 95:5) to give Compound 3 (4.25 g, crude) as colorless oil.

LCMS [M+1]⁺=348.2

1H NMR (400 MHz, CHLOROFORM-d) δ=4.07 (t, J=6.8 Hz, 2H), 3.42 (t, J=6.8 Hz, 2H), 2.33 (t, J=7.2 Hz, 2H), 1.94-1.84 (m, 2H), 1.71-1.58 (m, 4H), 1.55-1.43 (m, 2H), 1.36-1.23 (m, 16H), 0.93-0.85 (m, 3H)

### Preparation of Compound B:

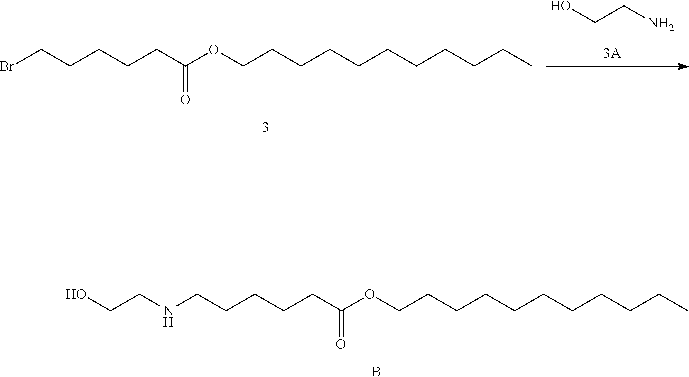

A mixture of Compound 3 (5 g, 14.31 mmol, 1 eq.), Compound 3A (4.37 g, 71.56 mmol, 4.32 mL, 5 eq.), K₂CO₃ (3.96 g, 28.62 mmol, 2 eq.) and KI (237.59 mg, 1.43 mmol, 0.1 eq.) in ACN (20 mL) was degassed and purged with N₂ for 3 times, and then the mixture was stirred at 80° C. for 16 hours under N₂ atmosphere. LCMS indicated Compound 3 was consumed completely and one main peak with desired MS was detected. The residue was diluted with H₂O (50 mL) and extracted with DCM (100 mL*3). The combined organic layers were dried over Na₂SO₄, filtered and concentrated under reduced pressure to give a residue. The residue was purified by column chromatography (SiO₂. DCM:MeOH=50:1 to 4:1) to give Compound B (3.13 g, 9.50 mmol, 66.37% yield) as white solid.

LCMS [M+1]⁺=330.4

1H NMR (400 MHz, CHLOROFORM-d) δ=4.06 (t, J=6.8 Hz, 2H), 3.69-3.60 (m, 2H), 2.82-2.76 (m, 2H), 2.65 (t, J=7.2 Hz, 2H), 2.31 (t, J=7.6 Hz, 2H), 1.70-1.58 (m, 4H), 1.53 (quin, J=7.2 Hz, 2H), 1.44-1.20 (m, 18H), 0.94-0.85 (m, 3H)

### Compound C:

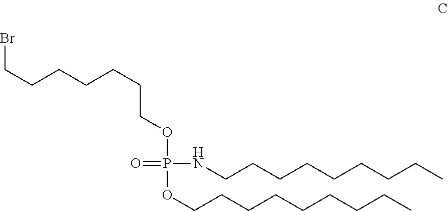 [SMILES: CCCCCCCCCNP(=O)(OCCCCCCCBr)OCCCCCCCCC]

### Compound D:

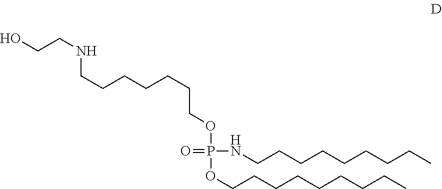 [SMILES: CCCCCCCCCNP(=O)(OCCCCCCCCC)OCCCCCCCNCCO]

### Synthesis of Compound D:

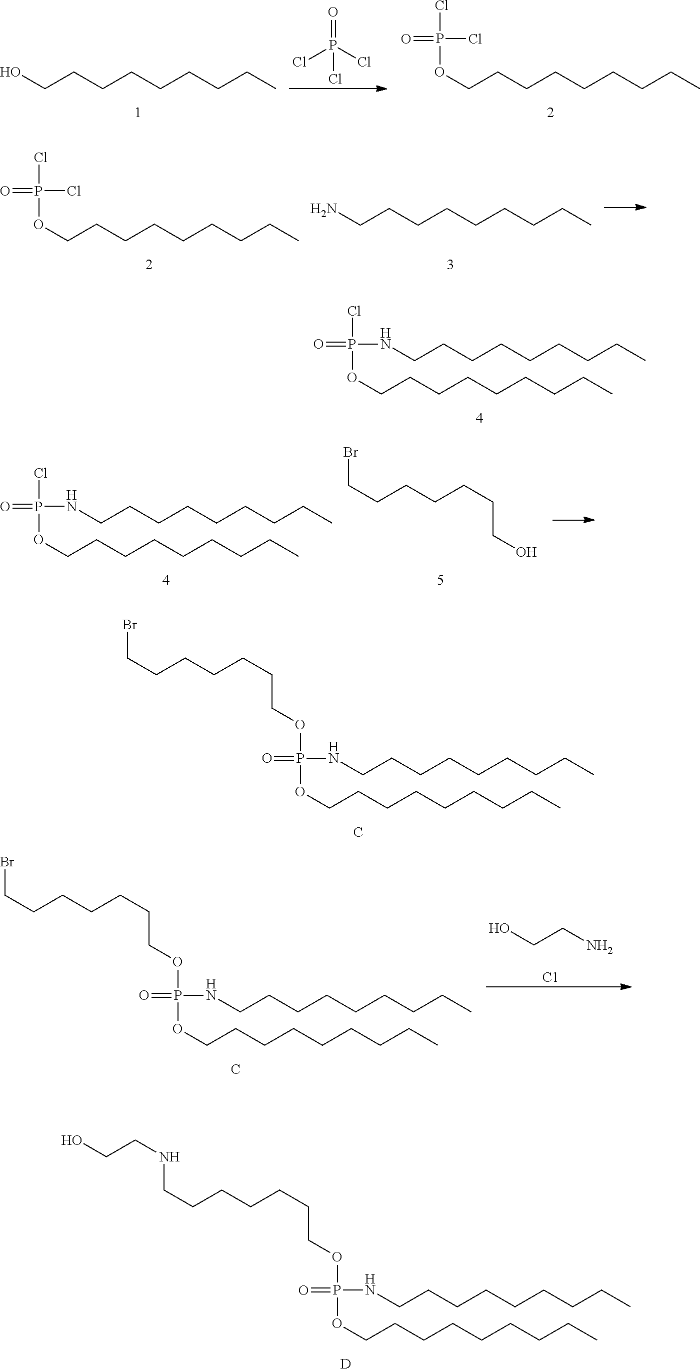

### Preparation of Compound 2:

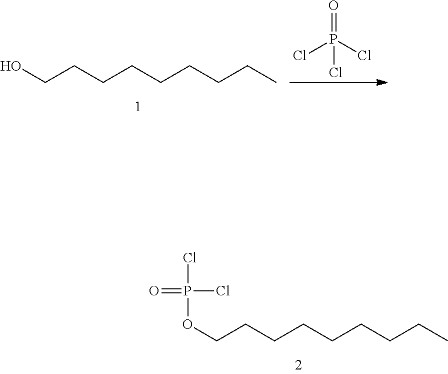

POCl₃ (10.63 g, 69.32 mmol, 6.46 mL, 2 eq.) was added dropwise to Compound 1 (5 g, 34.66 mmol, 1 eq.) at 20° C. After addition, the mixture was stirred at this temperature for 16 hours. TLC indicated Compound 1 was consumed, and one major new spot was detected. The reaction mixture was concentrated under reduced pressure to give Compound 2 (7 g, 26.81 mmol, 77.34% yield, 100% purity) as yellow oil, which was used without further purification)

¹H NMR (400 MHz, CHLOROFORM-d) δ=4.39-4.30 (m, 2H), 1.86-1.76 (m, 2H), 1.48-1.21 (m, 12H), 0.93-0.85 (m, 3H)

### Preparation of Compound 4:

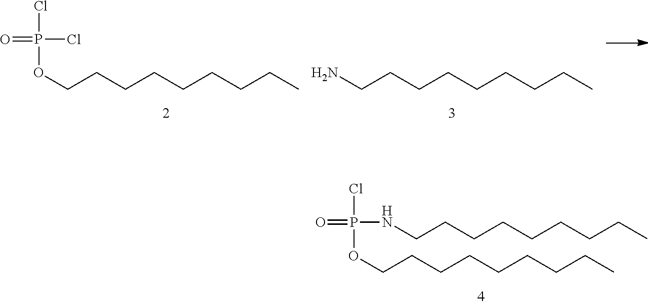

To a solution of Compound 2 (5 g, 19.15 mmol, 1 eq.) in Tol. (100 mL) was added Compound 3 (2.19 g, 15.32 mmol, 0.8 eq.) and TEA (1.55 g, 15.32 mmol, 2.13 mL, 0.8 eq.). The resulting mixture was stirred at 20° C. for 2 hours. TLC indicated Compound 2 was consumed, and one major new spot was detected. The reaction mixture was diluted with sat.NH₄Cl (50 mL) and extracted with ethyl acetate (50 mL*2), dried over Na₂SO₄, filtered and concentrated under reduced pressure to give a residue. The residue was purified by column chromatography (SiO₂, Petroleum ether: Ethyl acetate=100:1 to 10:1) to give Compound 4 (2.5 g, 6.79 mmol, 35.49% yield, 100% purity) as white solid.

LCMS [M+1]⁺=368.3

¹H NMR (400 MHz, CHLOROFORM-d) δ=4.06 (br s, 2H), 3.29-3.09 (m, 1H), 3.07-2.96 (m, 2H), 1.79-1.68 (m, 2H), 1.59-1.50 (m, 2H), 1.46-1.17 (m, 24H), 0.99-0.81 (m, 6H)

### Preparation of Compound C:

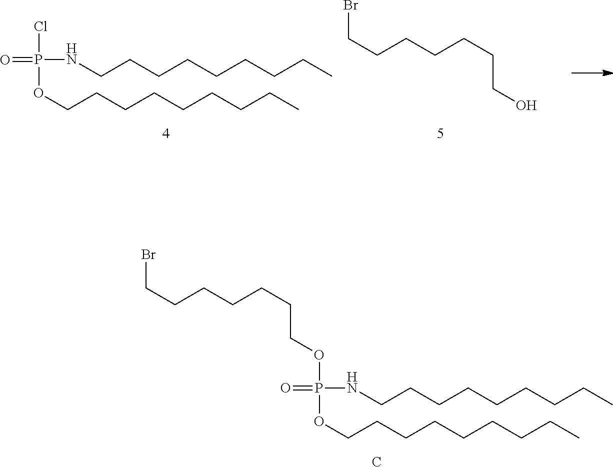

To a solution of Compound 4 (2.3 g, 6.25 mmol, 1 eq.) in DCM (14 mL) was added Compound 5 (1.34 g, 6.88 mmol, 1.1 eq.) and TEA (1.27 g, 12.50 mmol, 1.74 mL, 2 eq.). The resulting mixture was stirred at 30° C. for 16 hours. LCMS showed >90% of Compound 4 was consumed, and several new peaks were shown on LCMS. The reaction mixture was diluted with sat. NH₄Cl (20 mL) and extracted with DCM (10 mL*2), dried over Na₂SO₄, filtered, and concentrated under reduced pressure to give a residue. The residue was purified by reversed-phase HPLC (column: Welch Xtimate C1 100*30 mm*5 um; mobile phase: [H₂O (10 mM NH₄HCO₃)-THF:ACN=1:3]; gradient: 50/6-80% B over 20.0 min) to give Compound C (0.52 g, 986.04 mol, 15.77% yield, 99.85% purity) as yellow oil.

LCMS [M+1]⁺=527.3, LCMS [2M+1]⁺=1053.5

¹H NMR (400 MHz, CHLOROFORM-d) δ=4.06-3.91 (m, 4H), 3.41 (t, J=6.8 Hz, 2H), 2.94-2.83 (m, 2H), 2.51-2.40 (m, 1H), 1.91-1.77 (m, 2H), 1.73-1.64 (m, 4H), 1.54-1.39 (m, 6H), 1.38-1.21 (m, 26H), 0.89 (t, J=6.8 Hz, 6H)

### Preparation of Compound D:

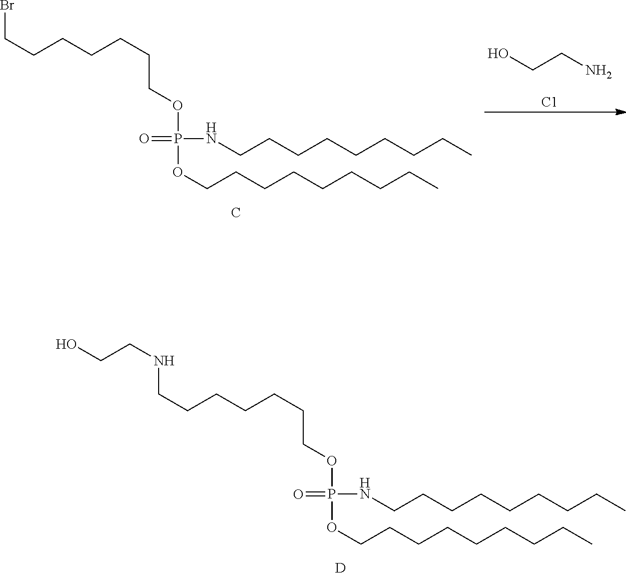

To a solution of Compound C (2 g, 3.80 mmol, 1 eq.) in EtOH (20 mL) was added Compound C1 (6.96 g, 113.95 mmol, 6.88 mL, 30 eq.). The mixture was heated to 80° C. for 16 hours. LCMS showed Compound C was consumed completely and one main peak with desired MS was detected. The residue was diluted with H₂O (15 mL) and extracted with ethyl acetate (10 mL*3), dried over Na₂SO₄, filtered and concentrated under reduced pressure to give a residue. The residue was purified by column chromatography (SiO₂, DCM:MeOH=50:1 to 3:1) to give Compound D (1.34 g, 2.64 mmol, 69.62% yield) as yellow oil.

LCMS [M+1]⁺=507.5

¹H NMR (400 MHz, CHLOROFORM-d) δ=4.04-3.89 (m, 4H), 3.72-3.57 (m, 2H), 2.93-2.76 (m, 4H), 2.71-2.53 (m, 6H), 1.72-1.62 (m, 4H), 1.58-1.44 (m, 4H), 1.42-1.21 (m, 30H), 0.88 (t, J=6.8 Hz, 6H)

### Compound E:

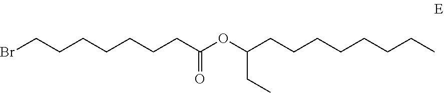 [SMILES: CCCCCCCCC(CC)OC(=O)CCCCCCCBr]

### Preparation of Compound E:

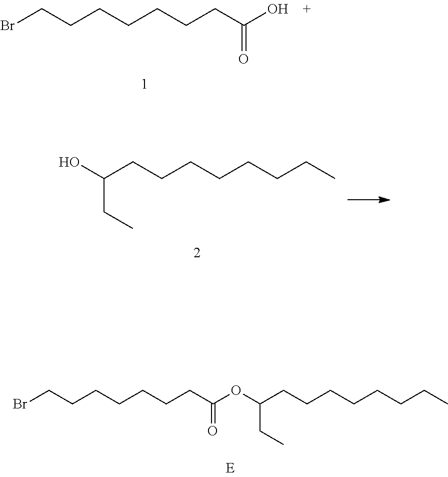 [SMILES: O=C(O)CCCCCCCBr ; CCCCCCCCC(O)CC ; CCCCCCCCC(CC)OC(=O)CCCCCCCBr]

To a solution of Compound 1 (10 g, 44.82 mmol, 1 eq.) and Compound 2 (9.27 g, 53.79 mmol, 1.2 eq.) in DCM (100 mL) was added EDCI (10.31 g, 53.79 mmol, 1.2 eq.), DMAP (1.10 g, 8.96 mmol, 0.2 eq.) and DIEA (11.59 g, 89.64 mmol, 15.61 mL, 2 eq.). The mixture was stirred at 20° C. for 16 hours. TLC (Petroleum ether: Ethyl acetate=10:1, R=0.5) indicated Compound 1 was consumed completely and one new spot formed. The reaction mixture was diluted with DCM (100 mL) and washed with H₂O (200 mL). The combined organic layers were dried over Na₂SO₄, filtered and concentrated under reduced pressure to give a residue. The residue was purified by column chromatography (SiO₂, Petroleum ether: Ethyl acetate=1:0 to 300:1) to give Compound E (8.7 g, 22.82 mmol, 50.92% yield, 99% purity) as colorless oil.

¹H NMR (400 MHz, CHLOROFORM-d) δ=4.83-4.67 (m, 1H), 3.37-3.30 (m, 2H), 2.25-2.18 (m, 2H), 1.78 (quin, J=7.2 Hz, 2H), 1.60-1.52 (m, 2H), 1.46-1.40 (m, 3H), 1.39 (s, 2H), 1.30 (s, 5H), 1.23-1.14 (m, 12H), 0.84-0.77 (m, 6H)

### Compound F:

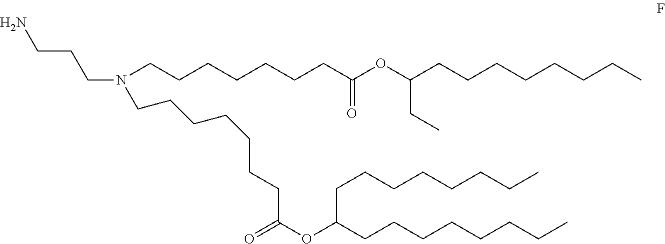 [SMILES: CCCCCCCCC(CC)OC(=O)CCCCCCCN(CCCN)CCCCCCCC(=O)OC(CCCCCCCC)CCCCCCCC]

### Synthesis of Compound F:

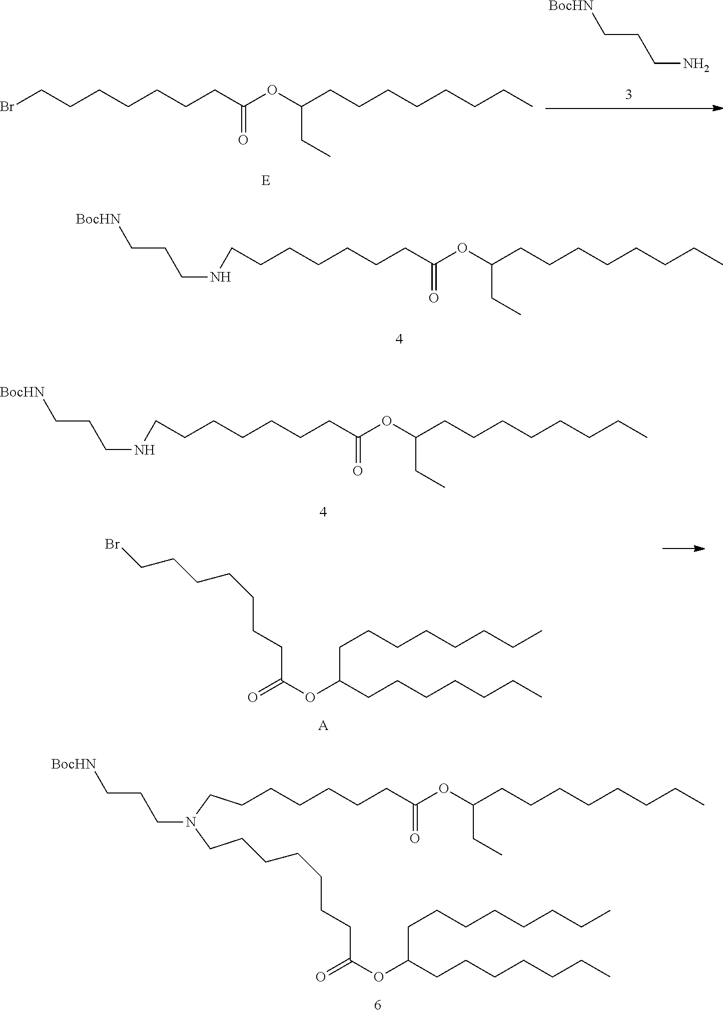

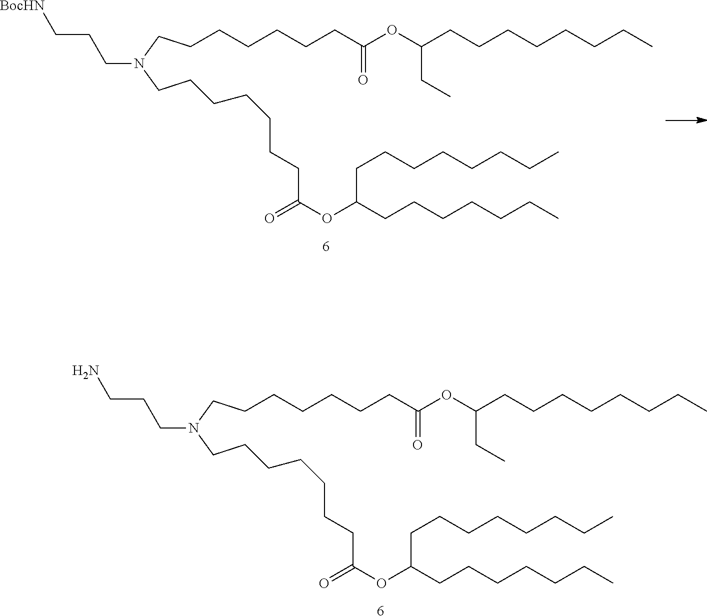

### Preparation of Compound 4:

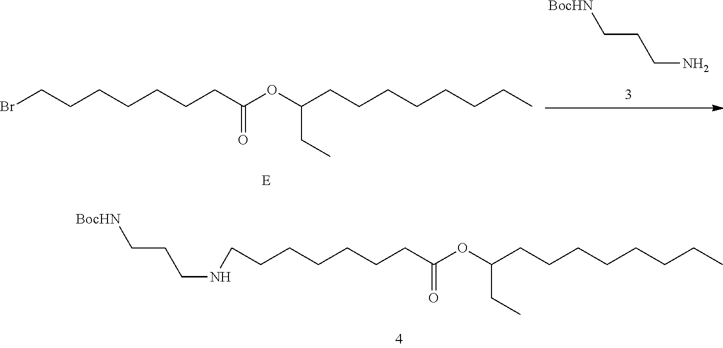 [SMILES: CC(C)(C)OC(=O)NCCCN ; CCCCCCCCC(CC)OC(=O)CCCCCCCBr ; CCCCCCCCC(CC)OC(=O)CCCCCCCNCCCNC(=O)OC(C)(C)C]

A mixture of Compound E (8.7 g, 23.05 mmol, 1 eq.), Compound 3 (20.08 g, 115.26 mmol, 20.12 mL, 5 eq.) in EtOH (70 mL) was degassed and purged with N₂ for 3 times, and then the mixture was stirred at 80° C. for 16 hours under N₂ atmosphere. LCMS showed Compound E was consumed completely and one main peak with desired MS was detected. The reaction mixture was partitioned between DCM (60 mL) and HCl (0.5 mol, 60 mL). The organic phase was separated, dried over Na₂SO₄, filtered and concentrated under reduced pressure to give a residue. The residue was purified by column chromatography (SiO₂, Dichloromethane:Methanol=50:1 to 8:1) to give Compound 4 (7.2 g, 15.14 mmol, 65.69% yield, 99% purity) as yellow solid.

LCMS [M+1]⁺=471.5

¹H NMR (400 MHz, CHLOROFORM-d) δ=4.86-4.73 (m, 1H), 3.40-3.22 (m, 2H), 3.08-2.82 (m, 4H), 2.33-2.22 (m, 2H), 2.16-1.98 (m, 2H), 1.91-1.83 (m, 2H), 1.82-1.68 (m, 3H), 1.66-1.57 (m, 3H), 1.57-1.47 (m, 4H), 1.47 (d, J=4.4 Hz, 9H), 1.31 (s, 6H), 1.30-1.24 (m, 11H), 0.92-0.84 (m, 6H)

### Preparation of Compound 6:

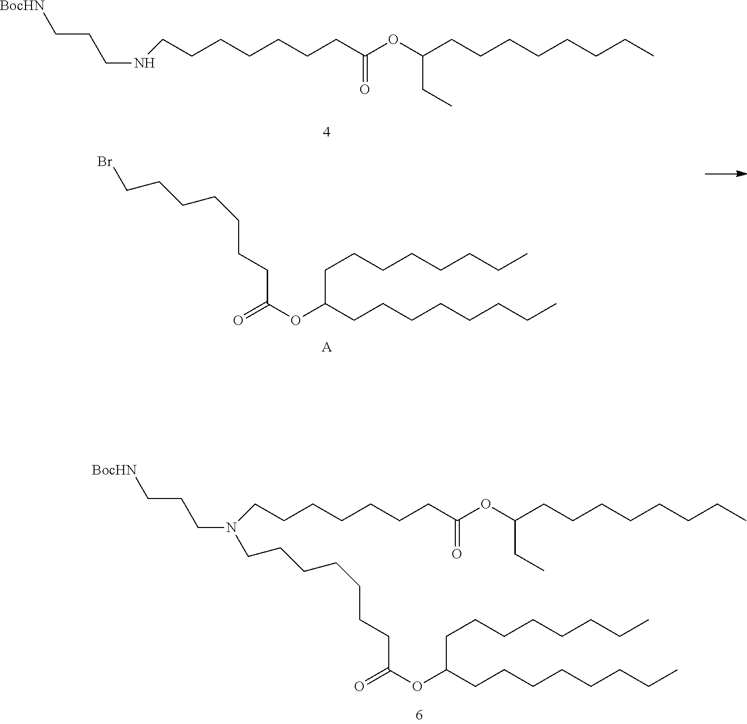

To a solution of Compound 4 (6.2 g, 13.17 mmol, 1 eq.) and Compound A (6.08 g, 13.17 mmol, 1 eq.) in ACN (130 mL) was added K₂CO₃ (5.46 g, 39.51 mmol, 3 eq.) and KI (2.19 g, 13.17 mmol, 1 eq.). The mixture was heated to 90° C. for 16 hours. LCMS showed Compound 4 was consumed completely and one main peak with desired MS was detected. The reaction mixture was concentrated to give a residue. The residue was diluted with DCM (20 mL) and washed with H₂O (30 mL). The organic layer were dried over Na₂SO₄, filtered and concentrated under reduced pressure to give crude product. The crude product was purified by column chromatography (SiO₂, Dichloromethane:Methanol=1:0 to 10:1) to give Compound 6 (10 g, 11.28 mmol, 85.61% yield, 96% purity) as yellow oil.

LCMS [M+1]⁺=851.7

¹H NMR (400 MHz, CHLOROFORM-d) δ=5.70-5.48 (m, 1H), 4.91-4.75 (m, 2H), 3.27-3.09 (m, 2H), 2.57-2.46 (m, 2H), 2.44-2.35 (m, 3H), 2.31 (s, 4H), 1.70-1.57 (m, 7H), 1.57-1.47 (m, 9H), 1.47-1.41 (m, 12H), 1.35-1.23 (m, 48H), 0.93-0.82 (m, 12H)

### Preparation of Compound F:

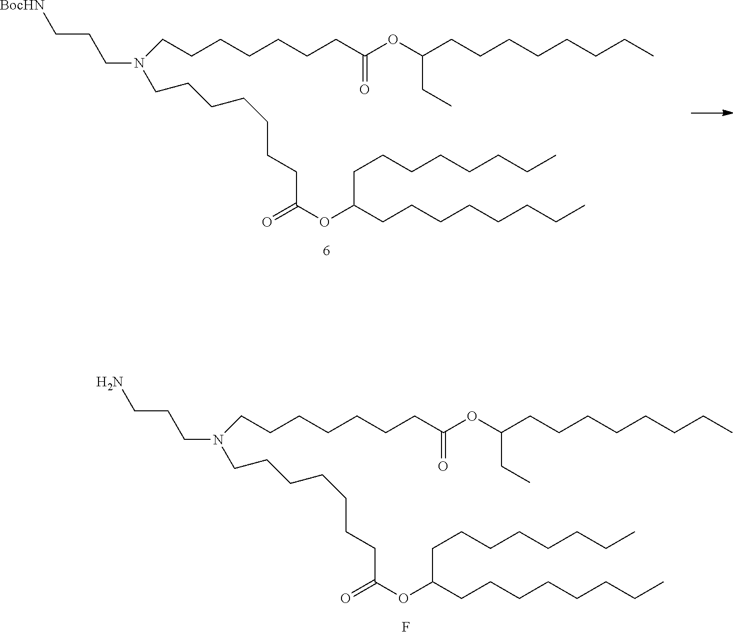

A mixture of Compound 6 (10 g, 11.75 mmol, 1 eq.) in HCl/dioxane (4 M, 50 mL) was degassed and purged with N₂ for 3 times, and then the mixture was stirred at 20° C. for 3 hours under N₂ atmosphere. LCMS showed Compound 6 was consumed completely and one main peak with desired MS was detected. The mixture was concentrated to afford the crude product. The crude product was dissolved in DCM (100 mL), and the mixture was washed with sat.NaHCO₃ (30 mL). The combined organic layers were dried over Na₂SO₄, filtered and concentrated under reduced pressure to give Compound F (8.6 g, 11.45 mmol, 90.17% yield, 100% purity) as colorless oil.

LCMS [M+1]⁺=751.8

¹H NMR (400 MHz, CHLOROFORM-d) δ=4.97-4.79 (m, 2H), 2.80-2.68 (m, 2H), 2.51-2.43 (m, 2H), 2.41-2.35 (m, 4H), 2.33-2.23 (m, 4H), 1.56 (d, 10H), 1.54-1.47 (m, 6H), 1.45-1.39 (m, 4H), 1.35-1.23 (m, 48H), 0.93-0.80 (m, 12H)

### Compound G:

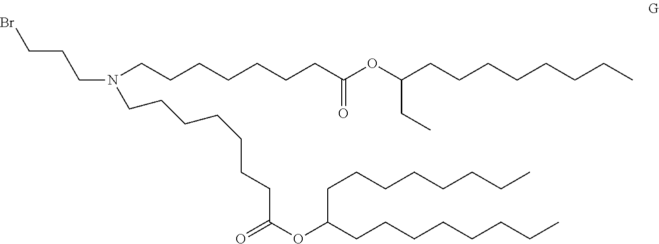 [SMILES: CCCCCCCCC(CC)OC(=O)CCCCCCCN(CCCBr)CCCCCCCC(=O)OC(CCCCCCCC)CCCCCCCC]

### Synthesis of Compound G:

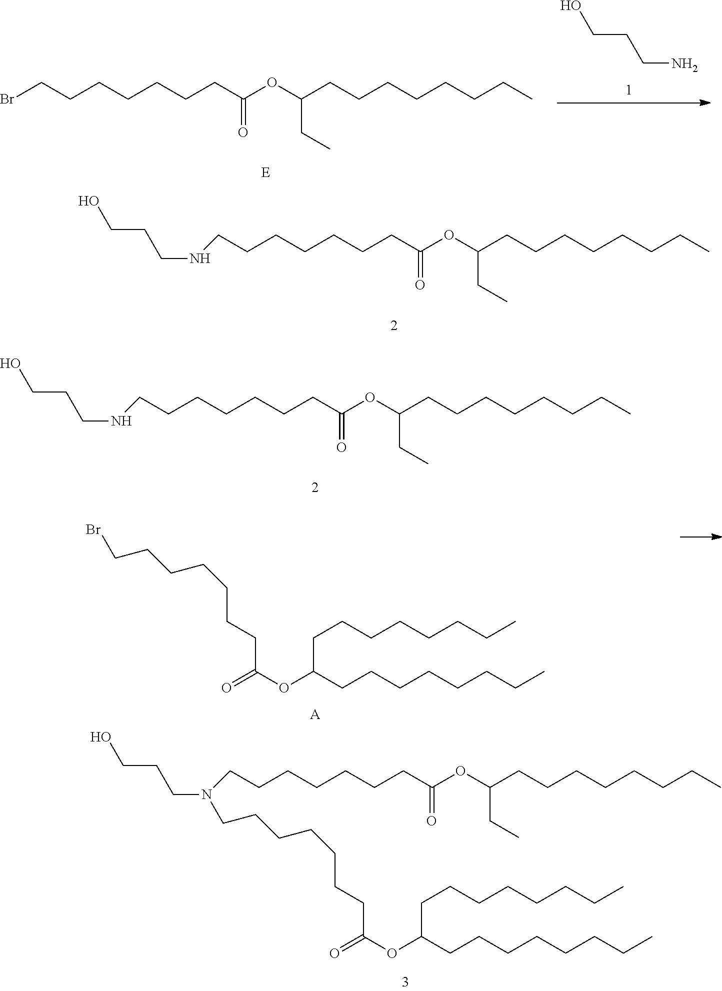

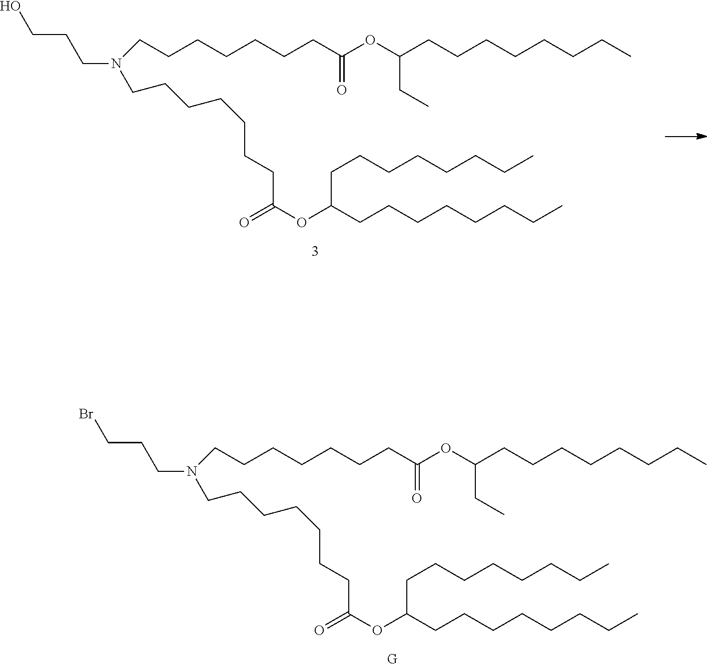 [SMILES: CCCCCCCCC(CC)OC(=O)CCCCCCCN(CCCO)CCCCCCCC(=O)OC(CCCCCCCC)CCCCCCCC ; CCCCCCCCC(CC)OC(=O)CCCCCCCN(CCCBr)CCCCCCCC(=O)OC(CCCCCCCC)CCCCCCCC]

### Preparation of Compound 2:

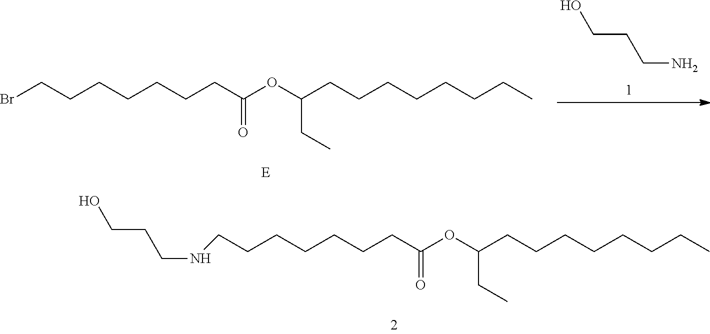 [SMILES: NCCCO ; CCCCCCCCC(CC)OC(=O)CCCCCCCBr ; CCCCCCCCC(CC)OC(=O)CCCCCCCNCCCO]

A mixture of Compound E (2.5 g, 6.62 mmol, 1 eq.), Compound 1 (9.95 g, 132.49 mmol, 10.22 mL, 20 eq.) in EtOH (50 mL) was degassed and purged with N₂ for 3 times, and then the mixture was stirred at 60° C. for 16 hours under N₂ atmosphere. LCMS indicated Compound E was consumed completely and one main peak with desired MS was detected. The residue was diluted with HCl (0.5 M, 50 mL) and extracted with DCM (60 mL*2). The combined organic layers were dried over Na₂SO₄, filtered and concentrated under reduced pressure to give a residue. The residue was purified by column chromatography (SiO₂, DCM:MeOH=99:1 to MeOH) to give Compound 2 (6.85 g, 18.43 mmol, 68.50% yield) as light yellow oil.

LCMS [M+1]⁺=372.4

¹H NMR (400 MHz, CHLOROFORM-d) δ=4.89-4.77 (m, 1H), 3.88-3.75 (m, 2H), 2.94-2.85 (m, 2H), 2.62 (t, J=7.2 Hz, 2H), 2.29 (t, J=7.6 Hz, 2H), 1.71 (td, J=5.6, 10.8 Hz, 2H), 1.65-1.58 (m, 2H), 1.57-1.44 (m, 5H), 1.66-1.43 (m, 1H), 1.41-1.14 (m, 18H), 0.98-0.82 (m, 6H)

### Preparation of Compound 3:

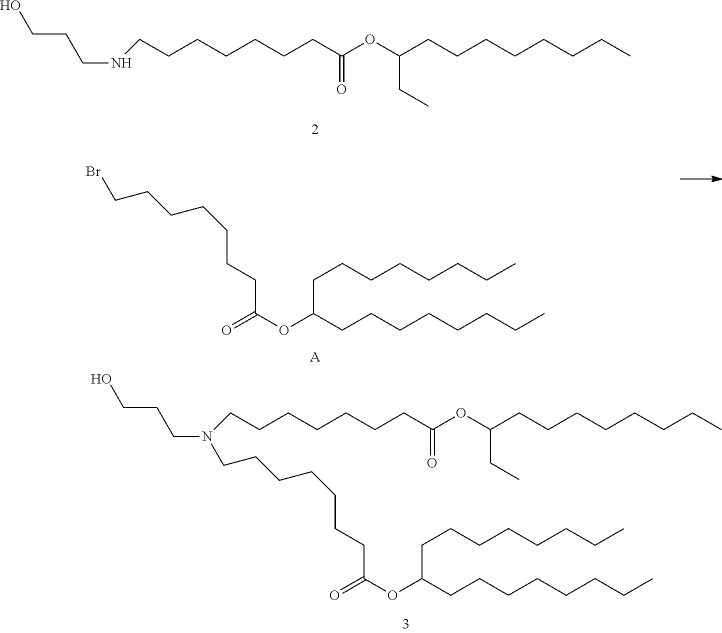

A mixture of Compound 2 (1 g, 2.69 mmol, I eq.), Compound A (1.24 g, 2.69 mmol, 1 eq.), K₂CO₃ (1.49 g, 10.76 mmol, 4 eq.), KI (446.73 mg, 2.69 mmol, 1 eq.) in ACN (20 mL) was degassed and purged with N₂ for 3 times, and then the mixture was stirred at 90° C. for 16 hours under N₂ atmosphere. LCMS indicated Compound A was consumed completely and one main peak with desired MS was detected. The reaction mixture was diluted with H₂O (30 mL) and extracted with DCM (40 mL*2). The combined organic layers were dried over Na₂SO₄, filtered and concentrated under reduced pressure to give a residue. The residue was purified by column chromatography (SiO₂, DCM:MeOH=98:2 to 96:4) to give Compound 3 (1.3 g, 1.73 mmol, 64.22% yield) as yellow oil.

LCMS [M+1]⁺=752.7

¹H NMR (400 MHz, CHLOROFORM-d) δ=4.91-4.76 (m, 2H), 3.81 (t, J=5.2 Hz, 2H), 2.72 (br s, 2H), 2.50 (br s, 4H), 2.29 (dt, J=4.0, 7.6 Hz, 4H), 1.73 (br s, 2H), 1.67-1.58 (m, 5H), 1.58-1.50 (m, 10H), 1.40-1.16 (m, 50H), 0.95-0.83 (m, 12H)

### Preparation of Compound G:

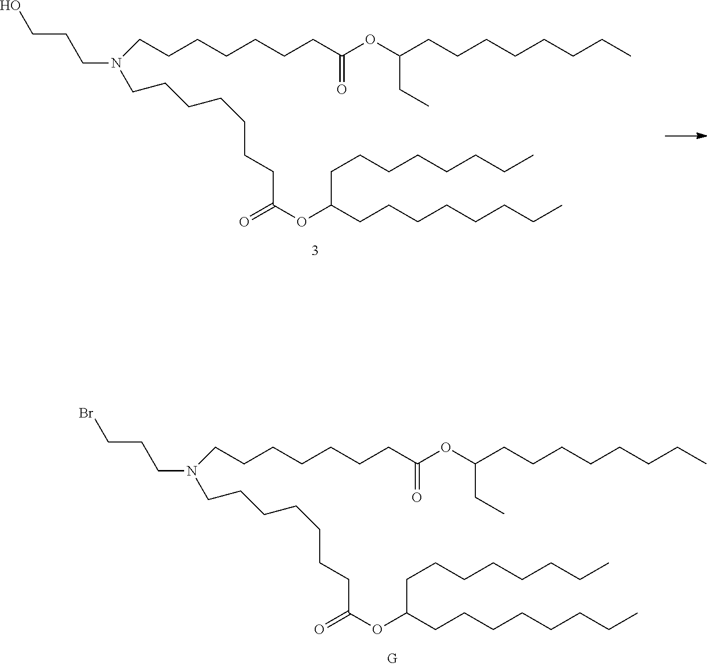 [SMILES: CCCCCCCCC(CC)OC(=O)CCCCCCCN(CCCO)CCCCCCCC(=O)OC(CCCCCCCC)CCCCCCCC ; CCCCCCCCC(CC)OC(=O)CCCCCCCN(CCCBr)CCCCCCCC(=O)OC(CCCCCCCC)CCCCCCCC]

A mixture of Compound 3 (1 g, 1.33 mmol, 1 eq.), PBr₃ (737.67 mg, 2.73 mmol, 2.05 eq.) in MeCN (10 mL) was degassed and purged with N₂ for 3 times, and then the mixture was stirred at 75° C. for 2 hours under N₂ atmosphere. LCMS indicated Compound 3 was consumed completely and one main peak with desired MS was detected. The residue was diluted with H₂O (80 mL) and extracted with DCM (100 mL*2). The combined organic layers were dried over Na₂SO₄, filtered and concentrated under reduced pressure to give Compound G (1.56 g, crude) as yellow oil.

LCMS [M+1]⁺=814.6

¹H NMR (400 MHz, CHLOROFORM-d) δ=11.36 (br d, J=2.0 Hz, JH), 4.84 (td, J=6.0, 20.0 Hz, 2H), 3.52 (t, J=5.6 Hz, 2H), 3.24-3.16 (m, 2H), 3.07-2.97 (m, 4H), 2.59-2.48 (m, 2H), 2.30 (dt, J=4.4, 7.6 Hz, 4H), 1.91-1.78 (m, 7H), 1.69-1.46 (m, 13H), 1.38 (br s, 12H), 1.27 (s, 40H), 0.97-0.79 (m, 12H)

### Example 6: Synthesis of Lipids 1-22

### Lipid 1:

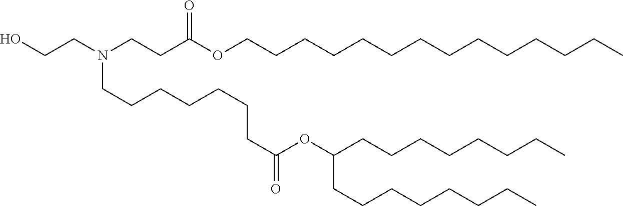 [SMILES: CCCCCCCCCCCCCCOC(=O)CCN(CCO)CCCCCCCC(=O)OC(CCCCCCCC)CCCCCCCC]

### Synthesis of Lipid 1:

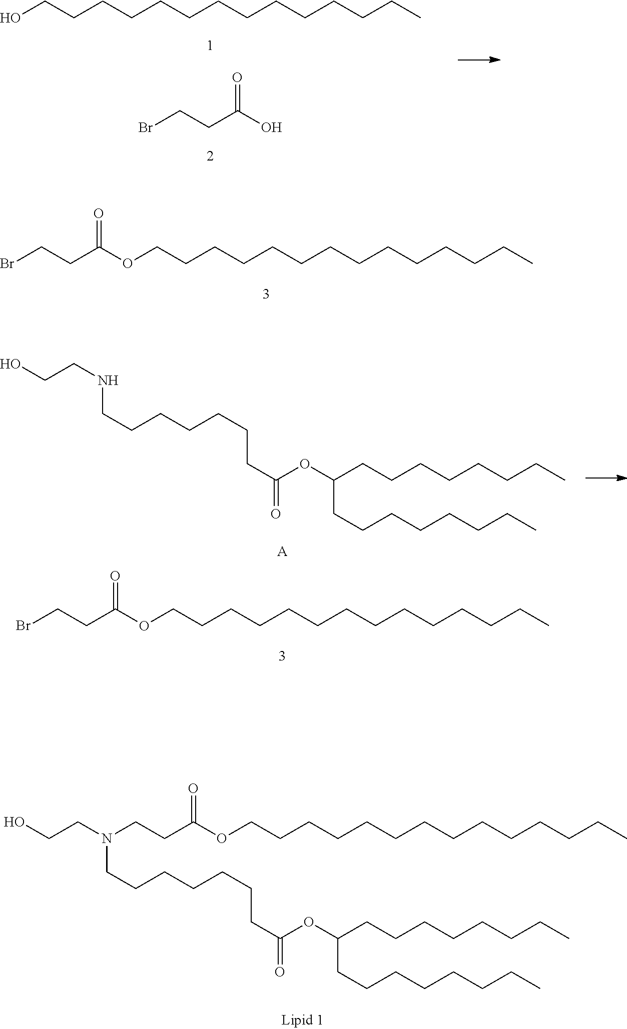

### Preparation of Compound 3:

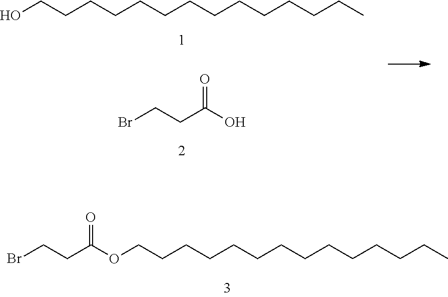 [SMILES: CCCCCCCCCCCCCCO ; O=C(O)CCBr ; CCCCCCCCCCCCCCOC(=O)CCBr]

To a solution of Compound 1 (2.00 g, 13.06 mmol, 1.35 mL, 1.4 eq.) in Tol. (20 mL) was added Compound 2 (2 g, 9.33 mmol, 1 eq.) and TsOH (803.23 mg, 4.66 mmol, 0.5 eq.) at 20° C. The resulting mixture was stirred at 50° C. for 16 hours. LCMS showed Compound 1 was consumed and desired MS was detected. The reaction mixture was diluted with H₂O (20 mL) and extracted with DCM (70 mL), dried over Na₂SO₄, filtered and concentrated under reduced pressure to give a residue. The residue was purified by column chromatography (SiO₂, Petroleum ether: Ethyl acetate=30:1 to 10:1) to give Compound 3 (2.6 g, 7.44 mmol, 79.78% yield, 100% purity) as white solid.

¹H NMR (400 MHz, DMSO-d₆) δ=4.08-4.02 (m, 2H), 3.67-3.61 (m, 2H), 2.97-2.91 (m, 2H), 1.61-1.52 (m, 2H), 1.35-1.17 (m, 22H), 0.88-0.83 (m, 3H)

### Preparation of Lipid 1:

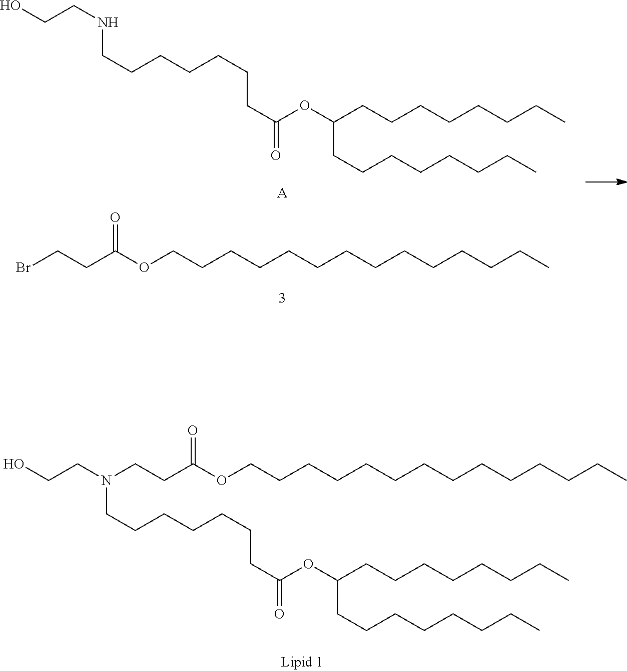

To a solution of Compound A (1.82 g, 5.21 mmol, 2.3 eq.) in ACN (10 mL) was added Compound 3 (1 g, 2.26 mmol, 1 eq.), K₂CO₃ (625.77 mg, 4.53 mmol, 2 eq.) and NaI (67.87 mg, 452.77 μmol, 0.2 eq.) at 20° C. The resulting mixture was stirred at 80° C. for 16 hours. LCMS showed Compound A was consumed and desired MS was detected. The mixture was diluted with ACN (10 mL) and filtered. The filtrate was concentrated under reduced pressure to give a residue. The residue was purified by column chromatography (SiO₂, Dichloromethane:Methanol=1:0 to 20:1) to give Lipid 1 (0.475 g, 668.86 μmol, 29.55% yield, 100% purity) as yellow oil.

LCMS [M+1]⁺=710.7

¹H NMR (400 MHz, CHLOROFORM-d) δ=4.91-4.83 (m, 1H), 4.11-4.05 (m, 2H), 3.60-3.54 (m, 2H), 2.83 (br t, J=6.8 Hz, 2H), 2.61 (br t, J=5.2 Hz, 2H), 2.47 (br t, J=6.8 Hz, 4H), 2.31-2.25 (m, 2H), 1.66-1.59 (m, 4H), 1.53-1.43 (m, 6H), 1.34-1.24 (m, 54H), 0.88 (t, J=6.8 Hz, 9H)

### Lipid 2:

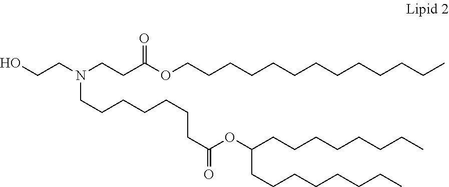 [SMILES: CCCCCCCCCCCCCOC(=O)CCN(CCO)CCCCCCCC(=O)OC(CCCCCCCC)CCCCCCCC]

### Synthesis of Lipid 2:

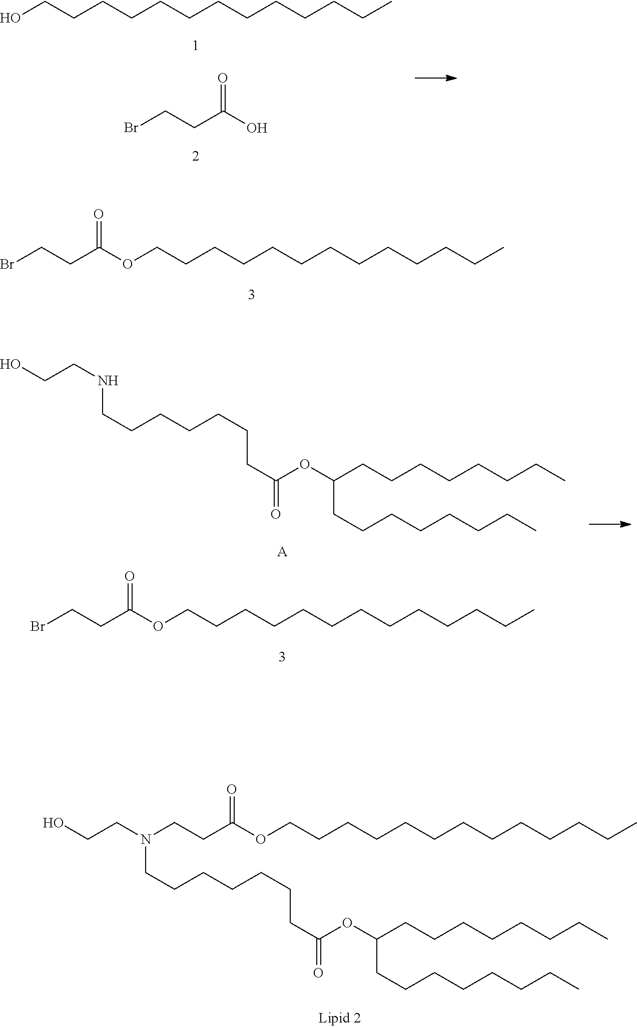

### Preparation of Compound 6:

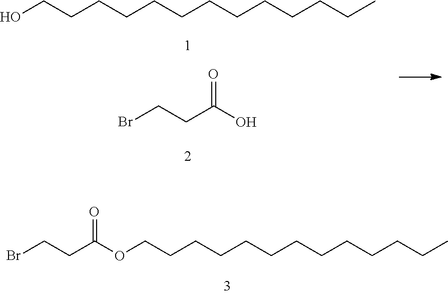 [SMILES: CCCCCCCCCCCCCO ; O=C(O)CCBr ; CCCCCCCCCCCCCOC(=O)CCBr]

To a solution of Compound 1 (916.20 mg, 5.99 mmol, 619.05 μL, 1.2 eq.) in toluene (20 mL) was added Compound 2 (1 g, 4.99 mmol, I eq.), TsOH (429.73 mg, 2.50 mmol, 0.5 eq.) at 20° C. The mixture was stirred at 50° C. for 16 hours. LCMS showed Compound 2 was consumed completely and one main peak with desired MS was detected. The mixture was diluted with DCM (30 mL) and washed with NH₄Cl (10 mL*3). The combined organic layers were washed with brine (10 mL*3), dried over Na₂SO₄, filtered and concentrated under reduced pressure to give a residue. The residue was purified by column chromatography (SiO₂, Petroleum ether: Ethyl acetate=100:1 to 80:1) to give Compound 3 (1.36 g, 4.06 mmol, 81.26% yield) as a yellow oil.

¹H NMR (400 MHz, DMSO-d₆) δ=4.09-4.01 (m, 2H), 3.68-3.60 (m, 2H), 2.99-2.90 (m, 2H), 1.61-1.51 (m, 2H), 1.34-1.19 (m, 20H), 0.88-0.82 (m, 3H)

### Preparation of Lipid 2:

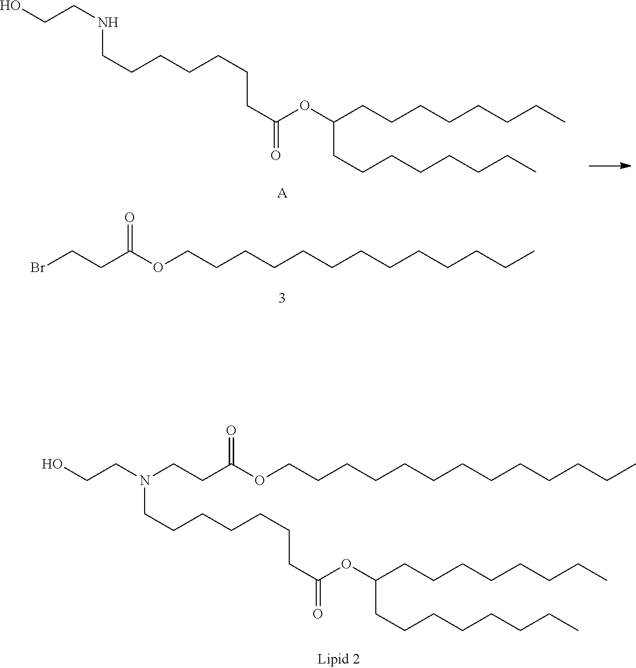

A mixture of Compound A (569.33 mg, 1.70 mmol, 1.5 eq.), Compound 3 (0.5 g, 1.13 mmol, 1 eq.), K₂CO₃ (312.88 mg, 2.26 mmol, 2 eq.) and NaI (16.97 mg, 113.19 μmol, 0.1 eq.) in ACN (5 mL) was degassed and purged with N₂ for 3 times, and then the mixture was stirred at 80° C. for 16 hours under N₂ atmosphere. LCMS showed Compound 3 was consumed completely and one main peak with desired MS was detected. The reaction was filtered and concentrated under reduced pressure to give a residue. The residue was purified by prep-TLC (SiO₂, DCM:MeOH=20:1) to give Lipid 2 (102.57 mg, 147.34 μmol, 13.02% yield, 100% purity) as yellow oil.

LCMS [M+1]⁺=696.7

¹H NMR (400 MHz, CHLOROFORM-d) δ=4.91-4.83 (m, 1H), 4.16 (t, J=6.8 Hz, 1H), 4.11-4.05 (m, 2H), 3.66-3.53 (m, 2H), 2.91-2.77 (m, 2H), 2.68-2.56 (m, 2H), 2.56-2.37 (m, 4H), 2.32-2.24 (m, 2H), 1.66-1.59 (m, 4H), 1.54-1.42 (m, 6H), 1.34-1.24 (m, 50H), 0.89 (t, J=6.8 Hz, 9H)

### Lipid 3:

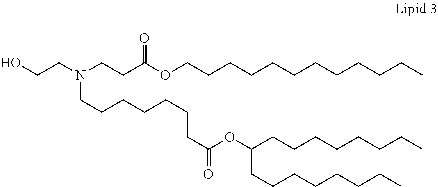 [SMILES: CCCCCCCCCCCCOC(=O)CCN(CCO)CCCCCCCC(=O)OC(CCCCCCCC)CCCCCCCC]

### Synthesis of Lipid 3:

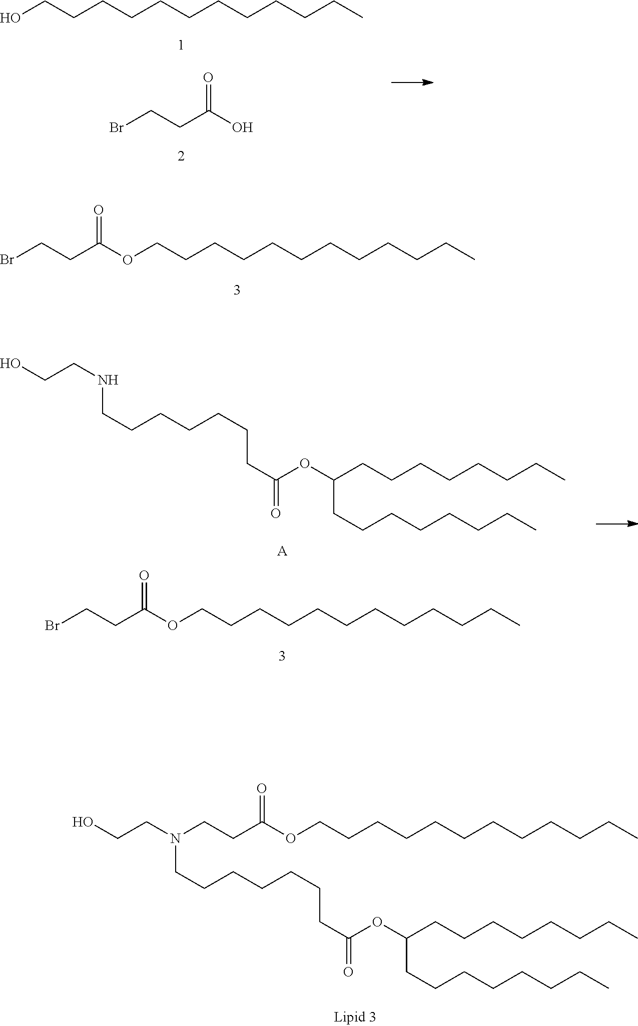

### Preparation of Compound 3:

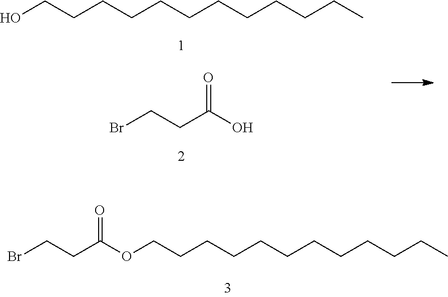 [SMILES: CCCCCCCCCCCCO ; O=C(O)CCBr ; CCCCCCCCCCCCOC(=O)CCBr]

To a solution of Compound 1 (985.17 mg, 6.44 mmol, 665.65 μL, 1.2 eq.) and Compound 2 (1 g, 5.37 mmol, 1 eq.) in Tol. (20 mL) was added TsOH (462.08 mg, 2.68 mmol, 0.5 eq.). The mixture was stirred at 50° C. for 16 hours. TLC (Petroleum ether: Ethyl acetate=10:1, R1: R_{f}=0.2, P1: R_{f}=0.6) indicated Compound 2 was consumed completely and one new spot formed. The reaction was clean according to TLC. The mixture was concentrated to give crude product. The residue was diluted with H₂O (20 mL) and extracted with EA (60 mL). The combined organic layers were dried over Na₂SO₄, filtered and concentrated under reduced pressure to give a residue to give Compound 3 (850 mg, 2.38 mmol, 44.37% yield, 90% purity) as colorless oil.

¹H NMR (400 MHz, DMSO-d₆) δ=4.11-4.00 (m, 2H), 3.68-3.58 (m, 2H), 2.99-2.88 (m, 2H), 1.63-1.48 (m, 2H), 1.36-1.19 (m, 18H), 0.93-0.78 (m, 3H)

### Preparation of Lipid 3:

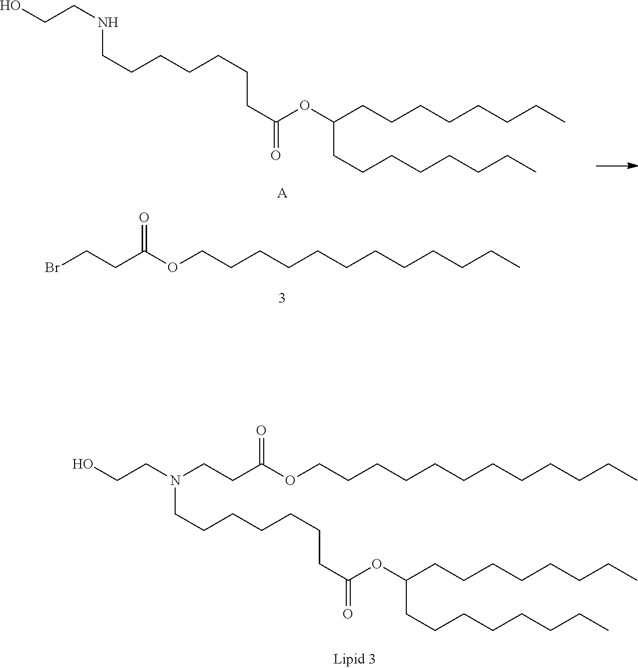

To a solution of Compound 3 (240.03 mg, 747.07 μmol, 1.5 eq.) and Compound A (220 mg, 498.04 μmol, 1 eq.) in ACN (3 mL) was added K₂CO₃ (103.25 mg, 747.07 μmol, 1.5 eq.) and NaI (7.47 mg, 49.80 μmol, 0.1 eq.). The mixture was stirred at 80° C. for 16 hours. LCMS showed Compound A was consumed completely and one main peak with desired MS was detected. The mixture was filtered through celatom, and the filtrate was concentrated to give a residue. The residue was purified by prep-HPLC (H₂O (0.04% HCl)-THF:ACN=1:3) to give Lipid 3 (117.95 mg, 73.30 μmol, 14.72% yield, 100% purity, HCl) as yellow oil.

LCMS [M+1]⁺=682.7

¹H NMR (400 MHz, DMSO-d₆) δ=9.32-9.12 (m, 1H), 5.39-5.26 (m, 1H), 4.83-4.70 (m, 1H), 4.15-3.97 (m, 2H), 3.79-3.64 (m, 2H), 3.46-3.33 (m, 2H), 3.22-3.15 (m, 2H), 3.12-3.03 (m, 2H), 2.88-2.78 (m, 2H), 2.29-2.23 (m, 2H), 1.67-1.41 (m, 10H), 1.32-1.20 (m, 48H), 0.91-0.78 (m, 9H)

### Lipid 4:

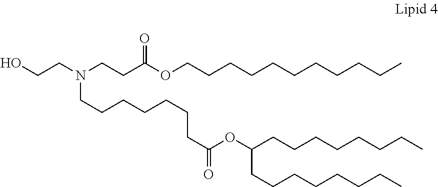 [SMILES: CCCCCCCCCCCOC(=O)CCN(CCO)CCCCCCCC(=O)OC(CCCCCCCC)CCCCCCCC]

### Synthesis of Lipid 4:

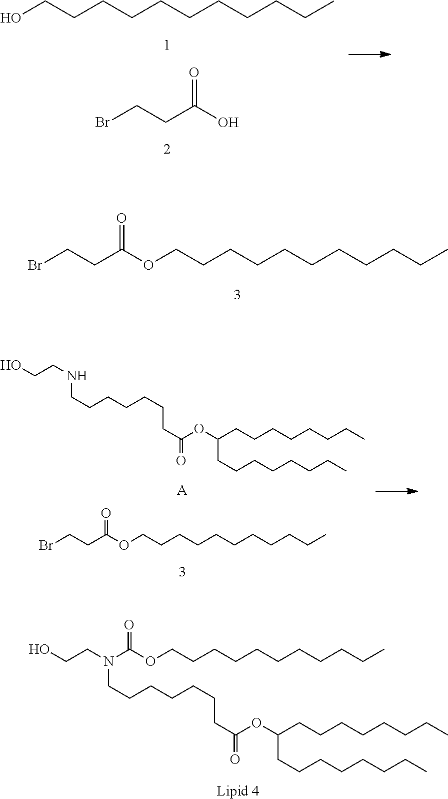

### Preparation of Compound 3:

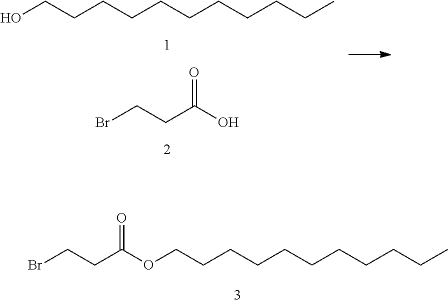 [SMILES: CCCCCCCCCCCO ; O=C(O)CCBr ; CCCCCCCCCCCOC(=O)CCBr]

To a solution of Compound 1 (2.13 g, 13.93 mmol, 1.44 mL, 1.2 eq.) and Compound 2 (2 g, 11.61 mmol, 1 eq.) in Tol. (40 mL) was added TsOH (999.39 mg, 5.80 mmol, 0.5 eq.). The mixture was stirred at 50° C. for 16 hours. TLC (Petroleum ether: Ethyl acetate=10:1, R1: R_{f}=0.2, P1: R_{f}=0.6) indicated Compound 2 was consumed completely and one new spot formed. The mixture was concentrated to give a residue. The residue was diluted with H₂O (30 mL) and extracted with EA (60 mL). The combined organic layers were dried over Na₂SO₄, filtered and concentrated under reduced pressure to give a residue to give Compound 3 (2.65 g, 7.76 mmol, 66.87% yield, 90% purity) as colorless oil.

¹H NMR (400 MHz, DMSO-d₆) δ=4.11-4.00 (m, 2H), 3.66-3.60 (m, 2H), 2.99-2.92 (m, 2H), 1.63-1.50 (m, 2H), 1.35-1.21 (m, 16H), 0.89-0.82 (m, 3H)

### Preparation of Lipid 4:

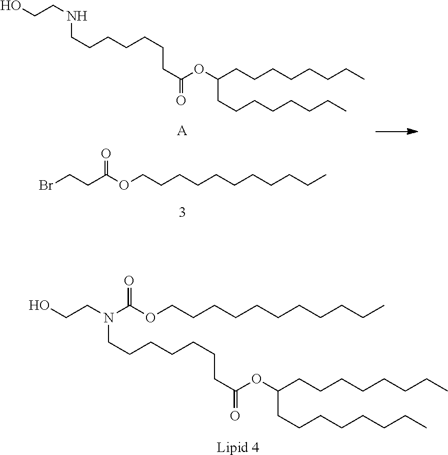

To a solution of Compound 3 (250.42 mg, 814.98 μmol, 1.2 eq.) and Compound A (300 mg, 679.15 μmol, 1 eq.) in ACN (3 mL) was added K₂CO₃ (112.63 mg, 814.98 μmol, 1.2 eq.) and NaI (10.18 mg, 67.92 μmol, 0.1 eq.). The mixture was stirred at 80° C. for 16 hours. LCMS showed Compound A was consumed completely and one main peak with desired MS was detected. The mixture was filtered through celatom, and the filtrate was concentrated to give crude product. The crude product was purified by prep-TLC (SiO₂, DCM:MeOH=8:1) to give Lipid 4 (101 mg, 151.18 μmol, 22.26% yield, 100/6 purity) as colorless oil.

LCMS [M+1]⁺=668.6

¹H NMR (400 MHz, DMSO-d₆) δ=4.85-4.69 (m, 1H), 4.25-4.18 (m, 1H), 4.01-3.94 (m, 2H), 3.42-3.35 (m, 2H), 2.72-2.64 (m, 2H), 2.46-2.41 (m, 2H), 2.38-2.32 (m, 4H), 2.27-2.21 (m, 2H), 1.60-1.41 (m, 8H), 1.36-1.16 (m, 48H), 0.91-0.78 (m, 9H)

### Lipid 5:

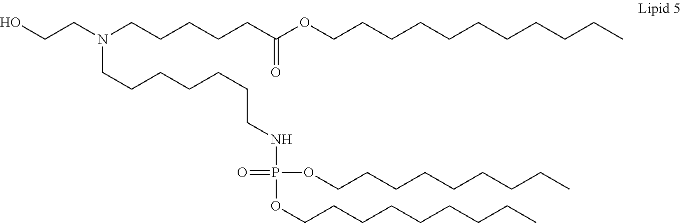 [SMILES: CCCCCCCCCCCOC(=O)CCCCCN(CCO)CCCCCCCNP(=O)(OCCCCCCCCC)OCCCCCCCCC]

### Synthesis of Lipid 5:

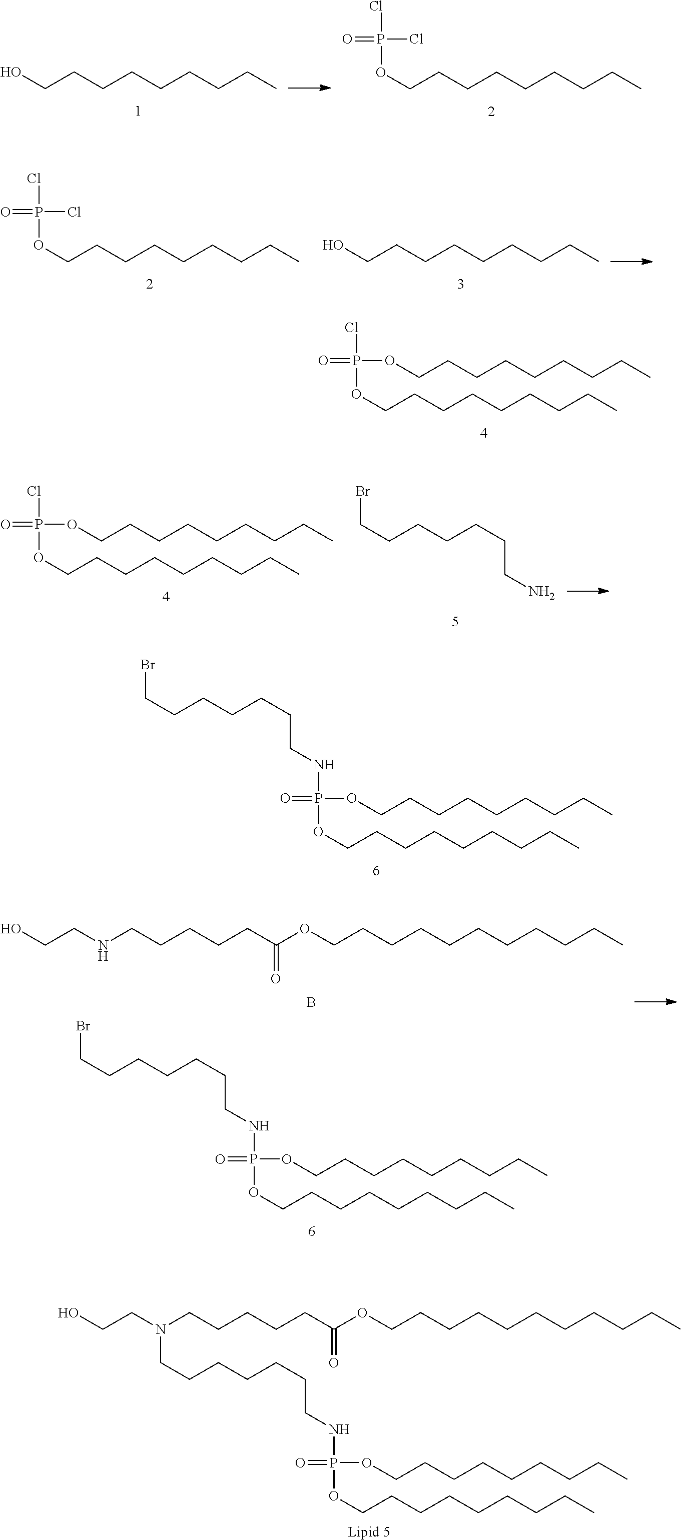

### Preparation of Compound 2:

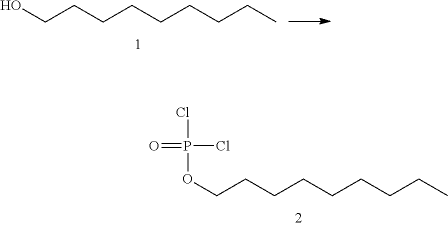

A mixture of Compound 1 (1 g, 6.93 mmol, 1 eq.) was added POCl₃ (2.13 g, 13.86 mmol, 1.29 mL, 2 eq.) dropwise at 20° C., and then the mixture was stirred at 20° C. for 3 hours under N₂ atmosphere. TLC (Petroleum ether: Ethyl acetate=5:1, Rt=0.56) indicated Compound I was consumed completely and one new spot formed. The reaction mixture was concentrated under reduced pressure to give Compound 2 (1.8 g, 6.89 mmol, 99.44% yield) as deep yellow oil, which was used without further purification.

### Preparation of Compound 4:

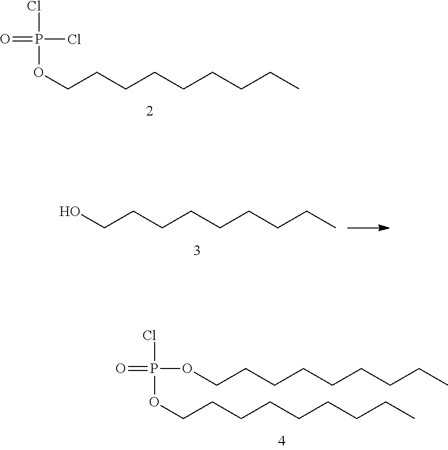

To a solution of Compound 3 (977.80 mg, 6.78 mmol, 1 eq.), TEA (685.90 mg, 6.78 mmol, 943.46 ILL, 1 eq.) in Tol. (10 mL) was added Compound 2 (1.77 g, 6.78 mmol, 1 eq.) in Tol. (30 mL) dropwise at 20° C. The mixture was stirred at 20° C. for 16 hours. TLC (Petroleum ether: Ethyl acetate=5:1, R_{f}=0.56) indicated Compound 2 was consumed completely and one new spot formed. The residue was diluted with H₂O (10 mL) and extracted with DCM (10 mL*2). The combined organic layers were dried over Na₂SO₄, filtered and concentrated under reduced pressure to give a residue. The residue was purified by column chromatography (SiO₂, Petroleum ether: Ethyl acetate=99:1 to 96:4) to give Compound 4 (2.8 g, 7.59 mmol, 55.99% yield) as colorless oil.

¹H NMR (400 MHz, CHLOROFORM-d) δ=4.27-4.11 (m, 4H), 1.74 (quin, J=6.8 Hz, 4H), 1.47-1.36 (m, 4H), 1.35-1.21 (m, 20H), 0.95-0.83 (m, 6H)

### Preparation of Compound 6.

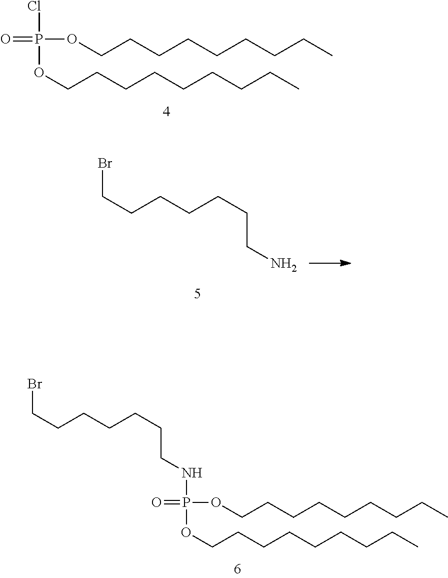 [SMILES: CCCCCCCCCOP(=O)(Cl)OCCCCCCCCC ; NCCCCCCCBr ; CCCCCCCCCOP(=O)(NCCCCCCCBr)OCCCCCCCCC]

To a solution of Compound 4 (606.55 mg, 1.65 mmol, 1 eq.) in DCM (9 mL) was added TEA (550.48 mg, 5.44 mmol, 757.20 μL, 3.3 eq.), Compound 5 (320.00 mg, 1.65 mmol, 1 eq.) was degassed and purged with N₂ for 3 times, and then the mixture was stirred at 20° C. for 16 hours under N₂ atmosphere. LCMS indicated Compound 4 was consumed completely and one main peak with desired MS was detected. The reaction mixture was diluted with H₂O (10 mL) and extracted with DCM (10 mL*3). The combined organic layers were dried. The residue was purified by column chromatography (SiO₂, Petroleum ether: Ethyl acetate=10:1 to 4:1) to give Compound 6 (618 mg, 1.18 mmol, 71.33% yield) as colorless oil.

LCMS [M+1]⁺=526.3

1H NMR (400 MHz, CHLOROFORM-d) δ=4.09-3.84 (m, 4H), 3.41 (t, J=6.8 Hz, 2H), 2.90 (q, J=7.7 Hz, 2H), 1.86 (quin, J=7.1 Hz, 2H), 1.73-1.63 (m, 4H), 1.54-1.41 (m, 4H), 1.41-1.20 (m, 27H), 0.92-0.85 (m, 6H)

### Preparation of Lipid 5:

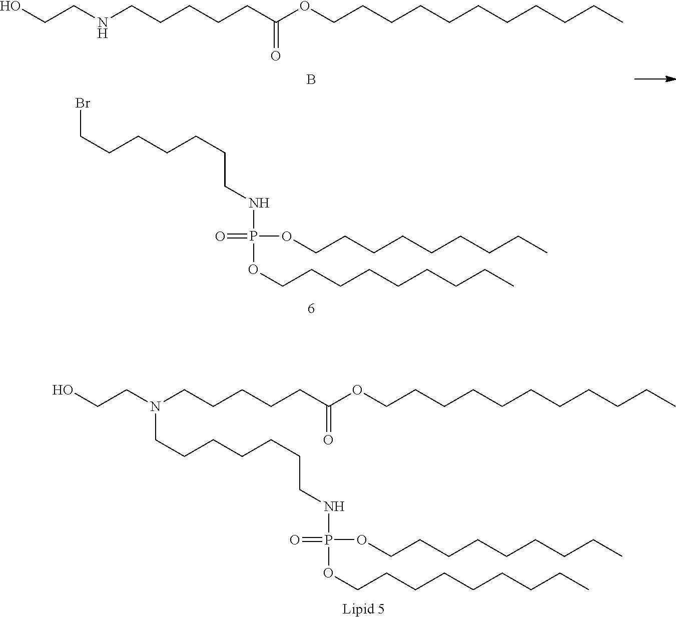

A mixture of Compound B (612.49 mg, 1.17 mmol, 1.2 eq.), Compound 6 (320 mg, 971.12 μmol, 1 eq.), K₂CO₃ (268.43 mg, 1.94 mmol, 2 eq.), NaI (14.56 mg, 97.11 μmol, 0.1 eq.) in ACN (8 mL) was degassed and purged with N₂ for 3 times, and then the mixture was stirred at 80° C. for 16 hours under N₂ atmosphere. LCMS indicated Compound 6 was consumed completely and one main peak with desired MS was detected. The residue was diluted with H₂O (10 mL) and extracted with DCM (10 mL*2). The combined organic layers were dried over Na₂SO₄, filtered and concentrated under reduced pressure to give a residue. The residue was purified by column chromatography (SiO₂, Dichloromethane:Methanol=80:1 to 3:1) to give Lipid 5 (400 mg, 516.01 μmol, 53.14% yield) as light red oil.

LCMS [M+1]⁺=775.7

1H NMR (400 MHz, DMSO-d₆) δ=4.84-4.72 (m, 1H), 4.23 (m, 1H), 3.99 (t, J=6.4 Hz, 2H), 3.80 (dq, J=3.2, 6.4 Hz, 4H), 3.52-3.34 (m, 4H), 2.75-2.68 (m, 2H), 2.34 (m, 2H), 2.27 (br t, J=7.2 Hz, 2H), 1.61-1.48 (m, 9H), 1.43-1.19 (m, 55H), 0.86 (br t, J=6.8 Hz, 9H)

### Lipid 6:

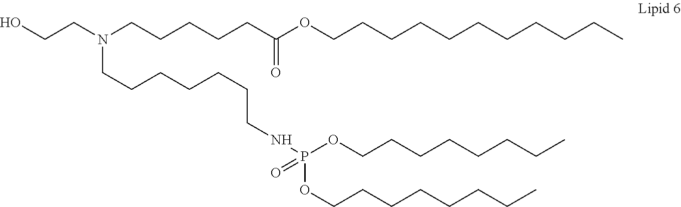 [SMILES: CCCCCCCCCCCOC(=O)CCCCCN(CCO)CCCCCCCNP(=O)(OCCCCCCCC)OCCCCCCCC]

### Synthesis of Lipid 6:

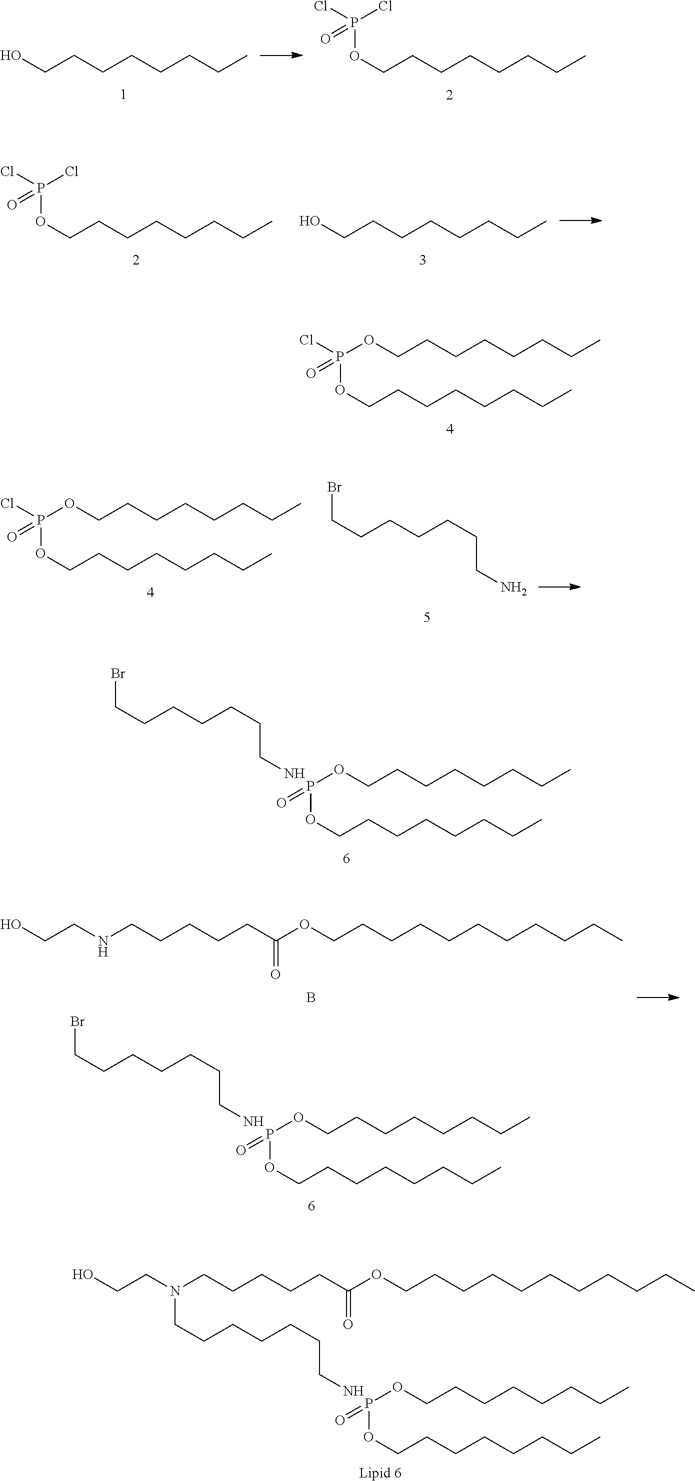

### Preparation of Compound 2:

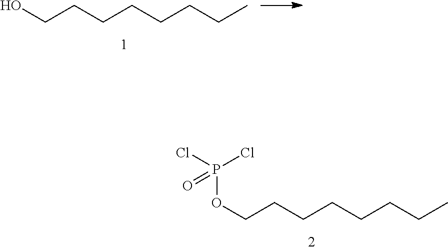

POCl₃ (11.77 g, 76.79 mmol, 7.16 mL, 2 eq.) in DCM (2.5 mL) was added dropwise to Compound 1 (5 g, 38.39 mmol, 6.07 mL, 1 eq.) at 20° C. After addition, the mixture was stirred at this temperature for 3 hours. TLC (Petroleum ether: Ethyl acetate=5:1, P: R_{f}=0.72) indicated trace of Compound 1 remained and new spots formed. The reaction mixture was concentrated under reduced pressure to remove POCl₃. And then the crude product was evaporated with toluene three times (3*2 mL) to give Compound 2 (9.49 g, crude) as colorless oil. The product was used in the next step directly.

¹H NMR (400 MHz, CHLOROFORM-d) δ=4.34 (td, J=6.4, 10.0 Hz, 2H), 1.93-1.66 (m, 2H), 1.50-1.37 (m, 2H), 1.37-1.21 (m, 8H), 1.00-0.75 (m, 3H)

### Preparation of Compound 4:

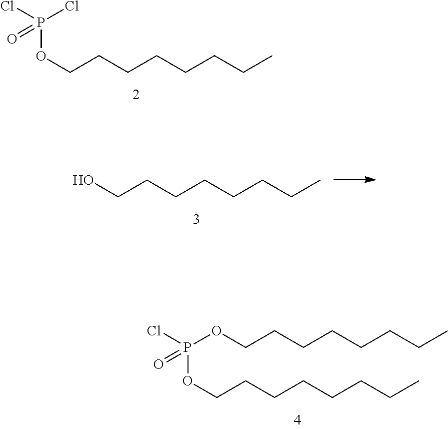

Compound 2 (4.75 g, 36.49 mmol, 5.77 mL, 0.95 eq.) and TEA (3.89 g, 38.41 mmol, 5.35 mL, 1 eq.) in Tol. (50 mL) was added dropwise to a solution of Compound 3 (9.49 g, 38.41 mmol, 1 eq.) in Tol. (150 mL). The mixture was stirred at 20° C. for 16 hours. TLC (Petroleum ether: Ethyl acetate=5:1, P: R_{f}=0.66) indicated Compound 2 was consumed and new spots formed. The reaction mixture was diluted with H₂O (40 mL) and extracted with DCM (200 mL). The combined organic layers were washed with brine (50 mL), dried over Na₂SO₄, filtered and concentrated under reduced pressure to give a residue. The residue was purified by column chromatography (SiO₂, Petroleum ether: Ethyl acetate=200:1 to 100:1) to give Compound 4 (7.3 g, 21.42 mmol, 55.76% yield) as light yellow oil.

¹H NMR (400 MHz, CHLOROFORM-d) δ=4.36-4.04 (m, 4H), 1.74 (quin, J=6.8 Hz, 4H), 1.46-1.36 (m, 4H), 1.36-1.22 (m, 16H), 1.02-0.77 (m, 6H)

### Preparation of Compound 6:

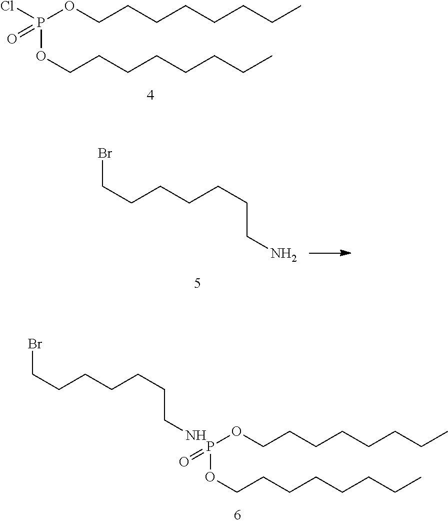

To a solution of Compound 4 (1.2 g, 3.52 mmol, 1 eq.) in DCM (18 mL) was added TEA (783.71 mg, 7.75 mmol, 1.08 mL, 2.2 eq.) and Compound 5 (683.36 mg, 3.52 mmol, 1 eq.) at 0° C. The mixture was stirred at 20° C. for 16 hours. LCMS showed desired MS was detected. TLC (Petroleum ether: Ethyl acetate=1:1, P: R_{f}=0.40) indicated Compound 4 was consumed and new spots formed. The reaction mixture was diluted with H₂O (30 mL) and extracted with DCM (80 mL). The combined organic layers were washed with brine (30 mL), dried over Na₂SO₄, filtered and concentrated under reduced pressure to give a residue. The residue was purified by column chromatography (SiO₂, Petroleum ether: Ethyl acetate=10:1 to 5:1) to give Compound 6 (1.5 g, 3.00 mmol, 85.21% yield, 99.7% purity) as light yellow oil.

LCMS [M+1]⁺=498.5

¹H NMR (400 MHz, CHLOROFORM-d) δ=4.08-3.87 (m, 4H), 3.41 (t, J=6.8 Hz, 2H), 2.99-2.80 (m, 2H), 2.46 (br d, J=5.6 Hz, 1H), 1.86 (quin, J=7.1 Hz, 2H), 1.67 (quin, J=7.2 Hz, 4H), 1.55-1.40 (m, 4H), 1.39-1.15 (m, 24H), 0.96-0.79 (m, 6H)

### Preparation of Lipid 6:

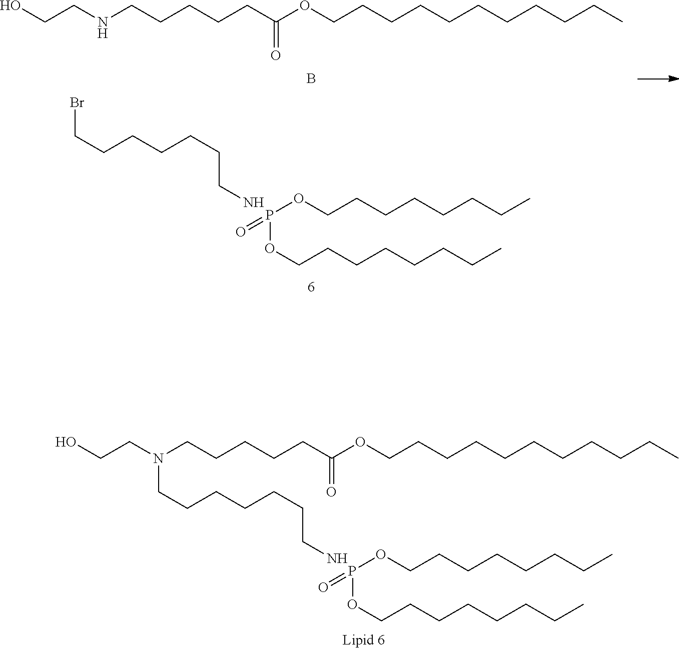

A mixture of Compound 6 (726.18 mg, 1.46 mmol, 1.2 eq.), K₂CO₃ (335.54 mg, 2.43 mmol, 2 eq.), NaI (18.20 mg, 121.39 μmol, 0.1 eq.) and Compound B (400 mg, 1.21 mmol, 1 eq.) in ACN (6 mL) was degassed and purged with N₂ for 3 times, and then the mixture was stirred at 80° C. for 16 hours under N₂ atmosphere. LCMS showed Compound 6 was consumed and desired MS was detected. The reaction mixture was concentrated under reduced pressure to give a residue. The residue was diluted with H₂O (20 mL) and extracted with DCM (60 mL). The combined organic layers were washed with brine (20 mL), dried over Na₂SO₄, filtered and concentrated under reduced pressure to give a residue. The residue was purified by column chromatography (SiO₂, DCM:MeOH=80:1 to 50:1) to give Lipid 6 (200 mg, 267.69 μmol, 22.05% yield, 100% purity) as colorless oil.

LCMS [M+1]⁺=747.8

¹H NMR (400 MHz, CHLOROFORM-d) δ=4.06 (t, J=6.8 Hz, 2H), 4.03-3.90 (m, 4H), 3.58 (br s, 2H), 2.89 (qd, J=7.2, 9.6 Hz, 2H), 2.63 (br s, 2H), 2.58-2.40 (m, 5H), 2.31 (t, J=7.6 Hz, 2H), 1.75-1.57 (m, 9H), 1.55-1.42 (m, 6H), 1.40-1.21 (m, 44H), 1.02-0.72 (m, 9H)

### Lipid 7:

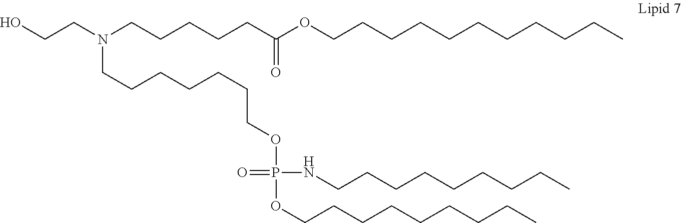 [SMILES: CCCCCCCCCCCOC(=O)CCCCCN(CCO)CCCCCCCOP(=O)(NCCCCCCCCC)OCCCCCCCCC]

### Preparation of Lipid 7:

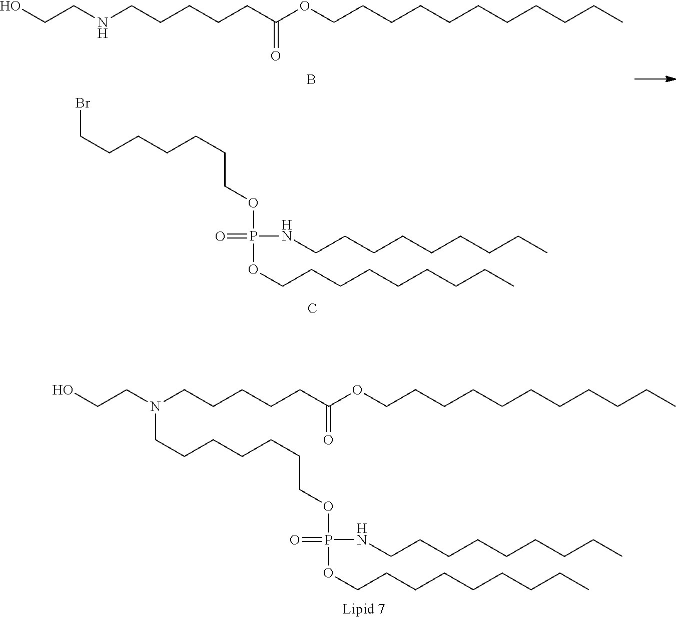

To a solution of Compound C (300 mg, 569.73 μmol, 1 eq.) in ACN (6 mL) was added Compound B (187.73 mg, 569.73 μmol, 1 eq.), K₂CO₃ (157.48 mg, 1.14 mmol, 2 eq.) and NaI (17.08 mg, 113.95 μmol, 0.2 eq.). The resulting mixture was heated to 80° C. for 16 hr. LCMS showed Compound C was consumed and desired MS was detected. The reaction mixture was filtered and the filtrate was diluted with H₂O (10 mL) and extracted with DCM (10 mL*2), dried over Na₂SO₄, filtered and concentrated under reduced pressure to give a residue. The residue was purified by column chromatography (SiO₂, Dichloromethane:Methanol=40:1 to 35:1) to give Lipid 7 (100 mg, 129.00 μmol, 22.64% yield, 100% purity) as brown oil.

LCMS [M+1]⁺=775.7

¹H NMR (400 MHz, DMSO-d₆) δ=4.84-4.71 (m, 1H), 4.05-3.95 (m, 2H), 3.87-3.70 (m, 4H), 3.61-3.41 (m, 2H), 2.53 (br s, 6H), 2.49-2.38 (m, 2H), 2.30-2.23 (m, 2H), 1.61-1.50 (m, 8H), 1.46-1.18 (m, 54H), 0.89-0.81 (m, 9H)

### Lipid 8:

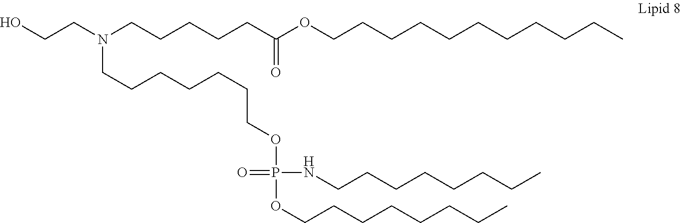 [SMILES: CCCCCCCCCCCOC(=O)CCCCCN(CCO)CCCCCCCOP(=O)(NCCCCCCCC)OCCCCCCCC]

### Synthesis of Lipid 8:

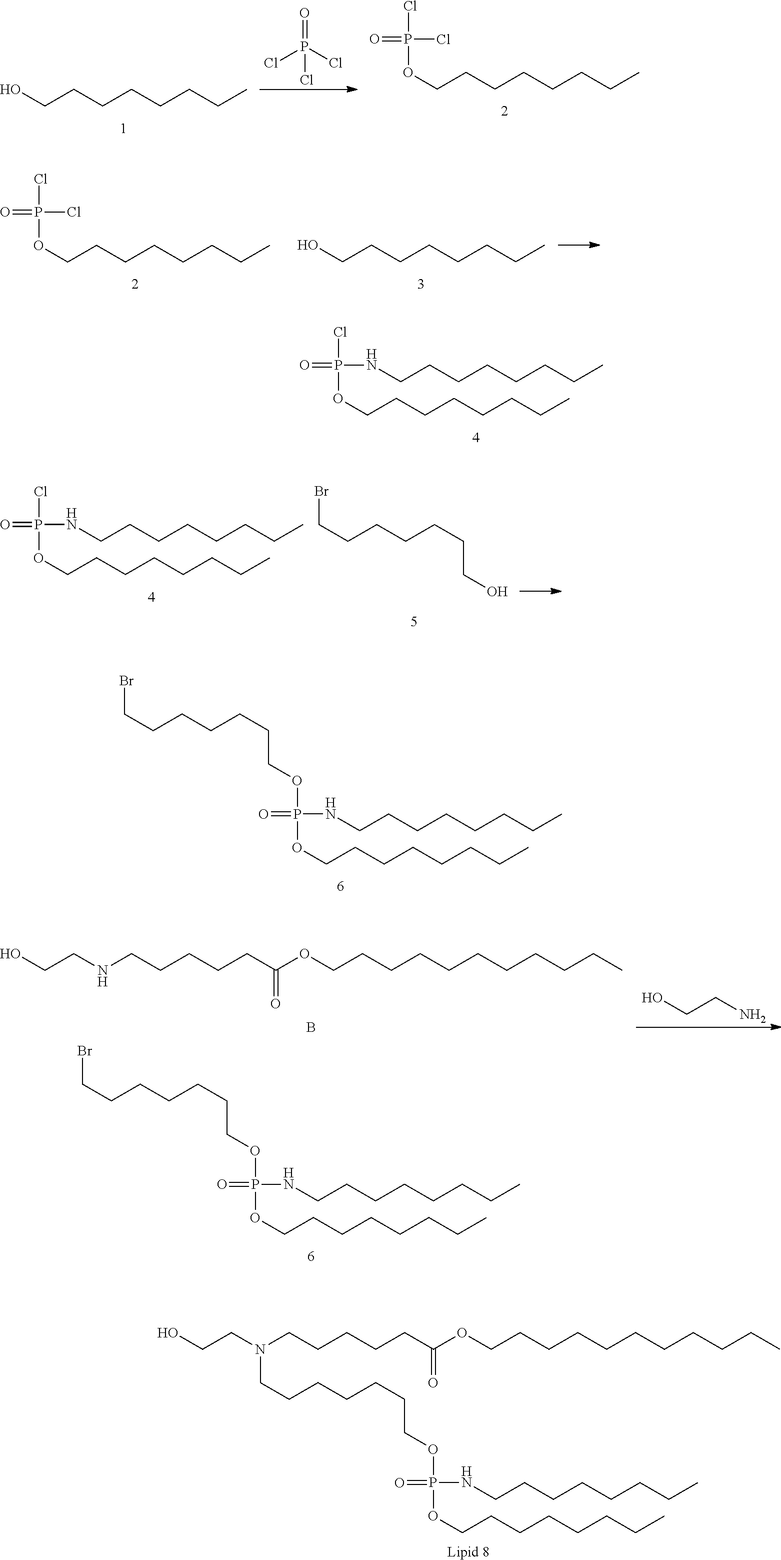

### Preparation of Compound 2:

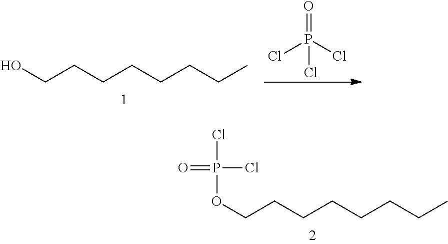

Phosphoryl trichloride (18.84 g, 122.86 mmol, 11.45 mL, 2 eq.) was added dropwise to Compound 1 (8 g, 61.43 mmol, 9.71 mL, 1 eq.) at 0° C., then the mixture was warmed to 20° C. for 2 hours. TLC indicated Compound 1 was consumed completely and one new spot formed. The mixture was concentrated to give Compound 2 (14 g, 56.66 mmol, 92.23% yield) as colorless oil. Which was used without further purification.

¹H NMR (400 MHz, CHLOROFORM-d) δ=4.34 (td, J=6.4, 9.6 Hz, 2H), 1.81 (q, J=6.8 Hz, 2H), 1.50-1.17 (m, 10H), 0.97-0.85 (m, 3H)

### Preparation of Compound 4:

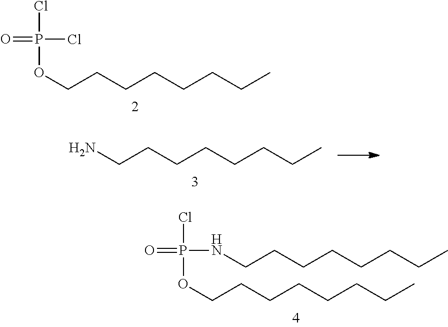

A mixture of Compound 3 (9 g, 36.42 mmol, 1 eq.), Compound 2 (3.77 g, 29.14 mmol, 4.82 mL, 0.8 eq.), TEA (2.95 g, 29.14 mmol, 4.06 mL, 0.8 eq.) in toluene (135 mL) was stirred at 20° C. for 2 hours. LCMS showed Compound 2 was consumed completely and one main peak with desired MS was detected. The mixture was quenched with H₂O (100 mL), extracted with EtOAc (200 mL*2), dried over Na₂SO₄, filtered, the filtrate was concentrated to give a residue. The residue was purified by column chromatography (SiO₂, Petroleum ether: Ethyl acetate=10:1 to 3:1) to give Compound 4 (10 g, 29.42 mmol, 80.78% yield) as yellow oil. LCMS [M+1]⁺=340.4

¹H NMR (400 MHz, CHLOROFORM-d) δ=4.28-4.03 (m, 2H), 3.23-3.09 (m, 1H), 3.09-2.96 (m, 2H), 1.79-1.49 (m, 5H), 1.47-1.18 (m, 20H), 1.02-0.77 (m, 6H)

### Preparation of Compound 6:

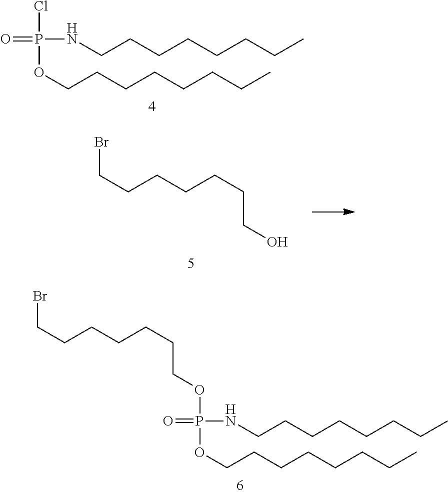 [SMILES: CCCCCCCCNP(=O)(Cl)OCCCCCCCC ; OCCCCCCCBr ; CCCCCCCCNP(=O)(OCCCCCCCC)OCCCCCCCBr]

A mixture of Compound 4 (1 g, 2.94 mmol, 1 eq.), Compound 5 (631.42 mg, 3.24 mmol, 1.1 eq.), TEA (595.45 mg, 5.88 mmol, 819.04 ILL, 2 eq.) in DCM (10 mL) was stirred at 30° C. for 12 hours. LCMS showed Compound 4 was consumed completely and one main peak with desired MS was detected. The mixture was quenched with H₂O (20 mL), extracted with ethyl acetate (20 mL*2), dried over Na₂SO₄, filtered, the filtrate was concentrated to give a residue. The residue was purified by column chromatography (SiO₂, Petroleum ether: Ethyl acetate=10:1 to 3:1) to give Compound 6 (0.48 g, 962.86 μmol, 32.73% yield) as yellow oil. LCMS [M+1]⁺=498

¹H NMR (400 MHz, CHLOROFORM-d) δ=4.09-3.89 (m, 4H), 3.41 (t, J=6.8 Hz, 2H), 2.97-2.84 (m, 2H), 1.87 (m, 2H), 1.73-1.58 (m, 5H), 1.56-1.21 (m, 29H), 0.89 (t, J=6.8 Hz, 6H)

### Preparation of Lipid 8:

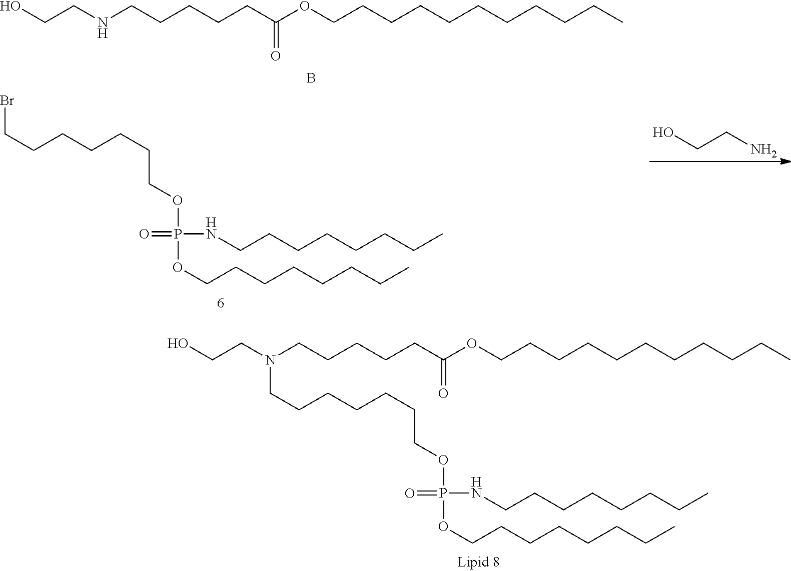

A mixture of Compound 6 (480 mg, 962.86 μmol, 1 eq.), Compound B (395.18 mg, 962.86 μmol, 1 eq.), K₂CO₃ (266.15 mg, 1.93 mmol, 2 eq.), NaI (72.16 mg, 481.43 μmol, 0.5 eq.) in ACN (5 mL) was stirred at 80° C. for 12 hours. LCMS showed Compound 6 was consumed completely and one main peak with desired MS was detected. The mixture was quenched with H₂O (10 mL), extracted with ethyl acetate (20 mL*2), dried over Na₂SO₄, filtered, the filtrate was concentrated to give a residue. The residue was purified by column chromatography (SiO₂, DCM:MeOH=50:1 to 10:1) to give Lipid 8 (0.2 g, 267.69 μmol, 27.80% yield) as yellow oil. LCMS [M+1]⁺=747.7

¹H NMR (400 MHz, CHLOROFORM-d) δ=4.08-3.95 (m, 6H), 3.62 (m, 2H), 2.93-2.85 (m, 2H), 2.57-2.47 (m, 7H), 2.33-2.29 (t, J=8.0 Hz, 2H), 1.69-1.59 (m, 8H), 1.57-1.45 (m, 6H), 1.45-1.17 (m, 44H), 0.89 (t, J=6.4 Hz, 9H)

### Lipid 9:

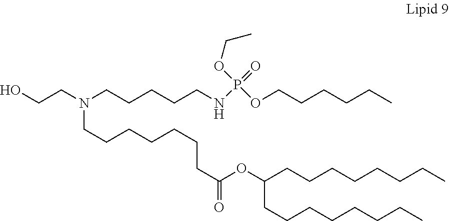 [SMILES: CCCCCCCCC(CCCCCCCC)OC(=O)CCCCCCCN(CCO)CCCCCNP(=O)(OCC)OCCCCCC]

### Synthesis of Lipid 9:

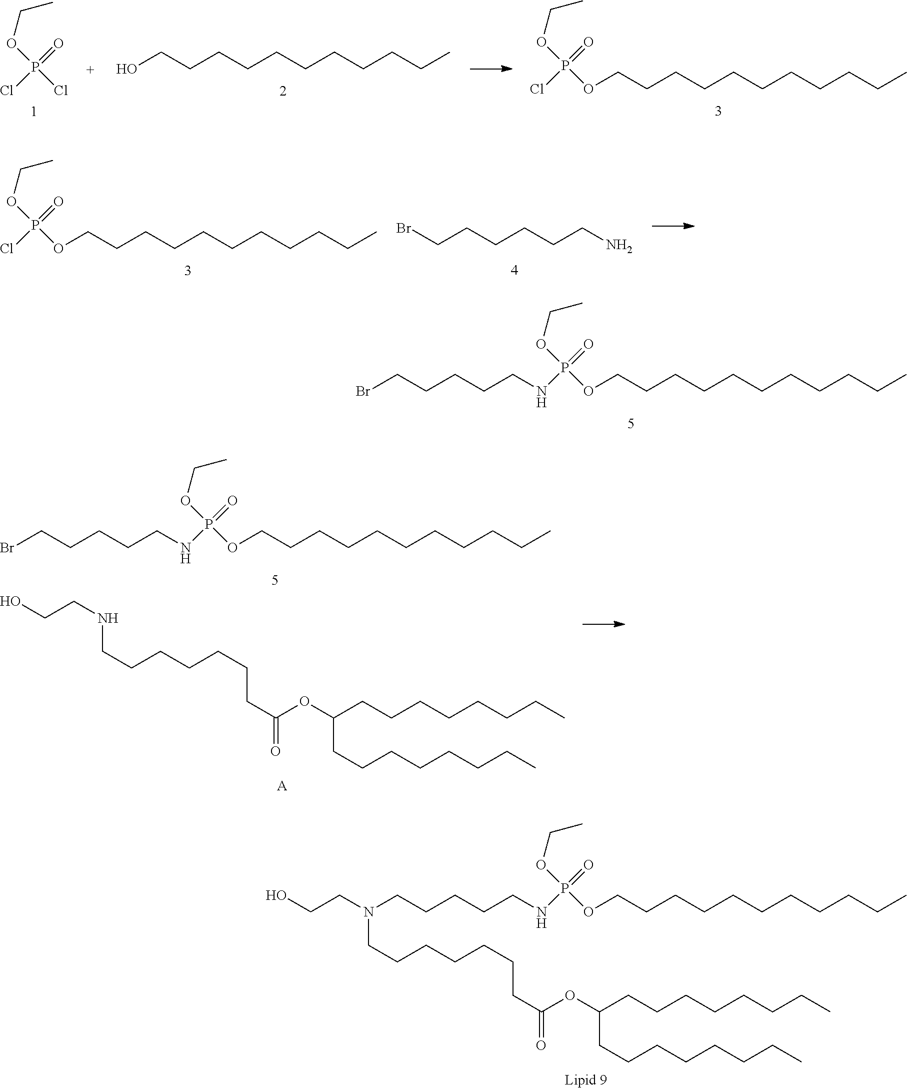

### Preparation of Compound 3:

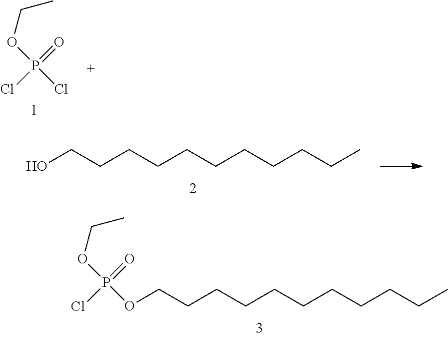

A solution of Compound 1 (8 g, 49.10 mmol, 5.83 mL, 1 eq.), Compound 2 (7.19 g, 41.73 mmol, 0.85 eq.) and TEA (7.45 g, 73.65 mmol, 10.25 mL, 1.5 eq.) in DCM (80 mL) was degassed and purged with N₂ for 3 times, and then the mixture was stirred at 20° C. for 2 hours under N₂ atmosphere. TLC (Dichloromethane:Methanol=25:1, P1: R_{f}=0.6) indicated Compound 1 was consumed and one new spot formed. The mixture was concentrated to give a residue that was purified by column chromatography (SiO₂, Petroleum ether: Ethyl acetate=100:1 to 50:1) to give Compound 3 (2.2 g, 6.63 mmol, 13.50% yield, 90% purity) as colorless oil.

¹H NMR (400 MHz, CHLOROFORM-d) δ=4.35-4.05 (m, 4H), 1.80-1.67 (m, 2H), 1.37 (s, 5H), 1.35-1.21 (m, 14H), 0.90-0.84 (m, 3H)

### Preparation of Compound 5:

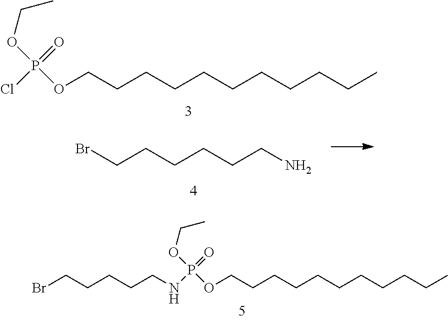 [SMILES: CCCCCCCCCCCOP(=O)(Cl)OCC ; NCCCCCCBr ; CCCCCCCCCCCOP(=O)(NCCCCCBr)OCC]

To a solution of Compound 3 (1.48 g, 4.94 mmol, 1 eq.) in DCM (5 mL) was added Compound 4 (1 g, 4.94 mmol, 1 eq., HCl) and TEA (2.00 g, 19.75 mmol, 2.75 mL, 4 eq.), and the mixture was stirred at 30° C. for 16 hours under N₂ atmosphere. LCMS showed Compound 3 was consumed completely and one main peak with desired MS was detected. The reaction mixture was diluted with sat. NH₄Cl (30 mL) and extracted with DCM (10 mL). The combined organic layers were dried over Na₂SO₄, filtered and concentrated under reduced pressure to give a residue. The residue was purified by column chromatography (SiO₂, Dichloromethane:Methanol=100:1 to 80:1) to give Compound 5 (1.1 g, 2.31 mmol, 46.80% yield, 90% purity) as yellow oil.

¹H NMR (400 MHz, CHLOROFORM-d) δ=4.15-3.89 (m, 4H), 3.47-3.35 (m, 2H), 3.12-3.06 (m, 11H), 2.98-2.85 (m, 2H), 2.57-2.39 (m, 1H), 1.91-1.85 (m, 1H), 1.72-1.61 (m, 2H), 1.57-1.45 (m, 4H), 1.40-1.20 (m, 19H), 0.94-0.80 (m, 3H)

### Preparation of Lipid 9

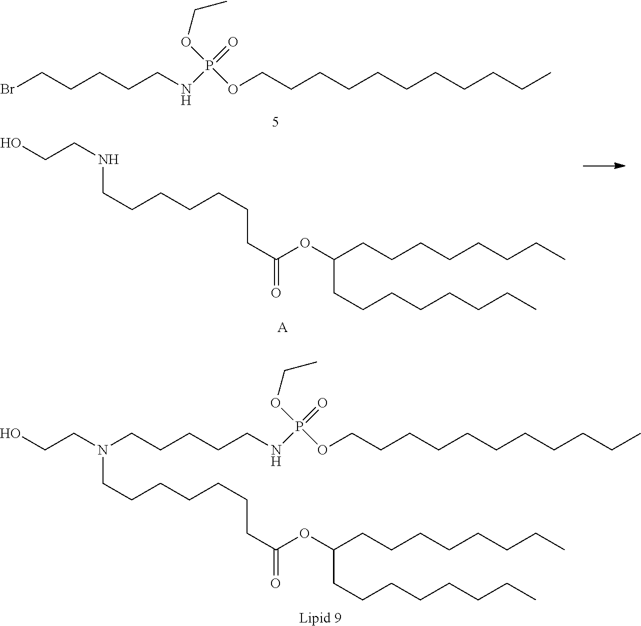

To a solution of Compound 5 (1.11 g, 2.58 mmol, 1 eq.), Compound A (1.14 g, 2.58 mmol, 1 eq.), K₂CO₃ (713.35 mg, 5.16 mmol, 2 eq.) and NaI (77.37 mg, 516.15 μmol, 0.2 eq.) in ACN (20 mL) was degassed and purged with N₂ for 3 times, and then the mixture was stirred at 80° C. for 16 hours under N₂ atmosphere. LCMS showed Compound 5 was consumed and one peak with desired MS was detected. The residue was diluted with H₂O (20 mL) and extracted with DCM (20 mL*2). The combined organic layers were dried over Na₂SO₄, filtered and concentrated under reduced pressure to give a residue. The residue was purified by column chromatography (SiO₂, Dichloromethane:Methanol=40:1) to give Lipid 9 (153.53 mg, 194.54 μmol, 7.54% yield, 100% purity) as yellow oil.

LCMS [M+1]⁺=789.7

¹H NMR (400 MHz, CHLOROFORM-d) δ=4.87-4.68 (m, 1H), 4.06-3.83 (m, 4H), 3.59-3.47 (m, 2H), 2.88-2.76 (m, 2H), 2.64-2.59 (m, 2H), 2.55-2.41 (m, 5H), 2.30-2.14 (m, 3H), 1.66-1.52 (m, 4H), 1.50 (s, 10H), 1.33-1.12 (m, 51H), 0.86-0.73 (m, 9H)

### Lipid 10:

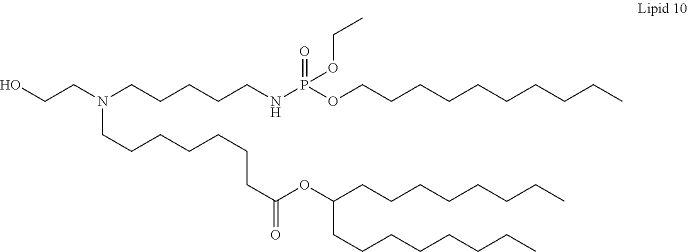 [SMILES: CCCCCCCCCCOP(=O)(NCCCCCN(CCO)CCCCCCCC(=O)OC(CCCCCCCC)CCCCCCCC)OCC]

### Synthesis of Lipid 10:

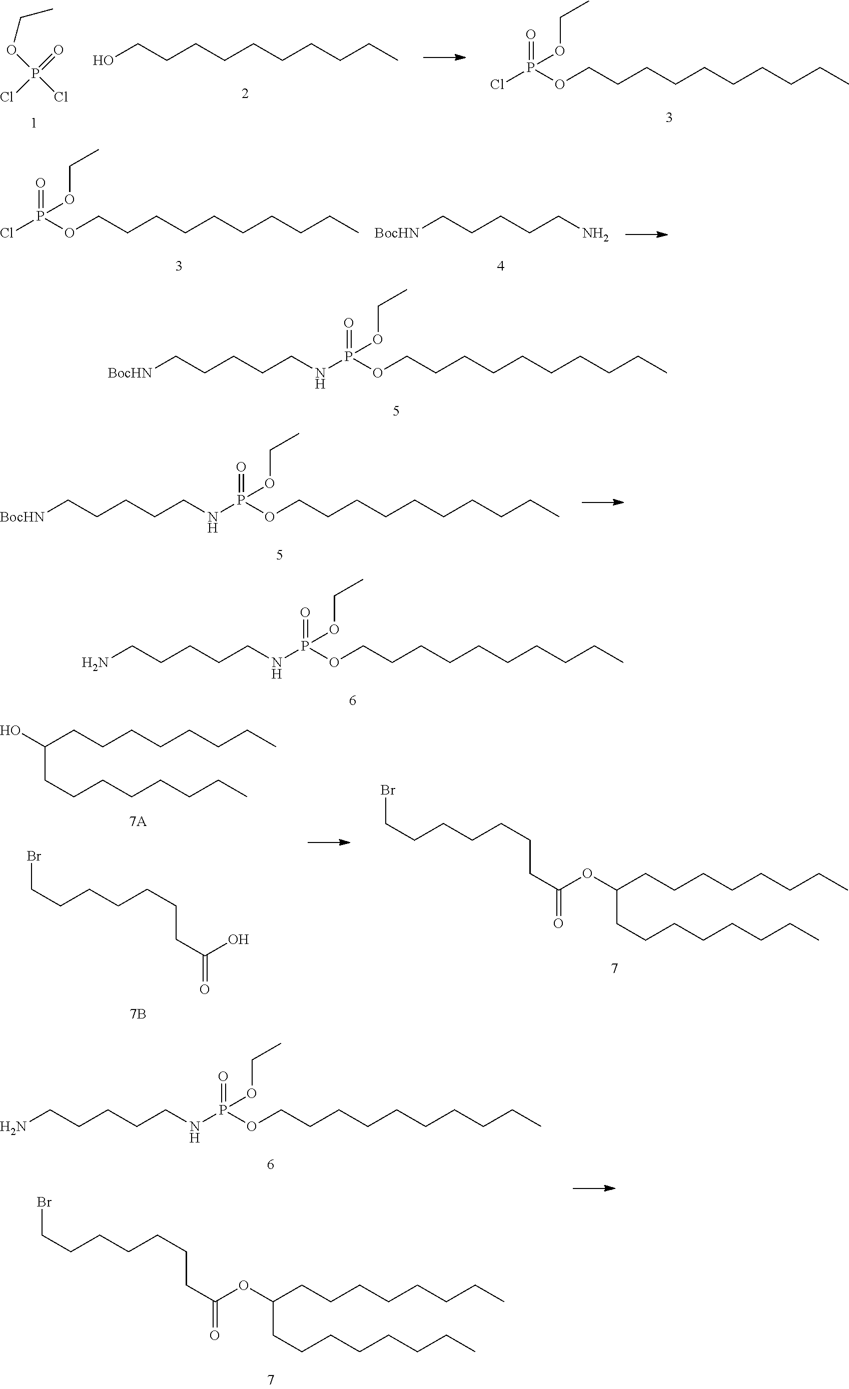

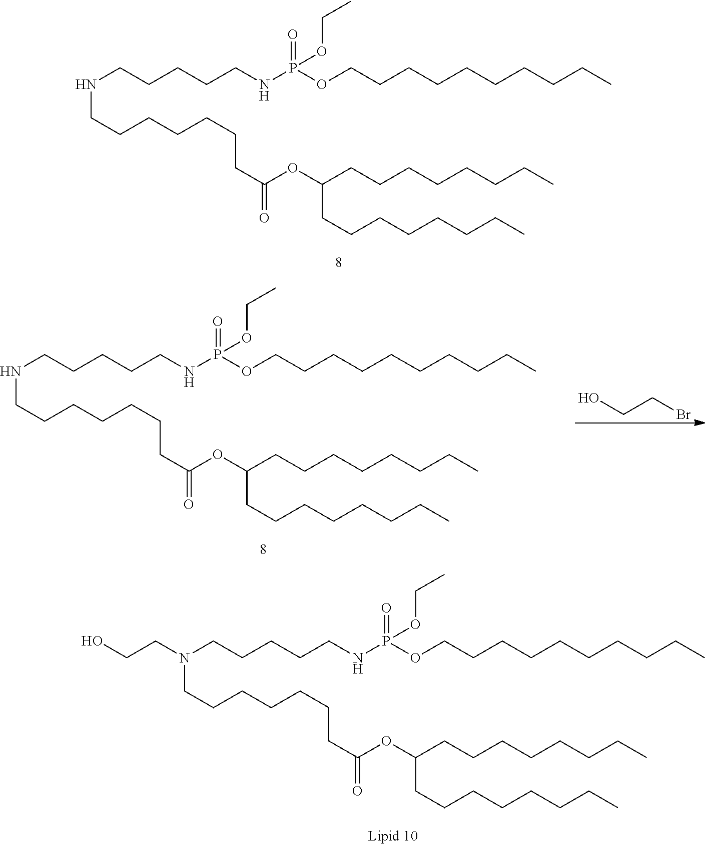

### Preparation of Compound 3:

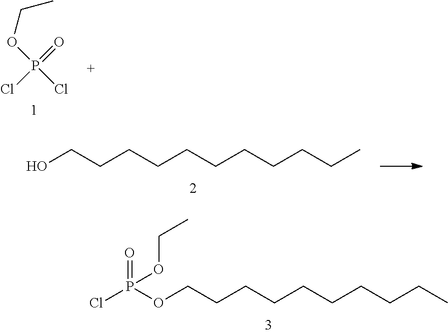

To a solution of Compound 2 (5 g, 30.69 mmol, 3.64 mL, I eq.) in DCM (50 mL) was added TEA (4.66 g, 46.03 mmol, 6.41 mL, 1.5 eq.), Compound 1 (4.13 g, 26.08 mmol, 4.98 mL, 0.85 eq.). The mixture was stirred at 20° C. for 2 hours. LCMS showed Compound 1 was consumed completely and one main peak with desired MS was detected. The residue was diluted with DCM (30 mL) and washed with sat. NH₄Cl (10 mL*3), dried over Na₂SO₄, filtered and concentrated under reduced pressure to give a residue which was then purified by column chromatography (SiO₂, Petroleum ether: Ethyl acetate=100:1 to 40:1) to give Compound 3 (3.29 g, 11.55 mmol, 37.65% yield) as yellow oil.

¹H NMR (400 MHz, CHLOROFORM-d) δ=4.42-4.09 (m, 4H), 1.79-1.70 (m, 2H), 1.45-1.36 (m, 5H), 1.35-1.23 (m, 12H), 0.92-0.87 (m, 3H)

### Preparation of Compound 5:

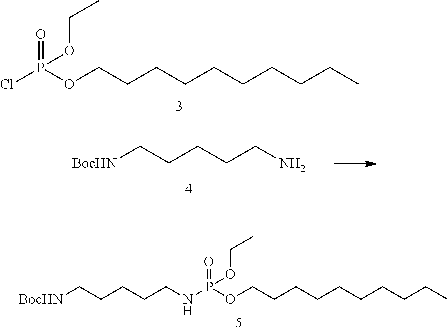 [SMILES: CCCCCCCCCCOP(=O)(Cl)OCC ; CC(C)(C)OC(=O)NCCCCCN ; CCCCCCCCCCOP(=O)(NCCCCCNC(=O)OC(C)(C)C)OCC]

To a solution of Compound 3 (1.5 g, 5.27 mmol, 1 eq.) in DCM (15 mL) was added TEA (1.17 g, 11.59 mmol, 1.61 mL, 2.2 eq.), and Compound 4 (1.07 g, 5.27 mmol, 1.10 mL, 1 eq.). The mixture was stirred at 30° C. for 16 hours. LCMS showed Compound 3 was consumed completely and one main peak with desired MS was detected. The reaction mixture was diluted with DCM (30 mL) and washed with sat.NH₄Cl (10 mL*3), dried over Na₂SO₄, filtered and concentrated under reduced pressure to give a residue. The residue was purified by column chromatography (SiO₂, Petroleum ether: Ethyl acetate=15:1 to 1:1) to give Compound 5 (2 g, 4.44 mmol, 84.26% yield) as yellow oil.

LCMS [M+1−100]⁺=351.3

¹H NMR (400 MHz, CHLOROFORM-d) δ=4.60-4.46 (m, 1H), 4.14-3.91 (m, 4H), 3.17-3.05 (m, 2H), 2.96-2.86 (m, 2H), 1.72-1.62 (m, 2H), 1.54-1.48 (m, 4H), 1.45 (s, 9H), 1.40-1.23 (m, 20H), 0.92-0.86 (m, 3H)

### Preparation of Compound 6:

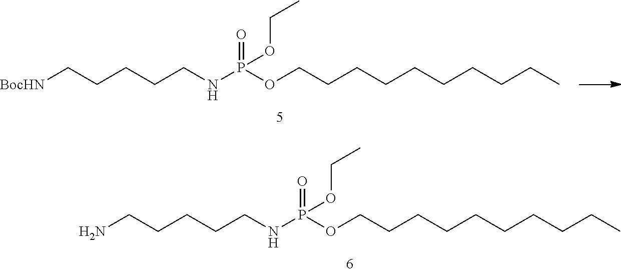 [SMILES: CCCCCCCCCCOP(=O)(NCCCCCNC(=O)OC(C)(C)C)OCC ; CCCCCCCCCCOP(=O)(NCCCCCN)OCC]

To a solution of Compound 5 (1.5 g, 3.33 mmol, 1 eq.) in DCM (15 mL) was added TFA (7.59 g, 66.58 mmol, 4.95 mL, 20 eq.). The mixture was stirred at 20° C. for 1 hour. LCMS showed Compound 5 was consumed completely and one main peak with desired MS was detected. The reaction mixture was diluted with DCM (40 mL) and washed with sat.NaHCO₃, dried over Na₂SO₄, filtered and concentrated under reduced pressure to give Compound 6 (1 g, 2.85 mmol, 85.71% yield) as yellow oil.

¹H NMR (400 MHz, CHLOROFORM-d) δ=8.76-8.48 (m, 2H), 4.84-4.51 (m, 1H), 4.11-3.86 (m, 3H), 3.86-3.77 (m, 1H), 3.00-2.83 (m, 2H), 1.79-1.68 (m, l H), 1.67-1.55 (m, 3H), 1.53-1.44 (m, 1H), 1.37-1.23 (m, 18H), 0.91-0.86 (m, 3H)

### Preparation of Compound 7:

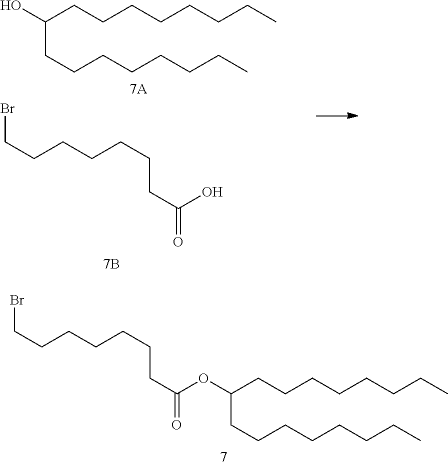 [SMILES: CCCCCCCCC(O)CCCCCCCC ; O=C(O)CCCCCCCBr ; CCCCCCCCC(CCCCCCCC)OC(=O)CCCCCCCBr]

A mixture of Compound 7A (10.44 g, 46.79 mmol, 1.2 eq.), Compound 7B (10 g, 38.99 mmol, 1 eq), DMAP (952.70 mg, 7.80 mmol, 0.2 eq.), EDCI (8.97 g, 46.79 mmol, 1.2 eq.) and DIEA (10.08 g, 77.98 mmol, 13.58 mL, 2 eq.) in DCM (100 mL) was degassed and purged with N₂ for 3 times, and then the mixture was stirred at 20° C. for 16 hours under N₂ atmosphere. TLC (Petroleum ether: Ethyl acetate=10:1, R_{f}=0.67) indicated Compound 7B was consumed completely and many new spots formed. The reaction mixture (2 batches) was partitioned between DCM (200 mL) and sat.NH₄Cl (100 mL). The organic phase was dried over Na₂SO₄, filtered and concentrated under reduced pressure to give a residue. The residue was purified by column chromatography (SiO₂, Petroleum ether: Ethyl acetate=100:1 to 20:1) to give Compound 7 (29 g, 62.83 mmol, 63.24% yield) as colorless oil.

LCMS [M+23]⁺=483.43

¹H NMR (400 MHz, CHLOROFORM-d) δ=4.87 (quin, J=6.4 Hz, 1H), 3.40 (t, J=6.8 Hz, 2H), 2.29 (t, J=7.6 Hz, 2H), 1.92-1.73 (m, 2H), 1.68-1.58 (m, 2H), 1.51 (br d, J=5.6 Hz, 4H), 1.47-1.40 (m, 2H), 1.39-1.30 (m, 6H), 1.32-1.18 (m, 21H), 0.88 (t, J=6.8 Hz, 6H)

### Preparation of Compound 8:

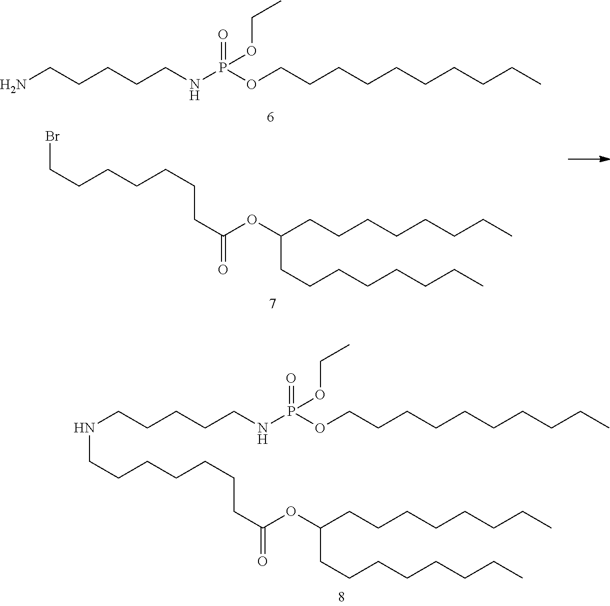 [SMILES: CCCCCCCCCCOP(=O)(NCCCCCN)OCC ; CCCCCCCCC(CCCCCCCC)OC(=O)CCCCCCCBr ; CCCCCCCCCCOP(=O)(NCCCCCNCCCCCCCC(=O)OC(CCCCCCCC)CCCCCCCC)OCC]

To a solution of Compound 6 (700 mg, 2.00 mmol, 1 eq.) in DMF (14 mL) was added Cs₂CO₃ (452.92 mg, 1.39 mmol, 0.696 eq.) and Compound 7 (921.86 mg, 2.00 mmol, 1 eq.). The mixture was stirred at 50° C. for 16 hours. LCMS showed Compound 7 was consumed completely and one main peak with desired MS was detected. The residue was diluted with Ethyl acetate (50 mL) and washed with H₂O (20 mL), brine (20 mL), dried over Na₂SO₄, filtered and concentrated under reduced pressure to give a residue. The residue was purified by column chromatography (SiO₂, DCM:MeOH=30:1 to 15:1) to give Compound 8 (0.6 g, 820.66 μmol, 41.09% yield) as yellow oil.

LCMS [M+1]⁺=731.7

¹H NMR (400 MHz, DMSO-d₆) δ=4.85-4.73 (m, 2H), 3.93-3.72 (m, 4H), 3.70-3.55 (m, 1H), 2.76 (br s, 6H), 2.28-2.20 (m, 2H), 1.58-1.36 (m, 14H), 1.30-1.18 (m, 48H), 1.14-1.06 (m, 2H), 0.90-0.80 (m, 9H)

### Preparation of Lipid 10.

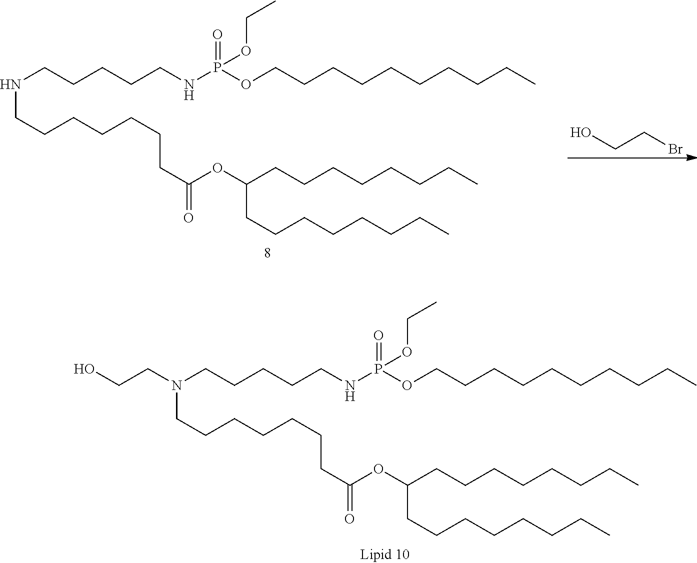

To a solution of Compound 8 (200 mg, 273.55 μmol, 1 eq.) in dioxane (10 mL) was added K₂CO₃ (56.71 mg, 410.33 μmol, 1.5 eq.), NaI (20.50 mg, 136.78 μmol, 0.5 eq.), 2-bromoethanol (170.92 mg, 1.37 mmol, 96.95 μL, 5 eq.). The mixture was stirred at 110° C. for 16 hours. LCMS showed Compound 8 was consumed completely and one main peak with desired MS was detected. The reaction mixture was concentrated under reduced pressure to give a residue. The residue was diluted with DCM (30 mL) and washed with sat.NH₄Cl (10 mL*3), dried over Na₂SO₄, filtered and concentrated under reduced pressure to give a residue. The residue was purified by column chromatography (SiO₂, DCM:MeOH=20:1 to 15:1) to give Lipid 10 (59 mg, 76.11 μmol, 27.82% yield, 100/6 purity) as yellow oil.

LCMS [M+1]⁺=775.7

¹H NMR (400 MHz, CHLOROFORM-d) δ=4.91-4.82 (m, 1H), 4.12-4.02 (m, 2H), 4.01-3.91 (m, 2H), 3.83-3.62 (m, 2H), 3.05-2.61 (m, 8H), 2.60-2.52 (m, 1H), 2.32-2.25 (m, 2H), 1.71-1.59 (m, 7H), 1.56-1.47 (m, 7H), 1.37-1.22 (m, 50H), 0.93-0.85 (m, 9H)

### Lipid 11:

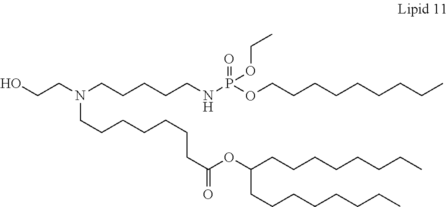 [SMILES: CCCCCCCCCOP(=O)(NCCCCCN(CCO)CCCCCCCC(=O)OC(CCCCCCCC)CCCCCCCC)OCC]

### Synthesis of Lipid 11:

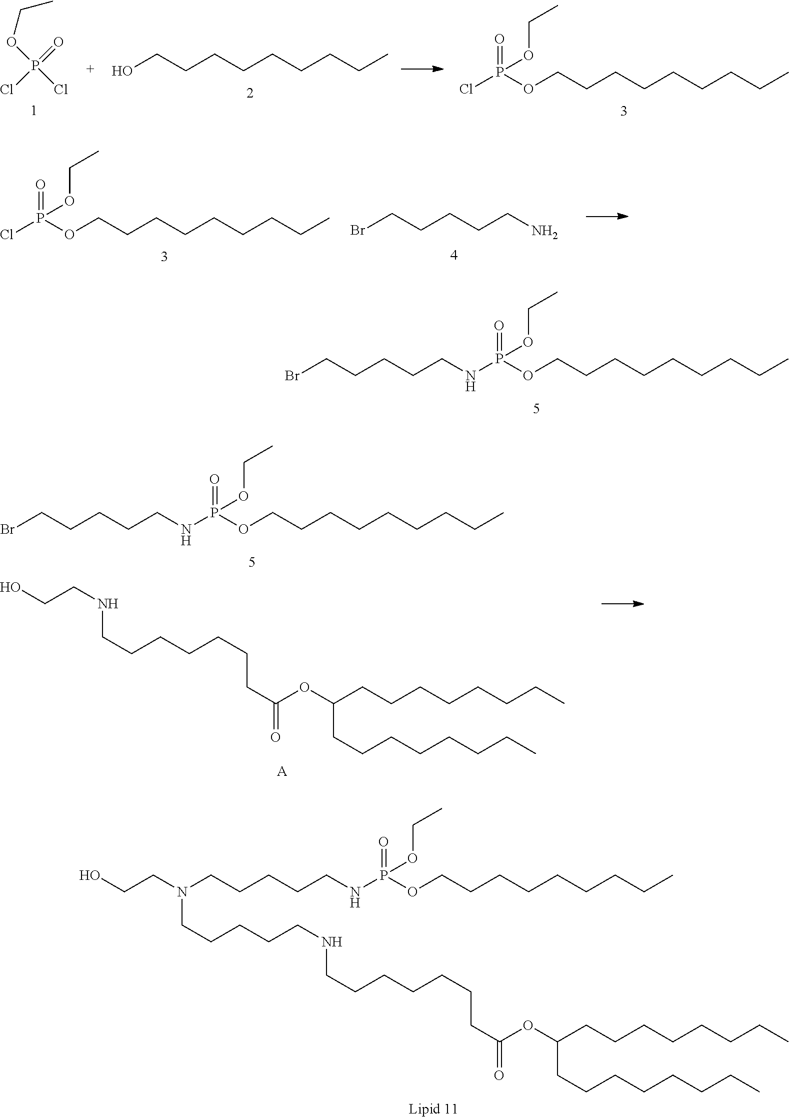

### Preparation of Compound 3

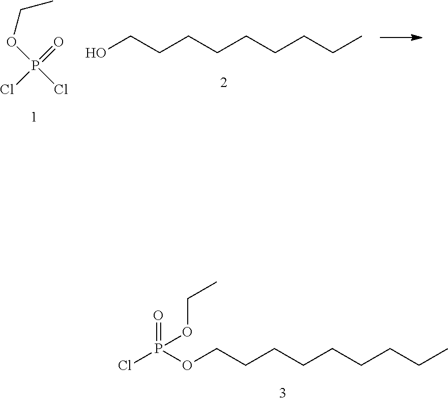

To a solution of Compound 1 (8 g, 49.10 mmol, 5.83 mL, 1 eq.) and Compound 2 (6.02 g, 41.73 mmol, 0.85 eq.) in DCM (80 mL) was added TEA (7.45 g, 73.65 mmol, 10.25 mL, 1.5 eq.). The mixture was stirred at 20° C. for 16 hours. TLC (Dichloromethane:Methanol=25:1, P1: R_{f}=0.6) indicated Compound 2 was consumed completely and one new spot formed. The residue was diluted with sat. NH₄Cl (10 mL) and extracted with DCM (20 mL). The combined organic layers were dried over Na₂SO₄, filtered and concentrated under reduced pressure to give a residue. The residue was purified by column chromatography (SiO₂, Petroleum ether: Ethyl acetate=100:1 to 50:1) to give Compound 3 (1.8 g, 6.65 mmol, 13.54% yield, 100% purity) as colorless oil.

¹H NMR (400 MHz, CHLOROFORM-d) δ=4.29-4.03 (m, 4H), 1.73-1.59 (m, 2H), 1.38-1.29 (m, 5H), 1.28-1.16 (m, 10H), 0.86-0.74 (m, 3H)

### Preparation of Compound 5

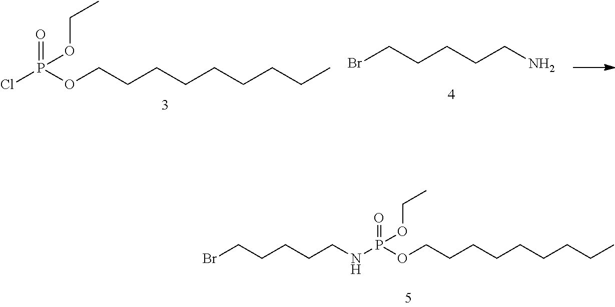

To a solution of Compound 3 (996.54 mg, 3.68 mmol, 1 eq.) in DCM (8.2 mL) was added TEA (1.49 g, 14.72 mmol, 2.05 mL, 4 eq.) and Compound 4 (0.82 g, 4.05 mmol, 1.1 eq.). The mixture was stirred at 30° C. for 4 hours. LCMS showed Compound 3 was consumed completely and one main peak with desired MS was detected. The residue was diluted with DCM (30 mL) and washed with sat. NH₄Cl (10 mL*3), dried over Na₂SO₄, filtered and concentrated under reduced pressure to give a residue. The residue was purified by column chromatography (SiO₂, DCM:MEOH=100:1 to 1:1) to give Compound 5 (1.07 g, 2.67 mmol, 72.61% yield) as yellow oil.

LCMS [M+1]⁺=400.2

¹H NMR (400 MHz, CHLOROFORM-d) δ=4.12-3.89 (m, 4H), 3.45-3.37 (m, 1H), 3.14-3.06 (m, 1H), 2.98-2.88 (m, 2H), 2.53-2.40 (m, 1H), 1.92-1.75 (m, 2H), 1.71-1.58 (m, 4H), 1.57-1.46 (m, 4H), 1.41-1.24 (m, 14H), 0.92-0.84 (m, 3H)

### Preparation of Lipid 11:

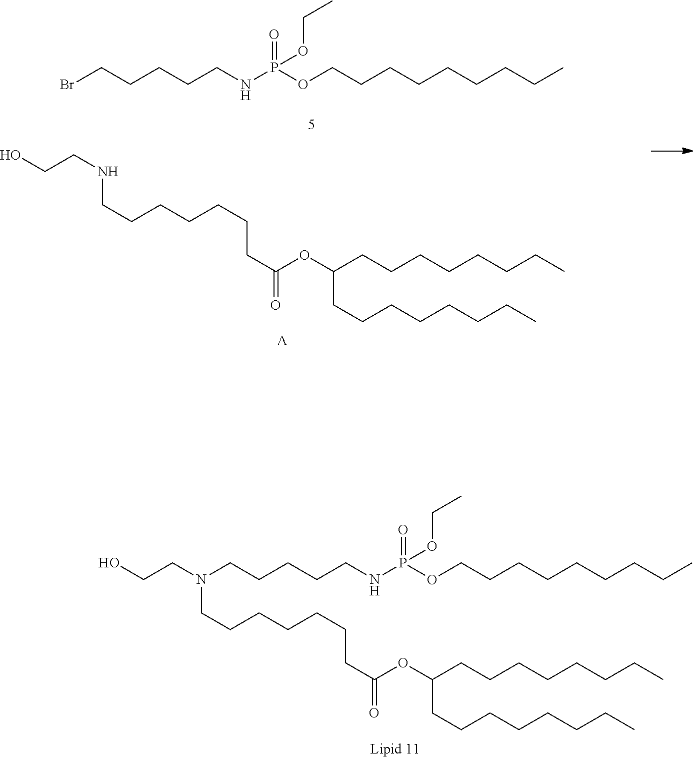

To a solution of Compound 5 (0.3 g, 749.38 μmol, 1 eq.) in ACN (6 mL) was added K₂COI (207.14 mg, 1.50 mmol, 2 eq.), NaI (22.46 mg, 149.88 μmol, 0.2 eq.), Compound A (331.02 mg, 749.38 μmol, 1 eq.). The mixture was stirred at 80° C. for 16 hours. LCMS showed Compound 5 was consumed completely and one main peak with desired MS was detected. The residue was diluted with DCM (30 mL) and washed with sat. NH₄Cl (10 mL*3), dried over Na₂SO₄, filtered and concentrated under reduced pressure to give a residue. The residue was purified by column chromatography (SiO₂, DCM:MEOH=50:1 to 40:1) to give Lipid 11 (109 mg, 141.77 μmol, 18.92% yield, 99% purity) as yellow oil.

LCMS [M+1]⁺=761.7

¹H NMR (400 MHz, DMSO-d₆) δ=9.37-9.24 (m, 1H), 5.36-5.25 (m, 1H), 4.87-4.73 (m, 2H), 3.94-3.76 (m, 4H), 3.75-3.68 (m, 2H), 3.19-2.99 (m, 6H), 2.79-2.69 (m, 2H), 2.29-2.23 (m, 2H), 1.65-1.38 (m, 14H), 1.30-1.17 (m, 47H), 0.89-0.81 (m, 9H).

### Lipid 12:

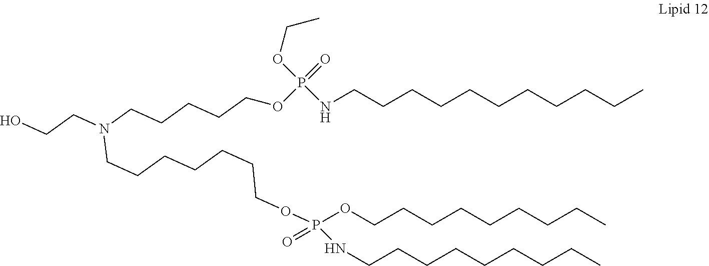 [SMILES: CCCCCCCCCCCNP(=O)(OCC)OCCCCCN(CCO)CCCCCCCOP(=O)(NCCCCCCCCC)OCCCCCCCCC]

### Synthesis of Lipid 12:

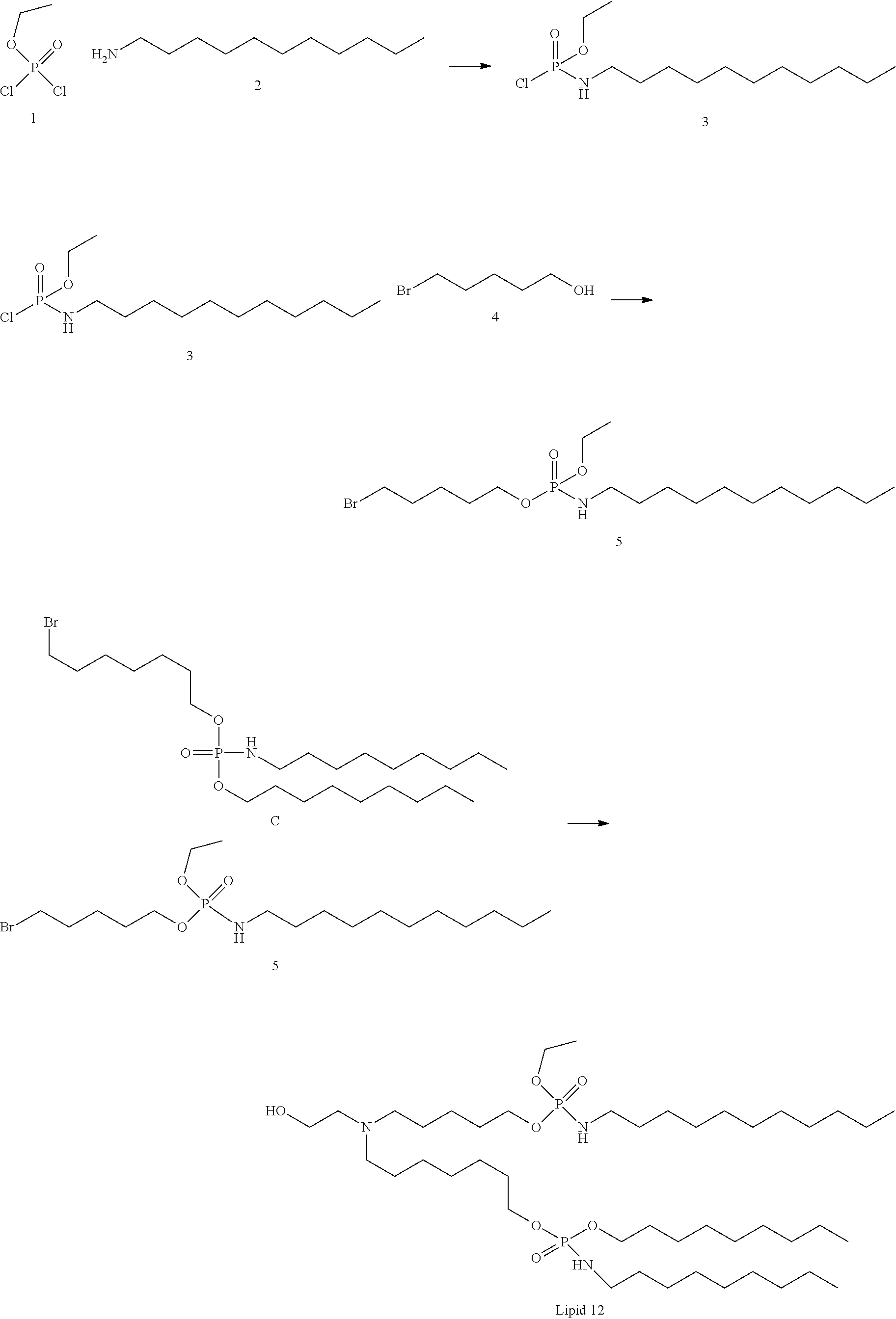

### Preparation of Compound 3:

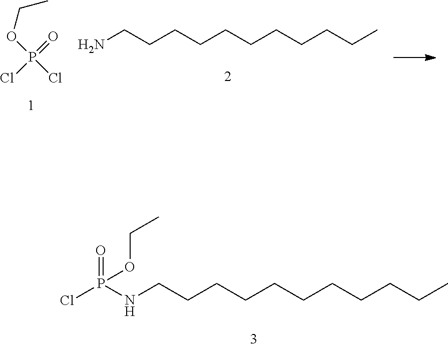

To a solution of Compound 1 (9.04 g, 55.45 mmol, 6.58 mL, 1 eq.) and TEA (5.89 g, 58.22 mmol, 8.10 mL, 1.05 eq.) in DCM (90 mL) was added Compound 2 (9.5 g, 55.45 mmol, 1 eq.) dropwise at 0° C. The resulting mixture was stirred at 20° C. for 2 hours. LCMS showed Compound 2 was consumed and desired MS was detected. The reaction mixture was concentrated to give a residue. The residue was purified by column chromatography (SiO₂, Petroleum ether: Ethyl acetate=10:1 to 5:1) to give Compound 3 (4.1 g, 6.79 mmol, 13.22% yield, 100% purity) as yellow oil.

LCMS [M+1]⁺=298.2

¹H NMR (400 MHz, CHLOROFORM-d) δ=4.32-4.13 (m, 2H), 3.56-3.44 (m, 1H), 3.08-2.92 (m, 2H), 1.59-1.49 (m, 2H), 1.43-1.21 (m, 19H), 0.89-0.85 (m, 3H)

### Preparation of Compound 5:

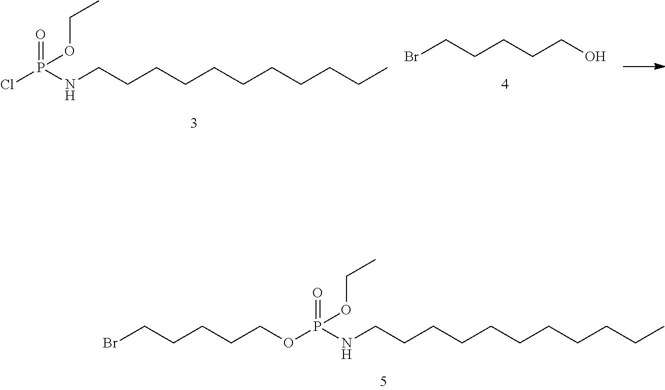

To a solution of Compound 3 (2 g, 6.72 mmol, 1 eq.) in DCM (20 mL) was added TEA (1.36 g, 13.43 mmol, 1.87 mL, 2 eq.) and Compound 4 (1.23 g, 7.39 mmol, 1.1 eq.). The resulting mixture was stirred at 20° C. for 16 hours. LCMS showed no Compound 3 remained and desired MS was detected. The reaction mixture was diluted with DCM (20 mL) and washed with sat.NH₄Cl (10 mL*3), dried over Na₂SO₄, filtered and concentrated under reduced pressure to give a residue. The residue was purified by reversed-phase HPLC (column: Welch Xtimate C1 250*50 mm*10 um; mobile phase: [H₂O (10 mM NH₄HCO₃)-ACN:THF=1:1]; gradient: 40%-80% B over 20.0 min) to give Compound 5 (0.55 g, 1.28 mmol, 19.12% yield, 100% purity) as white solid.

LCMS [M+1]⁺=428.3

¹H NMR (400 MHz, DMSO-d₆) δ=4.83-4.73 (m, 1H), 3.93-3.78 (m, 4H), 3.59-3.48 (m, 2H), 2.76-2.66 (m, 2H), 1.87-1.77 (m, 2H), 1.64-1.55 (m, 2H), 1.49-1.42 (m, 2H), 1.37 (br d, J=6.4 Hz, 1H), 1.30-1.17 (m, 20H), 0.89-0.82 (m, 3H)

### Preparation of Lipid 12:

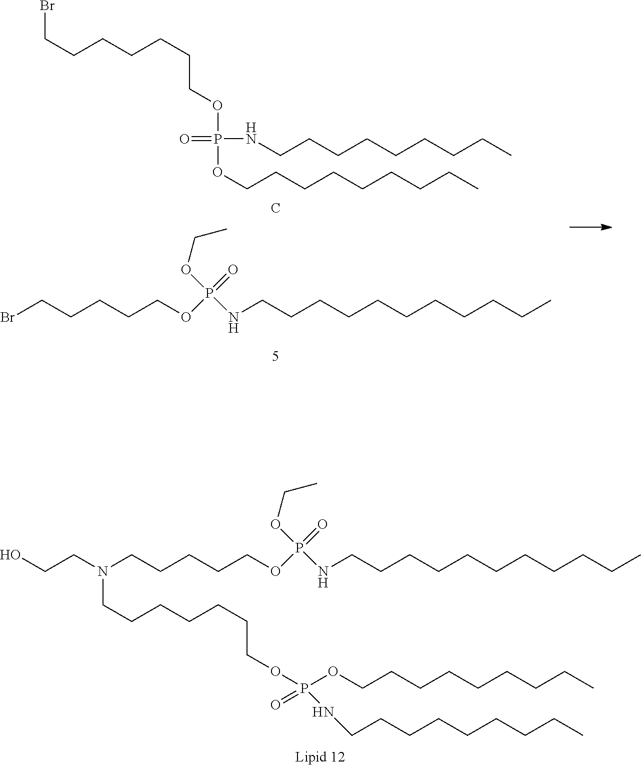

To a solution of Compound C (659.39 mg, 1.54 mmol, 1.3 eq.) in ACN (12 mL) was added Compound 5 (0.6 g, 1.18 mmol, 1 eq.), K₂CO₃ (327.28 mg, 2.37 mmol, 2 eq.) and NaI (35.50 mg, 236.81 μmol, 0.2 eq.). The resulting mixture was stirred at 80° C. for 16 hours. LCMS showed Compound C was consumed and desired MS was detected. The reaction mixture was diluted with DCM (20 mL) and washed with H₂O (6 mL), dried over Na₂SO₄, filtered and concentrated under reduced pressure to give a residue. The residue was purified by column chromatography (SiO₂, Dichloromethane:Methanol=40:1 to 4:1) to give Lipid 12 (102 mg, 119.41 μmol, 51.00% yield, 100% purity) as yellow oil.

LCMS [M+1]⁺=854.7

¹H NMR (400 MHz, CHLOROFORM-d) δ=4.10-3.92 (m, 8H), 3.81-3.60 (m, 2H), 2.95-2.56 (m, 12H), 1.70-1.63 (m, 6H), 1.56-1.19 (m, 59H), 0.89 (t, J=6.8 Hz, 9H)

### Lipid 13:

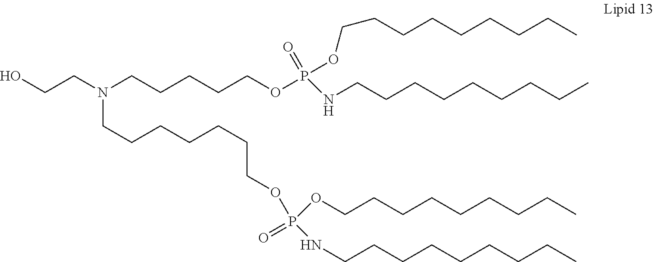 [SMILES: CCCCCCCCCNP(=O)(OCCCCCCCCC)OCCCCCCCN(CCO)CCCCCOP(=O)(NCCCCCCCCC)OCCCCCCCCC]

### Synthesis of Lipid 13:

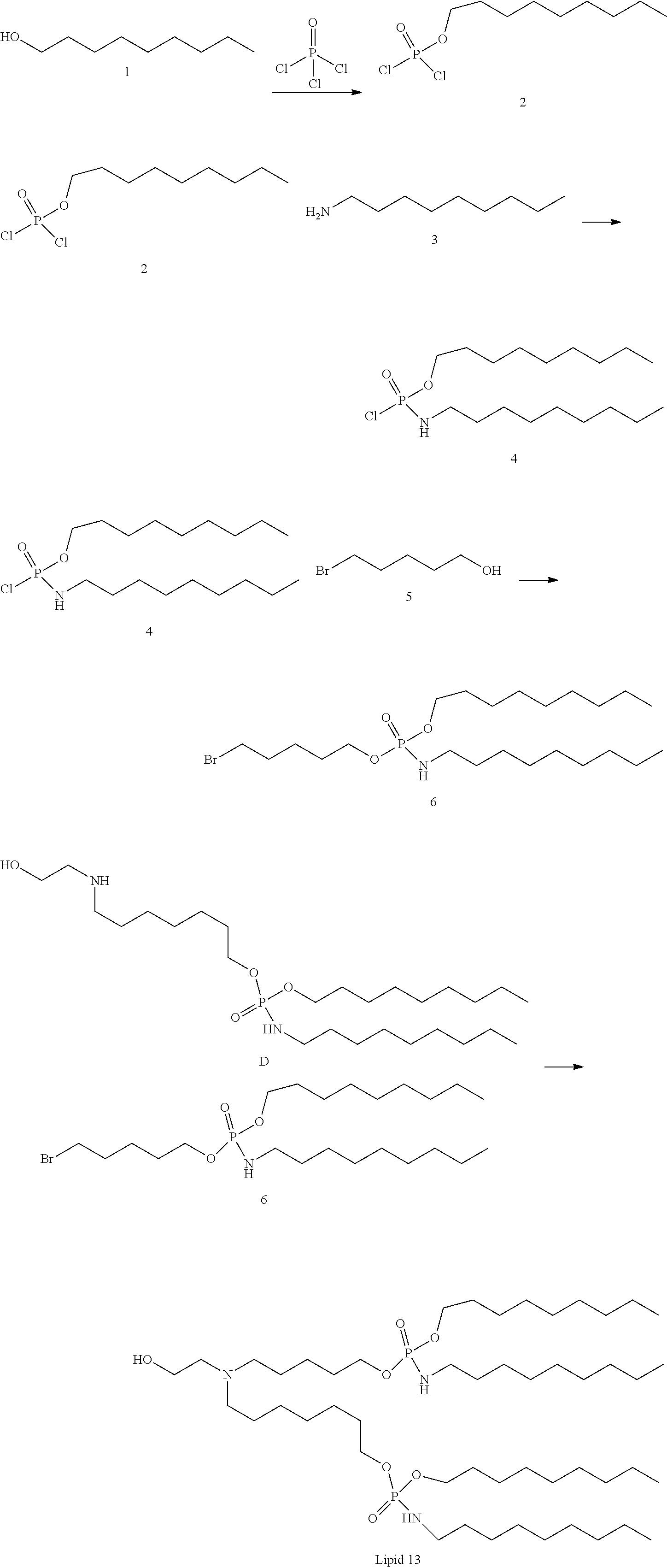

### Preparation of Compound 2:

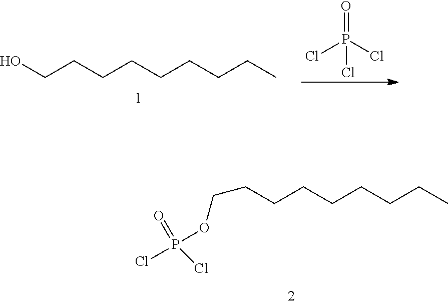

To a solution of Compound 1 (10 g, 69.32 mmol, 1 eq.) in DCM (2 mL) was added POCl₃ (21.26 g, 138.64 mmol, 12.92 mL, 2 eq.). The mixture was stirred at 20° C. for 3 hours. TLC (Petroleum ether: Ethyl acetate=3:1, R=0.76) indicated Compound 1 was consumed, and one major new spot was detected. The reaction mixture was evaporated with toluene (5 mL*3) three times to give Compound 2 (21.42 g, crude) as brown oil.

¹H NMR (400 MHz, CHLOROFORM-d) δ=4.42-4.29 (m, 2H), 1.88-1.71 (m, 2H), 1.49-1.24 (m, 10H), 0.95-0.87 (m, 3H)

### Preparation of Compound 4:

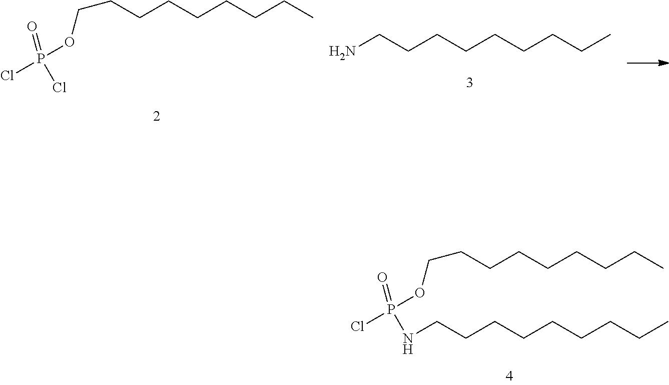

To a solution of Compound 2 (21.42 g, 82.01 mmol, 1 eq.) in toluene (460 mL) was added TEA (6.64 g, 65.61 mmol, 9.13 mL, 0.8 eq.) and Compound 3 (9.4 g, 65.61 mmol, 0.8 eq.). The mixture was stirred at 20° C. for 3 hours. LCMS showed Compound 2 was consumed completely and one main peak with desired MS was detected. The reaction mixture was diluted with sat.NH₄Cl (20 mL) and extracted with DCM (25 mL). The combined organic layers were washed with brine (20 mL), dried over Na₂SO₄, filtered and concentrated under reduced pressure to give a residue. The residue was purified by column chromatography (SiO₂, Petroleum ether: Ethyl acetate=100:1 to 10:1) to give Compound 4 (14 g, 38.05 mmol, 46.39% yield) as yellow oil.

LCMS [M+1]⁺=368.3

1H NMR (400 MHz, CHLOROFORM-d) δ=4.25-4.03 (m, 2H), 3.22-3.11 (m, 1H), 3.24-3.10 (m, 1H), 3.08-2.93 (m, 2H), 1.78-1.68 (m, 2H), 1.60-1.50 (m, 2H), 1.43-1.22 (m, 24H), 0.89 (br t, J=6.8 Hz, 6H)

### Preparation of Compound 6:

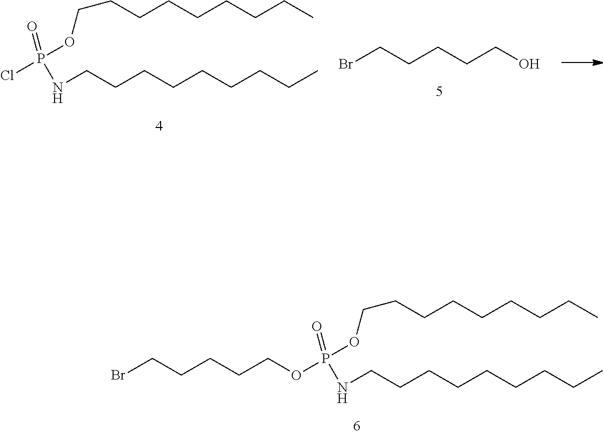

To a solution of Compound 4 (3 g, 8.15 mmol, 1 eq.) in toluene (30 mL) was added TEA (1.65 g, 16.31 mmol, 2.27 mL, 2 eq.) and Compound 5 (1.36 g, 8.15 mmol, 1 eq.). The mixture was stirred at 50° C. for 16 hours. LCMS showed Compound 4 was consumed completely and one main peak with desired MS was detected. The reaction mixture was concentrated under reduced pressure to give a residue. The residue was purified by prep-HPLC (column: Welch Xtimate C1 100*30 mm*5 um; mobile phase: [H₂O (10 mM NHaHCO₃)-THF:ACN=1:3]; gradient: 48%-88% B over 20.0 min) to give Compound 6 (1.7 g, 3.41 mmol, 28.33% yield) as yellow oil.

LCMS [M+1]⁺=498.3

1H NMR (400 MHz, CHLOROFORM-d) δ=4.06-3.92 (m, 4H), 3.57-3.52 (m, 1H), 3.42 (t, J=6.8 Hz, 2H), 2.92-2.83 (m, 2H), 2.53-2.36 (m, 1H), 1.96-1.78 (m, 2H), 1.76-1.62 (m, 5H), 1.61-1.53 (m, 4H), 1.40-1.21 (m, 24H), 0.89 (t, J=6.8 Hz, 6H)

### Preparation of Lipid 13:

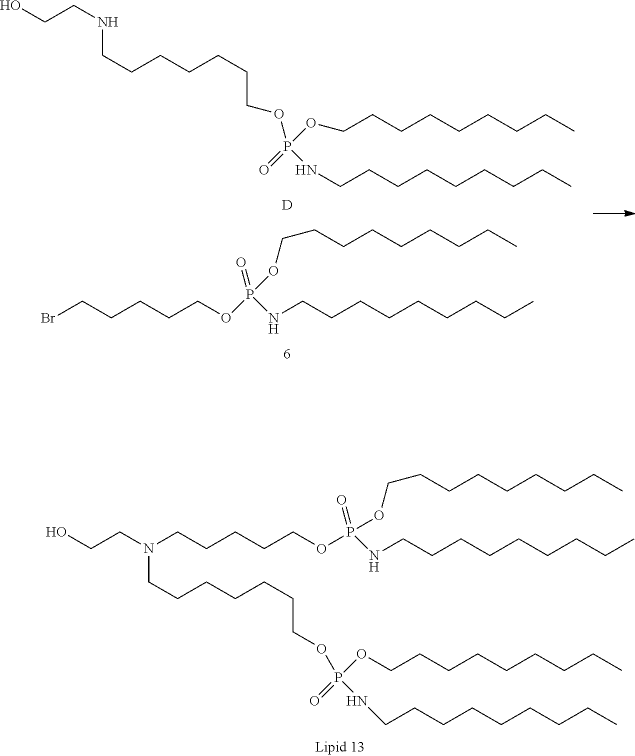

To a solution of Compound 6 (511.56 mg, 1.03 mmol, 1.3 eq.) in ACN (8 mL) was added NaI (23.66 mg, 157.87 μmol, 0.2 eq.), K₂CO₃ (218.19 mg, 1.58 mmol, 2 eq.) and Compound D (0.4 g, 789.36 μmol, 1 eq.). The mixture was heated 80° C. for 16 hours. LCMS showed Compound D was consumed completely and one main peak with desired MS was detected. The reaction mixture was poured into H₂O (10 mL) extracted with DCM (10 mL*3), dried over Na₂SO₄, filtered and concentrated under reduced pressure to give a residue. The residue was purified by column chromatography (SiO₂, DCM:MeOH=50:1 to 10:1) to give Lipid 13 (109.17 mg, 118.11 μmol, 15.60% yield) as white solid.

LCMS [M+1]⁺=924.7

¹H NMR (400 MHz, DMSO-d₆) δ=4.83-4.72 (m, 2H), 3.89-3.71 (m, 8H), 3.45 (br s, 2H), 2.79-2.54 (m, 6H), 2.49-2.25 (m, 4H), 1.62-1.49 (m, 8H), 1.45-1.15 (m, 64H), 0.85 (br t, J=6.8 Hz, 12H)

### Lipid 14:

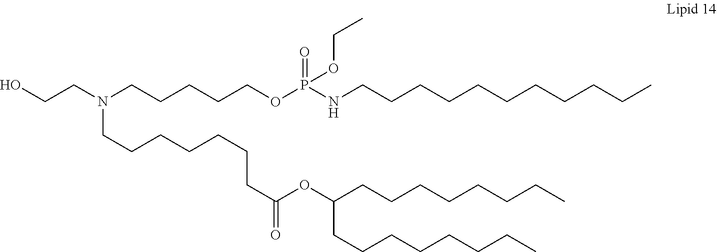 [SMILES: CCCCCCCCCCCNP(=O)(OCC)OCCCCCN(CCO)CCCCCCCC(=O)OC(CCCCCCCC)CCCCCCCC]

### Synthesis of Lipid 14:

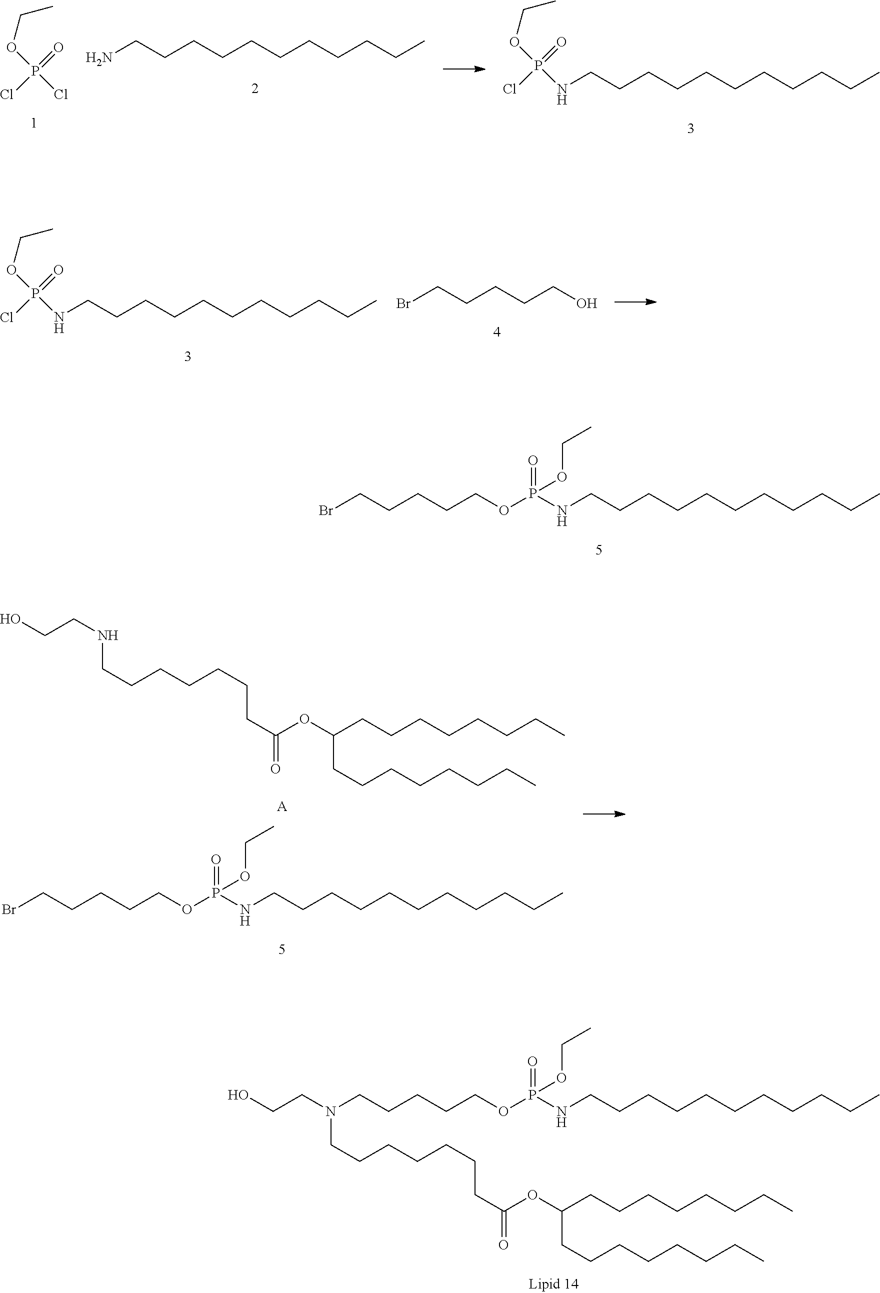

### Preparation of Compound 3

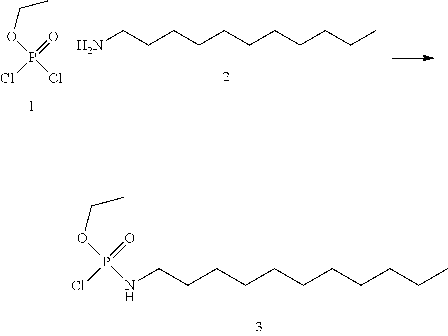

To a solution of Compound 1 (5 g, 30.69 mmol, 3.64 mL, 1 eq.) and TEA (3.26 g, 32.22 mmol, 4.48 mL, 1 eq.) in DCM (50 mL) was added Compound 2 (4.47 g, 26.08 mmol, 0.85 eq.) dropwise at 0° C. The resulting mixture was stirred at 20° C. for 2 hours. TLC indicated Compound 2 was consumed and one major new spot was formed. The reaction mixture was concentrated to give a residue. The residue was purified by column chromatography (SiO₂, Petroleum ether: Ethyl acetate=10:1 to 10:1) to give Compound 3 (3.5 g, 11.75 mmol, 38.30% yield, 100% purity) as yellow oil.

LCMS [M+1]⁺=298.2

¹H NMR (400 MHz, CHLOROFORM-d) δ=4.34-4.14 (m, 2H), 3.28-3.14 (m, 1H), 3.07-2.95 (m, 2H), 1.60-1.51 (m, 2H), 1.40-1.25 (m, 19H), 0.92-0.86 (m, 3H)

### Preparation of Compound 5

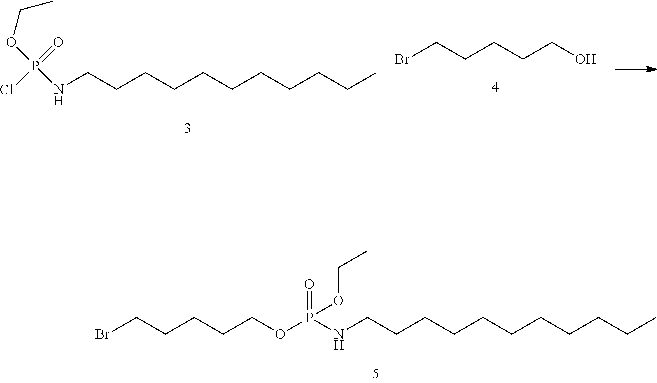

To a solution of Compound 3 (1.18 g, 3.96 mmol, 1 eq.) and TEA (801.90 mg, 7.92 mmol, 1.10 mL, 2 eq.) in DCM (20 mL) was added Compound 4 (728.08 mg, 4.36 mmol, 1.1 eq.). The resulting mixture was stirred at 20° C. for 16 hours. LCMS showed that Compound 3 was consumed and desired MS was detected. The reaction mixture was diluted with NH₄Cl (10 mL) and extracted with DCM (10 mL*3), dried over Na₂SO₄, filtered and concentrated under reduced pressure to give a residue. The residue was purified by reversed-phase HPLC (column: Welch Xtimate C1 250*50 mm*10 um; mobile phase: [H₂O (10 mM NH₄HCO₃)-ACN:THF=1:1]; gradient: 40/6-50% B over 20.0 min) to give Compound 5 (0.812 g, 1.90 mmol, 47.84% yield, 100% purity) as yellow oil.

LCMS [M+1]⁺=429.2

¹H NMR (400 MHz, CHLOROFORM-d) δ=4.13-3.95 (m, 4H), 3.58-3.52 (m, 1H), 3.55 (t, J=6.4 Hz, 1H), 2.94-2.84 (m, 2H), 2.59-2.43 (m, 1H), 1.94-1.80 (m, 2H), 1.75-1.67 (m, 2H), 1.59-1.52 (m, 2H), 1.37-1.23 (m, 20H), 0.89 (t, J=6.8 Hz, 3H)

### Preparation of Lipid 14:

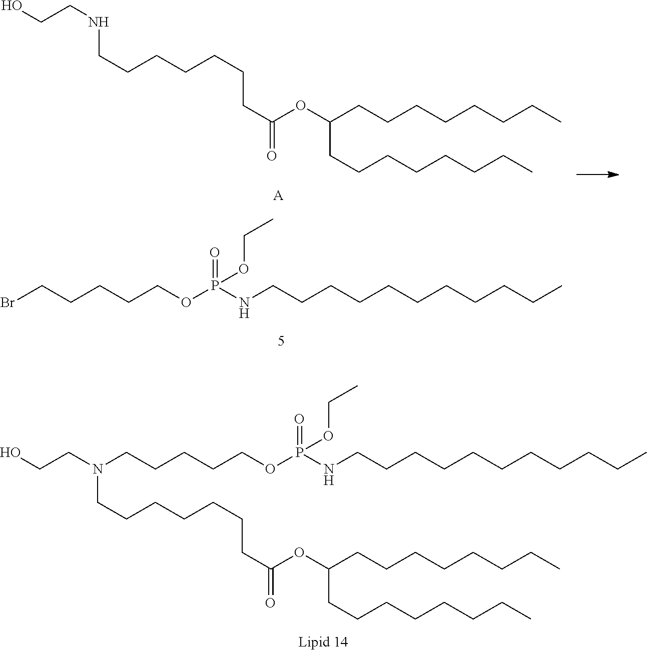

To a solution of Compound A (290.94 mg, 679.15 μmol, 1 eq.) in ACN (6 mL) was added Compound 5 (0.3 g, 679.15 μmol, 1 eq.), K₂CO₃ (187.72 mg, 1.36 mmol, 2 eq.) and NaI (20.36 mg, 135.83 μmol, 0.2 eq.). The resulting mixture was heated to 80° C. for 5 hours. TLC indicated Compound A was consumed and one major new spot was formed. The reaction mixture was diluted with DCM (20 mL) and washed with H₂O (10 mL*2), dried over Na₂SO₄, filtered and concentrated under reduced pressure to give a residue. The residue was purified by column chromatography (SiO₂, Dichloromethane:Methanol=100:1 to 20:1) to give Lipid 14 (0.101 g, 3.85 mmol, 32.24% yield, 98.02% purity) as brown oil.

LCMS [M+1]⁺=789.7

¹H NMR (400 MHz, CHLOROFORM-d) δ=4.90-4.82 (m, 1H), 4.20-3.83 (m, 1H), 3.00 (br d, J=6.0 Hz, 3H), 2.93-2.84 (m, 2H), 2.79 (br s, 1H), 2.31-2.26 (m, 2H), 1.80-1.70 (m, 4H), 1.69-1.56 (m, 4H), 1.54-1.46 (m, 8H), 1.41-1.20 (m, 52H), 0.94-0.83 (m, 9H)

### Lipid 15:

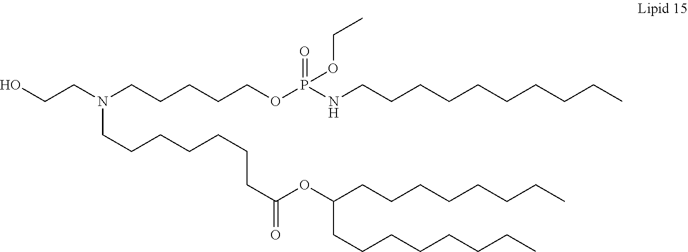 [SMILES: CCCCCCCCCCNP(=O)(OCC)OCCCCCN(CCO)CCCCCCCC(=O)OC(CCCCCCCC)CCCCCCCC]

### Synthesis of Lipid 15:

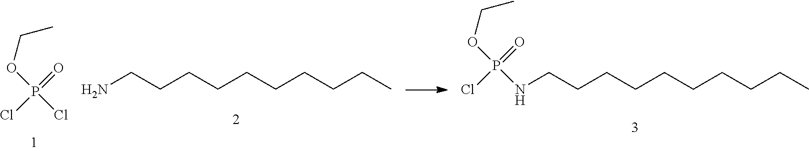

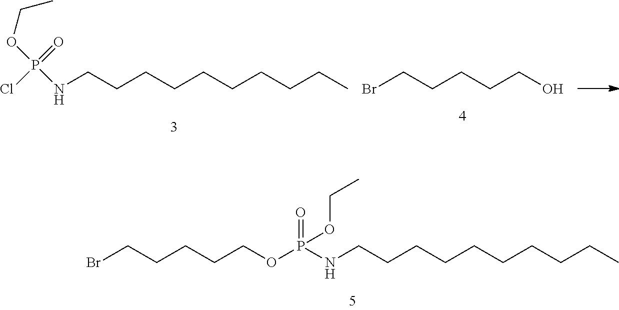

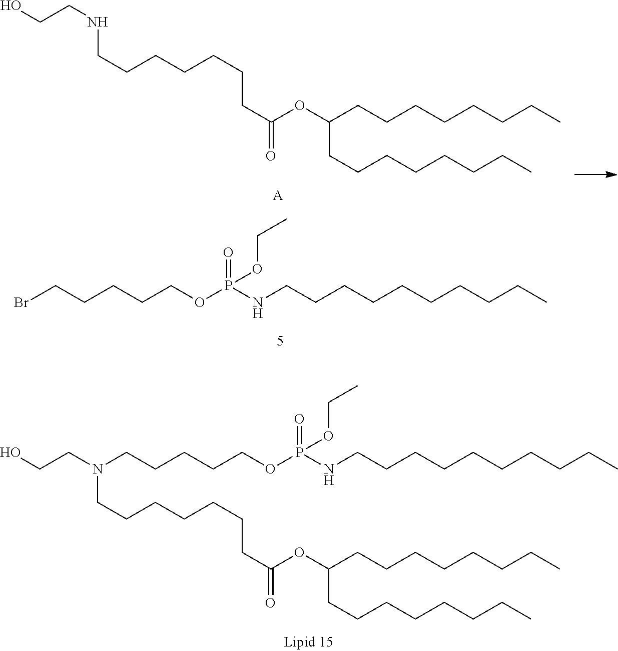

### Preparation of Compound 3:

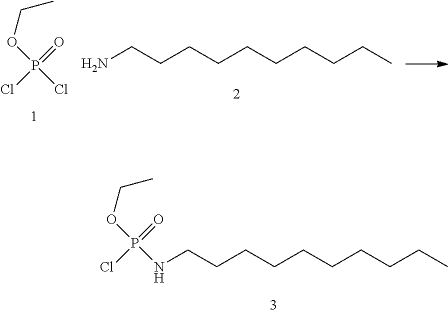

To a solution of Compound 1 (2 g, 12.27 mmol, 1.46 mL, 1 eq.) and TEA (1.30 g, 12.89 mmol, 1.79 mL, 1.05 eq.) in DCM (20 mL) was added Compound 2 (1.93 g, 12.27 mmol, 1 eq.) in DCM (10 mL) dropwise at 20° C. The mixture was stirred at 20° C. for 2 hours. TLC (Dichloromethane:Methanol=10:1, R_{f}=0.8) indicated Compound 1 was consumed completely and many new spots formed. The reaction mixture was concentrated under reduced pressure to give a residue. The residue was purified by column chromatography (SiO₂, Petroleum ether: Ethyl acetate=96:4 to 88:12) to give Compound 3 (2 g, 7.05 mmol, 57.42% yield, 100/6 purity) as white solid

LCMS [M+1]⁺=284.2

¹H NMR (400 MHz, CHLOROFORM-d) δ 4.33-4.12 (m, 2H), 3.29-3.15 (m, 1H), 3.06-2.97 (m, 2H), 1.59-1.53 (m, 2H), 1.44-1.40 (m, 3H), 1.34-1.24 (m, 14H), 0.91-0.88 (m, 3H)

### Preparation of Compound 5:

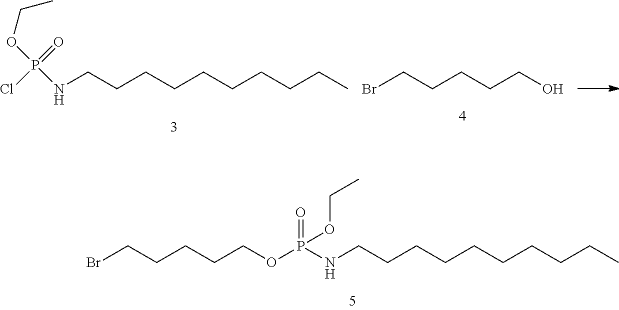

To a solution of Compound 3 (2 g, 7.05 mmol, 1.0 eq.) and TEA (1.43 g, 14.10 mmol, 1.96 mL, 2 eq.) in DCM (20 mL) was added Compound 4 (1.30 g, 7.75 mmol, 1.1 eq.) at 20° C. The mixture was stirred at 20° C. for 16 hours. LCMS showed Compound 3 was consumed completely and desired MS was detected. The reaction mixture was concentrated under reduced pressure to give a residue. The residue was purified by prep-HPLC (column: Welch X timate C1 250*50 mm*10 um; mobile phase: [H₂O (10 mM NH₄HCO₃)-ACN:THF=1:1]; gradient: 40%-80% B over 20.0 min) to give Compound 5 (460 mg, 1.11 mmol, 15.75% yield) as white solid.

LCMS[M+1]⁺=414.2

¹H NMR (400 MHz, CHLOROFORM-d) δ=4.15-3.91 (m, 4H), 3.42 (t, J=6.8 Hz, 2H), 2.89 (qd, J=7.2, 9.2 Hz, 2H), 2.59-2.45 (m, 1H), 1.97-1.85 (m, 2H), 1.77-1.66 (m, 2H), 1.61-1.52 (m, 2H), 1.48 (br t, J=6.8 Hz, 2H), 1.35-1.24 (m, 19H), 0.92-0.85 (m, 1H)

### Preparation of Lipid 15.

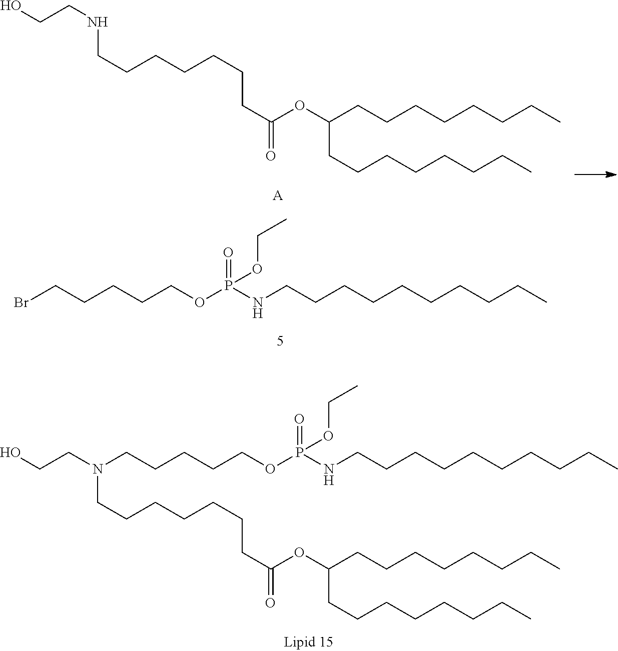

A mixture of Compound A (460 mg, 1.11 mmol, 1.05 eq.), Compound 5 (457.69 mg, 1.04 mmol, 1.0 eq.), K₂CO₃ (204.57 mg, 1.48 mmol, 2 eq.), NaI (11.09 mg, 74.01 μmol, 0.1 eq.) in ACN (5 mL) was degassed and purged with N₂ for 3 times, and then the mixture was stirred at 80° C. for 16 hours under N₂ atmosphere. LCMS showed the starting material was consumed completely. The reaction mixture was concentrated under reduced pressure to give a residue.

The residue was purified by column chromatography prep-TLC (SiO₂, DCM:MeOH=100:1 to 90:10) to give Lipid 15 (120 mg, 154.80 μmol, 20.92% yield, 100% purity) as white solid.

LCMS [M+1]⁺=775.7

¹H NMR (400 MHz, CHLOROFORM-d) δ 4.86 (t, J=6.4 Hz, 1H), 4.12-3.94 (m, 4H), 3.94-3.79 (m, 2H), 3.02 (br s, 2H), 3.11-2.81 (m, 7H), 2.75-2.58 (m, 1H), 2.28 (t, J=7.2 Hz, 2H), 1.73 (td, J=6.8, 13.6 Hz, 4H), 1.69-1.55 (m, 3H), 1.55-1.42 (m, 8H), 1.40-1.18 (m, 49H), 0.88 (t, J=6.8 Hz, 9H)

### Lipid 16:

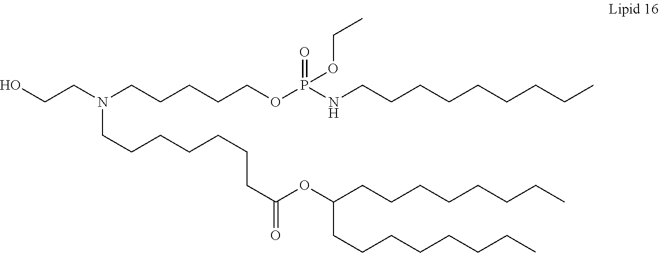 [SMILES: CCCCCCCCCNP(=O)(OCC)OCCCCCN(CCO)CCCCCCCC(=O)OC(CCCCCCCC)CCCCCCCC]

### Synthesis of Lipid 16:

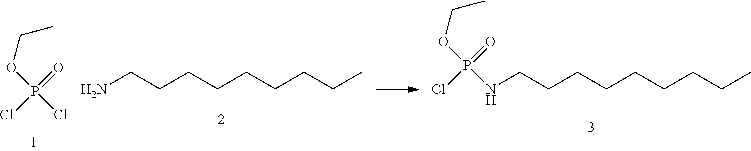

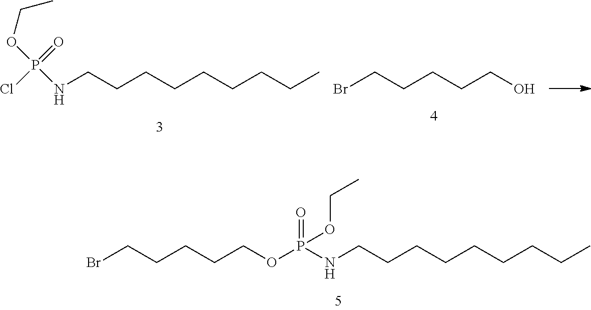

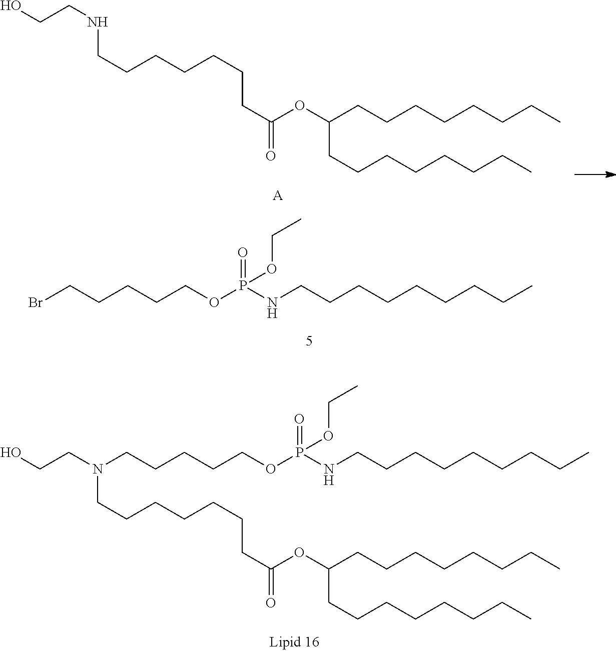

### Preparation of Compound 3:

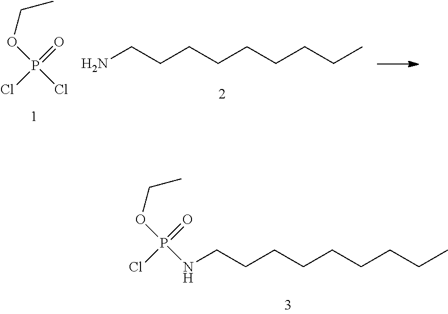

A solution of Compound 2 (2.11 g, 14.73 mmol, 0.8 eq.) and TEA (1.96 g, 19.33 mmol, 2.69 mL, 1.05 eq.) in Tol. (10 mL) was added dropwise to Compound 1 (3 g, 18.41 mmol, 2.18 mL, 1 eq.) in Tol. (20 mL). The mixture was stirred at 20° C. for 16 hours under N₂ atmosphere. TLC (Dichloromethane:Methanol=10:1, R_{f}=0.5) indicated Compound 2 was consumed completely and one new spot formed. The mixture was filtered through a Celite pad, and the filtrate was concentrated to give crude product. The crude product was diluted with H₂O (20 mL) and extracted with DCM (20 mL*2). The combined organic layers were dried over Na₂SO₄, filtered and concentrated under reduced pressure to give a residue. The residue was purified by column chromatography (SiO₂, Petroleum ether: Ethyl acetate=96:4 to 90:10) to give Compound 3 (2.96 g, 10.97 mmol, 59.60% yield) as light yellow oil.

¹H NMR (400 MHz, CHLOROFORM-d) δ=4.36-4.15 (m, 2H), 3.13 (br s, 1H), 3.08-2.96 (m, 2H), 1.60-1.50 (m, 2H), 1.40 (dt, J=0.8, 7.2 Hz, 3H), 1.37-1.18 (m, 12H), 0.89 (br t, 0.1=6.8 Hz, 3H)

### Preparation of Compound 5:

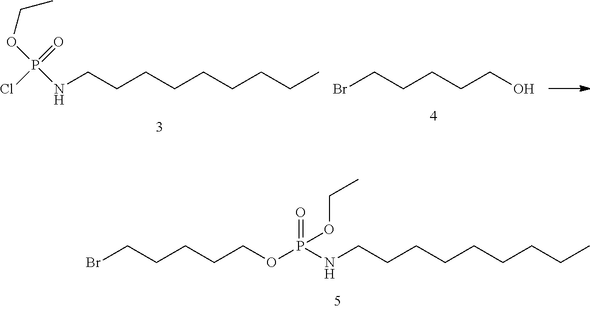

A mixture of Compound 3 (2.94 g, 10.90 mmol, 1 eq.), Compound 4 (2.37 g, 14.17 mmol, 1.3 eq.), TEA (2.21 g, 21.79 mmol, 3.03 mL, 2 eq.) in DCM (30 mL) was degassed and purged with N₂ for 3 times, and then the mixture was stirred at 20° C. for 16 hours under N₂ atmosphere. LCMS indicated Compound 3 was consumed completely and one main peak with desired MS was detected. The reaction mixture was concentrated under reduced pressure to give a residue. The residue was purified by prep-HPLC (Column: Welch Xtimate C1 100*30 mm*5 um Mobile phase: A: H₂O (0.05% HCl); B: THF:ACN=1:3 Gradient: B from 40.00% to 80.00% in 20.00 min Flow rate: 100.00 ml/min Monitor wavelength: 220&254 nm) to give Compound 5 (530 mg, 1.32 mmol, 12.15% yield) as white solid.

LCMS [M+1]⁺=400.4

¹H NMR (400 MHz, CHLOROFORM-d) δ=8.13 (m, 1H), 4.16-3.93 (m, 4H), 3.55 (t, J=6.8 Hz, 1H), 3.42 (t, J=6.8 Hz, 2H), 3.07-2.95 (m, 1H), 2.93-2.83 (m, 2H), 1.91 (quin, J=7.2 Hz, 2H), 1.81 (br dd, J=7.2, 14.4 Hz, 1H), 1.85-1.76 (m, 1H), 1.76-1.66 (m, 2H), 1.60-1.53 (m, 2H), 1.52-1.44 (m, 2H), 1.44-1.14 (m, 16H), 0.89 (t, J=6.8 Hz, 3H)

### Preparation of Lipid 16:

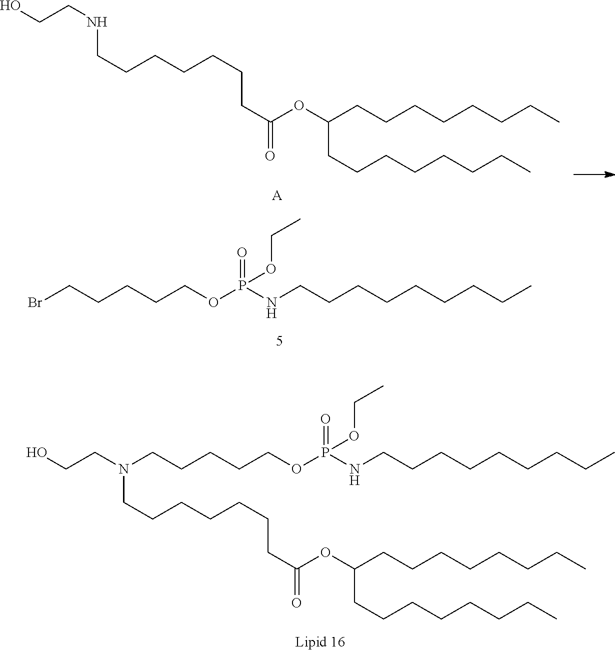

A mixture of Compound 5 (498.46 mg, 1.25 mmol, 1.1 eq.), K₂CO₃ (312.87 mg, 2.26 mmol, 2 eq.), NaI (16.97 mg, 113.19 μmol, 0.1 eq.) and Compound A (500 mg, 1.13 mmol, 1 eq.) in ACN (10 mL) was degassed and purged with N₂ for 3 times, and then the mixture was stirred at 80° C. for 16 hours under N₂ atmosphere. LCMS showed Compound A was consumed completely and one main peak with desired MS was detected. The reaction mixture was quenched by addition sat.NH₄Cl (5 mL) at 20° C., and then diluted with H₂O (5 mL) and extracted with DCM (10 mL*3). The combined organic layers were washed with brine (5 mL), dried over Na₂SO₄, filtered and concentrated under reduced pressure to give a residue. The residue was purified by column chromatography (SiO₂, DCM:MeOH=100:1 to 10:1) to give Lipid 16 (133.9 mg, 175.92 μmol, 14.88% yield, 100% purity) as colorless oil.

LCMS [M+1]⁺=761.7

¹H NMR (400 MHz, CHLOROFORM-d) δ=4.86 (t, J=6.0 Hz, 1H), 4.14-3.84 (m, 6H), 3.26-2.80 (m, 7H), 2.69-2.59 (m, 1H), 2.29 (t, J=7.6 Hz, 2H), 1.78-1.70 (m, 3H), 1.67-1.57 (m, 4H), 1.50 (br d, J=4.0 Hz, 9H), 1.41-1.16 (m, 46H), 0.89 (t, J=6.8 Hz, 9H).

### Lipid 17:

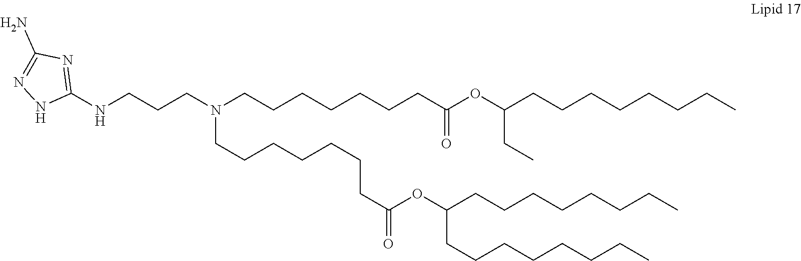 [SMILES: CCCCCCCCC(CC)OC(=O)CCCCCCCN(CCCCCCCC(=O)OC(CCCCCCCC)CCCCCCCC)CCCNc1nc(N)n[nH]1]

### Synthesis of Lipid 17:

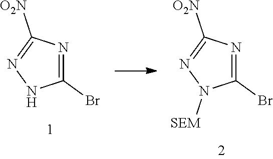 [SMILES: O=[N+]([O-])c1n[nH]c(Br)n1 ; C[Si](C)(C)CCOCn1nc([N+](=O)[O-])nc1Br]

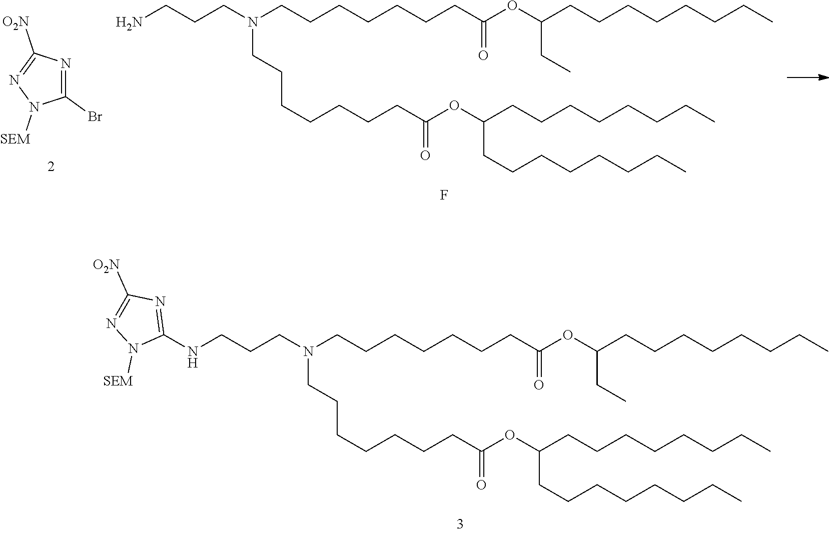

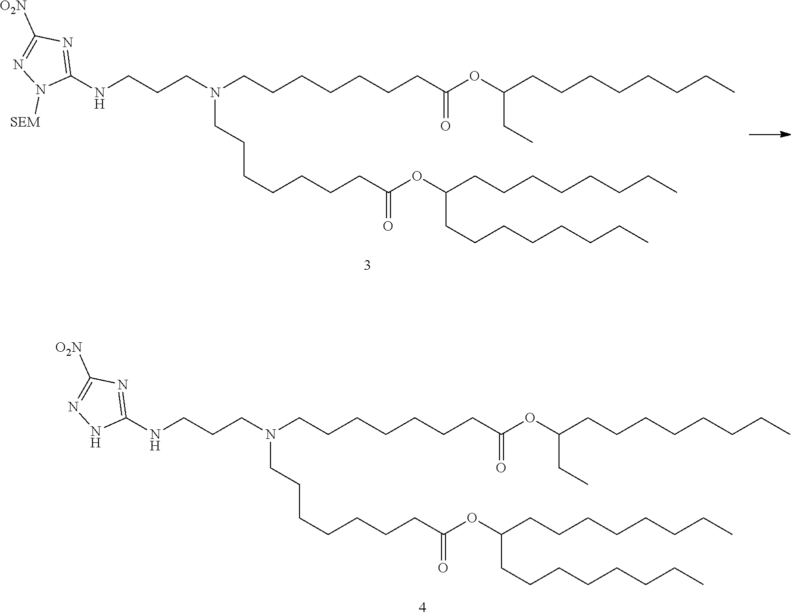 [SMILES: CCCCCCCCC(CC)OC(=O)CCCCCCCN(CCCCCCCC(=O)OC(CCCCCCCC)CCCCCCCC)CCCNc1nc([N+](=O)[O-])nn1COCC[Si](C)(C)C ; CCCCCCCCC(CC)OC(=O)CCCCCCCN(CCCCCCCC(=O)OC(CCCCCCCC)CCCCCCCC)CCCNc1nc([N+](=O)[O-])n[nH]1]

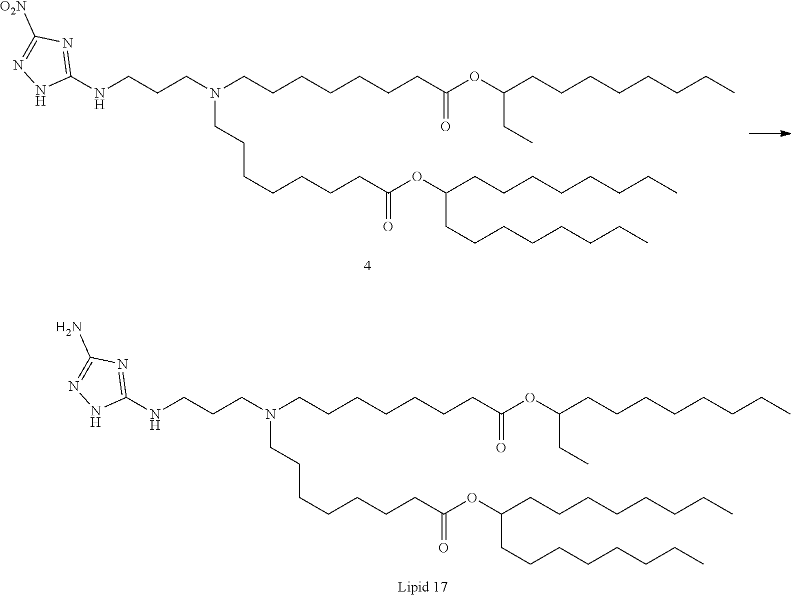 [SMILES: CCCCCCCCC(CC)OC(=O)CCCCCCCN(CCCCCCCC(=O)OC(CCCCCCCC)CCCCCCCC)CCCNc1nc([N+](=O)[O-])n[nH]1 ; CCCCCCCCC(CC)OC(=O)CCCCCCCN(CCCCCCCC(=O)OC(CCCCCCCC)CCCCCCCC)CCCNc1nc(N)n[nH]1]

### Preparation of Compound 2:

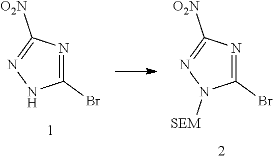 [SMILES: O=[N+]([O-])c1n[nH]c(Br)n1 ; C[Si](C)(C)CCOCn1nc([N+](=O)[O-])nc1Br]

To a solution of Compound 1 (2 g, 10.36 mmol, 1 eq.) in THF (20 mL) was added NaH (497.47 mg, 12.44 mmol, 60% purity, 1.2 eq.) at 0° C., and the mixture was stirred at 0° C. for 1 hour under N₂ atmosphere, then SEM-Cl (2.25 g, 13.47 mmol, 2.38 mL, 1.3 eq) was added, and the mixture was stirred at 20° C. for 2 hours under N₂ atmosphere. TLC (Petroleum ether: Ethyl acetate=3:1, R_{f}=0.42) Compound 1 was consumed completely and one new spot formed. The reaction mixture was quenched with sat.NH₄Cl (10 mL) and extracted with ethyl acetate (10 mL*3). The combined organic layers were concentrated under reduced pressure to give a residue. The residue was purified by column chromatography (SiO₂, Petroleum ether: Ethyl acetate=100:1 to 3:1) to give Compound 2 (5.9 g, 18.25 mmol, 88.06% yield) as colorless oil.

¹H NMR (400 MHz, DMSO-d₆) δ=5.64 (s, 2H), 3.66 (t, J=8.0 Hz, 2H), 0.95-0.74 (m, 2H), 0.03 (s, 9H)

### Preparation of Compound 3:

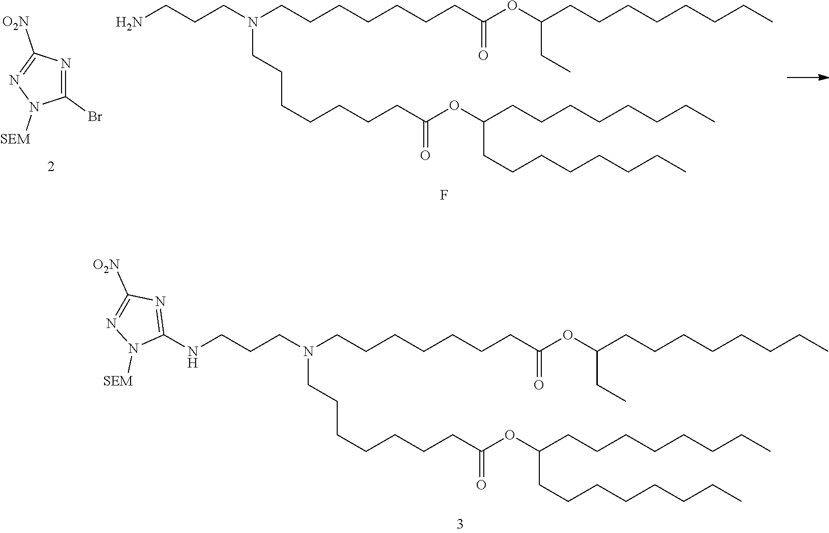

A mixture of Compound 2 (51.63 mg, 159.73 μmol, 1.2 eq.), Compound F (100 mg, 133.11 μmol, 1 eq.) in dioxane (1 mL), tBuXPhos Pd-G3 (31.72 mg, 39.93 μmol, 0.3 eq.), NaOt-Bu (2 M, 133.11 μL, 2 eq.) was degassed and purged with N₂ for 3 times, and then the mixture was stirred at 110° C. for 16 hours under N₂ atmosphere. LCMS indicated Compound F was consumed completely and one main peak with desired MS was detected. The reaction mixture (45 batches) was diluted with H₂O (80 mL) and extracted with DCM (80 mL*2). The combined organic layers were dried over Na₂SO₄, filtered and concentrated under reduced pressure to give a residue. The residue was purified by column chromatography (SiO₂, Dichloromethane:Methanol=100:1 to 20:1) to give Compound 3 (3 g, crude) as yellow oil.

LCMS [M+1]⁺=993.8

¹H NMR (400 MHz, CHLOROFORM-d) δ=5.42-5.22 (m, 2H), 4.93-4.66 (m, 2H), 3.70-3.42 (m, 4H), 3.40-2.85 (m, 2H), 2.71-2.56 (m, 1H), 2.52-2.38 (m, 2H), 2.29 (dt, J=4.0, 7.6 Hz, 4H), 1.90-1.68 (m, 2H), 1.67-1.46 (m, 18H), 1.36-1.21 (m, 48H), 0.94 (br d, J=8.8 Hz, 2H), 0.92-0.84 (m, 13H), 0.05-0.03 (m, 9H)

### Preparation of Compound 4:

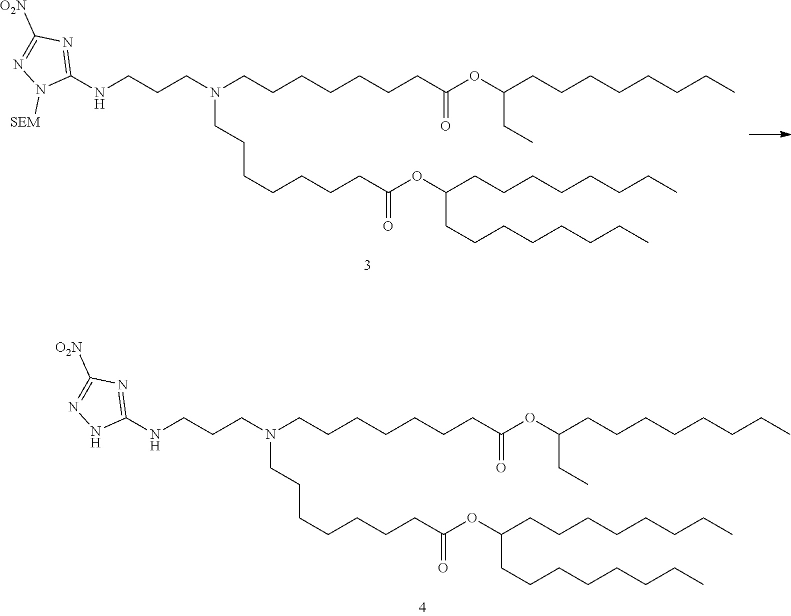 [SMILES: CCCCCCCCC(CC)OC(=O)CCCCCCCN(CCCCCCCC(=O)OC(CCCCCCCC)CCCCCCCC)CCCNc1nc([N+](=O)[O-])nn1COCC[Si](C)(C)C ; CCCCCCCCC(CC)OC(=O)CCCCCCCN(CCCCCCCC(=O)OC(CCCCCCCC)CCCCCCCC)CCCNc1nc([N+](=O)[O-])n[nH]1]

To a solution of Compound 3 (101 mg, 101.65 μmol, 1 eq.) in THE (0.5 mL) was TBAF (1 M, 1.02 mL, 10 eq.) at 20° C., and the mixture was heated to 80° C. for 3 hours under N₂ atmosphere. LCMS indicated Compound 3 was consumed completely and one main peak with desired MS was detected. The reaction mixture was concentrated under reduced pressure to give a residue. The residue was purified by column chromatography by prep-TLC (SiO₂, DCM:MeOH=96:4 to 9:1) to give Compound 4 (180 mg, 208.50 μmol, 41.02% yield) as yellow oil.

LCMS [M+1]⁺=863.7

¹H NMR (400 MHz, CHLOROFORM-d) δ=4.93-4.76 (m, 2H), 3.48 (br s, 1H), 2.96-2.72 (m, 5H), 2.29 (m, I 1H), 2.06-1.93 (m, 2H), 1.72-1.45 (m, 16H), 1.38-1.21 (m, 48H), 0.97-0.80 (m, 12H)

### Preparation of Lipid 17:

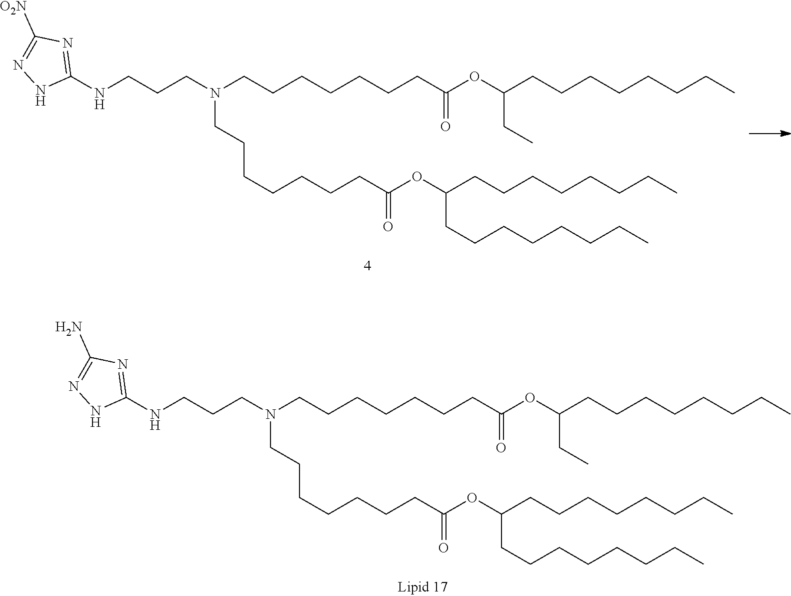 [SMILES: CCCCCCCCC(CC)OC(=O)CCCCCCCN(CCCCCCCC(=O)OC(CCCCCCCC)CCCCCCCC)CCCNc1nc([N+](=O)[O-])n[nH]1 ; CCCCCCCCC(CC)OC(=O)CCCCCCCN(CCCCCCCC(=O)OC(CCCCCCCC)CCCCCCCC)CCCNc1nc(N)n[nH]1]

To a solution of Compound 4 (100 mg, 115.83 μmol, 1 eq.) in TFE (2 mL) was added Pd/C (29.89 mg, 56.18 μmol, 20% purity) under N₂ atmosphere. The suspension was degassed and purged with H₂ for 3 times. The mixture was stirred under H₂ (15 Psi) at 30° C. for 2 hours. LCMS indicated Compound 4 was consumed completely and one main peak with desired MS was detected. The reaction mixture was filtered, and the filter cake was washed with DCM:IPA=1:1 (40 mL*3). The filtrate was concentrated under reduced pressure to give Lipid 17 (44.67 mg, 53.60 μmol, 46.28% yield) as yellow oil.

LCMS [M+1]⁺=833.5

1H NMR (400 MHz, DMSO-d₆) δ=5.70-5.36 (m, 1H), 5.32-5.00 (m, 1H), 4.89-4.53 (m, 2H), 3.51 (m, 2H), 2.98 (br s, 2H), 2.71 (br t, J=5.6 Hz, 1H), 2.41-2.19 (m, 8H), 2.19-2.03 (m, 2H), 1.64-1.42 (m, 11H), 1.34 (br s, 6H), 1.29-1.01 (m, 46H), 0.91-0.66 (m, 12H)

### Lipid 18:

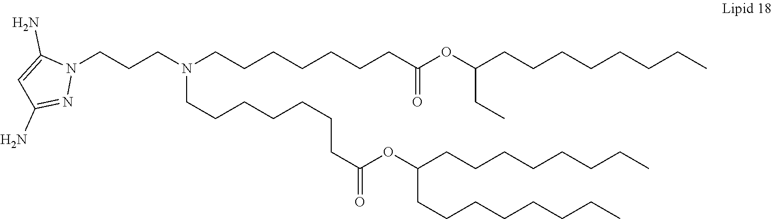 [SMILES: CCCCCCCCC(CC)OC(=O)CCCCCCCN(CCCCCCCC(=O)OC(CCCCCCCC)CCCCCCCC)CCCn1nc(N)cc1N]

### Synthesis of Lipid 18:

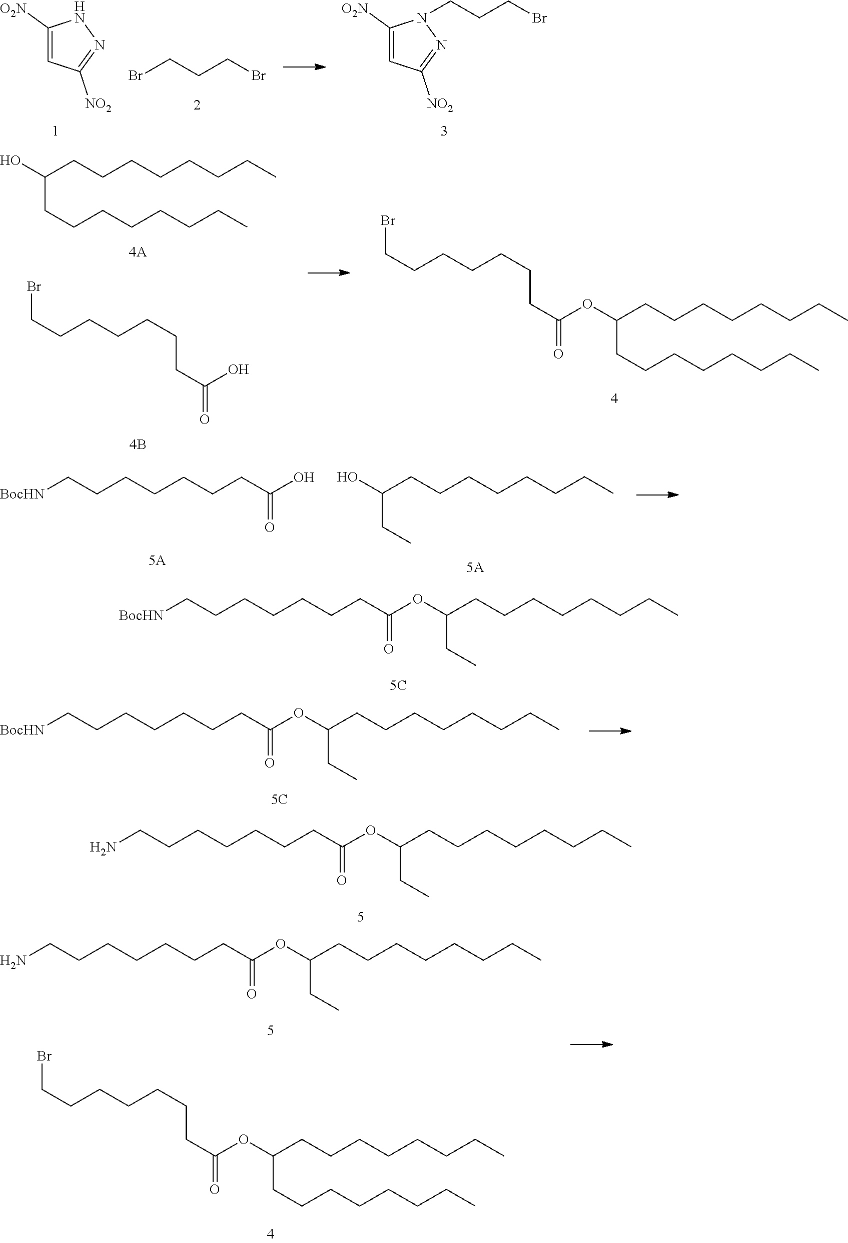

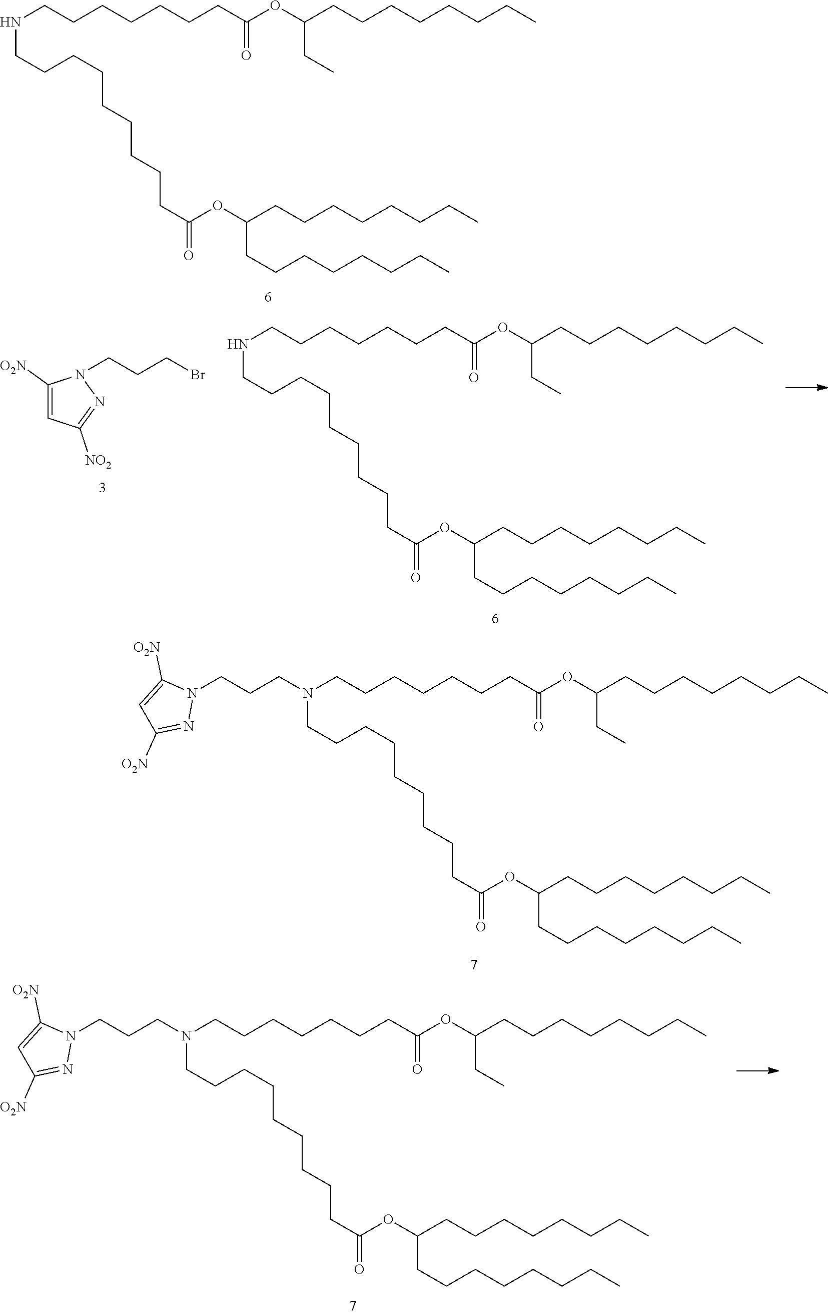 [SMILES: CCCCCCCCC(CC)OC(=O)CCCCCCCNCCCCCCCCCC(=O)OC(CCCCCCCC)CCCCCCCC ; O=[N+]([O-])c1cc([N+](=O)[O-])n(CCCBr)n1 ; CCCCCCCCC(CC)OC(=O)CCCCCCCNCCCCCCCCCC(=O)OC(CCCCCCCC)CCCCCCCC ; CCCCCCCCC(CC)OC(=O)CCCCCCCN(CCCCCCCCCC(=O)OC(CCCCCCCC)CCCCCCCC)CCCn1nc([N+](=O)[O-])cc1[N+](=O)[O-] ; CCCCCCCCC(CC)OC(=O)CCCCCCCN(CCCCCCCCCC(=O)OC(CCCCCCCC)CCCCCCCC)CCCn1nc([N+](=O)[O-])cc1[N+](=O)[O-]]

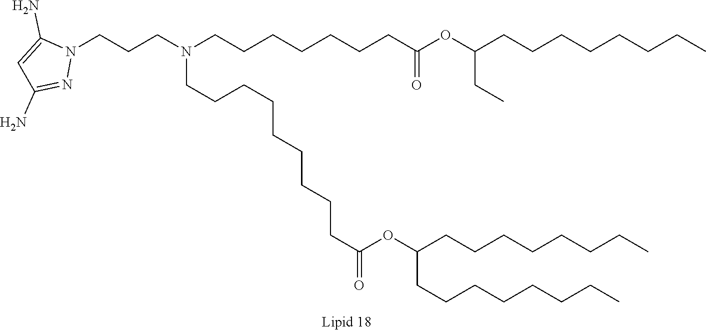 [SMILES: CCCCCCCCC(CC)OC(=O)CCCCCCCN(CCCCCCCCCC(=O)OC(CCCCCCCC)CCCCCCCC)CCCn1nc(N)cc1N]

### Preparation of Compound 3:

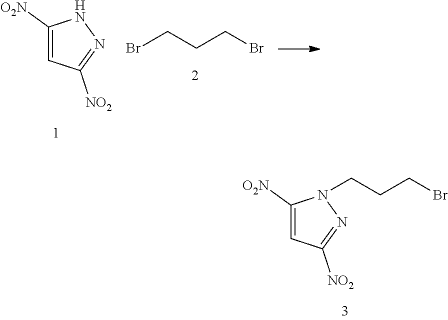

To a solution of Compound 1 (300 mg, 1.90 mmol, 1 eq.) in acetone (5 mL) was added K₂CO₃ (1.31 g, 9.49 mmol, 5 eq.) and Compound 2 (1.15 g, 5.69 mmol, 580.54 μL, 3 eq.). The mixture was heated to 60° C. for 2 hours. TLC (ethyl acetate, R₁=0.24) indicated ˜5% of Compound 1 was remained, and one major new spot was formed. The reaction mixture (3 batches) was filtered and concentrated under reduced pressure to give a residue. The residue was purified by column chromatography (SiO₂, Petroleum ether: Ethyl acetate=25:1 to 0:1) to give Compound 3 (640 mg, 2.29 mmol, 40.40% yield) as yellow oil.

¹H NMR (400 MHz, CHLOROFORM-d) δ=7.65 (s, 1H), 4.91 (t, J=7.2 Hz, 2H), 3.48 (t, J=6.4 Hz, 2H), 2.62-2.51 (m, 2H)

### Preparation of Compound 4:

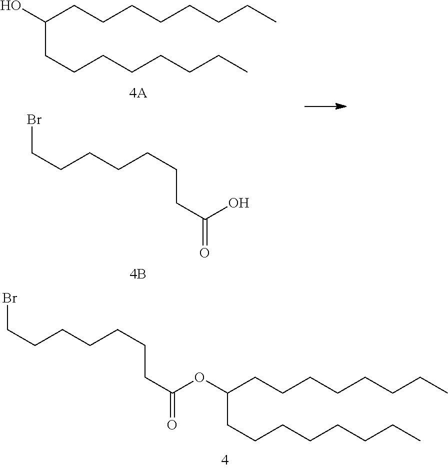 [SMILES: CCCCCCCCC(O)CCCCCCCC ; O=C(O)CCCCCCCBr ; CCCCCCCCC(CCCCCCCC)OC(=O)CCCCCCCBr]

A mixture of Compound 4A (10.44 g, 46.79 mmol, 1.2 eq.), Compound 4B (10 g, 38.99 mmol, 1 eq), DMAP (952.70 mg, 7.80 mmol, 0.2 eq.), EDCI (8.97 g, 46.79 mmol, 1.2 eq.) and DIEA (10.08 g, 77.98 mmol, 13.58 mL, 2 eq.) in DCM (100 mL) was degassed and purged with N₂ for 3 times, and then the mixture was stirred at 20° C. for 16 hours under N₂ atmosphere. TLC (Petroleum ether: Ethyl acetate=10:1, R=0.67) indicated Compound 4B was consumed completely and many new spots formed. The reaction mixture (2 batches) was partitioned between DCM (200 mL) and sat.NH₄Cl (100 mL). The organic phase was dried over Na₂SO₄, filtered and concentrated under reduced pressure to give a residue. The residue was purified by column chromatography (SiO₂, Petroleum ether: Ethyl acetate=100:1 to 20:1) to give Compound 4 (29 g, 62.83 mmol, 63.24% yield) as colorless oil.

LCMS [M+23]⁺=483.43

¹H NMR (400 MHz, CHLOROFORM-d) δ=4.87 (quin, J=6.4 Hz, 1H), 3.40 (t, J=6.8 Hz, 2H), 2.29 (t, J=7.6 Hz, 2H), 1.92-1.73 (m, 2H), 1.68-1.58 (m, 2H), 1.51 (br d, J=5.6 Hz, 4H), 1.47-1.40 (m, 2H), 1.39-1.30 (m, 6H), 1.32-1.18 (m, 21H), 0.88 (t, J=6.8 Hz, 6H)

### Preparation of Compound 5C:

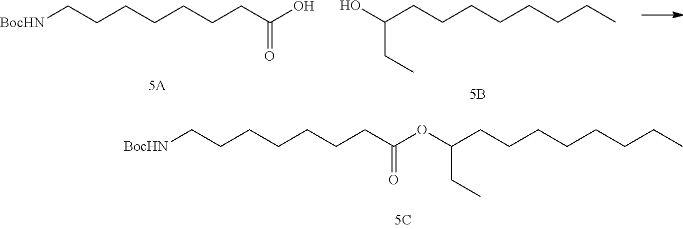 [SMILES: CC(C)(C)OC(=O)NCCCCCCCC(=O)O ; CCCCCCCCC(O)CC ; CCCCCCCCC(CC)OC(=O)CCCCCCCNC(=O)OC(C)(C)C]

A mixture of Compound 5A (13.36 g, 51.51 mmol, 1 eq.), Compound 5B (7.1 g, 41.21 mmol, 0.8 eq.), DMAP (1.26 g, 10.30 mmol, 0.2 eq.) EDCI (11.85 g, 61.81 mmol, 1.2 eq.) DIEA (13.31 g, 103.01 mmol, 17.94 mL, 2 eq.) in DCM (71 mL) was degassed and purged with N₂ for 3 times, and then the mixture was stirred at 20° C. for 16 hr under N₂ atmosphere. LCMS showed Compound 5B was consumed and desired MS was detected. The reaction mixture was diluted with DCM (71 mL) and washed with sat.NH₄Cl (50 mL*3), dried over Na₂SO₄, filtered and concentrated under reduced pressure to give a residue. The residue was purified by column chromatography (SiO₂, Petroleum ether: Ethyl acetate=1:0 to 9:1) to give Compound 5C (15 g, 36.26 mmol, 70.41% yield, 100% purity) as yellow oil.

¹H NMR (400 MHz, CHLOROFORM-d) δ=4.89-4.70 (m, 1H), 4.51 (br s, 1H), 3.18-2.98 (m, 2H), 2.28 (t, J=7.6 Hz, 2H), 1.69-1.40 (m, 17H), 1.38-1.18 (m, 18H), 0.93-0.83 (m, 6H)

### Preparation of Compound 5:

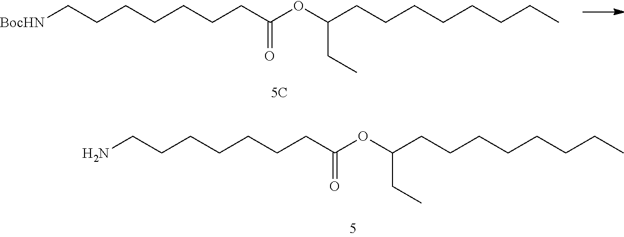 [SMILES: CCCCCCCCC(CC)OC(=O)CCCCCCCNC(=O)OC(C)(C)C ; CCCCCCCCC(CC)OC(=O)CCCCCCCN]

A mixture of Compound 5C (5 g, 12.09 mmol, 1 eq.) in HCl/dioxane (250 mL) was degassed and purged with N₂ for 3 times, and then the mixture was stirred at 20° C. for 2 hours under N₂ atmosphere. LCMS showed Compound 5C was consumed and desired MS was detected. The reaction solution was concentrated to give Compound 5 (8.1 g, 23.14 mmol, 95.73% yield, HCl) as yellow oil.

LCMS [M+1]⁺=314.4

¹H NMR (400 MHz, CHLOROFORM-d) δ=8.29 (br s, 3H), 4.81 (quin, J=6.4 Hz, 1H), 2.99 (br d, J=5.6 Hz, 2H), 2.28 (t, J=7.6 Hz, 2H), 1.90 (br s, 1H), 1.77 (br t, J=7.2 Hz, 2H), 1.70-1.47 (m, 6H), 1.46-1.14 (m, 18H), 0.94-0.80 (m, 6H)

### Preparation of Compound 6:

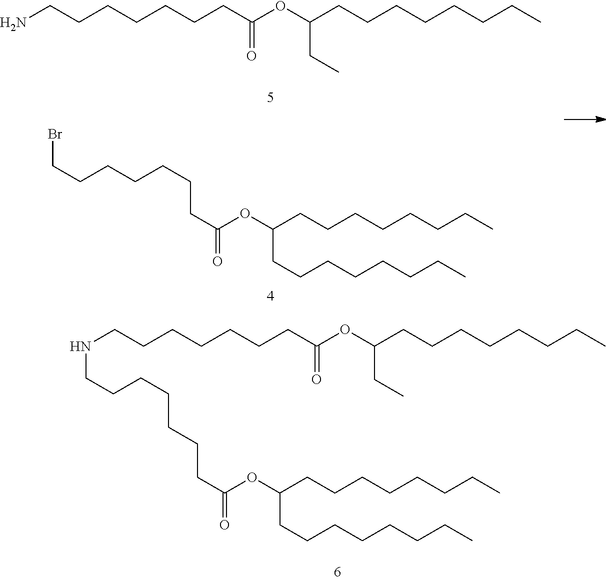 [SMILES: CCCCCCCCC(CC)OC(=O)CCCCCCCN ; CCCCCCCCC(CCCCCCCC)OC(=O)CCCCCCCBr ; CCCCCCCCC(CC)OC(=O)CCCCCCCNCCCCCCCC(=O)OC(CCCCCCCC)CCCCCCCC]

A mixture of Compound 5 (6.1 g, 17.43 mmol, 1 eq., HCl), Compound 4 (7.24 g, 15.69 mmol, 0.9 eq.), Cs₂CO₃ (3.95 g, 12.13 mmol, 0.9 eq.) in DMF (61 mL) was degassed and purged with N₂ for 3 times, and then the mixture was stirred at 20° C. for 2 hr under N₂ atmosphere. LCMS showed Compound 4 was consumed and desired MS was detected. The reaction mixture was diluted with ethyl acetate (60 mL) and washed with H₂O (30 mL*3), dried over Na₂SO₄, filtered and concentrated under reduced pressure to give a residue. The residue was purified by column chromatography (SiO₂, Petroleum ether: Ethyl acetate=80:1 to 8:1) to give Compound 6 (1.6 g, 2.30 mmol, 16.00% yield, 98.33% purity) as white solid.

LCMS [M+1]⁺=694.7

¹H NMR (400 MHz, CHLOROFORM-d) δ=4.96-4.71 (m, 2H), 2.61 (t, J=7.6 Hz, 4H), 2.28 (dt, J=3.6, 7.6 Hz, 4H), 1.67-1.59 (m, 4H), 1.55-1.43 (m, 11H), 1.40-1.09 (m, 50H), 0.95-0.78 (m, 12H)

### Preparation of Compound 7:

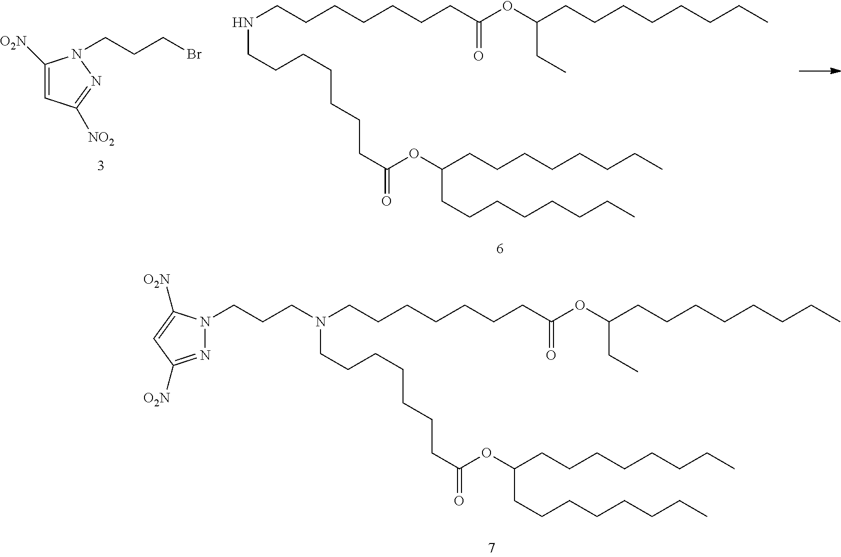 [SMILES: O=[N+]([O-])c1cc([N+](=O)[O-])n(CCCBr)n1 ; CCCCCCCCC(CC)OC(=O)CCCCCCCNCCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCn1nc([N+](=O)[O-])cc1[N+](=O)[O-]]

To a solution of Compound 3 (96.48 mg, 345.74 μmol, 1.2 eq.) in ACN (4 mL) was added K₂CO₃ (79.64 mg, 576.23 μmol, 2 eq.), NaI (8.64 mg, 57.62 μmol, 0.2 eq.) and Compound 6 (0.2 g, 288.12 μmol, 1 eq.). The mixture was stirred at 80° C. for 16 hours. LCMS showed Compound 6 was consumed completely and one main peak with desired MS was detected. The reaction mixture (2 batches) was concentrated under reduced pressure to give a residue. The residue was purified by prep-TLC (SiO₂, DCM:MeOH=15:1) to give Compound 7 (100 mg, 112.07 μmol, 19.23% yield) as yellow solid.

LCMS [M+1]⁺=892.8

¹H NMR (400 MHz, CHLOROFORM-d) δ=7.69-7.55 (m, 1H), 4.97-4.68 (m, 4H), 2.54-2.47 (m, 2H), 2.39-2.24 (m, 7H), 2.12-2.02 (m, 2H), 1.67-1.60 (m, 5H), 1.55-1.47 (m, 8H), 1.41-1.16 (m, 52H), 0.92-0.84 (m, 12H)

### Preparation of Lipid 18:

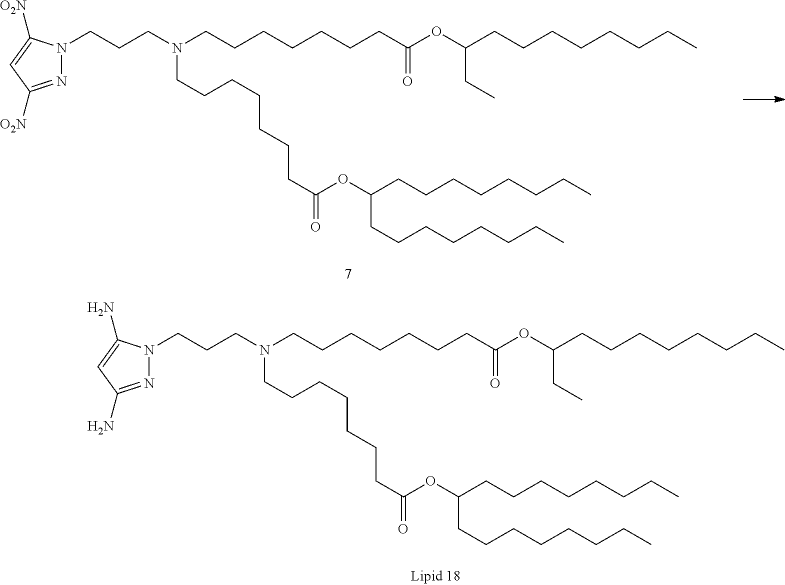 [SMILES: CCCCCCCCC(CC)OC(=O)CCCCCCCN(CCCCCCCC(=O)OC(CCCCCCCC)CCCCCCCC)CCCn1nc([N+](=O)[O-])cc1[N+](=O)[O-] ; CCCCCCCCC(CC)OC(=O)CCCCCCCN(CCCCCCCC(=O)OC(CCCCCCCC)CCCCCCCC)CCCn1nc(N)cc1N]

To a solution of Compound 7 (140 mg, 156.90 μmol, 1 eq.) in TFE (15 mL) was added Pd/C (312.24 mg, 293.40 μmol, 10% purity) N₂ atmosphere. The suspension was degassed and purged with H₂ for 3 times. The mixture was stirred under H₂ (15 Psi) at 30° C. for 2 hours. LCMS showed Compound 7 was consumed completely and one main peak with desired MS was detected. The reaction mixture was filtered, and the filter cake was washed with DCM:IPA=1:1 (40 mL*3). The filtrate was concentrated under reduced pressure to give Lipid 18 (113.69 mg, 136.59 μmol, 87.06% yield) as yellow oil.

LCMS [M+1]⁺=832.8

1H NMR (400 MHz, DMSO-d₆) δ=4.88-4.66 (m, 4H), 4.55 (s, 1H), 4.14-4.06 (m, 2H), 3.58-3.48 (m, 2H), 2.34-2.18 (m, 11H), 1.72-1.59 (m, 2H), 1.55-1.40 (m, 12H), 1.36-1.13 (m, 54H), 0.90-0.77 (m, 12H)

### Lipid 19:

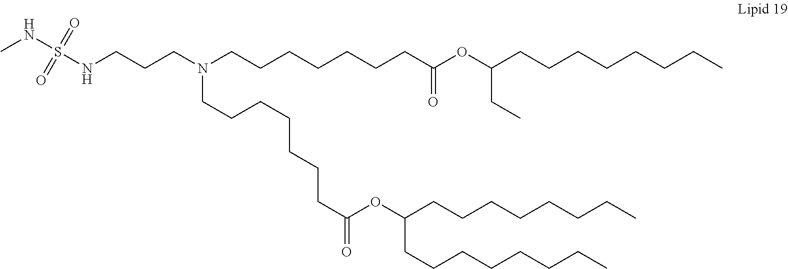 [SMILES: CCCCCCCCC(CC)OC(=O)CCCCCCCN(CCCCCCCC(=O)OC(CCCCCCCC)CCCCCCCC)CCCNS(=O)(=O)NC]

### Preparation of Lipid 19:

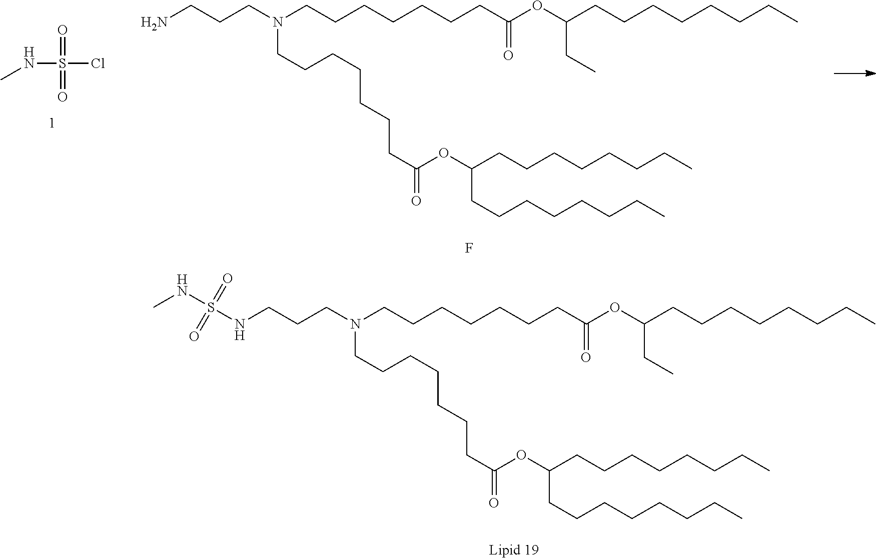

To a solution of Compound F (400 mg, 485.33 μmol, 1 eq., 2HCl) and TEA (392.89 mg, 3.88 mmol, 540.42 μL, 8 eq.) in DCM (8 mL) was added Compound 1 (150.92 mg, 1.16 mmol, 2.4 eq.) at 0° C. The reaction mixture was stirred at 20° C. for 2 hours. LCMS showed Compound F was consumed and one main peak with desired MS was detected. The reaction mixture was partitioned between sat.NaHCO₃ (15 mL) and DCM (30 mL). The organic phase was separated, dried over Na₂SO₄, filtered and concentrated under reduced pressure to give a residue. The residue was purified by prep-HPLC (column: X-Select CSH Phenyl-Hexyl 100*30 5u; mobile phase: [H₂O (0.04% HCl)-THF:ACN=1:3]; gradient: 40/0-80% B over 8.0 min) to give Lipid 19 (185 mg, 219.10 μmol, 45.14% yield) as colorless gum.

LCMS [M+1]⁺=844.8

¹H NMR (400 MHz, CHLOROFORM-d) δ=11.27 (br s, 1H), 4.90-4.75 (m, 2H), 3.32 (br s, 2H), 3.20 (br d, J=4.8 Hz, 2H), 3.12-2.92 (m, 5H), 2.74 (s, 3H), 2.29 (dt, J=4.4, 7.6 Hz, 4H), 2.11 (br s, 2H), 1.89-1.69 (m, 4H), 1.67-1.45 (m, 13H), 1.37 (br s, 12H), 1.32-1.18 (m, 36H), 0.92-0.83 (m, 12H)

### Lipid 20:

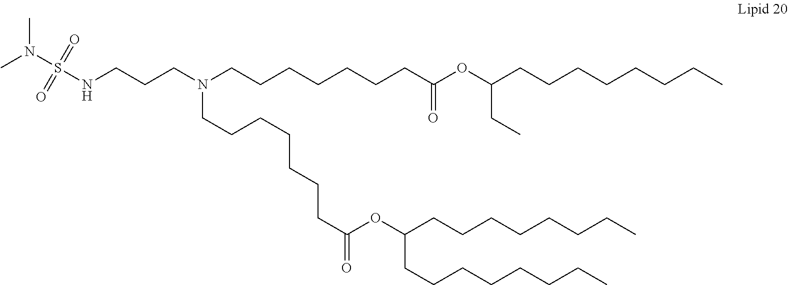 [SMILES: CCCCCCCCC(CC)OC(=O)CCCCCCCN(CCCCCCCC(=O)OC(CCCCCCCC)CCCCCCCC)CCCNS(=O)(=O)N(C)C]

### Preparation of Lipid 20:

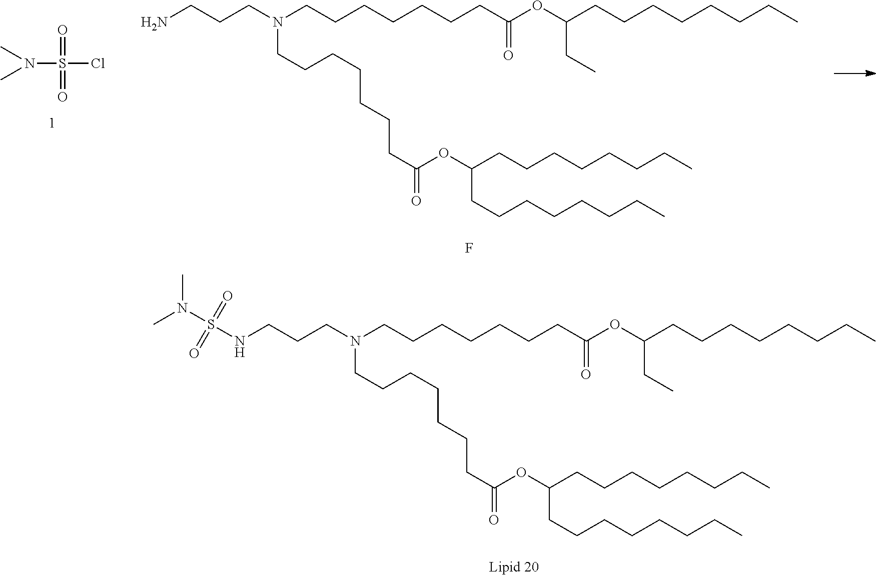

To a solution of Compound F (300 mg, 399.33 μmol, 1 eq.) in DCM (2 mL) was added TEA (121.22 mg, 1.20 mmol, 166.75 μL, 3 eq.) and Compound 1 (137.62 mg, 958.39 μmol, 102.93 L, 2.4 eq.) at 0° C. The mixture was stirred at 25° C. for 2 hours. LCMS showed Compound F was consumed completely and one main peak with desired MS was detected. The reaction mixture was concentrated under reduced pressure to give a residue. The residue was purified by prep-HPLC (column: X-Select CSHPhenyl-Hexyl100*305u; mobile phase: [H₂O (0.04% HCl)-THF:ACN=1:3]; gradient: 30/6-80% B over 10.0 min) to give Lipid 20 (120 mg, 139.80 μmol, 35.01% yield, HCl) as white solid.

LCMS [M+1]⁺=858.7

¹H NMR (DMSO-d₆, 400 MHz) δ=10.01 (br s, 1H), 7.30 (br t, J=5.6 Hz, 1H), 4.7-4.8 (m, 2H), 2.9-3.1 (m, 8H), 2.67 (s, 6H), 2.2-2.3 (m, 4H), 1.7-1.9 (m, 2H), 1.62 (br s, 4H), 1.4-1.6 (m, 13H), 1.2-1.3 (m, 50H), 0.8-0.9 (m, 12H)

### Lipid 21:

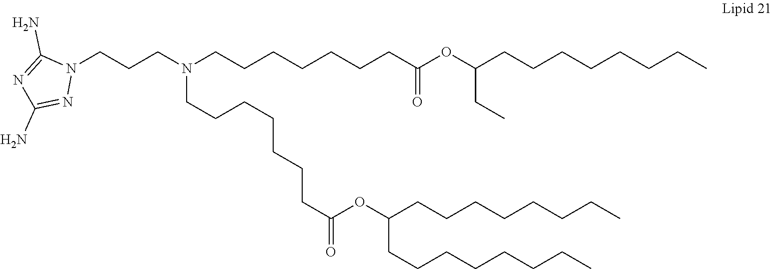 [SMILES: CCCCCCCCC(CC)OC(=O)CCCCCCCN(CCCCCCCC(=O)OC(CCCCCCCC)CCCCCCCC)CCCn1nc(N)nc1N]

### Preparation of Lipid 21:

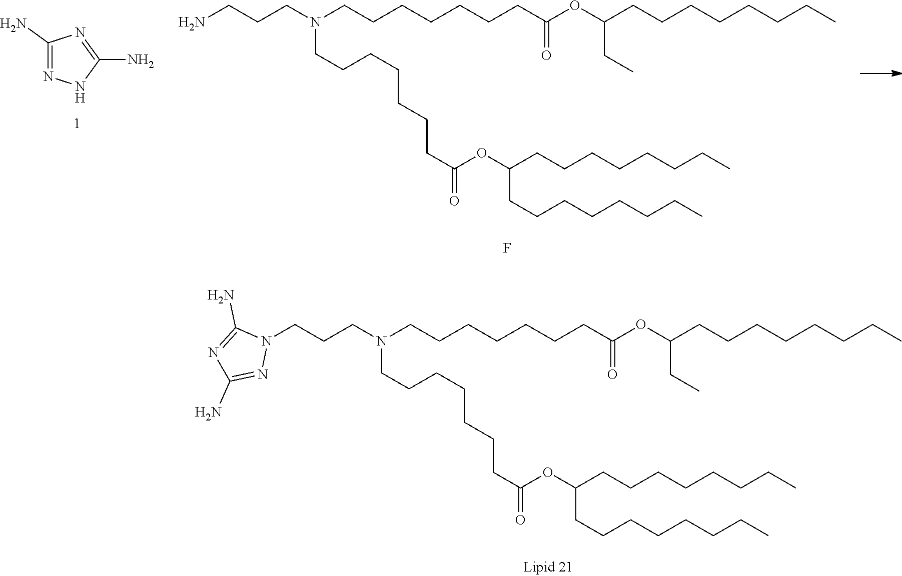

To a solution of Compound G (200 mg, 245.36 μmol, 1 eq.) in DMF (6 mL) was added Compound 1 (24.31 mg, 245.36 μmol, 1 eq.), NaI (7.36 mg, 49.07 μmol, 0.2 eq.), K₂CO₃ (67.82 mg, 490.71 μmol, 2 eq.), and the mixture was stirred at 80° C. for 3 hours under N₂ atmosphere. LCMS indicated Compound G was consumed completely and one main peak with desired MS was detected. The residue was diluted with H₂O (50 mL) and extracted with DCM (60 mL*2). The combined organic layers were dried over Na₂SO₄, filtered and concentrated under reduced pressure to give a residue. The residue was purified by prep-HPLC (column: X-Select CSH Phenyl-Hexyl 100*30 5u; mobile phase: [H₂O (0.04% HCl)-THF:ACN=1:3]; gradient: 45%-85% B over 8.0 min) to give Lipid 21 (120 mg, 144.00 μmol, 14.67% yield, HCl) as yellow oil. LCMS [M+1]⁺=833.7

¹H NMR (400 MHz, DMSO-d₆) δ=13.26-11.41 (m, 1H), 10.71 (br s, 1H), 8.21 (br s, 2H), 6.77-6.11 (m, I H), 4.86-4.65 (m, 2H), 3.95 (br t, J=6.4 Hz, 2H), 3.08 (br s, 2H), 2.98 (br s, 4H), 2.35-2.16 (m, 4H), 2.07 (br s, 2H), 1.65 (br s, 5H), 1.57-1.35 (m, 13H), 1.36-1.09 (m, 48H), 0.97-0.73 (m, 12H)

### Lipid 22:

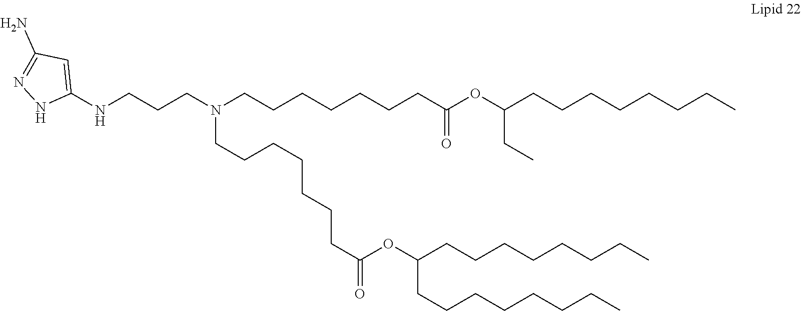 [SMILES: CCCCCCCCC(CC)OC(=O)CCCCCCCN(CCCCCCCC(=O)OC(CCCCCCCC)CCCCCCCC)CCCNc1cc(N)n[nH]1]

### Synthesis of Lipid 22:

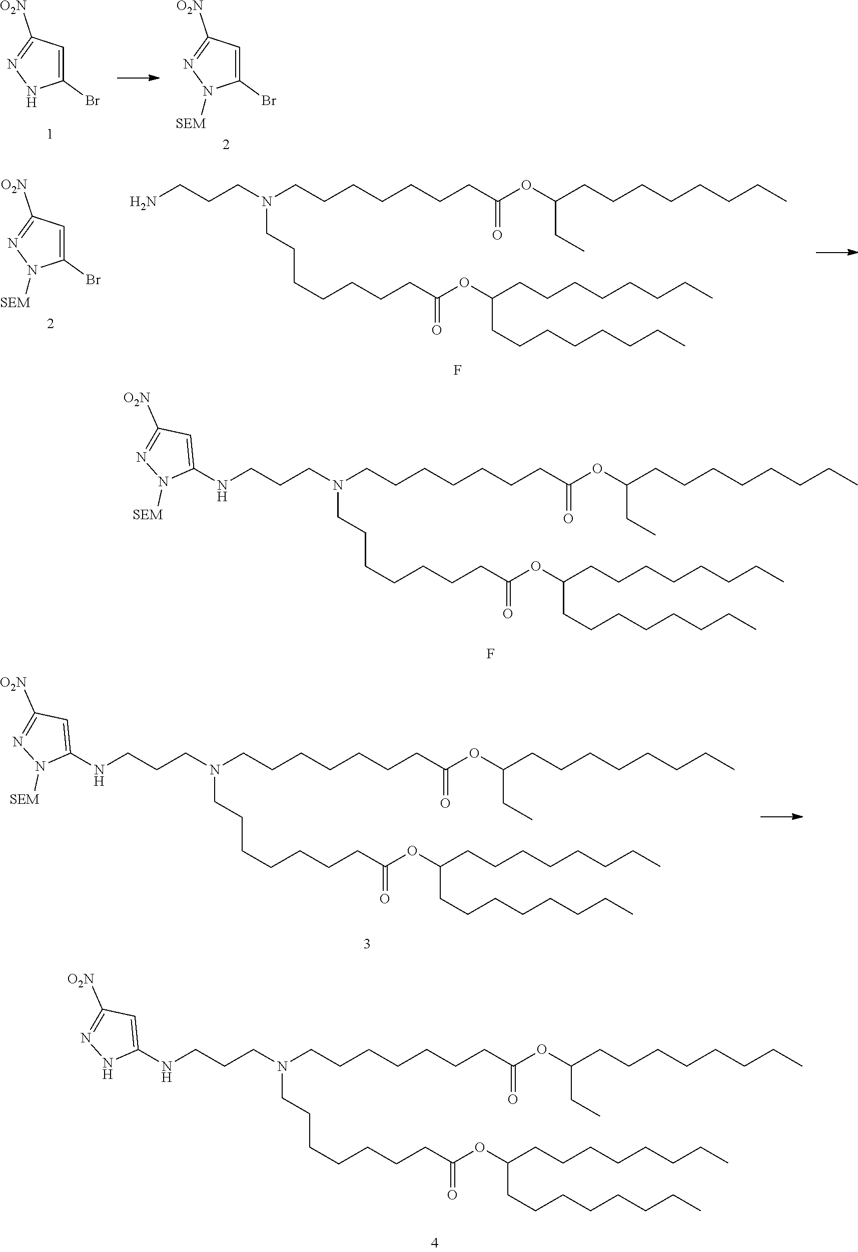

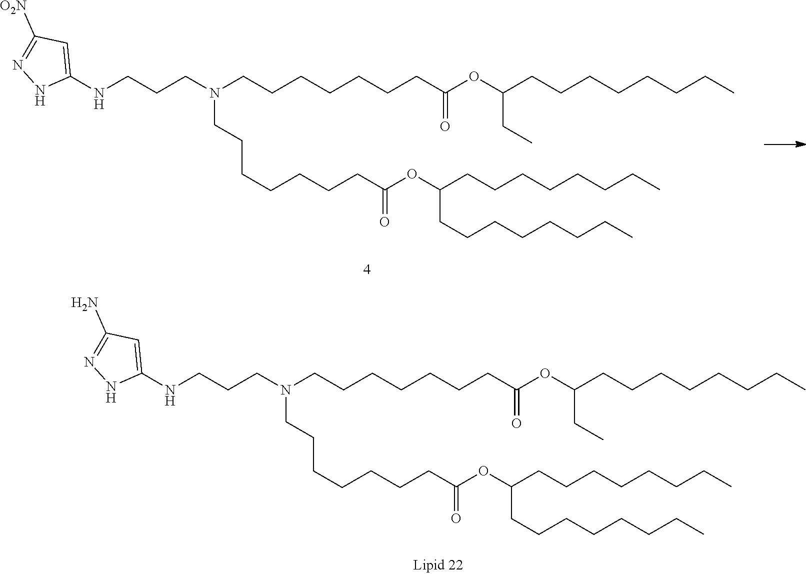 [SMILES: CCCCCCCCC(CC)OC(=O)CCCCCCCN(CCCCCCCC(=O)OC(CCCCCCCC)CCCCCCCC)CCCNc1cc([N+](=O)[O-])n[nH]1 ; CCCCCCCCC(CC)OC(=O)CCCCCCCN(CCCCCCCC(=O)OC(CCCCCCCC)CCCCCCCC)CCCNc1cc(N)n[nH]1]

### Preparation of Compound 2:

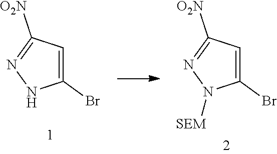 [SMILES: O=[N+]([O-])c1cc(Br)[nH]n1 ; C[Si](C)(C)CCOCn1nc([N+](=O)[O-])cc1Br]

To a solution of Compound 1 (4.5 g, 23.44 mmol, 1 eq.) in THF (45 mL) was added NaH (1.88 g, 46.88 mmol, 60% purity, 2 eq.) and SEM-Cl (5.08 g, 30.47 mmol, 5.39 mL, 1.3 eq.) at 0° C. The mixture was stirred at 20° C. for 16 hours. TLC (Petroleum ether: Ethyl acetate=3:1, R_{f}=0.5) indicated Compound 1 was consumed completely and one new spot formed. The residue was quenched with sat.NH₄Cl (50 mL) and extracted with EtOAc (50 mL*3). The combined organic layers were concentrated under reduced pressure to give a residue. The residue was purified by column chromatography (SiO₂, Petroleum ether: Ethyl acetate=200:1 to 10:1) to give Compound 2 (4.2 g, 11.73 mmol, 50.04% yield, 90% purity) as colorless oil.

¹H NMR (400 MHz, CHLOROFORM-d) δ=7.06-6.96 (m, 1H), 5.67-5.54 (m, 2H), 3.75-3.58 (m, 2H), 1.62-1.49 (m, 1H), 1.01-0.88 (m, 2H), 0.07-−0.06 (m, 9H)

### Preparation of Compound 3:

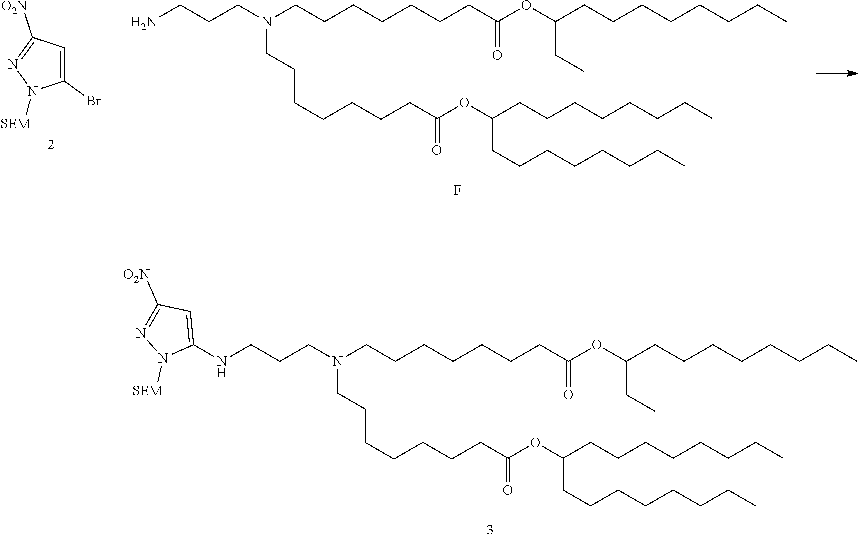

To a solution of Compound 2 (3.08 g, 9.583 mmol, 1.2 eq.) and Compound F (6 g, 7.986 mmol, 1 eq.) in dioxane (60 mL) was added tBuXPhos Pd-G3 (1.2 g, 1.597 mmol, 0.2 eq.) and NaOtBu (2 M, 7.98 mL, 2 eq.). The mixture was stirred at 110° C. for 16 hours. LCMS showed Compound 2 was consumed completely and one main peak with desired MS was detected. The residue was diluted with DCM (50 mL) and extracted with H₂O (25 mL). The combined organic layers were dried over Na₂SO₄, filtered and concentrated under reduced pressure to give a residue. The residue was purified by column chromatography (SiO₂, Dichloromethane:Methanol=500:1 to 125:1) to give Compound 3 (1.6 g, 1.60 mmol, 92.22% yield, 99% purity) as yellow oil.

LCMS [M+1]⁺=993.0

¹H NMR (400 MHz, CHLOROFORM-d) δ=5.97-5.89 (m, 2H), 5.42-5.31 (m, 2H), 4.90-4.77 (m, 2H), 3.68-3.53 (m, 2H), 3.25-3.14 (m, 2H), 2.97-2.56 (m, 5H), 2.34-2.21 (m, 5H), 1.89-1.71 (m, 2H), 1.66-1.55 (m, 8H), 1.54-1.46 (m, 8H), 1.35-1.23 (m, 52H), 0.95-0.84 (m, 15H), 0.06-−0.07 (m, 9H)

### Preparation of Compound 4:

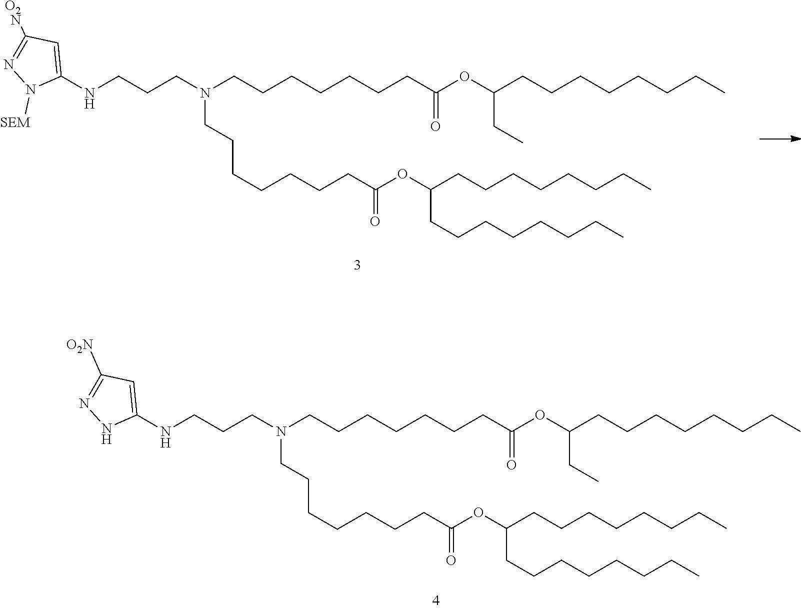 [SMILES: CCCCCCCCC(CC)OC(=O)CCCCCCCN(CCCCCCCC(=O)OC(CCCCCCCC)CCCCCCCC)CCCNc1cc([N+](=O)[O-])nn1COCC[Si](C)(C)C ; CCCCCCCCC(CC)OC(=O)CCCCCCCN(CCCCCCCC(=O)OC(CCCCCCCC)CCCCCCCC)CCCNc1cc([N+](=O)[O-])n[nH]1]

To a solution of Compound 3 (700 mg, 705.46 μmol, 1 eq.) in THF (3.5 mL) was added TBAF (1 M, 3.5 mL, 5 eq.). The mixture was stirred at 80° C. for 3 hours. LCMS showed Compound 10 was consumed completely and one main peak with desired MS was detected. The mixture was concentrated to give a residue. The residue was purified by prep-TLC (SiO₂, DCM:MeOH=10:1) to give Compound 4 (100 mg, 104.37 μmol, 14.80% yield, 90% purity) as yellow oil.

LCMS [M+1]⁺=862.8

¹H NMR (400 MHz, CHLOROFORM-d) δ=5.91-5.80 (m, 1H), 4.85-4.60 (m, 2H), 3.31-3.14 (m, 2H), 2.89-2.76 (m, 2H), 2.72-2.57 (m, 3H), 2.28-2.13 (m, 4H), 1.94-1.77 (m, 2H), 1.63-1.36 (m, 16H), 1.32-1.10 (m, 51H), 0.85-0.70 (m, 12H)

### Preparation of Lipid 22:

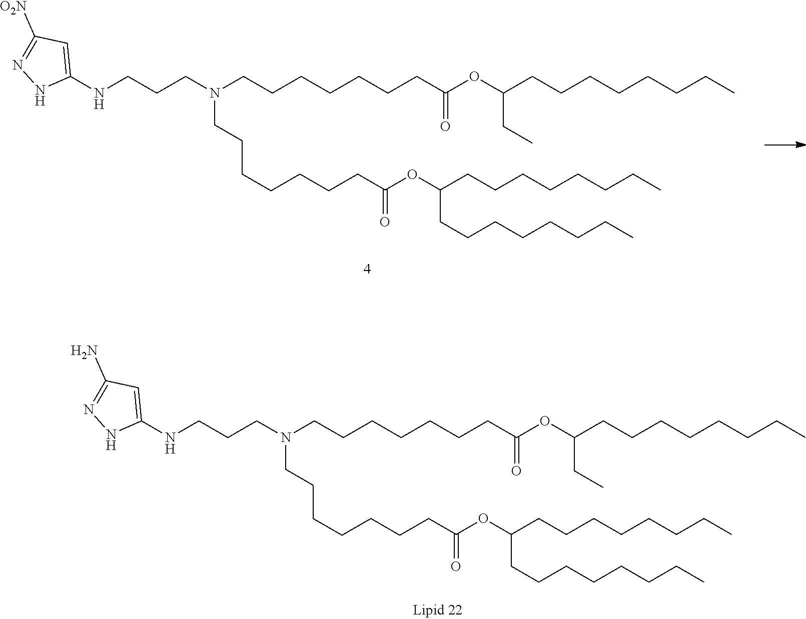 [SMILES: CCCCCCCCC(CC)OC(=O)CCCCCCCN(CCCCCCCC(=O)OC(CCCCCCCC)CCCCCCCC)CCCNc1cc([N+](=O)[O-])n[nH]1 ; CCCCCCCCC(CC)OC(=O)CCCCCCCN(CCCCCCCC(=O)OC(CCCCCCCC)CCCCCCCC)CCCNc1cc(N)n[nH]1]

To a solution of Compound 4 (100 mg, 120.14 μmol, 1 eq.) in 2,2,2-trifluoroethanol (1 mL) was added Pd/C (63.93 mg, 10% purity) under N₂ atmosphere. The suspension was degassed and purged with H₂ for 3 times. The mixture was stirred under H₂ (15 Psi) at 20° C. for 2 hours. LCMS showed Compound 4 was consumed and one main peak with desired MS was detected. The reaction mixture was filtered, and the filter cake was washed with DCM:IPA=1:1 (40 mL*3). The filtrate was concentrated under reduced pressure to give Lipid 22 (62.75 mg, 21.87 mol, 43.41% yield, 94.62% purity) as brown oil.

LCMS [M+1]⁺=832.9

¹H NMR (400 MHz, CHLOROFORM-d) δ=4.84-4.69 (m, 2H), 3.12 (m, 3H), 2.85-2.42 (m, 8H), 2.21 (m, 4H), 1.81-1.67 (m, 2H), 1.62-1.37 (m, 16H), 1.31-1.10 (m, 50H), 0.87-0.75 (m, 12H)

### Example 7. Lipid Screening by In Vivo Expression Evaluation in Lipid Nanoparticle Formulations

In this example, messenger RNA molecules encoding hEPO proteins were formulated in lipid nanoparticles for delivery in vivo. The lipid nanoparticle (LNP) formulations comprised of a lipid composition of ionizable lipid: helper lipid: cholesterol. DMG-PEG2k at 50:10:38.5:1.5 mol %. The lipid mixture in ethanol was mixed with hEPO mRNA in RNA acidifying buffer (10 mM citrate, pH 4) at an ionizable-lipid-nitrogen-to-RNA-phosphate ratio (N:P) of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Samples were then characterized and diluted as needed (3 replicates).

LNPs were characterized by dynamic light scattering (DLS) measurement for measuring its hydrodynamic radius and polydispersity index (PDI). Encapsulation efficiency (EE %) and total RNA concentration were quantitated using a fluorescence based Ribogreen assay. The measurements for each of the LNPs are shown in Table 1 below.

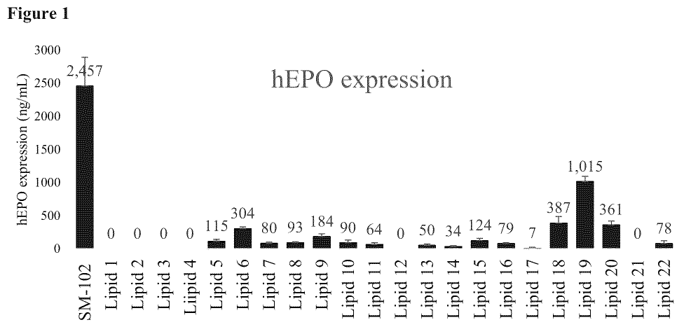

The in vivo studies were performed in C57BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs was compared to a control LNP comprising SM102 (DC Chemicals Cat. No. DC52025). (FIG. 1).

TABLE 1
LNP Measurements

| | | | | Size | | Zeta | EE | | hEPO | |
|---|---|---|---|---|---|---|---|---|---|---|
| Lipid ID | mRNA | Lipid:Cholesterol:DSPC:PEG-2k | N:P | (nm) | PDI | (mV) | (%) | pKa | (ng/mL) | St. Dev |
| SM-102 | hEPO | 50:38.5:10:1.5 | 6 | 78.70 | 0.106 | −3.1 | 96.7 | 7 | 2457.05 | 435.54 |
| Lipid 1 | hEPO | 50:38.5:10:1.5 | 6 | 65.8 | 0.267 | −8.8 | 97.9 | 5.1 | 0.00 | 0.00 |
| Lipid 2 | hEPO | 50:38.5:10:1.5 | 6 | 68.8 | 0.399 | 0.1 | 97.9 | 5.1 | 0.00 | 0.00 |
| Lipid 3 | hEPO | 50:38.5:10:1.5 | 6 | 62.8 | 0.48 | −9.2 | 97.6 | 5.1 | 0.00 | 0.00 |
| Lipid 4 | hEPO | 50:38.5:10:1.5 | 6 | 56.2 | 0.103 | −9.3 | 97.5 | 5.1 | 0.00 | 0.00 |
| Lipid 5 | hEPO | 50:38.5:10:1.5 | 6 | 87.4 | 0.358 | −1.0 | 95.3 | 6.8 | 114.89 | 28.88 |
| Lipid 6 | hEPO | 50:38.5:10:1.5 | 6 | 132.7 | 0.227 | −0.4 | 95.8 | 6.9 | 304.28 | 24.81 |
| Lipid 7 | hEPO | 50:38.5:10:1.5 | 6 | 145.3 | 0.296 | −2.0 | 97.2 | 6.9 | 80.47 | 18.45 |
| Lipid 8 | hEPO | 50:38.5:10:1.5 | 6 | 134.7 | 0.302 | 0.1 | 94.2 | 7.0 | 93.09 | 11.41 |
| Lipid 9 | hEPO | 50:38.5:10:1.5 | 6 | 83.9 | 0.101 | 0.7 | 99.7 | 7.0 | 184.44 | 40.48 |
| Lipid 10 | hEPO | 50:38.5:10:1.5 | 6 | 85.6 | 0.064 | 0.8 | 99.6 | 7.2 | 90.05 | 41.97 |
| Lipid 11 | hEPO | 50:38.5:10:1.5 | 6 | 94.4 | 0.08 | 2.6 | 99.7 | 7.3 | 63.81 | 27.89 |
| Lipid 12 | hEPO | 50:38.5:10:1.5 | 6 | 87.6 | 0.316 | 3.5 | 99.3 | 8.3 | 0.00 | 0.00 |
| Lipid 13 | hEPO | 50:38.5:10:1.5 | 6 | 67.1 | 0.108 | 2.4 | 96.7 | 7.8 | 49.71 | 22.47 |
| Lipid 14 | hEPO | 50:38.5:10:1.5 | 6 | 101.5 | 0.248 | 0.9 | 98.3 | 6.9 | 33.98 | 12.68 |
| Lipid 15 | hEPO | 50:38.5:10:1.5 | 6 | 83.1 | 0.165 | 0.2 | 94.8 | 7.0 | 123.69 | 33.46 |
| Lipid 16 | hEPO | 50:38.5:10:1.5 | 6 | 91.6 | 0.116 | −0.4 | 88.1 | 7.0 | 79.22 | 12.62 |
| Lipid 17 | hEPO | 50:38.5:10:1.5 | 6 | 75.1 | 0.169 | −14.1 | 98.8 | 6.1 | 6.59 | 14.74 |
| Lipid 18 | hEPO | 50:38.5:10:1.5 | 6 | 67.2 | 0.079 | −12.8 | 98.9 | 6.5 | 386.98 | 99.14 |
| Lipid 19 | hEPO | 50:38.5:10:1.5 | 6 | 60.9 | 0.183 | −27.6 | 96.9 | 6.3 | 1014.79 | 79.49 |
| Lipid 20 | hEPO | 50:38.5:10:1.5 | 6 | 58.6 | 0.162 | −14.5 | 95.2 | 6.6 | 361.46 | 58.04 |
| Lipid 21 | hEPO | 50:38.5:10:1.5 | 6 | 83.3 | 0.277 | −12.3 | 96.4 | 6.5 | 0.00 | 0.00 |
| Lipid 22 | hEPO | 50:38.5:10:1.5 | 6 | 62.7 | 0.159 | −15.3 | 99.9 | 5.8 | 78.29 | 42.32 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having the structure of formula (I-w): 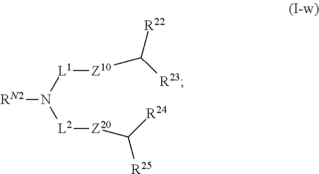
or a salt thereof;
wherein:
R^{N2} is 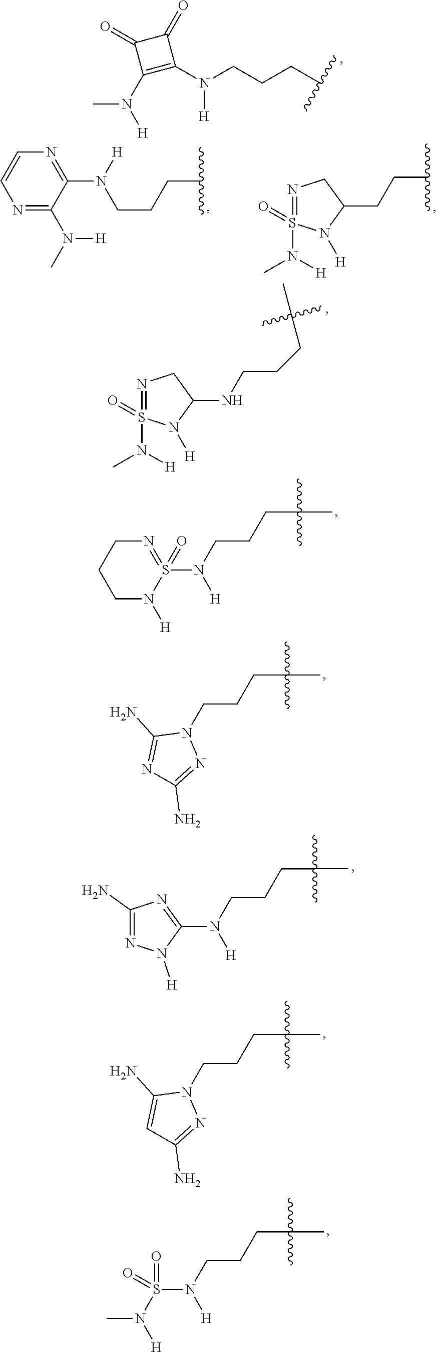 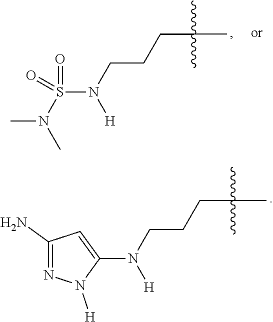 [SMILES: *CCCNS(=O)(=O)N(C)C ; *CCCNc1cc(N)n[nH]1]
L¹ and L² are each independently (C₁-C₁₅)alkylene;
Z¹⁰ and Z²⁰ are each independently 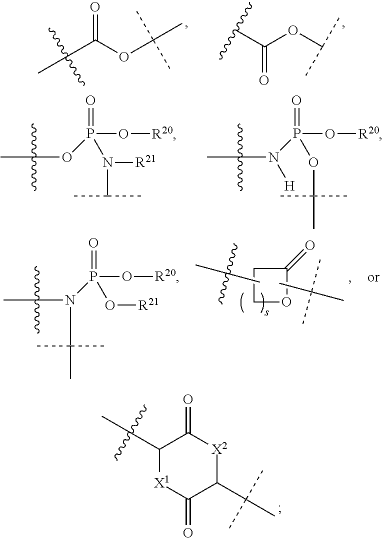
each of X¹ and X² is independently O, S, or N(R²¹);
R²⁰ is branched (C₁-C₁₅)alkyl or unbranched (C₁-C₁₅)alkyl;
R²¹ is H, (C₁-C₅)alkyl, or (C₃-C₈)cycloalkyl;
s is an integer from 1 to 4;
in Z¹⁰, [symbol] indicates the point of attachment to L¹;
in Z²⁰, [symbol] indicates the point of attachment to L²; and
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H.

2. A compound having the structure of formula (I-w-4), (I-w-5), or (I-w-6): 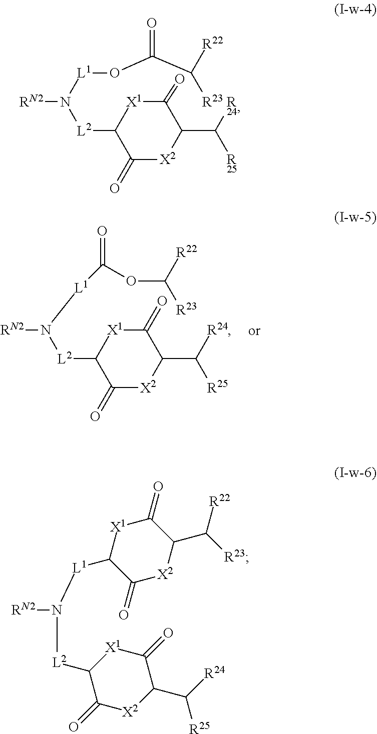
or a salt thereof;
wherein:
R^{N2} is —(CH₂)ₘ(NH)ₙQ²;
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3;
n is an integer from 0-1;
L¹ and L² are each independently (C₁-C₁₅)alkylene;
each of X¹ and X² is independently O, S, or N(R²¹);
R²¹ is H, (C₁-C₅)alkyl, or (C₃-C₈)cycloalkyl; and
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H.

3. A compound having the structure of formula (I-w-7), (I-w-8), or (I-w-9): 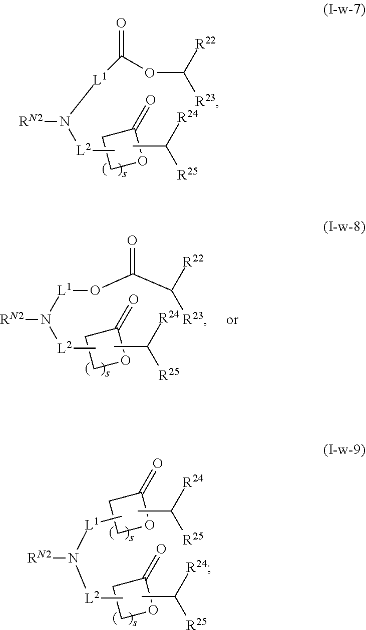
or a salt thereof:
wherein:
R^{N2} is —(CH₂)ₘ(NH)ₙQ²;
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3;
n is an integer from 0-1;
L¹ and L² are each independently (C₁-C₁₅)alkylene;
s is an integer from 1 to 4; and
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H.

4. A compound having the structure of formula (I-w-10), (I-w-11), (I-w-12), (I-w-13), (I-w-14), or (I-w-15): 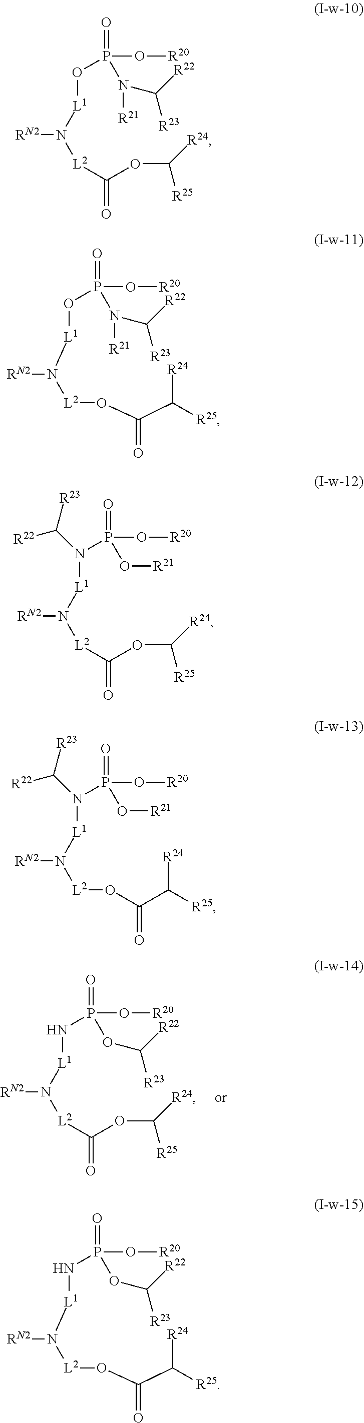
or a salt thereof:
wherein:
R^{N2} is —(CH₂)ₘ(NH)ₙQ²;
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3;
n is an integer from 0-1;
L¹ and L² are each independently (C₁-C₁₅)alkylene;
R²⁰ is branched (C₁-C₁₅)alkyl or unbranched (C₁-C₁₅)alkyl;
R²¹ is H, (C₁-C₅)alkyl, or (C₃-C₈)cycloalkyl; and
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H.

5. A compound having the structure of formula (Ia): 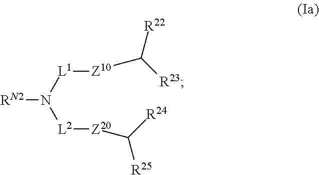
or a salt thereof;
wherein:
R^{N2} is —(CH₂)ₘ(NH)ₙQ²,
Q² is —OH, —SO₂NH(alkyl), —SO₂N(alkyl)₂, optionally substituted cycloalkyl, optionally substituted aryl, optionally substituted heteroaryl, optionally substituted heterocycloalkyl, or optionally substituted heterocycloalkenyl;
m is an integer from 2-3;
n is an integer from 0-1;
L¹ and L² are each independently (C₁-C₁₅)alkylene;
Z¹⁰ and Z²⁰ are each independently 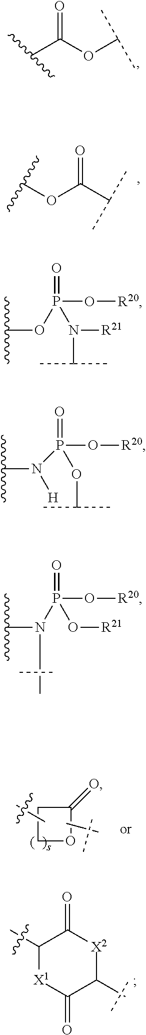
each of X¹ and X² is independently O, S, or N(R²¹);
R²⁰ is branched (C₁-C₁₅)alkyl or unbranched (C₁-C₁₅)alkyl;
R²¹ is H, (C₁-C₅)alkyl, or (C₃-C₈)cycloalkyl;
s is an integer from 1 to 4;
in Z¹⁰, [symbol] indicates the point of attachment to L¹;
in Z²⁰, [symbol] indicates the point of attachment to L²;
each of R²², R²³, R²⁴, and R²⁵ is independently H, branched (C₁-C₁₅)alkyl, or unbranched (C₁-C₁₅)alkyl; provided that at least one of R²² and R²³ is not H, and at least one of R²⁴ and R²⁵ is not H;
wherein when Q² is —OH, then at least one of (i) or (ii) applies:
(i) at least one of Z¹⁰ and Z²⁰ is 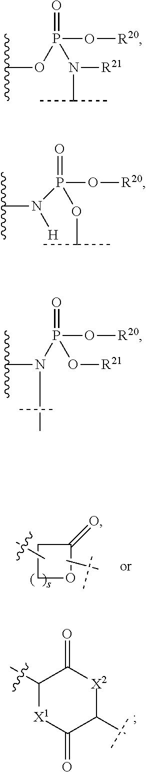
or
(ii) L¹ and L² are not identical.

6. A compound, or a salt thereof, having one of the following structures: 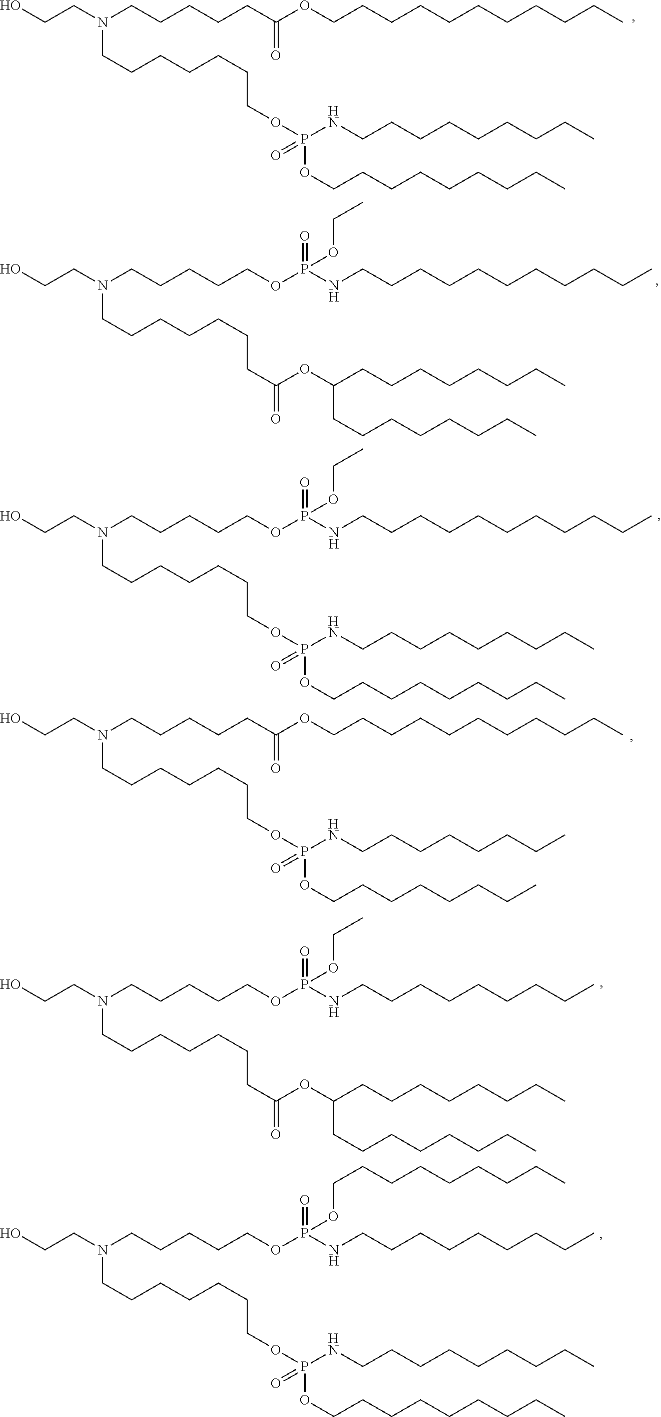 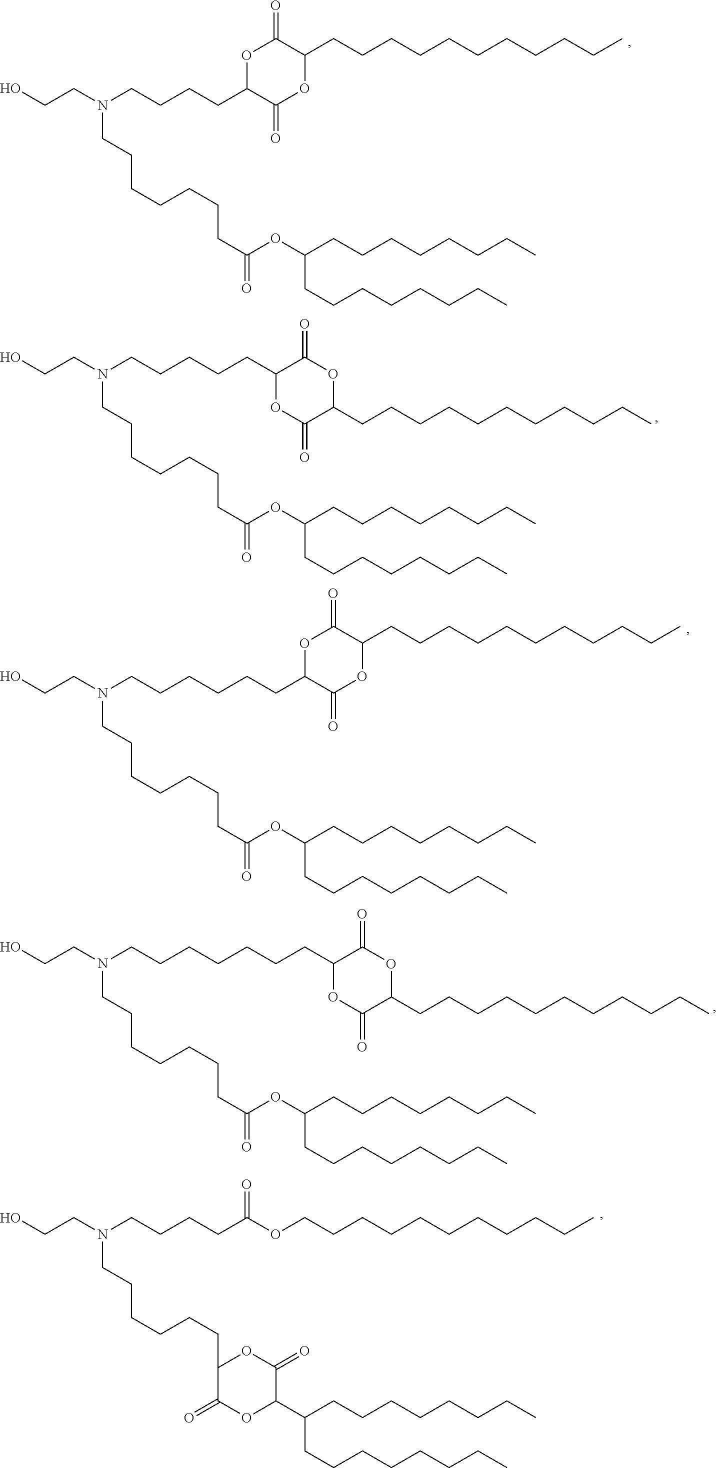 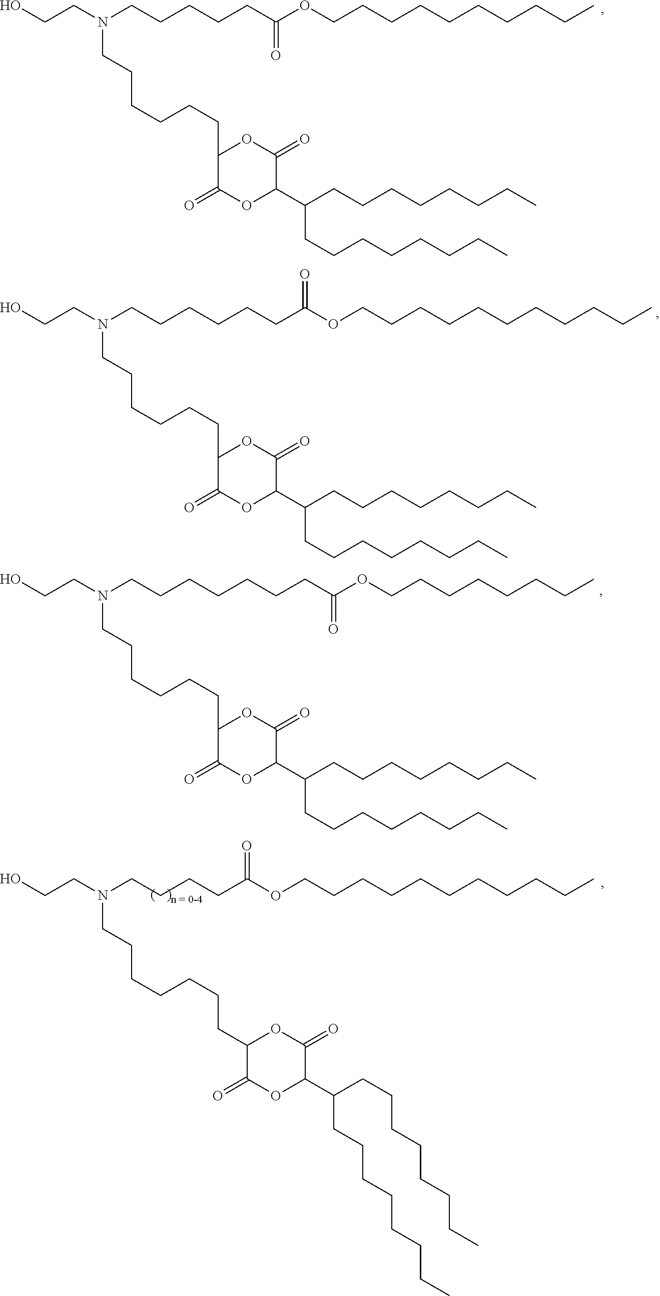 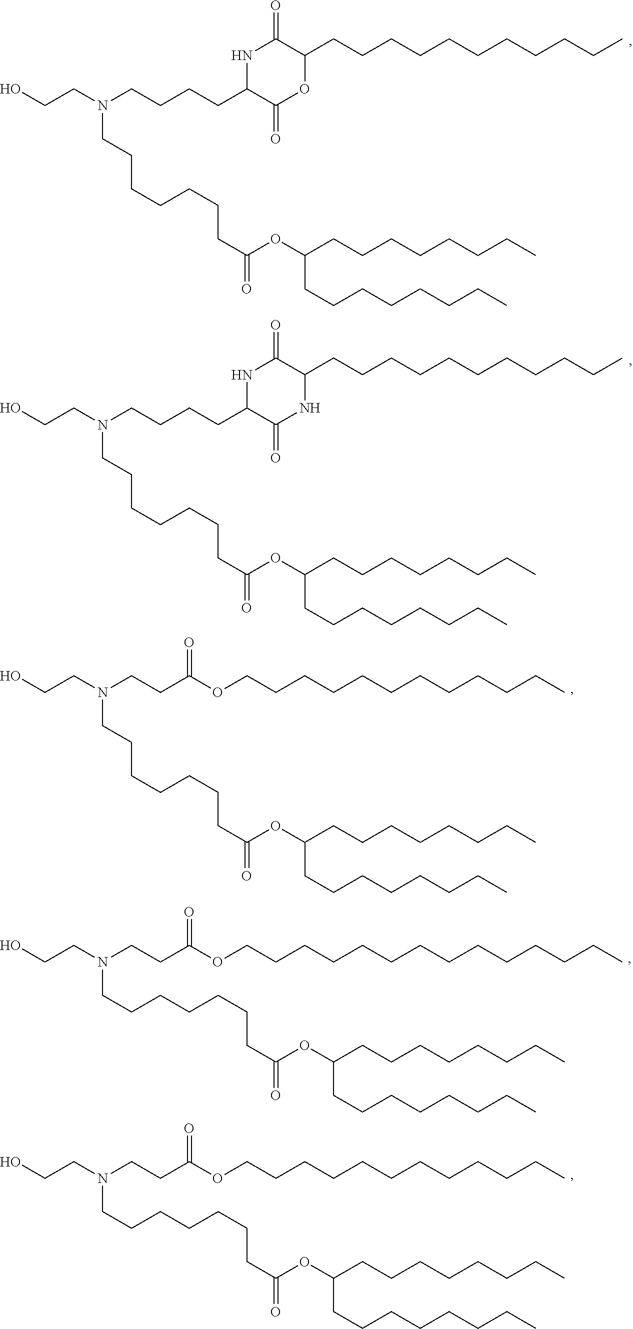 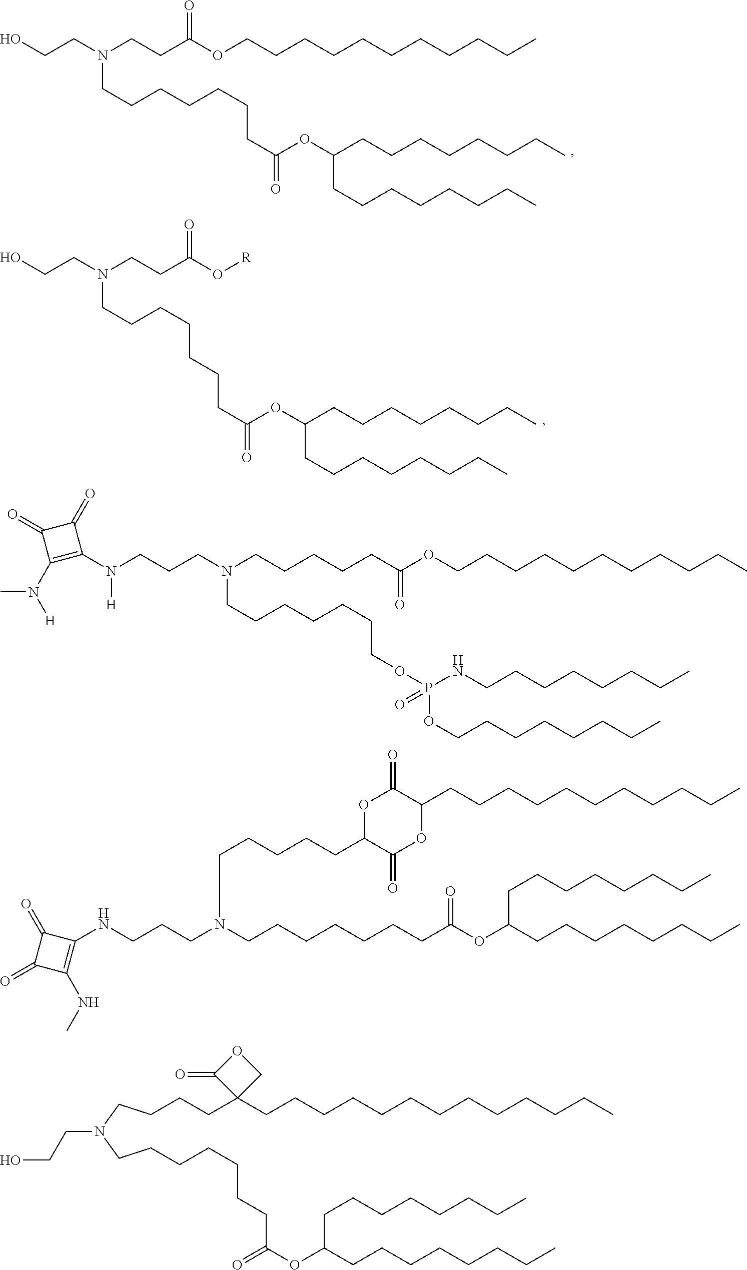 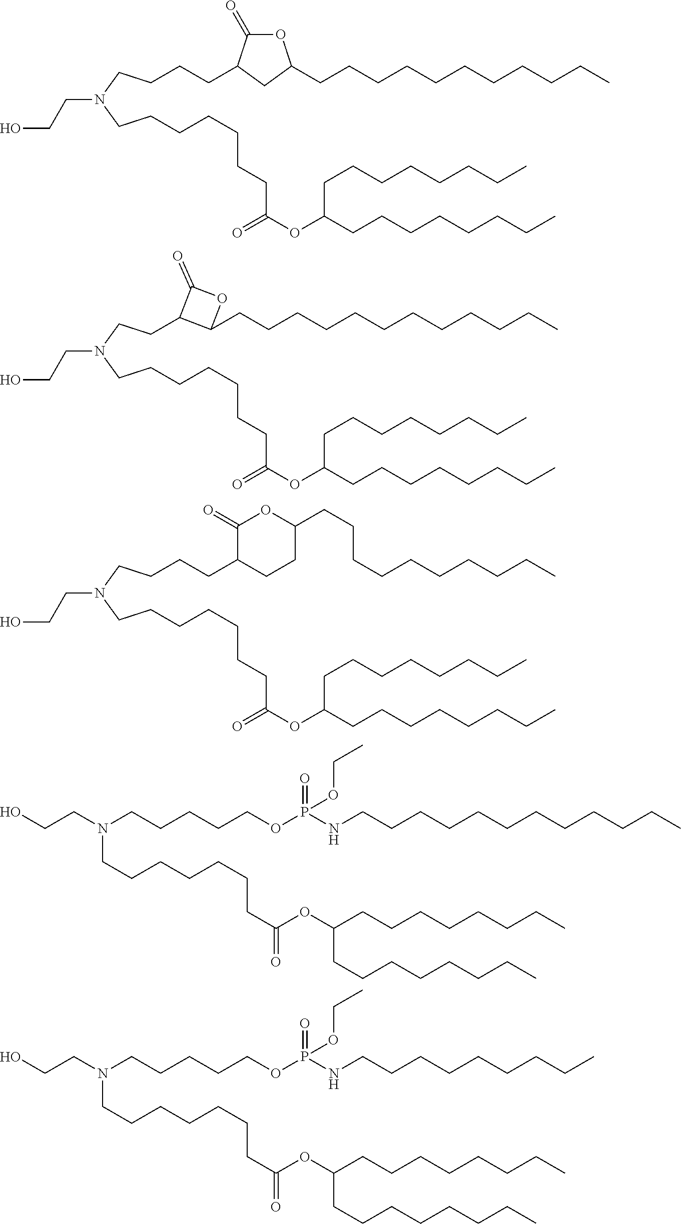 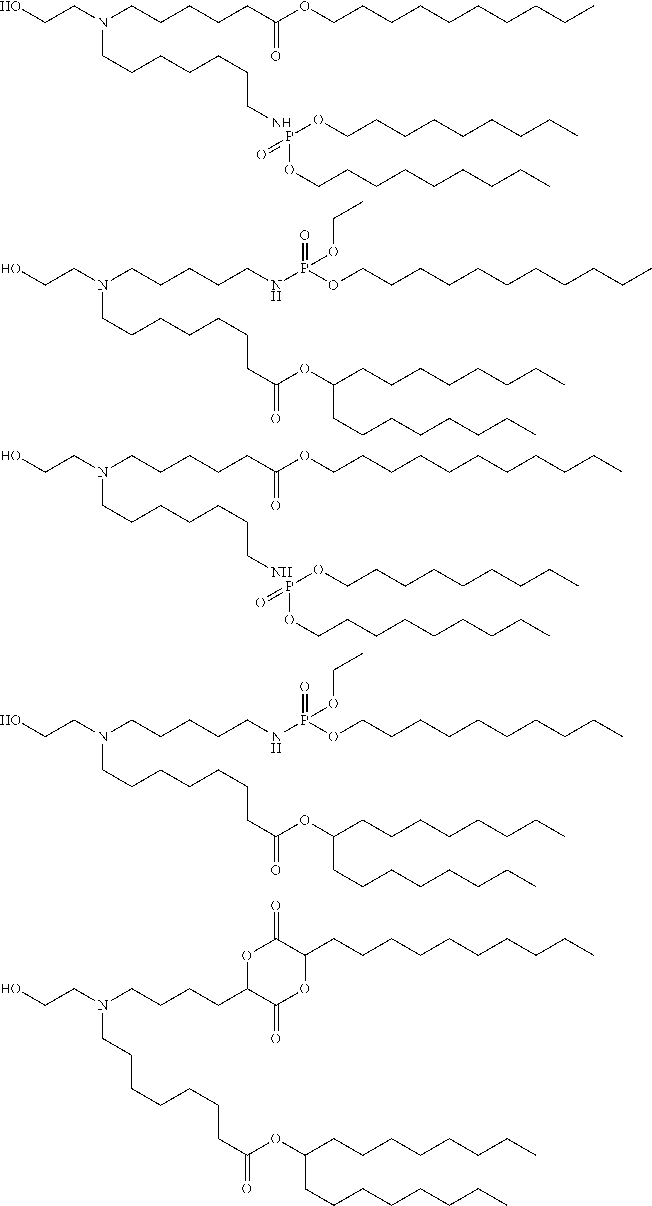 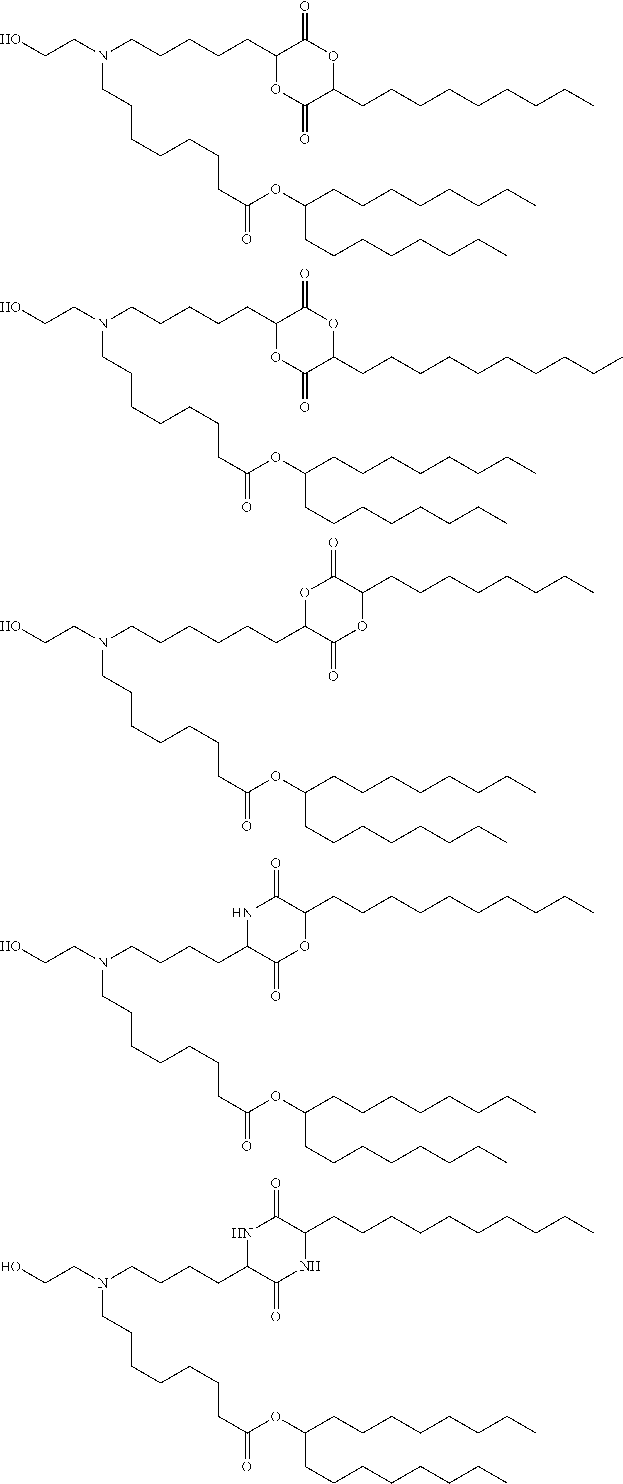 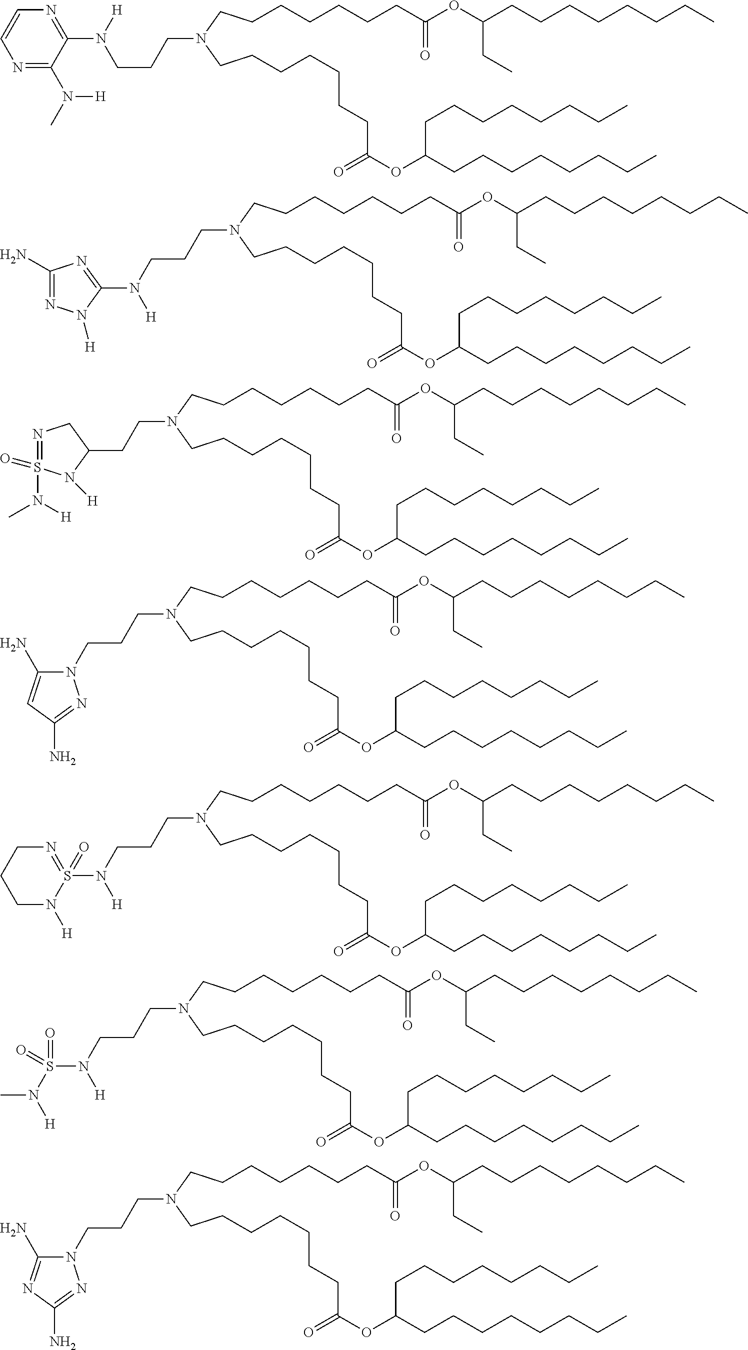 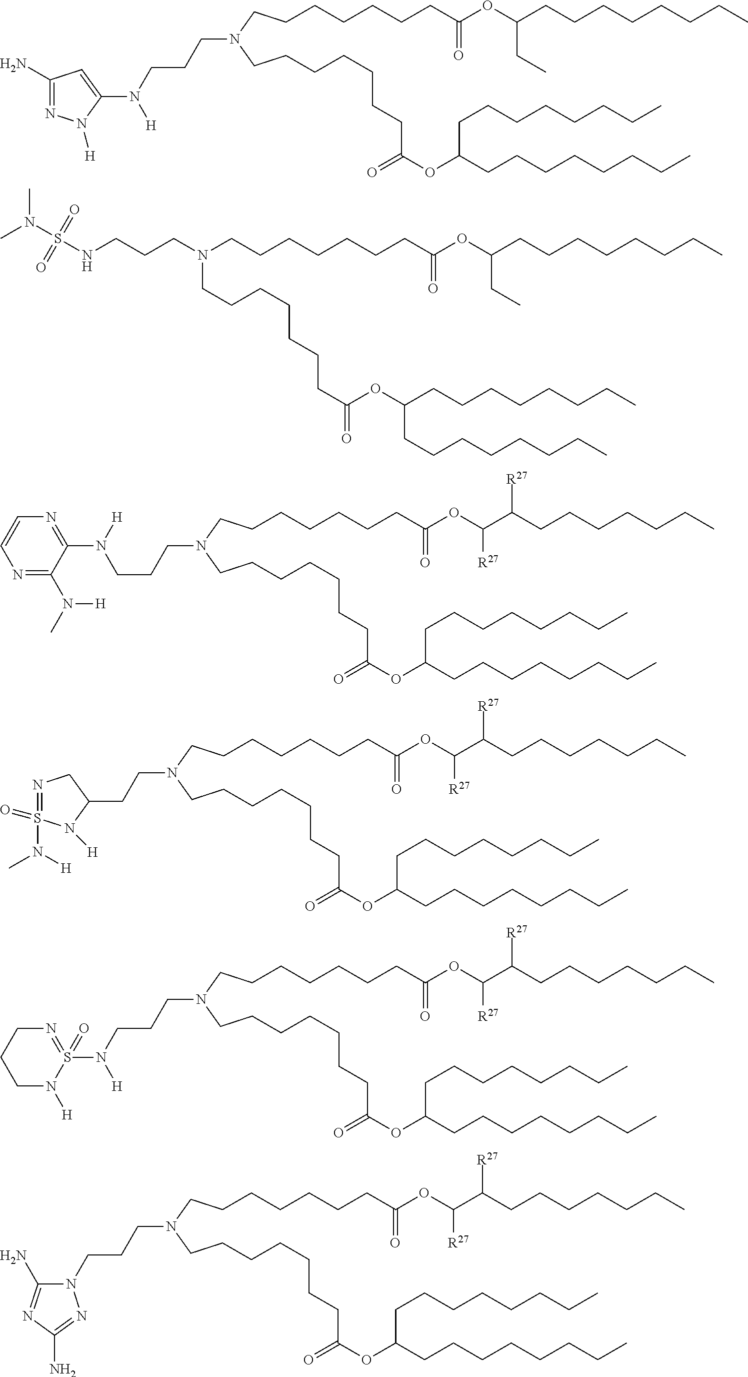 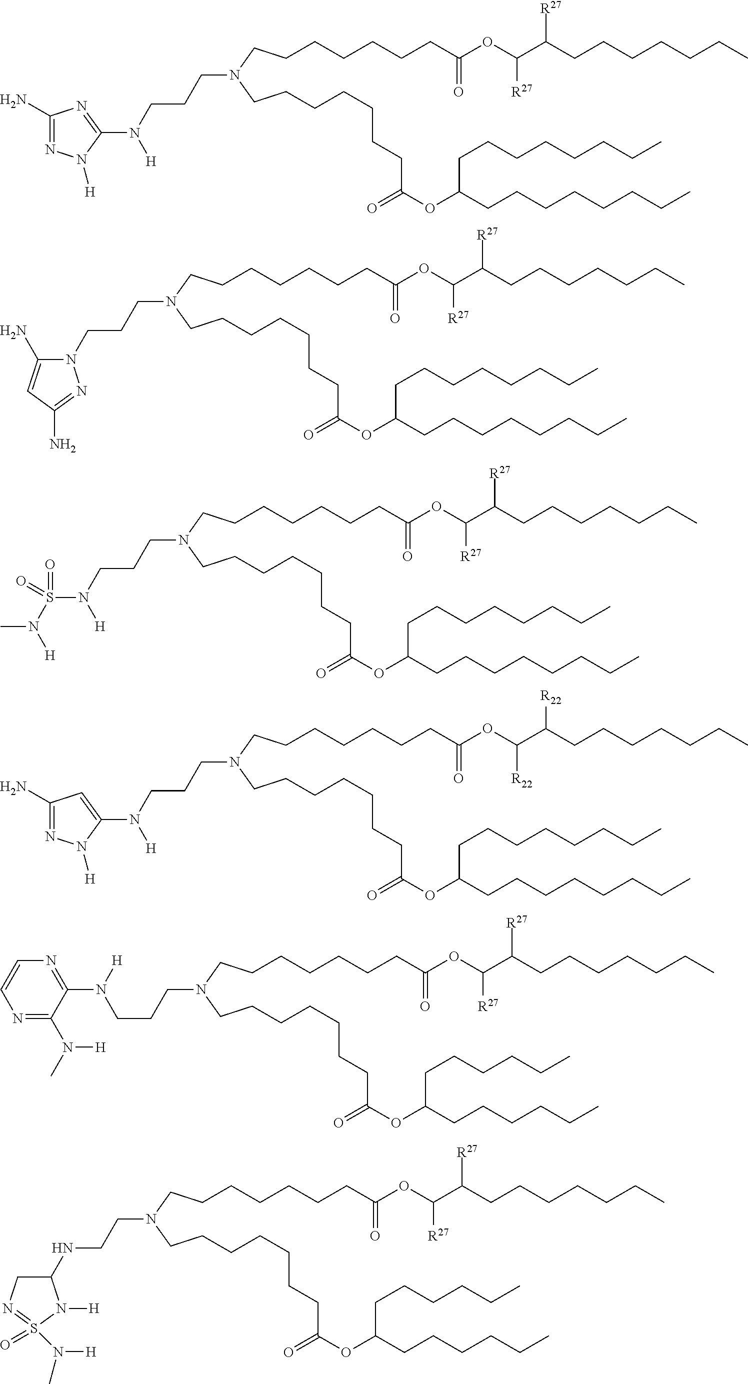 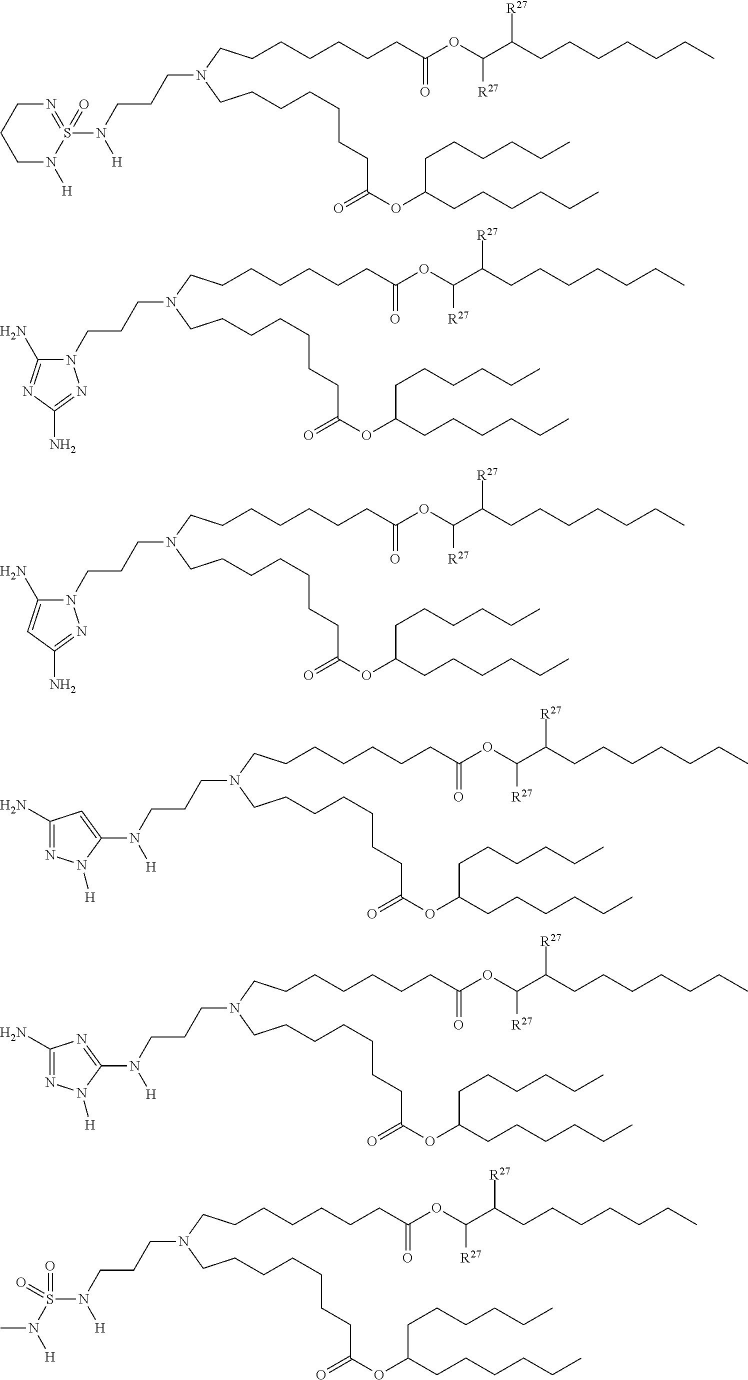 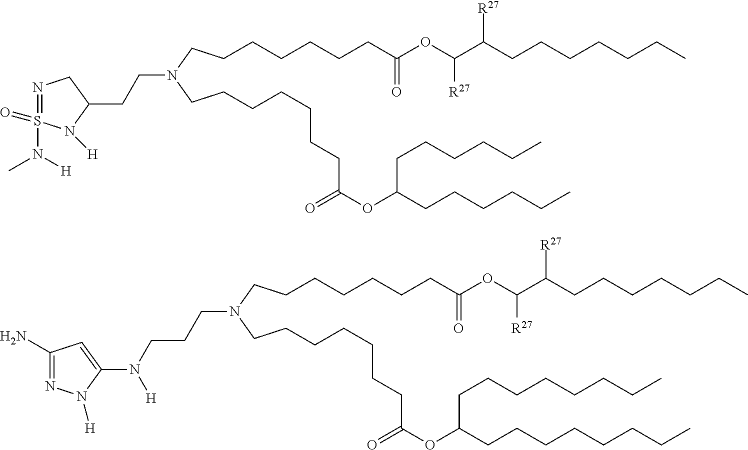
wherein R= 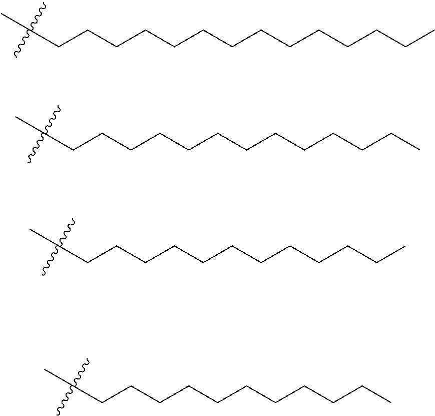 [SMILES: *CCCCCCCCCCCCCC ; *CCCCCCCCCCCCC ; *CCCCCCCCCCCC ; *CCCCCCCCCCC]
and each R²⁷ is independent H, C₁-C₁₅ alkyl, C₂-C₈ alkenyl, or C₂-C₈ alkynyl.

7. A lipid-based carrier comprising a compound of claim 1, wherein the lipid-based carrier is a lipid nanoparticle.

8. A lipid-based carrier comprising a compound of claim 2, wherein the lipid-based carrier is a lipid nanoparticle.

9. A lipid-based carrier comprising a compound of claim 3, wherein the lipid-based carrier is a lipid nanoparticle.

10. A lipid-based carrier comprising a compound of claim 4, wherein the lipid-based carrier is a lipid nanoparticle.

11. A lipid-based carrier comprising a compound of claim 5, wherein the lipid-based carrier is a lipid nanoparticle.

12. A lipid-based carrier comprising a compound of claim 6, wherein the lipid-based carrier is a lipid nanoparticle.

13. The lipid-based carrier of claim 7, wherein the lipid nanoparticle further comprises an effector, e.g., a therapeutic agent.

14. The lipid-based carrier of claim 8, wherein the lipid nanoparticle further comprises an effector, e.g., a therapeutic agent.

15. The lipid-based carrier of claim 9, wherein the lipid nanoparticle further comprises an effector, e.g., a therapeutic agent.

16. The lipid-based carrier of claim 10, wherein the lipid nanoparticle further comprises an effector, e.g., a therapeutic agent.

17. The lipid-based carrier of claim 11, wherein the lipid nanoparticle further comprises an effector, e.g., a therapeutic agent.

18. The lipid-based carrier of claim 12, wherein the lipid nanoparticle further comprises an effector, e.g., a therapeutic agent.

19. The lipid-based carrier of claim 7, wherein the lipid nanoparticle comprises an antigen.

20. The lipid-based carrier of claim 8, wherein the lipid nanoparticle comprises an antigen.

21. The lipid-based carrier of claim 9, wherein the lipid nanoparticle comprises an antigen.

22. The lipid-based carrier of claim 10, wherein the lipid nanoparticle comprises an antigen.

23. The lipid-based carrier of claim 11, wherein the lipid nanoparticle comprises an antigen.

24. The lipid-based carrier of claim 12, wherein the lipid nanoparticle comprises an antigen.

25. A method of delivering an effector to a subject, the method comprising administering to the subject the lipid-based carrier of claim 13.

26. A method of delivering an effector to a subject, the method comprising administering to the subject the lipid-based carrier of claim 14.

27. A method of delivering an effector to a subject, the method comprising administering to the subject the lipid-based carrier of claim 15.

28. A method of delivering an effector to a subject, the method comprising administering to the subject the lipid-based carrier of claim 16.

29. A method of delivering an effector to a subject, the method comprising administering to the subject the lipid-based carrier of claim 17.

30. A method of delivering an effector to a subject, the method comprising administering to the subject the lipid-based carrier of claim 18.

31. A method of vaccinating a subject in need thereof, comprising administering to the subject an effective amount of the lipid-based carrier of claim 19.

32. A method of vaccinating a subject in need thereof, comprising administering to the subject an effective amount of the lipid-based carrier of claim 20.

33. A method of vaccinating a subject in need thereof, comprising administering to the subject an effective amount of the lipid-based carrier of claim 21.

34. A method of vaccinating a subject in need thereof, comprising administering to the subject an effective amount of the lipid-based carrier of claim 22.

35. A method of vaccinating a subject in need thereof, comprising administering to the subject an effective amount of the lipid-based carrier of claim 23.

36. A method of vaccinating a subject in need thereof, comprising administering to the subject an effective amount of the lipid-based carrier of claim 24.

37. A pharmaceutical composition comprising the lipid-based carrier of claim 1, and a pharmaceutically acceptable excipient.
